(19) United States
(12) Patent Application Publication
(10) Pub. No.: US 2024/0252643 A1
(43) Pub. Date: Aug. 1, 2024

(54) GUCY2C T CELL-ANTIGEN COUPLERS AND USES THEREOF

(71) Applicant: Triumvira Immunologics USA, Inc., Austin, TX (US)

(72) Inventors: Andreas Bader, Austin, TX (US); Christopher W. Helsen, Oakville (CA); Philbert Ip, Hamilton (CA); Tania Benatar, Thornhill (CA)

(21) Appl. No.: 18/577,219

(22) PCT Filed: Jun. 30, 2022
(86) PCT No.: PCT/US2022/035871

§ 371 Date: Jan. 5, 2024

Related U.S. Application Data
(60) Provisional application No. 63/261,930, filed on Sep. 30, 2021.
(60) Provisional application No. 63/203,106, filed on Jul. 8, 2021.

(51) Int. Cl. A61K 39/00; A61P 35/00; C07K 14/725; C07K 16/40

(52) U.S. Cl. CPC A61K 39/464454; A61K 39/4611; A61K 39/4632; A61P 35/00; C07K 14/7051; C07K 16/40; C07K 2317/569; C07K 2317/622; C07K 2319/30

(57) ABSTRACT

GUCY2C T cell antigen coupler (TAC) polypeptides having (i) an antigen-binding domain that binds GUCY2C, (ii) an antigen-binding domain that binds a protein associated with a TCR complex, and (iii) a T cell receptor signaling domain polypeptide are provided.

### CROSS-REFERENCE TO RELATED APPLICATIONS

This application claims the benefit of and priority to U.S. Provisional Patent Application No. 63/203,106, filed Jul. 8, 2021; and U.S. Provisional Patent Application No. 63/261,930, filed Sep. 30, 2021, the disclosures of each of which are hereby incorporated by reference in their entireties for all purposes.

### SEQUENCE LISTING

The instant application contains a Sequence Listing which has been submitted electronically in ASCII format and is hereby incorporated by reference in its entirety. Said ASCII copy, created on xxxx xx, 2022, is named xxxxxxxxx.txt and is ______ bytes in size.

### SUMMARY

Disclosed herein, in certain embodiments, are polynucleotides encoding a GUCY2C (Guanylate Cyclase 2C) T cell-antigen coupler (GUCY2C-TAC) polypeptide.

Disclosed herein, in certain embodiments, are Guanylate Cyclase 2C (GUCY2C) T cell-antigen coupler (GUCY2C-TAC) proteins, comprising: (a) a first polypeptide encoding an antigen-binding domain that binds GUCY2C; (b) a second polypeptide encoding an antigen-binding domain that binds a protein associated with a TCR complex; and (c) a third polypeptide encoding a TCR co-receptor cytosolic domain and transmembrane domain; wherein components encoded by (a), components encoded by (b), and components encoded by (c) are fused directly to each other, or joined by at least one linker. In some embodiments, the first polynucleotide, the second polynucleotide, and the third polynucleotide are in order. In some embodiments, the antigen-binding domain that binds GUCY2C is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody. In some embodiments, the antigen-binding domain that binds GUCY2C is a designed ankyrin repeat (DARPin) polypeptide, a single chain variable fragment (scFv), or a nanobody. In some embodiments, the antigen-binding domain that binds GUCY2C is a nanobody. In some embodiments, the protein associated with the TCR complex is a CD3 protein. In some embodiments, the CD3 protein is a CD3γ protein, CD3δ protein and/or CD3ε protein. In some embodiments, the CD3 protein is a CD3ε protein. In some embodiments, the CD3 protein is a CD3ε protein. In some embodiments, the antigen-binding domain that binds the protein associated with the TCR complex is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody. In some embodiments, the antigen-binding domain that binds the protein associated with the TCR complex is derived from an antibody selected from UCHT1 OKT3, F6A, and L2K. In some embodiments, the antigen-binding domain that binds the protein associated with the TCR complex is a UCHT1 antigen-binding domain. In some embodiments, the UCHT1 antigen-binding domain is an scFv of UCHT1. In some embodiments, the UCHT1 antigen-binding domain comprises a Y to T mutation at a position corresponding to amino acid 182 of SEQ ID NO: 32 (Y182T). In some embodiments, the UCHT1 antigen-binding domain comprises a humanized variant of UCHT1 (huUCHT1). In some embodiments, the UCHT1 antigen-binding domain comprises a humanized variant of UCHT1 comprising a Y to T mutation at a position corresponding to amino acid 177 of SEQ ID NO: 40 (huUCHT1 (Y177T)). In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)). In some embodiments, the antigen-binding domain that binds the protein associated with the TCR complex is an OKT3 antigen-binding domain. In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 34 (OKT3). In some embodiments, the antigen-binding domain that binds the protein associated with the TCR complex is a F6A antigen-binding domain. In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 36 (F6A). In some embodiments, the antigen-binding domain that binds the protein associated with the TCR complex is a L2K antigen-binding domain. In some embodiments, the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 38 (L2K). In some embodiments, the transmembrane domain is a CD4 transmembrane domain and the cytosolic domain is a CD4 cytosolic domain. In some embodiments, the transmembrane and cytosolic domain comprise an amino acid sequence having at least 80%, at least 85%, at least 90%, at least 95%, at least 96%, at least 97%, at least 98%, at least 99%, or 100% sequence identity with the amino acid sequence of SEQ ID NO: 46 (CD4 transmembrane and cytosolic domain). In some embodiments, the transmembrane domain is a CD8 transmembrane domain and the cytosolic domain is a CD8 cytosolic domain. In some embodiments, the component encoded by (a) and the component encoded by (c) are fused to the component encoded by (b). In some embodiments, the component encoded by (b) and the component encoded by (c) are fused to the component encoded by (a). In some embodiments, the at least one linker joins the component encoded by (a) to the component encoded by (b). In some embodiments, the at least one linker is a glycine and/or serine-rich linker, a large protein domain, a long helix structure, or a short helix structure. In some embodiments, at least one linker comprises an amino acid sequence having at least 80%, at least 85%, at least 90%, at least 95%, at least 96%, at least 97%, at least 98%, at least 99%, or 100% sequence identity with the amino acid sequence of SEQ ID NO: 26 ((G4S)4-based linker), SEQ ID NO: 28 (G4S-based linker), SEQ ID NO: 14 (CD4 based linker), SEQ ID NO: 12 (short helix connector), SEQ ID NO: 14 (long helix connector), SEQ ID NO: 16 (large domain connector), or SEQ ID NO: 24 ((G4S)3 flexible linker). In some embodiments, the GUYC2C antigen binding domain is selected from an amino acid sequence according to any one of SEQ ID NOs: 53-127 or 514-521. In some embodiments, the GUCY2C antigen-binding domain comprises a heavy chain variable region having an amino acid sequence having at least 80% sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 80% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203. In some embodiments, the GUYC2C antigen binding domain comprises a heavy chain variable region having an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203. In some embodiments, the GUCY2C antigen-binding domain comprises a heavy chain variable region comprising (a) a CDR1 having an amino acid selected from the group consisting of SEQ ID NO: 204, 210, 216, 222, 228, 234, 240, 246, 252, 258, 264, 270, 276, 282, 288, 294, 300, 306, 312, 318, 324, 330, 336, 342, 348, 354, 393, 399, 405, 411, 417, 423, 429, 435, 441, 447, 453, 459, 522, 525, 528, 531, 534, 537, 540, and 543, (b) a CDR2 having an amino acid selected from the group consisting of SEQ ID NO: 205, 211, 217, 223, 229, 235, 241, 247, 253, 259, 265, 271, 277, 283, 289, 295, 301, 307, 313, 319, 325, 331, 337, 343, 349, 355, 394, 400, 406, 412, 418, 424, 430, 436, 442, 448, 454 460, 523, 526, 529, 532, 535, 538, 541, and 544, and (c) a CDR3 having an amino acid selected from the group consisting of SEQ ID NO: 206, 212, 218, 224, 230, 236, 242, 248, 254, 260, 266, 272, 278, 284, 290, 296, 302, 308, 314, 320, 326, 332, 338, 344, 350, 356, 395, 401, 407, 413, 419, 425, 431, 437, 443, 449, 455, 461, 524, 527, 530, 533, 536, 539, 542, and 545; and a light chain variable region comprising (a) a CDR1 having an amino acid selected from the group consisting of SEQ ID NO: 207, 213, 219, 225, 231, 237, 243, 249, 255, 261, 267, 273, 279, 285, 291, 297, 303, 309, 315, 321, 327, 333, 339, 345, 351, 357, 396, 402, 408, 414, 420, 426, 432, 438, 444, 450, 456 and 462, (b) a CDR2 having an amino acid selected from the group consisting of SEQ ID NO: 208, 214, 220, 226, 232, 238, 244, 250, 256, 262, 268, 274, 280, 286, 292, 298, 304, 310, 316, 322, 328, 334, 340, 346, 352, 358, 397, 403, 409, 415, 421, 427, 433, 439, 445, 451, 457 and 463, and (c) a CDR3 having an amino acid selected from the group consisting of SEQ ID NO: 209, 215, 221, 227, 233, 239, 245, 251, 257, 263, 269, 275, 281, 287, 293, 299, 305, 311, 317, 323, 329, 335, 341, 347, 353, 359, 398, 404, 410, 416, 422, 428, 434, 440, 446, 452, 458 and 464. In some embodiments, the GUCY2C antigen-binding domain is a nanobody and comprises (a) a VHH CDR1 having an amino acid selected from the group consisting of SEQ ID NO: 360, 363, 366, 369, 372, 375, 378, 381, 384, 387, and 390; (b) a VHH CDR2 having an amino acid selected from the group consisting of SEQ ID NO: 361, 364, 367, 370, 373, 376, 379, 382, 385, 388, and 391; and (c) a VHH CDR3 having an amino acid selected from the group consisting of SEQ ID NO: 362, 365, 368, 371, 374, 377, 380, 383, 386, 389, and 392. In some embodiments, the GUCY2C-TAC protein does not comprise a co-stimulatory domain. In some embodiments, the GUCY2C-TAC protein does not comprise an activation domain. In some embodiments, the GUCY2C-TAC protein further comprises a leader sequence. In some embodiments, the leader sequence comprises an amino acid sequence having at least 80%, at least 85%, at least 90%, at least 95%, at least 96%, at least 97%, at least 98%, at least 99%, or 100% sequence identity with the amino acid sequence of SEQ ID NO: 2 (muIgG leader), SEQ ID NO: 18 (huIgG leader), SEQ ID NO: 20 (huCD8a-1 leader) or SEQ ID NO: 30 (huCD8a-2 leader).

Disclosed herein, in certain embodiments, are GUCY2C TAC proteins comprising an amino acid sequence having at least 80% sequence identity with the amino acid sequence of any one of SEQ ID NOs: 465-513, 546-590, or 686.

Disclosed herein, in certain embodiments, are GUCY2C TAC protein comprising an amino acid sequence of any one of SEQ ID NOs: 465-513, 546-590, or 686.

Disclosed herein, in certain embodiments, are nucleic acid sequences encoding a GUCY2C-TAC protein described herein. In some embodiments, the nucleic acid sequence has at least 80% sequence identity with the nucleic acid sequence of any one of SEQ ID NOs: 591-685. In some embodiments, the nucleic acid sequence comprises the nucleic acid sequence of any one of SEQ ID NOs: 591-685.

Disclosed herein, in certain embodiments, are T cells expressing a GUCY2C-TAC protein described herein. Disclosed herein, in certain embodiments, are T cells comprising a nucleic acid described herein.

Disclosed herein, in certain embodiments, are pharmaceutical compositions comprising a T cell described herein, and a pharmaceutically acceptable excipient.

Disclosed herein, in certain embodiments, are method of treating a GUCY2C-expressing cancer in an individual in need thereof, comprising administering to the individual a pharmaceutical composition described herein. In some embodiments, the cancer is a solid cancer. In some embodiments, the cancer is a primary colorectal cancer, a primary gastric cancer, a primary gastroesophageal junction cancer, a primary esophageal cancer, or a primary pancreatic cancer. In some embodiments, the cancer is a metastatic colorectal cancer, a metastatic gastric cancer, a metastatic gastroesophageal junction cancer, a metastatic esphageal cancer, or a metastatic pancreatic cancer.

### BRIEF DESCRIPTION OF THE DRAWINGS

The invention can be more completely understood with reference to the following drawings.

FIG. 1 depicts a graph showing surface expression level of indicated GUCY2C-TACs as measured by flow cytometry.

FIGS. 2A-2B depict graphs showing activation of T cells expressing the indicated TACs as measured by up-regulation of CD69 following co-culture with NALM6^{GUCY2C} (FIG. 2A) or N87^{GUCY2C} (FIG. 2B) target cells.

FIGS. 3A-3B depict a cell trace assay. FIG. 3A depicts the normalized division indices of T cells expressing the indicated TACs following co-culture with NALM6^{GUCY2C} target cells.

FIG. 3B depicts representative graphs of cell trace violet (CTV) staining of T cells expressing indicated TACs from FIG. 3A.

FIG. 4 depicts a graph showing cytotoxicity of NALM6^{GUCY2C-GFP} target cells following co-culture with T cells expressing indicated TACs.

FIG. 5 depicts a graph showing activation of T cells expressing the indicated TACs as measured by up-regulation of CD69 following co-culture with indicated target cells.

FIG. 6 depicts a graph showing the normalized division indices of T cells expressing the indicated TACs following co-culture with indicated target cells.

FIG. 7 depicts the relative cell counts of target NALM6^{GUCY2C-GFPeLuc} cells as measured by detection of fluorescence signal from target cells following co-culture with T cells expressing indicated TACs at indicated effector:target (E:T) ratios.

FIGS. 8A-8B depict results of an assay measuring activation of GUCY2C-TAC T cells against cell lines with varying expression of GUCY2C. FIG. 8A depicts graphs showing relative GUCY2C expression (horizontal axes) with cell counts shown on the vertical axes. FIG. 8B depicts a graph showing activation of T cells expressing the indicated TACs as measured by up-regulation of CD69 following co-culture with indicated target cells.

### DETAILED DESCRIPTION

Cancer is a major health challenge. According to the American Cancer Society, more than one million people in the United States are diagnosed with cancer each year. While patients with early stage disease are sometimes treated effectively by conventional therapies (surgery, radiation, chemotherapy), few options are available to patients with advanced disease, and those options are typically palliative in nature.

Active immunotherapy seeks to employ the patient's immune system to clear tumors and offers an option to patients who have failed conventional therapies. Generally, this treatment involves infusing patients with large numbers of tumor-specific T cells. To this point, most engineered T cell therapies involving genetic modification of the T cells yield: (i) forced expression of T cell receptor (TCR); or (ii) a chimeric antigen receptor (CAR) specific for antigen targets on the tumor. To date, the chimeric antigen receptors used for engineering T cells consist of: (i) a targeting domain, usually a single-chain fragment variable (scFv); (ii) a transmembrane domain; and (iii) a cytosolic domain that contains signaling elements from the T cell receptor and associated proteins. Such chimeric antigen receptors have also been referred to as “T-body” or “Chimeric Immune Receptor” (CIR), but currently, most researchers use the term “CAR”. One advantage of the CAR approach is that it allows any patient's immune cells to be targeted against any desirable target in a major histocompatibility complex (MHC) independent manner. This is appealing as MHC presentation is often defective in tumor cells.

CARs are considered in modular terms and scientists have spent considerable time investigating the influence of different cytoplasmic signaling domains on CAR function. Conventional CARs generally share two main components: (i) the CD3 zeta cytoplasmic domain, which contains immunotyrosine activation motifs (ITAMs) critical for T cell activation; and (ii) components of costimulatory receptors that trigger important survival pathways such as the Akt pathway.

The first-generation CARs employed a single signaling domain from either CD3ζ or FcεRIγ. Second-generation CARs combined the signaling domain of CD3ζ with the cytoplasmic domain of costimulatory receptors from either the CD28 or TNFR family of receptors. Most CAR-engineered T cells that are currently being tested in the clinic employ second-generation CARs where CD3ζ is coupled to the cytoplasmic domain of either CD28 or CD137. These second generation CARs have demonstrated anti-tumor activity in CD19-positive tumors. Third-generation CARs combined multiple costimulatory domains, but there is concern that third-generation CARs may lose antigen-specificity.

While CAR-engineered T cells have shown considerable promise in clinical application, they rely on a synthetic method for replacing the native activation signal that is provided by the T cell receptor (TCR). Since this synthetic receptor does not deliver all of the signaling components associated with the TCR (ex. ITAMs on CD3γ, CD3δ, CD3ε), it remains unclear whether the T cells are optimally activated by the CAR or how the CAR activation affects T cell differentiation (ex. progression to memory). Furthermore, since the CAR signaling domains are disconnected from their natural regulatory partners by the very nature of the CAR structure, there is an inherent risk that CARs may lead to a low-level of constitutive activation, which could result in off-target toxicities. Therefore, the synthetic nature of the prototypic CAR may disrupt canonical mechanisms that limit TCR activation, and may underpin the severe toxicity often associated with therapeutic doses of conventional CAR T cells.

Given these limitations, it is preferable to re-direct T cells to attack tumors via their natural TCR. An alternate chimeric receptor, termed a T cell Antigen Coupler (TAC or TAC) receptor, has been developed which employs a distinct biology to direct the T cell to attack tumors. While the CAR is a fully synthetic receptor that stitches together components of T cell receptor (TCR) signaling complex, the TAC receptor re-directs the TCR towards tumor targets and recapitulates the native TCR signaling structure. For example, in some embodiments, the TACs disclosed herein activate natural Major Histocompatibility complex (MHC) signaling through the T cell receptor (TCR), while retaining MHC-unrestricted targeting. Further, the TACs disclosed herein recruit the T Cell Receptor (TCR) in combination with co-receptor stimulation. Moreover, in some embodiments, TACs disclosed herein show enhanced activity and safety.

### Certain Terminology

The term “antigen-binding domain,” refers to any substance or molecule that binds, directly or indirectly, to a target (e.g., GUCY2C). Antigen-binding domains include antibodies or fragments thereof, peptides, peptidomimetics, proteins, glycoproteins, proteoglycans, carbohydrates, lipids, nucleic acids, or small molecules that bind to a target.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L}) and one constant region (C_{L}). The heavy chain consists of one variable region (V_{H}) and at least three constant regions (CH₁, CH₂ and CH₃). The variable regions determine the binding specificity of the antibody. Each variable region contains three hypervariable regions known as complementarity determining regions (CDRs) flanked by four relatively conserved regions known as framework regions (FRs). The extent of the FRs and CDRs has been defined (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242; and Chothia, C. et al. (1987) J. MOL. BIOL. 196:901-917). CDRs can also be identified by alignment of the amino acid sequences. FRs contain conserved amino acid sequences, thus CDR sequences can be identified by identification of non-conserved amino acid residues between variable regions with conserved FRs. The three CDRs, referred to as CDR₁, CDR₂, and CDR₃, contribute to the antibody binding specificity. Naturally occurring antibodies have been used as starting material for engineered antibodies, such as chimeric antibodies and humanized antibodies. Examples of antibody-based antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

The term “T cell” as used herein refers to a type of lymphocyte that plays a central role in cell-mediated immunity. T cells, also referred to as T lymphocytes, are distinguished from other lymphocytes, such as B cells and natural killer cells, by the presence of a T-cell receptor (TCR) on the cell surface. There are several subsets of T cells with distinct functions, including but not limited to, T helper cells, cytotoxic T cells, memory T cells, regulatory T cells and natural killer T cells.

The term “γδ T cell” or “gamma delta T cell” or “gd T cell” as used herein refers to any lymphocyte having a γδ T cell receptor (TCR) on its surface, including one γ-chain and one δ-chain.

The term “T cell antigen coupler” or TAC is used interchangeably with “trifunctional T cell antigen coupler” or Tri-TAC and refers to an engineered nucleic acid construct or polypeptide comprising (a) an antigen-binding domain that binds a target, (b) an antigen-binding domain that binds a protein associated with a T cell receptor (TCR) complex, and (c) a T cell receptor signaling domain.

The term “polynucleotide” and/or “nucleic acid sequence” and/or “nucleic acid” as used herein refers to a sequence of nucleoside or nucleotide monomers consisting of bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acids of the present disclosure may be isolated from biological organisms, formed by laboratory methods of genetic recombination or obtained by chemical synthesis or other known protocols for creating nucleic acids.

The term “isolated polynucleotide” or “isolated nucleic acid sequence” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e., sequences located at the 5′ and 3′ ends of the nucleic acid) from which the nucleic acid is derived. The term “nucleic acid” is intended to include DNA and RNA and is either double stranded or single stranded, and represents the sense or antisense strand. Further, the term “nucleic acid” includes the complementary nucleic acid sequences.

The term “recombinant nucleic acid” or “engineered nucleic acid” as used herein refers to a nucleic acid or polynucleotide that is not found in a biological organism. For example, recombinant nucleic acids may be formed by laboratory methods of genetic recombination (such as molecular cloning) to create sequences that would not otherwise be found in nature. Recombinant nucleic acids may also be created by chemical synthesis or other known protocols for creating nucleic acids.

The terms “peptide”, “polypeptide,” and “protein” as used herein mean a chain of amino acids. The term protein as used herein further means a large molecule comprising one or more chains of amino acids and, in some embodiments, is a fragment or domain of a protein or a full length protein. Furthermore, as used herein, the term protein either refers to a linear chain of amino acids or to a chain of amino acids that has been processed and folded into a functional protein. The protein structure is divided into four distinct levels: (1) primary structure—referring to the sequence of amino acids in the polypeptide chain, (2) secondary structure—referring to the regular local sub-structures on the polypeptide backbone chain, such as α-helix and β-sheets, (3) tertiary structure—referring to the three-dimensional structure if monomeric and multimeric protein molecules, and (4) quaternary structure—referring to the three-dimensional structure comprising the aggregation of two or more individual polypeptide chains that operate as a single functional unit. The use of peptide or polypeptide herein does not mean that the chain of amino acids is not also a protein (i.e., a chain of amino acids having a secondary, tertiary or quaternary structure).

The term “isolated polypeptide” refers to a polypeptide substantially free of cellular material or culture medium when produced by recombinant DNA techniques, or chemical precursors or other chemicals when chemically synthesized.

The term “vector” as used herein refers to a polynucleotide that is used to deliver a nucleic acid to the inside of a cell. In some embodiments, a vector is an expression vector comprising expression control sequences (for example, a promoter) operatively linked to a nucleic acid to be expressed in a cell. Vectors known in the art include, but are not limited to, plasmids, phages, cosmids and viruses.

The term “tumor antigen” or “tumor associated antigen” as used herein refers to an antigenic substance produced in tumor cells that triggers an immune response in a host (e.g., which is presented by MHC complexes). In some embodiments, a tumor antigen is on the surface of a tumor cell.

As used herein, the term “transmembrane and cytosolic domain” refers to a polypeptide that comprises a transmembrane domain and a cytosolic domain of a protein associated with the T cell receptor (TCR) complex. In some embodiments, such transmembrane and cytosolic domain may include, but is not limited to, protein domains that (a) associate with the lipid raft and/or (b) bind Lck.

A “TCR co-receptor” as used herein, refers to a molecule that assists the T cell receptor (TCR) in communicating with an antigen-presenting cell and may be considered part of the first signal that leads to the activation of the TCR. Examples of TCR co-receptors include, but are not limited to, CD4, LAG3, and CD8.

A “TCR co-stimulator” or “co-stimulatory domain” as used herein, refers to a molecule that enhances the response of a T cell to an antigen and may be considered as the second signal that leads to the activation of the TCR. Examples of TCR co-stimulators include, but are not limited to, ICOS, CD27, CD28, 4-1BB (CD 137), OX40 (CD134), CD30, CD40, lymphocyte fiction-associated antigen 1 (LFA-1), CD2, CD7, LIGHT, NKG2C, B7-H3, and a ligand that specifically binds CD83.

The terms “recipient”, “individual”, “subject”, “host”, and “patient”, are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

As used herein, the terms “treatment,” “treating,” and the like, in some embodiments, refer to administering an agent, or carrying out a procedure, for the purposes of obtaining an effect. The effect may be prophylactic in terms of completely or partially preventing a disease or symptom thereof and/or may be therapeutic in terms of affecting a partial or complete cure for a disease and/or symptoms of the disease. “Treatment,” as used herein, may include treatment of a disease or disorder (e.g., cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms; or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent, delay, alleviate, arrest or inhibit development of the symptoms or conditions associated with diseases (e.g., cancer). The term “therapeutic effect” refers to the reduction, elimination, or prevention of the disease, symptoms of the disease, or side effects of the disease in the subject.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 96%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

As used herein, the term “selective binding” refers to the higher affinity with which a molecule (e.g., protein such as an antigen-binding domain of TAC) binds its target molecule (e.g., target antigen such as GUCY2C) over other molecules. Unless indicated otherwise, the terms “selective binding” and “specific binding” are used interchangeably herein.

As used herein, the term GUCY2C means the enzyme Guanylate Cyclase 2C. GUCY2C is a transmembrane protein that functions as a receptor for endogenous peptides guanylin and uroguanylin, and the heat-stable E. coli enterotoxin. The encoded protein activates the cystic fibrosis transmembrane conductance regulator. GUCY2C produces the cGMP following activation by the binding of guanylin or uroguanylin, regulating intestinal homeostasis, tumorigenesis, and obesity. Cell surface expression of GUCY2C is found on luminal surfaces of the intestinal epithelium and certain hypothalamic neurons. Over-expression of GUCY2C is found in tumors that evolve from intestinal metaplasia, including colorectal, esophageal, gastric, and pancreatic cancers. Over-expression is maintained in >95% of colorectal cancer metastases.

### T Cell Antigen Couplers (TACs)

Disclosed herein, in certain embodiments, are nucleic acids encoding GUCY2C T cell-antigen coupler (TAC) polypeptides. In some embodiments, the nucleic acids encoding the GUCY2C TAC comprise: (a) a first polynucleotide encoding an antigen-binding domain that binds GUCY2C; (b) a second polynucleotide encoding an antigen-binding domain that binds the TCR complex; and (c) a third polynucleotide encoding a transmembrane domain and cytosolic domain. In some embodiments, the nucleic acids comprise, in order (e.g., from 5′ to 3′): (a) the first polynucleotide; (b) the second polynucleotide; and (c) the third polynucleotide encoding a TCR co-receptor cytosolic domain and transmembrane domain. In some embodiments, the nucleic acids encoding the GUCY2C TAC do not encode a co-stimulatory domain. In some embodiments, the nucleic acids encoding the GUCY2C TAC do not encode a co-activation domain.

Further disclosed herein, in certain embodiments, are GUCY2C T cell-antigen coupler (TAC) polypeptides. In some embodiments, the GUCY2C TAC polypeptides comprise: (a) an antigen-binding domain that binds GUCY2C; (b) an antigen-binding domain that binds the TCR complex; and (c) a transmembrane domain and cytosolic domain. In some embodiments, the GUCY2C TAC polypeptides comprise, in order (e.g., from N-terminus to C-terminus) (a) the antigen-binding domain that binds GUCY2C; (b) the antigen-binding domain that binds the TCR complex; and (c) the transmembrane domain and cytosolic domain. In some embodiments, the GUCY2C TAC polypeptides do not include a co-stimulatory domain. In some embodiments, the GUCY2C TAC polypeptides do not include a co-activation domain.

Further disclosed herein, in certain embodiments, are expression vectors comprising a nucleic acid encoding a GUCY2C TAC polypeptide as described herein.

Further disclosed herein, in certain embodiments, are T cells comprising a nucleic acid encoding a GUCY2C TAC polypeptide as described herein, T cells comprising an expression vector encoding a GUCY2C TAC polypeptide as described herein, or T cells comprising a GUCY2C TAC polypeptide as described herein.

Further disclosed herein, in certain embodiments, are methods of treating a cancer in an individual in need thereof, comprising administering to the individual a T cell comprising a GUCY2C T cell-antigen coupler (TAC) polypeptide as described herein.

### TCR Complex Protein Antigen-Binding Domain

In certain embodiments, the GUCY2C TAC comprises an antigen-binding domain that binds a protein associated with the TCR complex. A “TCR complex protein antigen-binding domain,” also referred to as a “TCR complex antigen-binding domain,” “antigen-binding domain that binds the TCR complex,” or “antigen-binding domain that binds a protein associated with the TCR complex,” refers to any substance or molecule that binds, directly or indirectly, to a protein associated with a TCR complex. In some embodiments, the antigen-binding domain that binds a protein associated with a TCR complex selectively binds to a protein of the TCR. In some embodiments, the antigen-binding domain that binds a protein associated with a TCR complex comprises a substance that specifically binds to a protein of the TCR.

In some embodiments, the TCR complex protein antigen-binding domain is selected from antibodies or fragments thereof, for example, single chain antibodies (e.g., single-chain fragment variable antibodies (scFvs)), single domain antibodies (e.g., heavy-chain-only antibodies (VHH), shark heavy-chain-only antibodies (VNAR)), nanobodies, diabodies, minibodies, Fab fragments, Fab′ fragments, F(ab′)₂ fragments, or Fv fragments that bind to a protein of the TCR. In some embodiments, the TCR complex protein antigen-binding domain is selected from ankyrin repeat proteins (DARPins), affibodies, adnectins, affilins, phylomers; fynomers, affimers, peptide aptamers, lectins, knottins, centyrins, anticalins, peptides, peptidomimetics, proteins, glycoproteins, or proteoglycans that bind to a protein of the TCR, or naturally occurring ligands for a protein of the TCR. In some embodiments, the TCR complex protein antigen-binding domain is a non-protein compound that binds to a protein of the TCR, including but not limited to carbohydrates, lipids, nucleic acids, or small molecules. In some embodiments, the TCR complex protein antigen-binding domain is a designed ankyrin repeat (DARPin) targeted to a protein of the TCR. In some embodiments, the TCR complex protein antigen-binding domain is a single-chain variable fragment (scFv) targeted to a protein of the TCR. In some embodiments, the TCR complex protein antigen-binding domain is a nanobody targeted to a protein of the TCR.

Proteins associated with the TCR include, but are not limited, to the TCR alpha (a) chain, TCR beta (β) chain, TCR gamma (γ) chain, TCR delta (δ) chain, CD3γ chain, CD3δ chain and CD3ε chains. In some embodiments, an antigen-binding domain that binds a protein associated with the TCR complex is an antibody to the TCR alpha (α) chain, TCR beta (β) chain, TCR gamma (γ) chain, TCR delta (δ) chain, CD3γ chain, CD3δ chain and/or CD3ε chain. In some embodiments, the protein associated with a TCR complex is CD3. In some embodiments, the protein associated with a TCR complex is CD3ε. In some embodiments, the antigen-binding domain that binds CD3 is an antibody, for example, a single chain antibody, for example a single-chain variable fragment (scFv). Examples of CD3 antibodies, include, but are not limited to, UCHT1, OKT3, F6A, L2K, muromonab, otelixizumab, teplizumab, visilizumab, CD3-12, MEM-57, 4D10A6, CD3D, or TR66.

In some embodiments, the antigen-binding domain that binds the TCR complex is UCHT1, or a variant thereof. In some embodiments, the UCHT1 antigen-binding domain is encoded by SEQ ID NO: 31. In some embodiments, the UCHT1 antigen-binding domain comprises SEQ ID NO: 32. In some embodiments, the UCHT1 antigen-binding domain is mutated. In some embodiments, the UCHT1 antigen-binding domain comprises a Y to T mutation at a position corresponding to amino acid 182 of SEQ ID NO: 32 (Y182T). In some embodiments, the UCHT1 (Y182T) antigen-binding domain is encoded by SEQ ID NO: 43. In some embodiments, the UCHT1 (Y182T) antigen-binding domain comprises SEQ ID NO: 44. In some embodiments, the antigen-binding domain that binds the TCR complex is a humanized UCHT1 (huUCHT1). In some embodiments, the huUCHT1 antigen-binding domain is encoded by SEQ ID NO: 39. In some embodiments, the huUCHT1 antigen-binding domain comprises SEQ ID NO: 40. In some embodiments, the huUCHT1 has a Y to T mutation at a position corresponding to amino acid 177 of SEQ ID NO: 40 (Y177T). In some embodiments, the huUCHT1 (Y177T) antigen-binding domain is encoded by SEQ ID NO: 41. In some embodiments, the huUCHT1 antigen-binding domain comprises SEQ ID NO: 42.

In some embodiments, the polynucleotide encoding the antigen-binding domain that binds the protein associated with the TCR complex comprises a nucleotide sequence having at least 70% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 75% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 80% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 85% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 90% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 95% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 96% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 97% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 98% sequence identity with the nucleotide sequence of SEQ ID NO: 31 (UCHT1). In some embodiments, the polynucleotide encoding the antigen-binding domain that binds the protein associated with the TCR complex comprises a nucleotide sequence having at least 99% sequence identity with the nucleotide sequence of SEQ ID NO: 31 (UCHT1). In some embodiments, the polynucleotide encoding the antigen-binding domain that binds the protein associated with the TCR complex comprises the nucleotide sequence of SEQ ID NO: 31 (UCHT1).

In some embodiments, the antigen-binding domain that binds the protein associated with the TCR complex comprises an amino acid sequence having at least 70% sequence identity with the amino acid sequence of SEQ ID NO: 32 (UCHT1). In some embodiments, the antigen-binding domain that binds the protein associated with the TCR complex comprises an amino acid sequence having at least 75% sequence identity with the amino acid sequence of SEQ ID NO: 32 (UCHT1). In some embodiments, the antigen-binding domain that binds the protein associated with the TCR complex comprises an amino acid sequence having at least 80% sequence identity with the amino acid sequence of SEQ ID NO: 32 (UCHT1). In some embodiments, the antigen-binding domain that binds the protein associated with the TCR complex comprises an amino acid sequence having at least 85% sequence identity with the amino acid sequence of SEQ ID NO: 32 (UCHT1). In some embodiments, the antigen-binding domain that binds the protein associated with the TCR complex comprises an amino acid sequence having at least 90% sequence identity with the amino acid sequence of SEQ ID NO: 32 (UCHT1). In some embodiments, the antigen-binding domain that binds the protein associated with the TCR complex comprises an amino acid sequence having at least 95% sequence identity with the amino acid sequence of SEQ ID NO: 32 (UCHT1). In some embodiments, the antigen-binding domain that binds the protein associated with the TCR complex comprises an amino acid sequence having at least 96% sequence identity with the amino acid sequence of SEQ ID NO: 32 (UCHT1). In some embodiments, the antigen-binding domain that binds the protein associated with the TCR complex comprises an amino acid sequence having at least 97% sequence identity with the amino acid sequence of SEQ ID NO: 32 (UCHT1). In some embodiments, the antigen-binding domain that binds the protein associated with the TCR complex comprises an amino acid sequence having at least 98% sequence identity with the amino acid sequence of SEQ ID NO: 32 (UCHT1). In some embodiments, the antigen-binding domain that binds the protein associated with the TCR complex comprises an amino acid sequence having at least 99% sequence identity with the amino acid sequence of SEQ ID NO: 32 (UCHT1). In some embodiments, the antigen-binding domain that binds the protein associated with the TCR complex comprises the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 32 (UCHT1). In some embodiments, the CDR sequences of the antigen-binding domain that binds the protein associated with the TCR complex have 100% identity with the CDR sequences of the amino acid sequence of SEQ ID NO: 32 (UCHT1), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 32 (UCHT1).

In some embodiments, the polynucleotide encoding the antigen-binding domain that binds the protein associated with the TCR complex comprises a nucleotide sequence having at least 70% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 75% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 80% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 85% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 90% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 95% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 96% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 97% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 98% sequence identity with the nucleotide sequence of SEQ ID NO: 43 (UCHT1 (Y182T)). In some embodiments, the polynucleotide encoding the antigen-binding domain that binds the protein associated with the TCR complex comprises a nucleotide sequence having at least 99% sequence identity with the nucleotide sequence of SEQ ID NO: 43 (UCHT1 (Y182T)). In some embodiments, the polynucleotide encoding the antigen-binding domain that binds the protein associated with the TCR complex comprises the nucleotide sequence of SEQ ID NO: 43 (UCHT1 (Y182T)).

In some embodiments, the antigen-binding domain that binds the protein associated with the TCR complex comprises an amino acid sequence having at least 70% sequence identity with the amino acid sequence of SEQ ID NO: 44 (UCHT1 (Y182T)). In some embodiments, the antigen-binding domain that binds the protein associated with the TCR complex comprises an amino acid sequence having at least 75% sequence identity with the amino acid sequence of SEQ ID NO: 44 (UCHT1 (Y182T)). In some embodiments, the antigen-binding domain that binds the protein associated with the TCR complex comprises an amino acid sequence having at least 80% sequence identity with the amino acid sequence of SEQ ID NO: 44 (UCHT1 (Y182T)). In some embodiments, the antigen-binding domain that binds the protein associated with the TCR complex comprises an amino acid sequence having at least 85% sequence identity with the amino acid sequence of SEQ ID NO: 44 (UCHT1 (Y182T)). In some embodiments, the antigen-binding domain that binds the protein associated with the TCR complex comprises an amino acid sequence having at least 90% sequence identity with the amino acid sequence of SEQ ID NO: 44 (UCHT1 (Y182T)). In some embodiments, the antigen-binding domain that binds the protein associated with the TCR complex comprises an amino acid sequence having at least 95% sequence identity with the amino acid sequence of SEQ ID NO: 44 (UCHT1 (Y182T)). In some embodiments, the antigen-binding domain that binds the protein associated with the TCR complex comprises an amino acid sequence having at least 96% sequence identity with the amino acid sequence of SEQ ID NO: 44 (UCHT1 (Y182T)). In some embodiments, the antigen-binding domain that binds the protein associated with the TCR complex comprises an amino acid sequence having at least 97% sequence identity with the amino acid sequence of SEQ ID NO: 44 (UCHT1 (Y182T)). In some embodiments, the antigen-binding domain that binds the protein associated with the TCR complex comprises an amino acid sequence having at least 98% sequence identity with the amino acid sequence of SEQ ID NO: 44 (UCHT1 (Y182T)). In some embodiments, the antigen-binding domain that binds the protein associated with the TCR complex comprises an amino acid sequence having at least 99% sequence identity with the amino acid sequence of SEQ ID NO: 44 (UCHT1 (Y182T)). In some embodiments, the antigen-binding domain that binds the protein associated with the TCR complex comprises the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 44 (UCHT1 (Y182T)). In some embodiments, the CDR sequences of the antigen-binding domain that binds the protein associated with the TCR complex have 100% identity with the CDR sequences of the amino acid sequence of SEQ ID NO: 44 (UCHT1 (Y182T)), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 44 (UCHT1 (Y182T)).

In some embodiments, the polynucleotide encoding the antigen-binding domain that binds the protein associated with the TCR complex comprises a nucleotide sequence having at least 70% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 75% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 80% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 85% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 90% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 95% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 96% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 97% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 98% sequence identity with the nucleotide sequence of SEQ ID NO: 39 (huUCHT1). In some embodiments, the polynucleotide encoding the antigen-binding domain that binds the protein associated with the TCR complex comprises a nucleotide sequence having at least 99% sequence identity with the nucleotide sequence of SEQ ID NO: 39 (huUCHT1). In some embodiments, the polynucleotide encoding the antigen-binding domain that binds the protein associated with the TCR complex comprises the nucleotide sequence of SEQ ID NO: 39 (huUCHT1).

In some embodiments, the antigen-binding domain that binds the protein associated with the TCR complex comprises an amino acid sequence having at least 70% sequence identity with the amino acid sequence of SEQ ID NO: 40 (huUCHT1). In some embodiments, the antigen-binding domain that binds the protein associated with the TCR complex comprises an amino acid sequence having at least 75% sequence identity with the amino acid sequence of SEQ ID NO: 40 (huUCHT1). In some embodiments, the antigen-binding domain that binds the protein associated with the TCR complex comprises an amino acid sequence having at least 80% sequence identity with the amino acid sequence of SEQ ID NO: 40 (huUCHT1). In some embodiments, the antigen-binding domain that binds the protein associated with the TCR complex comprises an amino acid sequence having at least 85% sequence identity with the amino acid sequence of SEQ ID NO: 40 (huUCHT1). In some embodiments, the antigen-binding domain that binds the protein associated with the TCR complex comprises an amino acid sequence having at least 90% sequence identity with the amino acid sequence of SEQ ID NO: 40 (huUCHT1). In some embodiments, the antigen-binding domain that binds the protein associated with the TCR complex comprises an amino acid sequence having at least 95% sequence identity with the amino acid sequence of SEQ ID NO: 40 (huUCHT1). In some embodiments, the antigen-binding domain that binds the protein associated with the TCR complex comprises an amino acid sequence having at least 96% sequence identity with the amino acid sequence of SEQ ID NO: 40 (huUCHT1). In some embodiments, the antigen-binding domain that binds the protein associated with the TCR complex comprises an amino acid sequence having at least 97% sequence identity with the amino acid sequence of SEQ ID NO: 40 (huUCHT1). In some embodiments, the antigen-binding domain that binds the protein associated with the TCR complex comprises an amino acid sequence having at least 98% sequence identity with the amino acid sequence of SEQ ID NO: 40 (huUCHT1). In some embodiments, the antigen-binding domain that binds the protein associated with the TCR complex comprises an amino acid sequence having at least 99% sequence identity with the amino acid sequence of SEQ ID NO: 40 (huUCHT1). In some embodiments, the antigen-binding domain that binds the protein associated with the TCR complex comprises the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 40 (huUCHT1). In some embodiments, the CDR sequences of the antigen-binding domain that binds the protein associated with the TCR complex have 100% identity with the CDR sequences of the amino acid sequence of SEQ ID NO: 40 (huUCHT1), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 40 (huUCHT1).

In some embodiments, the polynucleotide encoding the antigen-binding domain that binds the protein associated with the TCR complex comprises a nucleotide sequence having at least 70% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 75% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 80% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 85% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 90% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 95% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 96% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 97% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 98% sequence identity with the nucleotide sequence of SEQ ID NO: 41 (huUCHT1 (Y177T)). In some embodiments, the polynucleotide encoding the antigen-binding domain that binds the protein associated with the TCR complex comprises a nucleotide sequence having at least 99% sequence identity with the nucleotide sequence of SEQ ID NO: 41 (huUCHT1 (Y177T)). In some embodiments, the polynucleotide encoding the antigen-binding domain that binds the protein associated with the TCR complex comprises the nucleotide sequence of SEQ ID NO: 41 (huUCHT1 (Y177T)).

In some embodiments, the antigen-binding domain that binds the protein associated with the TCR complex comprises an amino acid sequence having at least 70% sequence identity with the amino acid sequence of SEQ ID NO: 42 (huUCHT1 (Y177T)). In some embodiments, the antigen-binding domain that binds the protein associated with the TCR complex comprises an amino acid sequence having at least 75% sequence identity with the amino acid sequence of SEQ ID NO: 42 (huUCHT1 (Y177T)). In some embodiments, the antigen-binding domain that binds the protein associated with the TCR complex comprises an amino acid sequence having at least 80% sequence identity with the amino acid sequence of SEQ ID NO: 42 (huUCHT1 (Y177T)). In some embodiments, the antigen-binding domain that binds the protein associated with the TCR complex comprises an amino acid sequence having at least 85% sequence identity with the amino acid sequence of SEQ ID NO: 42 (huUCHT1 (Y177T)). In some embodiments, the antigen-binding domain that binds the protein associated with the TCR complex comprises an amino acid sequence having at least 90% sequence identity with the amino acid sequence of SEQ ID NO: 42 (huUCHT1 (Y177T)). In some embodiments, the antigen-binding domain that binds the protein associated with the TCR complex comprises an amino acid sequence having at least 95% sequence identity with the amino acid sequence of SEQ ID NO: 42 (huUCHT1 (Y177T)). In some embodiments, the antigen-binding domain that binds the protein associated with the TCR complex comprises an amino acid sequence having at least 96% sequence identity with the amino acid sequence of SEQ ID NO: 42 (huUCHT1 (Y177T)). In some embodiments, the antigen-binding domain that binds the protein associated with the TCR complex comprises an amino acid sequence having at least 97% sequence identity with the amino acid sequence of SEQ ID NO: 42 (huUCHT1 (Y177T)). In some embodiments, the antigen-binding domain that binds the protein associated with the TCR complex comprises an amino acid sequence having at least 98% sequence identity with the amino acid sequence of SEQ ID NO: 42 (huUCHT1 (Y177T)). In some embodiments, the antigen-binding domain that binds the protein associated with the TCR complex comprises an amino acid sequence having at least 99% sequence identity with the amino acid sequence of SEQ ID NO: 42 (huUCHT1 (Y177T)). In some embodiments, the antigen-binding domain that binds the protein associated with the TCR complex comprises the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 42 (huUCHT1 (Y177T)). In some embodiments, the CDR sequences of the antigen-binding domain that binds the protein associated with the TCR complex have 100% identity with the CDR sequences of the amino acid sequence of SEQ ID NO: 42 (huUCHT1 (Y177T)), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 42 (huUCHT1 (Y177T)).

In some embodiments, the antigen-binding domain that binds to the protein associated with the TCR complex is OKT3. In some embodiments, the murine OKT3 antigen-binding domain is encoded by SEQ ID NO: 33. In some embodiments, the OKT3 antigen-binding domain comprises SEQ ID NO: 34.

In some embodiments, the polynucleotide encoding the antigen-binding domain that binds the protein associated with the TCR complex comprises a nucleotide sequence having at least 70% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 75% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 80% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 85% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 90% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 95% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 96% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 97% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 98% sequence identity with the nucleotide sequence of SEQ ID NO: 33 (OKT3). In some embodiments, the polynucleotide encoding the antigen-binding domain that binds the protein associated with the TCR complex comprises a nucleotide sequence having at least 99% sequence identity with the nucleotide sequence of SEQ ID NO: 33 (OKT3). In some embodiments, the polynucleotide encoding the antigen-binding domain that binds the protein associated with the TCR complex comprises the nucleotide sequence of SEQ ID NO: 33 (OKT3).

In some embodiments, the antigen-binding domain that binds the protein associated with the TCR complex comprises an amino acid sequence having at least 70% sequence identity with the amino acid sequence of SEQ ID NO: 34 (OKT3). In some embodiments, the antigen-binding domain that binds the protein associated with the TCR complex comprises an amino acid sequence having at least 75% sequence identity with the amino acid sequence of SEQ ID NO: 34 (OKT3). In some embodiments, the antigen-binding domain that binds the protein associated with the TCR complex comprises an amino acid sequence having at least 80% sequence identity with the amino acid sequence of SEQ ID NO: 34 (OKT3). In some embodiments, the antigen-binding domain that binds the protein associated with the TCR complex comprises an amino acid sequence having at least 85% sequence identity with the amino acid sequence of SEQ ID NO: 34 (OKT3). In some embodiments, the antigen-binding domain that binds the protein associated with the TCR complex comprises an amino acid sequence having at least 90% sequence identity with the amino acid sequence of SEQ ID NO: 34 (OKT3). In some embodiments, the antigen-binding domain that binds the protein associated with the TCR complex comprises an amino acid sequence having at least 95% sequence identity with the amino acid sequence of SEQ ID NO: 34 (OKT3). In some embodiments, the antigen-binding domain that binds the protein associated with the TCR complex comprises an amino acid sequence having at least 96% sequence identity with the amino acid sequence of SEQ ID NO: 34 (OKT3). In some embodiments, the antigen-binding domain that binds the protein associated with the TCR complex comprises an amino acid sequence having at least 97% sequence identity with the amino acid sequence of SEQ ID NO: 34 (OKT3). In some embodiments, the antigen-binding domain that binds the protein associated with the TCR complex comprises an amino acid sequence having at least 98% sequence identity with the amino acid sequence of SEQ ID NO: 34 (OKT3). In some embodiments, the antigen-binding domain that binds the protein associated with the TCR complex comprises an amino acid sequence having at least 99% sequence identity with the amino acid sequence of SEQ ID NO: 34 (OKT3). In some embodiments, the antigen-binding domain that binds the protein associated with the TCR complex comprises the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 34 (OKT3). In some embodiments, the CDR sequences of the antigen-binding domain that binds the protein associated with the TCR complex have 100% identity with the CDR sequences of the amino acid sequence of SEQ ID NO: 34 (OKT3), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 34 (OKT3).

In some embodiments, the antigen-binding domain that binds to the protein associated with the TCR complex is F6A. In some embodiments, the murine F6A antigen-binding domain is encoded by SEQ ID NO: 35. In some embodiments, the F6A antigen-binding domain comprises SEQ ID NO: 36.

In some embodiments, the polynucleotide encoding the antigen-binding domain that binds the protein associated with the TCR complex comprises a nucleotide sequence having at least 70% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 75% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 80% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 85% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 90% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 95% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 96% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 97% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 98% sequence identity with the nucleotide sequence of SEQ ID NO: 35 (F6A). In some embodiments, the polynucleotide encoding the antigen-binding domain that binds the protein associated with the TCR complex comprises a nucleotide sequence having at least 99% sequence identity with the nucleotide sequence of SEQ ID NO: 35 (F6A). In some embodiments, the polynucleotide encoding the antigen-binding domain that binds the protein associated with the TCR complex comprises the nucleotide sequence of SEQ ID NO: 35 (F6A).

In some embodiments, the antigen-binding domain that binds the protein associated with the TCR complex comprises an amino acid sequence having at least 70% sequence identity with the amino acid sequence of SEQ ID NO: 36 (F6A). In some embodiments, the antigen-binding domain that binds the protein associated with the TCR complex comprises an amino acid sequence having at least 75% sequence identity with the amino acid sequence of SEQ ID NO: 36 (F6A). In some embodiments, the antigen-binding domain that binds the protein associated with the TCR complex comprises an amino acid sequence having at least 80% sequence identity with the amino acid sequence of SEQ ID NO: 36 (F6A). In some embodiments, the antigen-binding domain that binds the protein associated with the TCR complex comprises an amino acid sequence having at least 85% sequence identity with the amino acid sequence of SEQ ID NO: 36 (F6A). In some embodiments, the antigen-binding domain that binds the protein associated with the TCR complex comprises an amino acid sequence having at least 90% sequence identity with the amino acid sequence of SEQ ID NO: 36 (F6A). In some embodiments, the antigen-binding domain that binds the protein associated with the TCR complex comprises an amino acid sequence having at least 95% sequence identity with the amino acid sequence of SEQ ID NO: 36 (F6A). In some embodiments, the antigen-binding domain that binds the protein associated with the TCR complex comprises an amino acid sequence having at least 96% sequence identity with the amino acid sequence of SEQ ID NO: 36 (F6A). In some embodiments, the antigen-binding domain that binds the protein associated with the TCR complex comprises an amino acid sequence having at least 97% sequence identity with the amino acid sequence of SEQ ID NO: 36 (F6A). In some embodiments, the antigen-binding domain that binds the protein associated with the TCR complex comprises an amino acid sequence having at least 98% sequence identity with the amino acid sequence of SEQ ID NO: 36 (F6A). In some embodiments, the antigen-binding domain that binds the protein associated with the TCR complex comprises an amino acid sequence having at least 99% sequence identity with the amino acid sequence of SEQ ID NO: 36 (F6A). In some embodiments, the antigen-binding domain that binds the protein associated with the TCR complex comprises the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 36 (F6A). In some embodiments, the CDR sequences of the antigen-binding domain that binds the protein associated with the TCR complex have 100% identity with the CDR sequences of the amino acid sequence of SEQ ID NO: 36 (F6A), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 36 (F6A).

In some embodiments, the antigen-binding domain that binds to the protein associated with the TCR complex is L2K. In some embodiments, the murine L2K antigen-binding domain is encoded by SEQ ID NO: 37. In some embodiments, the L2K antigen-binding domain comprises SEQ ID NO: 38.

In some embodiments, the polynucleotide encoding the antigen-binding domain that binds the protein associated with the TCR complex comprises a nucleotide sequence having at least 70% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 75% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 80% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 85% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 90% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 95% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 96% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 97% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 98% sequence identity with the nucleotide sequence of SEQ ID NO: 37 (L2K). In some embodiments, the polynucleotide encoding the antigen-binding domain that binds the protein associated with the TCR complex comprises a nucleotide sequence having at least 99% sequence identity with the nucleotide sequence of SEQ ID NO: 37 (L2K). In some embodiments, the polynucleotide encoding the antigen-binding domain that binds the protein associated with the TCR complex comprises the nucleotide sequence of SEQ ID NO: 37 (L2K).

In some embodiments, the antigen-binding domain that binds the protein associated with the TCR complex comprises an amino acid sequence having at least 70% sequence identity with the amino acid sequence of SEQ ID NO: 38 (L2K). In some embodiments, the antigen-binding domain that binds the protein associated with the TCR complex comprises an amino acid sequence having at least 75% sequence identity with the amino acid sequence of SEQ ID NO: 38 (L2K). In some embodiments, the antigen-binding domain that binds the protein associated with the TCR complex comprises an amino acid sequence having at least 80% sequence identity with the amino acid sequence of SEQ ID NO: 38 (L2K). In some embodiments, the antigen-binding domain that binds the protein associated with the TCR complex comprises an amino acid sequence having at least 85% sequence identity with the amino acid sequence of SEQ ID NO: 38 (L2K). In some embodiments, the antigen-binding domain that binds the protein associated with the TCR complex comprises an amino acid sequence having at least 90% sequence identity with the amino acid sequence of SEQ ID NO: 38 (L2K). In some embodiments, the antigen-binding domain that binds the protein associated with the TCR complex comprises an amino acid sequence having at least 95% sequence identity with the amino acid sequence of SEQ ID NO: 38 (L2K). In some embodiments, the antigen-binding domain that binds the protein associated with the TCR complex comprises an amino acid sequence having at least 96% sequence identity with the amino acid sequence of SEQ ID NO: 38 (L2K). In some embodiments, the antigen-binding domain that binds the protein associated with the TCR complex comprises an amino acid sequence having at least 97% sequence identity with the amino acid sequence of SEQ ID NO: 38 (L2K). In some embodiments, the antigen-binding domain that binds the protein associated with the TCR complex comprises an amino acid sequence having at least 98% sequence identity with the amino acid sequence of SEQ ID NO: 38 (L2K). In some embodiments, the antigen-binding domain that binds the protein associated with the TCR complex comprises an amino acid sequence having at least 99% sequence identity with the amino acid sequence of SEQ ID NO: 38 (L2K). In some embodiments, the antigen-binding domain that binds the protein associated with the TCR complex comprises the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K) (i.e., the antigen-binding domain that binds the protein associated with the TCR complex comprises an amino acid sequence comprising a CDRH1, CDRH2, CDRH3, CDRL1, CDRL2, and CDRL3, each having 100% identity to the corresponding CDR in the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 80% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 85% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 90% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 95% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 96% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 97% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 98% sequence identity with the non-CDR (e.g., framework) sequences of the amino acid sequence of SEQ ID NO: 38 (L2K). In some embodiments, the CDR sequences of the antigen-binding domain that binds the protein associated with the TCR complex have 100% identity with the CDR sequences of the amino acid sequence of SEQ ID NO: 38 (L2K), and the non-CDR (e.g., framework) sequences of the antigen-binding domain that binds the protein associated with the TCR complex have at least 99% sequence identity with the non-CDR (e.g., framework) sequences of the amino acid sequence of SEQ ID NO: 38 (L2K).

Amino acid and nucleotide sequences of exemplary antigen-binding domains that bind a protein associated with the TCR complex are provided in Table 1.

TABLE 1
Table of Sequences

| SEQ ID NO | Description | Nucleotide/Amino Acid |
|---|---|---|
| SEQ ID NO: 31 | UCHT1¹ | Nucleotide |
| SEQ ID NO: 32 | UCHT1² | Amino Acid |
| SEQ ID NO: 33 | OKT3 | Nucleotide |
| SEQ ID NO: 34 | OKT3 | Amino Acid |
| SEQ ID NO: 35 | F6A | Nucleotide |
| SEQ ID NO: 36 | F6A | Amino Acid |
| SEQ ID NO: 37 | L2K | Nucleotide |
| SEQ ID NO: 38 | L2K | Amino Acid |
| SEQ ID NO: 39 | huUCHT1 | Nucleotide |
| SEQ ID NO: 40 | huUCHT1 | Amino Acid |
| SEQ ID NO: 41 | huUCHT1 (Y177T) | Nucleotide |
| SEQ ID NO: 42 | huUCHT1 (Y177T) | Amino Acid |
| SEQ ID NO: 43 | UCHT1 (Y182T) | Nucleotide |
| SEQ ID NO: 44 | UCHT1 (Y182T) | Amino Acid |

¹Light chain, nucleotides 1-324; Linker, nucleotides 325-387; Heavy chain, nucleotides 388-750
²Light chain, amino acids 1-108; Linker, amino acids 109-128; Heavy chain, amino acids 129-250

### Transmembrane Domain and Cytosolic Domain

In some embodiments, a GUCY2C T cell antigen coupler polypeptide comprises a T cell receptor signaling domain polypeptide. In some embodiments, a GUCY2C T cell antigen coupler polypeptide comprises a transmembrane domain of a TCR signaling domain. In some embodiments, a GUCY2C T cell antigen coupler polypeptide comprises a cytosolic domain of a TCR signaling domain polypeptide. In some embodiments, a GUCY2C T cell antigen coupler polypeptide comprises a transmembrane domain and a cytosolic domain of a TCR signaling domain polypeptide.

In some embodiments, the T cell receptor signaling domain polypeptide comprises a TCR co-receptor domain. In some embodiments, the TCR signaling domain polypeptide comprises a transmembrane domain and/or a cytosolic domain of a TCR co-receptor. In some embodiments, the TCR co-receptor is CD4, CD8, LAG3, or a chimeric variation thereof.

In some embodiments, the TCR co-receptor is CD4. In some embodiments, the GUCY2C TAC comprises a transmembrane domain and a cytosolic domain of a CD4 co-receptor. In some embodiments, the polynucleotide encoding the cytosolic and transmembrane domain comprises a nucleotide sequence having at least 70% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 75% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 80% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 85% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 90% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 95% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 96% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 97% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 98% sequence identity with the nucleotide sequence of SEQ ID NO: 45 (CD4 transmembrane and cytosolic domain). In some embodiments, the polynucleotide encoding the cytosolic and transmembrane domain comprises a nucleotide sequence having at least 99% sequence identity with the nucleotide sequence of SEQ ID NO: 45 (CD4 transmembrane and cytosolic domain). In some embodiments, the polynucleotide encoding the cytosolic and transmembrane domain comprises the nucleotide sequence of SEQ ID NO: 45 (CD4 transmembrane and cytosolic domain). In some embodiments, the cytosolic and transmembrane domain comprise an amino acid sequence having at least 70% sequence identity with the amino acid sequence of SEQ ID NO: 46 (CD4 transmembrane and cytosolic domain). In some embodiments, the cytosolic and transmembrane domain comprise an amino acid sequence having at least 75% sequence identity with the amino acid sequence of SEQ ID NO: 46 (CD4 transmembrane and cytosolic domain). In some embodiments, the cytosolic and transmembrane domain comprise an amino acid sequence having at least 80% sequence identity with the amino acid sequence of SEQ ID NO: 46 (CD4 transmembrane and cytosolic domain). In some embodiments, the cytosolic and transmembrane domain comprise an amino acid sequence having at least 85% sequence identity with the amino acid sequence of SEQ ID NO: 46 (CD4 transmembrane and cytosolic domain). In some embodiments, the cytosolic and transmembrane domain comprise an amino acid sequence having at least 90% sequence identity with the amino acid sequence of SEQ ID NO: 46 (CD4 transmembrane and cytosolic domain). In some embodiments, the cytosolic and transmembrane domain comprise an amino acid sequence having at least 95% sequence identity with the amino acid sequence of SEQ ID NO: 46 (CD4 transmembrane and cytosolic domain). In some embodiments, the cytosolic and transmembrane domain comprise an amino acid sequence having at least 96% sequence identity with the amino acid sequence of SEQ ID NO: 46 (CD4 transmembrane and cytosolic domain). In some embodiments, the cytosolic and transmembrane domain comprise an amino acid sequence having at least 97% sequence identity with the amino acid sequence of SEQ ID NO: 46 (CD4 transmembrane and cytosolic domain). In some embodiments, the cytosolic and transmembrane domain comprise an amino acid sequence having at least 98% sequence identity with the amino acid sequence of SEQ ID NO: 46 (CD4 transmembrane and cytosolic domain). In some embodiments, the cytosolic and transmembrane domain comprise an amino acid sequence having at least 99% sequence identity with the amino acid sequence of SEQ ID NO: 46 (CD4 transmembrane and cytosolic domain). In some embodiments, the cytosolic and transmembrane domain comprise the amino acid sequence of SEQ ID NO: 46 (CD4 transmembrane and cytosolic domain).

In some embodiments, the TCR co-receptor is CD8. In some embodiments, the TCR co-receptor is CD8a. In some embodiments, the polynucleotide encoding the cytosolic and transmembrane domain comprises a nucleotide sequence having at least 70% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 75% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 80% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 85% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 90% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 95% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 96% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 97% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 98% sequence identity with the nucleotide sequence of SEQ ID NO: 47 (CD8 transmembrane and cytosolic domain). In some embodiments, the polynucleotide encoding the cytosolic and transmembrane domain comprises a nucleotide sequence having at least 99% sequence identity with the nucleotide sequence of SEQ ID NO: 47 (CD8 transmembrane and cytosolic domain). In some embodiments, the polynucleotide encoding the cytosolic and transmembrane domain comprises the nucleotide sequence of SEQ ID NO: 47 (CD8 transmembrane and cytosolic domain). In some embodiments, the cytosolic and transmembrane domain comprise an amino acid sequence having at least 70% sequence identity with the amino acid sequence of SEQ ID NO: 48 (CD8 transmembrane and cytosolic domain). In some embodiments, the cytosolic and transmembrane domain comprise an amino acid sequence having at least 75% sequence identity with the amino acid sequence of SEQ ID NO: 48 (CD8 transmembrane and cytosolic domain). In some embodiments, the cytosolic and transmembrane domain comprise an amino acid sequence having at least 80% sequence identity with the amino acid sequence of SEQ ID NO: 48 (CD8 transmembrane and cytosolic domain). In some embodiments, the cytosolic and transmembrane domain comprise an amino acid sequence having at least 85% sequence identity with the amino acid sequence of SEQ ID NO: 48 (CD8 transmembrane and cytosolic domain). In some embodiments, the cytosolic and transmembrane domain comprise an amino acid sequence having at least 90% sequence identity with the amino acid sequence of SEQ ID NO: 48 (CD8 transmembrane and cytosolic domain). In some embodiments, the cytosolic and transmembrane domain comprise an amino acid sequence having at least 95% sequence identity with the amino acid sequence of SEQ ID NO: 48 (CD8 transmembrane and cytosolic domain). In some embodiments, the cytosolic and transmembrane domain comprise an amino acid sequence having at least 96% sequence identity with the amino acid sequence of SEQ ID NO: 48 (CD8 transmembrane and cytosolic domain). In some embodiments, the cytosolic and transmembrane domain comprise an amino acid sequence having at least 97% sequence identity with the amino acid sequence of SEQ ID NO: 48 (CD8 transmembrane and cytosolic domain). In some embodiments, the cytosolic and transmembrane domain comprise an amino acid sequence having at least 98% sequence identity with the amino acid sequence of SEQ ID NO: 48 (CD8 transmembrane and cytosolic domain). In some embodiments, the cytosolic and transmembrane domain comprise an amino acid sequence having at least 99% sequence identity with the amino acid sequence of SEQ ID NO: 48 (CD8 transmembrane and cytosolic domain). In some embodiments, the cytosolic and transmembrane domain comprise the amino acid sequence of SEQ ID NO: 48 (CD8 transmembrane and cytosolic domain).

In some embodiments, the TCR signaling domain polypeptide comprises a chimera of sequences or domains from co-receptors. In some embodiments, the TCR signaling domain polypeptide comprises a chimera of CD8α and CD8β, wherein the CD8α arginine rich region is replaced with the CD8β arginine rich region (CD8α+R(β) chimera). In some embodiments, the polynucleotide encoding the cytosolic and transmembrane domain comprises a nucleotide sequence having at least 70% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 75% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 80% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 85% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 90% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 95% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 96% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 97% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 98% sequence identity with the nucleotide sequence of SEQ ID NO: 49 (CD8α+R(β) chimera). In some embodiments, the polynucleotide encoding the cytosolic and transmembrane domain comprises a nucleotide sequence having at least 99% sequence identity with the nucleotide sequence of SEQ ID NO: 49 (CD8α+R(β) chimera). In some embodiments, the polynucleotide encoding the cytosolic and transmembrane domain comprises the nucleotide sequence of SEQ ID NO: 49 (CD8α+R(β) chimera). In some embodiments, the cytosolic and transmembrane domain comprise an amino acid sequence having at least 70% sequence identity with the amino acid sequence of SEQ ID NO: 50 (CD8α+R(β) chimera). In some embodiments, the cytosolic and transmembrane domain comprise an amino acid sequence having at least 75% sequence identity with the amino acid sequence of SEQ ID NO: 50 (CD8α+R(β) chimera). In some embodiments, the cytosolic and transmembrane domain comprise an amino acid sequence having at least 80% sequence identity with the amino acid sequence of SEQ ID NO: 50 (CD8α+R(β) chimera). In some embodiments, the cytosolic and transmembrane domain comprise an amino acid sequence having at least 85% sequence identity with the amino acid sequence of SEQ ID NO: 50 (CD8α+R(β) chimera). In some embodiments, the cytosolic and transmembrane domain comprise an amino acid sequence having at least 90% sequence identity with the amino acid sequence of SEQ ID NO: 50 (CD8α+R(β) chimera). In some embodiments, the cytosolic and transmembrane domain comprise an amino acid sequence having at least 95% sequence identity with the amino acid sequence of SEQ ID NO: 50 (CD8α+R(β) chimera). In some embodiments, the cytosolic and transmembrane domain comprise an amino acid sequence having at least 96% sequence identity with the amino acid sequence of SEQ ID NO: 50 (CD8α+R(β) chimera). In some embodiments, the cytosolic and transmembrane domain comprise an amino acid sequence having at least 97% sequence identity with the amino acid sequence of SEQ ID NO: 50 (CD8α+R(β) chimera). In some embodiments, the cytosolic and transmembrane domain comprise an amino acid sequence having at least 98% sequence identity with the amino acid sequence of SEQ ID NO: 50 (CD8α+R(β) chimera). In some embodiments, the cytosolic and transmembrane domain comprise an amino acid sequence having at least 99% sequence identity with the amino acid sequence of SEQ ID NO: 50 (CD8α+R(β) chimera). In some embodiments, the cytosolic and transmembrane domain comprise the amino acid sequence of SEQ ID NO: 50 (CD8α+R(β) chimera).

In some embodiments, the TCR signaling domain polypeptide comprises a chimera of CD8α and CD8β, where the CD8α CXCP domain, which contains an Lck binding motif, is appended to the C-terminus of the CD8β cytosolic domain (CD8β+Lck chimera). In some embodiments, the polynucleotide encoding the cytosolic and transmembrane domain comprises a nucleotide sequence having at least 70% sequence identity with the nucleotide sequence of SEQ ID NO: 51 (CD8β+Lck chimera). In some embodiments, the polynucleotide encoding the cytosolic and transmembrane domain comprises a nucleotide sequence having at least 75% sequence identity with the nucleotide sequence of SEQ ID NO: 51 (CD8β+Lck chimera). In some embodiments, the polynucleotide encoding the cytosolic and transmembrane domain comprises a nucleotide sequence having at least 80% sequence identity with the nucleotide sequence of SEQ ID NO: 51 (CD8β+Lck chimera). In some embodiments, the polynucleotide encoding the cytosolic and transmembrane domain comprises a nucleotide sequence having at least 85% sequence identity with the nucleotide sequence of SEQ ID NO: 51 (CD8β+Lck chimera). In some embodiments, the polynucleotide encoding the cytosolic and transmembrane domain comprises a nucleotide sequence having at least 90% sequence identity with the nucleotide sequence of SEQ ID NO: 51 (CD8β+Lck chimera). In some embodiments, the polynucleotide encoding the cytosolic and transmembrane domain comprises a nucleotide sequence having at least 95% sequence identity with the nucleotide sequence of SEQ ID NO: 51 (CD8β+Lck chimera). In some embodiments, the polynucleotide encoding the cytosolic and transmembrane domain comprises a nucleotide sequence having at least 96% sequence identity with the nucleotide sequence of SEQ ID NO: 51 (CD8β+Lck chimera). In some embodiments, the polynucleotide encoding the cytosolic and transmembrane domain comprises a nucleotide sequence having at least 97% sequence identity with the nucleotide sequence of SEQ ID NO: 51 (CD8β+Lck chimera). In some embodiments, the polynucleotide encoding the cytosolic and transmembrane domain comprises a nucleotide sequence having at least 98% sequence identity with the nucleotide sequence of SEQ ID NO: 51 (CD8β+Lck chimera). In some embodiments, the polynucleotide encoding the cytosolic and transmembrane domain comprises a nucleotide sequence having at least 99% sequence identity with the nucleotide sequence of SEQ ID NO: 51 (CD8β+Lck chimera). In some embodiments, the polynucleotide encoding the cytosolic and transmembrane domain comprises the nucleotide sequence of SEQ ID NO: 51 (CD8β+Lck chimera). In some embodiments, the cytosolic and transmembrane domain comprise an amino acid sequence having at least 70% sequence identity with the amino acid sequence of SEQ ID NO: 52 (CD8β+Lck chimera). In some embodiments, the cytosolic and transmembrane domain comprise an amino acid sequence having at least 75% sequence identity with the amino acid sequence of SEQ ID NO: 52 (CD8β+Lck chimera). In some embodiments, the cytosolic and transmembrane domain comprise an amino acid sequence having at least 80% sequence identity with the amino acid sequence of SEQ ID NO: 52 (CD8β+Lck chimera). In some embodiments, the cytosolic and transmembrane domain comprise an amino acid sequence having at least 85% sequence identity with the amino acid sequence of SEQ ID NO: 52 (CD8β+Lck chimera). In some embodiments, the cytosolic and transmembrane domain comprise an amino acid sequence having at least 90% sequence identity with the amino acid sequence of SEQ ID NO: 52 (CD8β+Lck chimera). In some embodiments, the cytosolic and transmembrane domain comprise an amino acid sequence having at least 95% sequence identity with the amino acid sequence of SEQ ID NO: 52 (CD8β+Lck chimera). In some embodiments, the cytosolic and transmembrane domain comprise an amino acid sequence having at least 96% sequence identity with the amino acid sequence of SEQ ID NO: 52 (CD8β+Lck chimera). In some embodiments, the cytosolic and transmembrane domain comprise an amino acid sequence having at least 97% sequence identity with the amino acid sequence of SEQ ID NO: 52 (CD8β+Lck chimera). In some embodiments, the cytosolic and transmembrane domain comprise an amino acid sequence having at least 98% sequence identity with the amino acid sequence of SEQ ID NO: 52 (CD8β+Lck chimera). In some embodiments, the cytosolic and transmembrane domain comprise an amino acid sequence having at least 99% sequence identity with the amino acid sequence of SEQ ID NO: 52 (CD8β+Lck chimera). In some embodiments, the cytosolic and transmembrane domain comprise the amino acid sequence of SEQ ID NO: 52 (CD8β+Lck chimera).

In some embodiments, the TCR signaling domain polypeptide includes both a cytosolic domain and a transmembrane domain of a TCR co-receptor protein. In some embodiments, the cytosolic domain and transmembrane domain are from the same co-receptor or from different co-receptors.

Amino acid and nucleotide sequences of exemplary transmembrane and cytosolic domains are provided in Table 2.

TABLE 2
Table of Sequences

| SEQ ID NO | Description | Nucleotide/Amino Acid |
|---|---|---|
| SEQ ID NO: 45 | CD4 Domain¹ | Nucleotide |
| SEQ ID NO: 46 | CD4 Domain² | Amino Acid |
| SEQ ID NO: 47 | CD8α Domain | Nucleotide |
| SEQ ID NO: 48 | CD8α Domain | Amino Acid |
| SEQ ID NO: 49 | CD8α + R(β) Domain | Nucleotide |
| SEQ ID NO: 50 | CD8α + R(β) Domain | Amino Acid |
| SEQ ID NO: 51 | CD8 α + Lck Domain | Nucleotide |
| SEQ ID NO: 52 | CD8 α + Lck Domain | Amino Acid |

¹Extracellular linker, nucleotides 1-66; Transmembrane domain, nucleotides 67-132; Cytosolic domain, nucleotides 133-254
²Extracellular linker, amino acids 1-22; Transmembrane domain, amino acids 23-44; Cytosolic domain, amino acids 45-84

### Configurations, Linkers, and Connectors

In some embodiments, a nucleic acid disclosed herein is in an order of (1) a first polynucleotide encoding an antigen-binding domain that binds GUCY2C; (2) a second polynucleotide encoding an antigen-binding domain that binds a TCR complex; (3) a third polynucleotide encoding a transmembrane domain and a cytosolic domain. In some embodiments, a nucleic acid disclosed herein is in an order of (1) a first polynucleotide encoding an antigen-binding domain that binds GUCY2C; (2) a second polynucleotide encoding an antigen-binding domain that binds a TCR complex; (3) a third polynucleotide encoding a transmembrane domain and a cytosolic domain, wherein the order is 5′ end to 3′ end. In some embodiments, a nucleic acid disclosed herein is in an order of (1) a first polynucleotide encoding an antigen-binding domain that binds GUCY2C; (2) a second polynucleotide encoding an antigen-binding domain that binds a TCR complex; (3) a third polynucleotide encoding a transmembrane domain and a cytosolic domain, wherein the order is 3′ end to 5′ end. In some embodiments, a nucleic acid described herein is in an order of (1) a first polynucleotide encoding an antigen-binding domain that binds a TCR complex; (2) a second polynucleotide encoding an antigen-binding domain that binds GUCY2C; (3) a third polynucleotide encoding a transmembrane domain and a cytosolic domain. In some embodiments, a nucleic acid described herein is in an order of (1) a first polynucleotide encoding an antigen-binding domain that binds a TCR complex; (2) a second polynucleotide encoding an antigen-binding domain that binds GUCY2C; (3) a third polynucleotide encoding a transmembrane domain and a cytosolic domain, wherein the order is 5′ end to 3′ end. In some embodiments, a nucleic acid described herein is in an order of (1) a first polynucleotide encoding an antigen-binding domain that binds a TCR complex; (2) a second polynucleotide encoding an antigen-binding domain that binds GUCY2C; (3) a third polynucleotide encoding a transmembrane domain and a cytosolic domain, wherein the order is 3′ end to 5′ end.

In some embodiments, a GUCY2C TAC polypeptide disclosed herein is in an order of (1) an antigen-binding domain that binds GUCY2C; (2) an antigen-binding domain that binds a TCR complex; (3) a transmembrane domain and a cytosolic domain, wherein the order is N-terminus to C-terminus. In some embodiments, a GUCY2C TAC polypeptide disclosed herein is in an order of (1) an antigen-binding domain that binds GUCY2C; (2) an antigen-binding domain that binds a TCR complex; (3) a transmembrane domain and a cytosolic domain, wherein the order is C-terminus to N-terminus. In some embodiments, a GUCY2C TAC polypeptide described herein is in an order of (1) an antigen-binding domain that binds a TCR complex; (2) an antigen-binding domain that binds GUCY2C; (3) a transmembrane domain and a cytosolic domain, wherein the order is N-terminus to C-terminus. In some embodiments, a GUCY2C TAC polypeptide described herein is in an order of (1) an antigen-binding domain that binds a TCR complex; (2) an antigen-binding domain that binds GUCY2C; (3) a transmembrane domain and a cytosolic domain, wherein the order is C-terminus to N-terminus.

In some embodiments, the antigen-binding domain that binds GUCY2C, the antigen-binding domain that binds the TCR complex, and/or the transmembrane domain and cytosolic domain are directly fused. For example, the antigen-binding domain that binds GUCY2C and the transmembrane domain and cytosolic domain are both fused to the antigen-binding domain that binds the TCR complex. In some embodiments, the antigen-binding domain that binds GUCY2C, the antigen-binding domain that binds the TCR complex, and/or the transmembrane domain and cytosolic domain are joined by at least one linker. In some embodiments, the antigen-binding domain that binds GUCY2C and the antigen-binding domain that binds the TCR complex are directly fused, and joined to the transmembrane domain and cytosolic domain by a linker. In some embodiments, the antigen-binding domain that binds the TCR complex and the transmembrane domain and cytosolic domain are directly fused, and joined to the antigen-binding domain that binds GUCY2C by a linker.

In some embodiments, the linker is a peptide linker. In some embodiments, the peptide linker comprises 1 to 40 amino acids. In some embodiments, the peptide linker comprises 1 to 30 amino acids. In some embodiments, the peptide linker comprises 1 to 15 amino acids. In some embodiments, the peptide linker comprises 1 to 10 amino acids. In some embodiments, the peptide linker comprises 1 to 6 amino acids. In some embodiments, the peptide linker comprises 30 to 40 amino acids. In some embodiments, the peptide linker comprises 32 to 36 amino acids. In some embodiments, the peptide linker comprises 5 to 30 amino acids. In some embodiments, the peptide linker comprises 5 amino acids. In some embodiments, the peptide linker comprises 10 amino acids. In some embodiments, the peptide linker comprises 15 amino acids. In some embodiments, the peptide linker comprises 20 amino acids. In some embodiments, the peptide linker comprises 25 amino acids. In some embodiments, the peptide linker comprises 30 amino acids. In some embodiments, the peptide linker comprises a glycine and/or serine-rich linker.

In some embodiments, the at least one linker comprises an amino acid sequence having at least 80% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 85% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 90% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 95% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 96% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 97% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 98% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises an amino acid sequence having at least 99% identity with the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker). In some embodiments, the at least one linker comprises the amino acid sequence of SEQ ID NO: 26 ((G4S)4-based linker), SEQ ID NO: 28 (G4S-based linker), SEQ ID NO: 6 (linker 1), SEQ ID NO: 8 (linker 2), SEQ ID NO: 10 (CD4 based linker), SEQ ID NO: 12 (short helix connector), SEQ ID NO: 14 (long helix connector), SEQ ID NO: 16 (large domain connector), or SEQ ID NO: 24 (G4S3 linker).

In some embodiments, the peptide linker that joins the antigen-binding domain that binds GUCY2C to the antigen-binding domain that binds a TCR complex (e.g., UCHT1) is known as the connector to distinguish this protein domain from other linkers in the TAC. The connector may be of any size. In some embodiments, the connector between the antigen-binding domain that binds a TCR complex and the antigen-binding domain that binds GUCY2C is a short helix comprising SEQ ID NO: 12. In some embodiments, the connector between the antigen-binding domain that binds a TCR complex and the antigen-binding domain that binds GUCY2C is a short helix encoded by SEQ ID NO: 11. In some embodiments, the connector between the antigen-binding domain that binds a TCR complex and the antigen-binding domain that binds GUCY2C is a long helix comprising SEQ ID NO: 14. In some embodiments, the connector between the antigen-binding domain that binds a TCR complex and the antigen-binding domain that binds GUCY2C is a long helix encoded by SEQ ID NO: 13. In some embodiments, the connector between the antigen-binding domain that binds a TCR complex and the antigen-binding domain that binds GUCY2C is a large domain comprising SEQ ID NO: 16. In some embodiments, the connector between the antigen-binding domain that binds a TCR complex and the antigen-binding domain that binds GUCY2C is a large domain encoded by SEQ ID NO: 15.

In some embodiments, a nucleic acid or TAC disclosed herein comprises a leader sequence. In some embodiments, the leader sequence is encoded by a nucleotide sequence having at least 80% sequence identity with the nucleotide sequence of SEQ ID NO: 1 (muIgG leader), SEQ ID NO: 17 (huIgG leader), SEQ ID NO: 19 (huCD8α leader), or SEQ ID NO: 29 (huCD8α leader). In some embodiments, the leader sequence is encoded by a nucleotide sequence having at least 85% sequence identity with the nucleotide sequence of SEQ ID NO: 1 (muIgG leader), SEQ ID NO: 17 (huIgG leader), SEQ ID NO: 19 (huCD8α leader), or SEQ ID NO: 29 (huCD8α leader). In some embodiments, the leader sequence is encoded by a nucleotide sequence having at least 90% sequence identity with the nucleotide sequence of SEQ ID NO: 1 (muIgG leader), SEQ ID NO: 17 (huIgG leader), SEQ ID NO: 19 (huCD8α leader), or SEQ ID NO: 29 (huCD8α leader). In some embodiments, the leader sequence is encoded by a nucleotide sequence having at least 95% sequence identity with the nucleotide sequence of SEQ ID NO: 1 (muIgG leader), SEQ ID NO: 17 (huIgG leader), SEQ ID NO: 19 (huCD8α leader), or SEQ ID NO: 29 (huCD8α leader). In some embodiments, the leader sequence is encoded by a nucleotide sequence having at least 96% sequence identity with the nucleotide sequence of SEQ ID NO: 1 (muIgG leader), SEQ ID NO: 17 (huIgG leader), SEQ ID NO: 19 (huCD8α leader), or SEQ ID NO: 29 (huCD8α leader). In some embodiments, the leader sequence is encoded by a nucleotide sequence having at least 97% sequence identity with the nucleotide sequence of SEQ ID NO: 1 (muIgG leader), SEQ ID NO: 17 (huIgG leader), SEQ ID NO: 19 (huCD8α leader), or SEQ ID NO: 29 (huCD8α leader). In some embodiments, the leader sequence is encoded by a nucleotide sequence having at least 98% sequence identity with the nucleotide sequence of SEQ ID NO: 1 (muIgG leader), SEQ ID NO: 17 (huIgG leader), SEQ ID NO: 19 (huCD8α leader), or SEQ ID NO: 29 (huCD8α leader). In some embodiments, the leader sequence is encoded by a nucleotide sequence having at least 99% sequence identity with the nucleotide sequence of SEQ ID NO: 1 (muIgG leader), SEQ ID NO: 17 (huIgG leader), SEQ ID NO: 19 (huCD8α leader), or SEQ ID NO: 29 (huCD8α leader). In some embodiments, the leader sequence comprises the nucleotide sequence of SEQ ID NO: 1 (muIgG leader), SEQ ID NO: 17 (huIgG leader), SEQ ID NO: 19 (huCD8α leader), or SEQ ID NO: 29 (huCD8α leader).

In some embodiments, a nucleic acid or TAC disclosed herein comprises a leader sequence. In some embodiments, the leader sequence comprises an amino acid sequence having at least 80% sequence identity with the amino acid sequence of SEQ ID NO: 2 (muIgG leader), SEQ ID NO: 18 (huIgG leader), or SEQ ID NO: 20 (huCD8α leader). In some embodiments, the leader sequence comprises an amino acid sequence having at least 85% sequence identity with the amino acid sequence of SEQ ID NO: 2 (muIgG leader), SEQ ID NO: 18 (huIgG leader), or SEQ ID NO: 20 (huCD8α leader). In some embodiments, the leader sequence comprises an amino acid sequence having at least 90% sequence identity with the amino acid sequence of SEQ ID NO: 2 (muIgG leader), SEQ ID NO: 18 (huIgG leader), or SEQ ID NO: 20 (huCD8α leader). In some embodiments, the leader sequence comprises an amino acid sequence having at least 95% sequence identity with the amino acid sequence of SEQ ID NO: 2 (muIgG leader), SEQ ID NO: 18 (huIgG leader), or SEQ ID NO: 20 (huCD8α leader). In some embodiments, the leader sequence comprises an amino acid sequence having at least 96% sequence identity with the amino acid sequence of SEQ ID NO: 2 (muIgG leader), SEQ ID NO: 18 (huIgG leader), or SEQ ID NO: 20 (huCD8α leader). In some embodiments, the leader sequence comprises an amino acid sequence having at least 97% sequence identity with the amino acid sequence of SEQ ID NO: 2 (muIgG leader), SEQ ID NO: 18 (huIgG leader), or SEQ ID NO: 20 (huCD8α leader). In some embodiments, the leader sequence comprises an amino acid sequence having at least 98% sequence identity with the amino acid sequence of SEQ ID NO: 2 (muIgG leader), SEQ ID NO: 18 (huIgG leader), or SEQ ID NO: 20 (huCD8α leader). In some embodiments, the leader sequence comprises an amino acid sequence having at least 99% sequence identity with the amino acid sequence of SEQ ID NO: 2 (muIgG leader), SEQ ID NO: 18 (huIgG leader), or SEQ ID NO: 20 (huCD8α leader). In some embodiments, the leader sequence comprises the amino acid sequence of SEQ ID NO: 2 (muIgG leader), SEQ ID NO: 18 (huIgG leader), or SEQ ID NO: 20 (huCD8α leader).

In some embodiments, a GUCY2C T cell antigen coupler polypeptide comprises a tag, e.g., a Myc tag. In some embodiments, the tag comprises an amino acid sequence having at least 80% identity with the amino acid sequence of SEQ ID NO: 4 (Myc Tag). In some embodiments, the tag comprises an amino acid sequence having at least 85% identity with the amino acid sequence of SEQ ID NO: 4 (Myc Tag). In some embodiments, the tag comprises an amino acid sequence having at least 90% identity with the amino acid sequence of SEQ ID NO: 4 (Myc Tag). In some embodiments, the tag comprises an amino acid sequence having at least 95% identity with the amino acid sequence of SEQ ID NO: 4 (Myc Tag). In some embodiments, the tag comprises an amino acid sequence having at least 96% identity with the amino acid sequence of SEQ ID NO: 4 (Myc Tag). In some embodiments, the tag comprises an amino acid sequence having at least 97% identity with the amino acid sequence of SEQ ID NO: 4 (Myc Tag). In some embodiments, the tag comprises an amino acid sequence having at least 98% identity with the amino acid sequence of SEQ ID NO: 4 (Myc Tag). In some embodiments, the tag comprises an amino acid sequence having at least 99% identity with the amino acid sequence of SEQ ID NO: 4 (Myc Tag). In some embodiments, the tag comprises the amino acid sequence of SEQ ID NO: 4 (Myc Tag).

Amino acid and nucleotide sequences of exemplary linkers, connectors, tags, and leader sequences are provided in Table 3.

TABLE 3
Table of Sequences

| SEQ ID NO | Description | Nucleotide/Amino Acid |
|---|---|---|
| SEQ ID NO: 1 | muIgG leader (secretion signal) | Nucleotide |
| SEQ ID NO: 2 | muIgG leader (secretion signal) | Amino Acid |
| SEQ ID NO: 3 | Myc Tag | Nucleotide |
| SEQ ID NO: 4 | Myc Tag | Amino Acid |
| SEQ ID NO: 5 | Linker 1 | Nucleotide |
| SEQ ID NO: 6 | Linker 1 | Amino Acid |
| SEQ ID NO: 7 | Linker 2 | Nucleotide |
| SEQ ID NO: 8 | Linker 2 | Amino Acid |
| SEQ ID NO: 9 | CD4 linker | Nucleotide |
| SEQ ID NO: 10 | CD4 linker | Amino Acid |
| SEQ ID NO: 11 | Short Helix connector | Nucleotide |
| SEQ ID NO: 12 | Short Helix connector | Amino Acid |
| SEQ ID NO: 13 | Long Helix connector | Nucleotide |
| SEQ ID NO: 14 | Long Helix connector | Amino Acid |
| SEQ ID NO: 15 | Large domain connector | Nucleotide |
| SEQ ID NO: 16 | Large domain connector | Amino Acid |
| SEQ ID NO: 17 | huIgG | Nucleotide |
| SEQ ID NO: 18 | huIgG | Amino Acid |
| SEQ ID NO: 19 | huCD8a-1 | Nucleotide |
| SEQ ID NO: 20 | huCD8a-1 | Amino Acid |
| SEQ ID NO: 21 | Whitlow Linker | Nucleotide |
| SEQ ID NO: 22 | Whitlow Linker | Amino Acid |
| SEQ ID NO: 23 | (G4S)3 linker | Nucleotide |
| SEQ ID NO: 24 | (G4S)3 linker | Amino Acid |
| SEQ ID NO: 25 | (G4S)4 linker | Nucleotide |
| SEQ ID NO: 26 | (G4S)4 linker | Amino Acid |
| SEQ ID NO: 27 | G4S linker | Nucleotide |
| SEQ ID NO: 28 | G4S linker | Amino Acid |
| SEQ ID NO: 29 | huCD8a-2 | Nucleotide |
| SEQ ID NO: 30 | huCD8a-2 | Amino Acid |

### GUCY2C Antigen-Binding Domain

In certain embodiments, the GUCY2C TAC polypeptide comprises a GUCY2C antigen-binding domain. In some embodiments, the GUCY2C antigen-binding domain selectively binds GUCY2C. In some embodiments, the GUCY2C antigen-binding domain binds to GUCY2C on a target cell. In some embodiments, a target cell is a cell associated with a disease state, including, but not limited to, cancer. In some embodiments, a target cell is a tumor cell.

In some embodiments, the GUCY2C antigen-binding domain is an antibody or a fragment thereof. In some embodiments, the GUCY2C antigen-binding domain is selected from single chain antibodies (e.g., single-chain fragment variable antibodies (scFvs)), single domain antibodies (e.g., heavy-chain-only antibodies (VHIH), shark heavy-chain-only antibodies (VNAR)), nanobodies, diabodies, minibodies, Fab fragments, Fab′ fragments, F(ab′)₂ fragments, or Fv fragments that bind to GUCY2C.

In some embodiments, the GUCY2C antigen-binding domain is selected from ankyrin repeat proteins (DARPins), affibodies, adnectins, affilins, phylomers, fynomers, affimers, peptide aptamers, lectins, knottins, centyrins, anticalins, peptides, peptidomimetics, proteins, glycoproteins, or proteoglycans that bind to GUCY2C, or naturally occurring ligands for GUCY2C. In some embodiments, the GUCY2C antigen-binding domain is a non-protein compound that binds to GUCY2C, including but not limited to carbohydrates, lipids, nucleic acids, or small molecules.

In some embodiments, the GUCY2C antigen-binding domain is a designed ankyrin repeat (DARPin) targeted to GUCY2C. In some embodiments, the GUCY2C antigen-binding domain is a single-chain variable fragment (scFv) targeted to GUCY2C. In some embodiments, the GUCY2C antigen-binding domain is a nanobody targeted to GUCY2C.

In some embodiments, the GUCY2C antigen-binding domain is selected from an amino acid sequence according to any one of SEQ ID NOs: 53-127 or 514-521.

In some embodiments, the GUCY2C antigen-binding domain comprises an amino acid sequence having at least 80% sequence identity with an amino acid sequence according to any one of SEQ ID NOs: 53-127 or 514-521. In some embodiments, the GUCY2C antigen-binding domain comprises an amino acid sequence having at least 85% sequence identity with an amino acid sequence according to any one of SEQ ID NOs: 53-127 or 514-521. In some embodiments, the GUCY2C antigen-binding domain comprises an amino acid sequence having at least 90% sequence identity an amino acid sequence according to any one of SEQ ID NOs: 53-127 or 514-521. In some embodiments, the GUCY2C antigen-binding domain comprises an amino acid sequence having at least 95% sequence identity with an amino acid sequence according to any one of SEQ ID NOs: 53-127 or 514-521. In some embodiments, the GUCY2C antigen-binding domain comprises an amino acid sequence having at least 96% sequence identity an amino acid sequence according to any one of SEQ ID NOs: 53-127 or 514-521. In some embodiments, the GUCY2C antigen-binding domain comprises an amino acid sequence having at least 97% sequence identity with an amino acid sequence according to any one of SEQ ID NOs: 53-127 or 514-521. In some embodiments, the GUCY2C antigen-binding domain comprises an amino acid sequence having at least 98% sequence identity with an amino acid sequence according to any one of SEQ ID NOs: 53-127 or 514-521. In some embodiments, the GUCY2C antigen-binding domain comprises an amino acid sequence having at least 99% sequence identity with an amino acid sequence according to any one of SEQ ID NOs: 53-127 or 514-521.

In some embodiments, the GUCY2C antigen-binding domain comprises an amino acid sequence at least 80% identical to an amino acid sequence according to any one of SEQ ID NOs: 53-127 or 514-521, wherein the CDR sequences of the GUCY2C antigen-binding domain sequence have 100% sequence identity to the CDR sequences of the sequence of the SEQ ID NO. In some embodiments, the GUCY2C antigen-binding domain comprises an amino acid sequence at least 85% identical to an amino acid sequence according to any one of SEQ ID NOs: 53-127 or 514-521, wherein the CDR sequences of the GUCY2C antigen-binding domain sequence have 100% sequence identity to the CDR sequences of the sequence of the SEQ ID NO. In some embodiments, the GUCY2C antigen-binding domain comprises an amino acid sequence at least 90% identical to an amino acid sequence according to any one of SEQ ID NOs: 53-127 or 514-521, wherein the CDR sequences of the GUCY2C antigen-binding domain sequence have 100% sequence identity to the CDR sequences of the sequence of the SEQ ID NO. In some embodiments, the GUCY2C antigen-binding domain comprises an amino acid sequence at least 95% identical to an amino acid sequence according to any one of SEQ ID NOs: 53-127 or 514-521, wherein the CDR sequences of the GUCY2C antigen-binding domain sequence have 100% sequence identity to the CDR sequences of the sequence of the SEQ ID NO. In some embodiments, the GUCY2C antigen-binding domain comprises an amino acid sequence at least 96% identical to an amino acid sequence according to any one of SEQ ID NOs: 53-127 or 514-521, wherein the CDR sequences of the GUCY2C antigen-binding domain sequence have 100% sequence identity to the CDR sequences of the sequence of the SEQ ID NO. In some embodiments, the GUCY2C antigen-binding domain comprises an amino acid sequence at least 97% identical to an amino acid sequence according to any one of SEQ ID NOs: 53-127 or 514-521, wherein the CDR sequences of the GUCY2C antigen-binding domain sequence have 100% sequence identity to the CDR sequences of the sequence of the SEQ ID NO. In some embodiments, the GUCY2C antigen-binding domain comprises an amino acid sequence at least 98% identical to an amino acid sequence according to any one of SEQ ID NOs: 53-127 or 514-521, wherein the CDR sequences of the GUCY2C antigen-binding domain sequence have 100% sequence identity to the CDR sequences of the sequence of the SEQ ID NO. In some embodiments, the GUCY2C antigen-binding domain comprises an amino acid sequence at least 99% identical to an amino acid sequence according to any one of SEQ ID NOs: 53-127 or 514-521, wherein the CDR sequences of the GUCY2C antigen-binding domain sequence have 100% sequence identity to the CDR sequences of the sequence of the SEQ ID NO.

Amino acid sequences of exemplary GUCY2C antigen-binding domains are provided in Table 4.

TABLE 4
Table of Sequences

| SEQ ID NO. | Description | Amino Acid/Nucleic Acid |
|---|---|---|
| SEQ ID NO: 53 | YU652-B06 | Amino Acid |
| SEQ ID NO: 54 | YU652-C01 | Amino Acid |
| SEQ ID NO: 55 | YU652-F02 | Amino Acid |
| SEQ ID NO: 56 | YU652-H02 | Amino Acid |
| SEQ ID NO: 57 | YU653-B12 | Amino Acid |
| SEQ ID NO: 58 | YU653-D11 | Amino Acid |
| SEQ ID NO: 59 | YU653-F12 | Amino Acid |
| SEQ ID NO: 60 | YU654-E01 | Amino Acid |
| SEQ ID NO: 61 | YU667-A01 | Amino Acid |
| SEQ ID NO: 62 | YU667-A02 | Amino Acid |
| SEQ ID NO: 63 | YU667-A04 | Amino Acid |
| SEQ ID NO: 64 | YU667-B01 | Amino Acid |
| SEQ ID NO: 65 | YU667-B02 | Amino Acid |
| SEQ ID NO: 66 | YU667-B03 | Amino Acid |
| SEQ ID NO: 67 | YU667-C04 | Amino Acid |
| SEQ ID NO: 68 | YU667-C06 | Amino Acid |
| SEQ ID NO: 69 | YU667-D05 | Amino Acid |
| SEQ ID NO: 70 | YU667-D06 | Amino Acid |
| SEQ ID NO: 71 | YU667-E01 | Amino Acid |
| SEQ ID NO: 72 | YU667-E05 | Amino Acid |
| SEQ ID NO: 73 | YU667-F02 | Amino Acid |
| SEQ ID NO: 74 | YU667-F06 | Amino Acid |
| SEQ ID NO: 75 | YU667-G02 | Amino Acid |
| SEQ ID NO: 76 | YU667-G04 | Amino Acid |
| SEQ ID NO: 77 | YU667-G06 | Amino Acid |
| SEQ ID NO: 78 | YU667-H06 | Amino Acid |
| SEQ ID NO: 79 | YU652-B06-Whitlow | Amino Acid |
| SEQ ID NO: 80 | YU652-C01-Whitlow | Amino Acid |
| SEQ ID NO: 81 | YU652-F02-Whitlow | Amino Acid |
| SEQ ID NO: 82 | YU652-H02-Whitlow | Amino Acid |
| SEQ ID NO: 83 | YU653-B12-Whitlow | Amino Acid |
| SEQ ID NO: 84 | YU653-D11-Whitlow | Amino Acid |
| SEQ ID NO: 85 | YU653-F12-Whitlow | Amino Acid |
| SEQ ID NO: 86 | YU654-E01-Whitlow | Amino Acid |
| SEQ ID NO: 87 | YU667-A01-Whitlow | Amino Acid |
| SEQ ID NO: 88 | YU667-A02-Whitlow | Amino Acid |
| SEQ ID NO: 89 | YU667-A04-Whitlow | Amino Acid |
| SEQ ID NO: 90 | YU667-B01-Whitlow | Amino Acid |
| SEQ ID NO: 91 | YU667-B02-Whitlow | Amino Acid |
| SEQ ID NO: 92 | YU667-B03-Whitlow | Amino Acid |
| SEQ ID NO: 93 | YU667-C04-Whitlow | Amino Acid |
| SEQ ID NO: 94 | YU667-C06-Whitlow | Amino Acid |
| SEQ ID NO: 95 | YU667-D05-Whitlow | Amino Acid |
| SEQ ID NO: 96 | YU667-D06-Whitlow | Amino Acid |
| SEQ ID NO: 97 | YU667-E01-Whitlow | Amino Acid |
| SEQ ID NO: 98 | YU667-E05-Whitlow | Amino Acid |
| SEQ ID NO: 99 | YU667-F02-Whitlow | Amino Acid |
| SEQ ID NO: 100 | YU667-F06-Whitlow | Amino Acid |
| SEQ ID NO: 101 | YU667-G02-Whitlow | Amino Acid |
| SEQ ID NO: 102 | YU667-G04-Whitlow | Amino Acid |
| SEQ ID NO: 103 | YU667-G06-Whitlow | Amino Acid |
| SEQ ID NO: 104 | YU667-H06-Whitlow | Amino Acid |
| SEQ ID NO: 105 | 6293-R4A-C2 | Amino Acid |
| SEQ ID NO: 106 | 6293-R4A-E3 | Amino Acid |
| SEQ ID NO: 107 | 6293-R4A-G4 | Amino Acid |
| SEQ ID NO: 108 | 6293-R5A-A5 | Amino Acid |
| SEQ ID NO: 109 | 6293-R5A-B6 | Amino Acid |
| SEQ ID NO: 110 | 6293-R5A-E7 | Amino Acid |
| SEQ ID NO: 111 | 6293-R5A-F6 | Amino Acid |
| SEQ ID NO: 112 | 6293-R6A-A10 | Amino Acid |
| SEQ ID NO: 113 | 6293-R6A-B11 | Amino Acid |
| SEQ ID NO: 114 | 6293-R6A-C9 | Amino Acid |
| SEQ ID NO: 115 | 6293-R6A-G9 | Amino Acid |
| SEQ ID NO: 116 | 6293-R6A-H11 | Amino Acid |
| SEQ ID NO: 117 | TI001-A23 | Amino Acid |
| SEQ ID NO: 118 | TI001-A25 | Amino Acid |
| SEQ ID NO: 119 | TI001-A28 | Amino Acid |
| SEQ ID NO: 120 | TI001-A30 | Amino Acid |
| SEQ ID NO: 121 | TI001-A48 | Amino Acid |
| SEQ ID NO: 122 | TI001-V20 | Amino Acid |
| SEQ ID NO: 123 | TI001-V30 | Amino Acid |
| SEQ ID NO: 124 | TI001-V54 | Amino Acid |
| SEQ ID NO: 125 | TI001-V65 | Amino Acid |
| SEQ ID NO: 126 | TI001-V67 | Amino Acid |
| SEQ ID NO: 127 | TI001-V68 | Amino Acid |
| SEQ ID NO: 514 | T1001-E8 | Amino Acid |
| SEQ ID NO: 515 | T1001-E16 | Amino Acid |
| SEQ ID NO: 516 | T1001-E6 | Amino Acid |
| SEQ ID NO: 517 | T1001-E64 | Amino Acid |
| SEQ ID NO: 518 | T1001-E55 | Amino Acid |
| SEQ ID NO: 519 | T1001-E17 | Amino Acid |
| SEQ ID NO: 520 | T1001-E24 | Amino Acid |
| SEQ ID NO: 521 | T1001-E25 | Amino Acid |

In some embodiments, the GUCY2C antigen-binding domain comprises a heavy chain variable region having an amino acid sequence according to any one of SEQ TD NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence according to any one of SEQ TD NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203.

In some embodiments, the GUCY2C antigen-binding domain comprises a heavy chain variable region having an amino acid sequence having at least 8000 sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 80% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203. In some embodiments, the GUCY2C antigen-binding domain comprises a heavy chain variable region having an amino acid sequence having at least 85% sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 85% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203. In some embodiments, the GUCY2C antigen-binding domain comprises a heavy chain variable region having an amino acid sequence having at least 90% sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 90% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203. In some embodiments, the GUCY2C antigen-binding domain comprises a heavy chain variable region having an amino acid sequence having at least 95% sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 95% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203. In some embodiments, the GUCY2C antigen-binding domain comprises a heavy chain variable region having an amino acid sequence having at least 96% sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 96% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203. In some embodiments, the GUCY2C antigen-binding domain comprises a heavy chain variable region having an amino acid sequence having at least 97% sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 97% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203. In some embodiments, the GUCY2C antigen-binding domain comprises a heavy chain variable region having an amino acid sequence having at least 98% sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 98% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203. In some embodiments, the GUCY2C antigen-binding domain comprises a heavy chain variable region having an amino acid sequence having at least 99% sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 99% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203.

In some embodiments, the GUCY2C antigen-binding domain comprises (a) a heavy chain variable region having an amino acid sequence at least 80% identical to a sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202, wherein the CDR sequences of the heavy chain variable region sequence have 100% sequence identity to the CDR sequences of the sequence of the SEQ ID NO.; and (b) a light chain variable region having an amino acid sequence at least 80% identical to a sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203, wherein the CDR sequences of the light chain variable region sequence have 100% sequence identity to the CDR sequences of the sequence of the SEQ ID NO.

In some embodiments, the GUCY2C antigen-binding domain comprises (a) a heavy chain variable region having an amino acid sequence at least 85% identical to a sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202, wherein the CDR sequences of the heavy chain variable region sequence have 100% sequence identity to the CDR sequences of the sequence of the SEQ ID NO.; and (b) a light chain variable region having an amino acid sequence at least 85% identical to a sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203, wherein the CDR sequences of the light chain variable region sequence have 100% sequence identity to the CDR sequences of the sequence of the SEQ ID NO.

In some embodiments, the GUCY2C antigen-binding domain comprises (a) a heavy chain variable region having an amino acid sequence at least 90% identical to a sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202, wherein the CDR sequences of the heavy chain variable region sequence have 100% sequence identity to the CDR sequences of the sequence of the SEQ ID NO.; and (b) a light chain variable region having an amino acid sequence at least 90% identical to a sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203, wherein the CDR sequences of the light chain variable region sequence have 100% sequence identity to the CDR sequences of the sequence of the SEQ ID NO.

In some embodiments, the GUCY2C antigen-binding domain comprises (a) a heavy chain variable region having an amino acid sequence at least 95% identical to a sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202, wherein the CDR sequences of the heavy chain variable region sequence have 100% sequence identity to the CDR sequences of the sequence of the SEQ ID NO.; and (b) a light chain variable region having an amino acid sequence at least 95% identical to a sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203, wherein the CDR sequences of the light chain variable region sequence have 100% sequence identity to the CDR sequences of the sequence of the SEQ ID NO.

In some embodiments, the GUCY2C antigen-binding domain comprises (a) a heavy chain variable region having an amino acid sequence at least 96% identical to a sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202, wherein the CDR sequences of the heavy chain variable region sequence have 100% sequence identity to the CDR sequences of the sequence of the SEQ ID NO.; and (b) a light chain variable region having an amino acid sequence at least 96% identical to a sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203, wherein the CDR sequences of the light chain variable region sequence have 100% sequence identity to the CDR sequences of the sequence of the SEQ ID NO.

In some embodiments, the GUCY2C antigen-binding domain comprises (a) a heavy chain variable region having an amino acid sequence at least 97% identical to a sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202, wherein the CDR sequences of the heavy chain variable region sequence have 100% sequence identity to the CDR sequences of the sequence of the SEQ ID NO.; and (b) a light chain variable region having an amino acid sequence at least 97% identical to a sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203, wherein the CDR sequences of the light chain variable region sequence have 100% sequence identity to the CDR sequences of the sequence of the SEQ ID NO.

In some embodiments, the GUCY2C antigen-binding domain comprises (a) a heavy chain variable region having an amino acid sequence at least 98% identical to a sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202, wherein the CDR sequences of the heavy chain variable region sequence have 100% sequence identity to the CDR sequences of the sequence of the SEQ ID NO.; and (b) a light chain variable region having an amino acid sequence at least 98% identical to a sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203, wherein the CDR sequences of the light chain variable region sequence have 100% sequence identity to the CDR sequences of the sequence of the SEQ ID NO.

In some embodiments, the GUCY2C antigen-binding domain comprises (a) a heavy chain variable region having an amino acid sequence at least 99% identical to a sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202, wherein the CDR sequences of the heavy chain variable region sequence have 100% sequence identity to the CDR sequences of the sequence of the SEQ ID NO.; and (b) a light chain variable region having an amino acid sequence at least 99% identical to a sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203, wherein the CDR sequences of the light chain variable region sequence have 100% sequence identity to the CDR sequences of the sequence of the SEQ ID NO.

Amino acid sequences of exemplary GUCY2C antigen-binding domain heavy chain variable regions and light chain variable regions are provided in Table 5.

TABLE 5
Table of Sequences

| SEQ ID NO | Description | Amino Acid/Nucleic Acid |
|---|---|---|
| SEQ ID NO: 128 | 6293-R4A-C2 VH | Amino Acid |
| SEQ ID NO: 129 | 6293-R4A-C2 VL | Amino Acid |
| SEQ ID NO: 130 | 6293-R4A-E3 VH | Amino Acid |
| SEQ ID NO: 131 | 6293-R4A-E3 VL | Amino Acid |
| SEQ ID NO: 132 | 6293-R4A-G4 VH | Amino Acid |
| SEQ ID NO: 133 | 6293-R4A-G4 VL | Amino Acid |
| SEQ ID NO: 134 | 6293-R5A-A5 VH | Amino Acid |
| SEQ ID NO: 135 | 6293-R5A-A5 VL | Amino Acid |
| SEQ ID NO: 136 | 6293-R5A-B6 VH | Amino Acid |
| SEQ ID NO: 137 | 6293-R5A-B6 VL | Amino Acid |
| SEQ ID NO: 138 | 6293-R5A-E7 VH | Amino Acid |
| SEQ ID NO: 139 | 6293-R5A-E7 VL | Amino Acid |
| SEQ ID NO: 140 | 6293-R5A-F6 VH | Amino Acid |
| SEQ ID NO: 141 | 6293-R5A-F6 VL | Amino Acid |
| SEQ ID NO: 142 | 6293-R6A-A10 VH | Amino Acid |
| SEQ ID NO: 143 | 6293-R6A-A10 VL | Amino Acid |
| SEQ ID NO: 144 | 6293-R6A-B11 VH | Amino Acid |
| SEQ ID NO: 145 | 6293-R6A-B11 VL | Amino Acid |
| SEQ ID NO: 146 | 6293-R6A-C9 VH | Amino Acid |
| SEQ ID NO: 147 | 6293-R6A-C9 VL | Amino Acid |
| SEQ ID NO: 148 | 6293-R6A-G9 VH | Amino Acid |
| SEQ ID NO: 149 | 6293-R6A-G9 VL | Amino Acid |
| SEQ ID NO: 150 | 6293-R6A-H11 VH | Amino Acid |
| SEQ ID NO: 151 | 6293-R6A-H11 VL | Amino Acid |
| SEQ ID NO: 152 | YU652-B06 VH | Amino Acid |
| SEQ ID NO: 153 | YU652-B06 VL | Amino Acid |
| SEQ ID NO: 154 | YU652-C01 VH | Amino Acid |
| SEQ ID NO: 155 | YU652-C01 VL | Amino Acid |
| SEQ ID NO: 156 | YU652-F02 VH | Amino Acid |
| SEQ ID NO: 157 | YU652-F02 VL | Amino Acid |
| SEQ ID NO: 158 | YU652-H02 VH | Amino Acid |
| SEQ ID NO: 159 | YU652-H02 VL | Amino Acid |
| SEQ ID NO: 160 | YU653-B12 VH | Amino Acid |
| SEQ ID NO: 161 | YU653-B12 VL | Amino Acid |
| SEQ ID NO: 162 | YU653-D11 VH | Amino Acid |
| SEQ ID NO: 163 | YU653-D11 VL | Amino Acid |
| SEQ ID NO: 164 | YU653-F12 VH | Amino Acid |
| SEQ ID NO: 165 | YU653-F12 VL | Amino Acid |
| SEQ ID NO: 166 | YU654-E01 VH | Amino Acid |
| SEQ ID NO: 167 | YU654-E01 VL | Amino Acid |
| SEQ ID NO: 168 | YU667-A01 VH | Amino Acid |
| SEQ ID NO: 169 | YU667-A01 VL | Amino Acid |
| SEQ ID NO: 170 | YU667-A02 VH | Amino Acid |
| SEQ ID NO: 171 | YU667-A02 VL | Amino Acid |
| SEQ ID NO: 172 | YU667-A04 VH | Amino Acid |
| SEQ ID NO: 173 | YU667-A04 VL | Amino Acid |
| SEQ ID NO: 174 | YU667-B01 VH | Amino Acid |
| SEQ ID NO: 175 | YU667-B01 VL | Amino Acid |
| SEQ ID NO: 176 | YU667-B02 VH | Amino Acid |
| SEQ ID NO: 177 | YU667-B02 VL | Amino Acid |
| SEQ ID NO: 178 | YU667-B03 VH | Amino Acid |
| SEQ ID NO: 179 | YU667-B03 VL | Amino Acid |
| SEQ ID NO: 180 | YU667-C04 VH | Amino Acid |
| SEQ ID NO: 181 | YU667-C04 VL | Amino Acid |
| SEQ ID NO: 182 | YU667-C06 VH | Amino Acid |
| SEQ ID NO: 183 | YU667-C06 VL | Amino Acid |
| SEQ ID NO: 184 | YU667-D05 VH | Amino Acid |
| SEQ ID NO: 185 | YU667-D05 VL | Amino Acid |
| SEQ ID NO: 186 | YU667-D06 VH | Amino Acid |
| SEQ ID NO: 187 | YU667-D06 VL | Amino Acid |
| SEQ ID NO: 188 | YU667-E01 VH | Amino Acid |
| SEQ ID NO: 189 | YU667-E01 VL | Amino Acid |
| SEQ ID NO: 190 | YU667-E05 VH | Amino Acid |
| SEQ ID NO: 191 | YU667-E05 VL | Amino Acid |
| SEQ ID NO: 192 | YU667-F02 VH | Amino Acid |
| SEQ ID NO: 193 | YU667-F02 VL | Amino Acid |
| SEQ ID NO: 194 | YU667-F06 VH | Amino Acid |
| SEQ ID NO: 195 | YU667-F06 VL | Amino Acid |
| SEQ ID NO: 196 | YU667-G02 VH | Amino Acid |
| SEQ ID NO: 197 | YU667-G02 VL | Amino Acid |
| SEQ ID NO: 198 | YU667-G04 VH | Amino Acid |
| SEQ ID NO: 199 | YU667-G04 VL | Amino Acid |
| SEQ ID NO: 200 | YU667-G06 VH | Amino Acid |
| SEQ ID NO: 201 | YU667-G06 VL | Amino Acid |
| SEQ ID NO: 202 | YU667-H06 VH | Amino Acid |
| SEQ ID NO: 203 | YU667-H06 VL | Amino Acid |

In some embodiments, the GUCY2C antigen-binding domain comprises a heavy chain variable region comprising (a) a CDR1 having an amino acid selected from the group consisting of SEQ TD NO: 204, 210, 216, 222, 228, 234, 240, 246, 252, 258, 264, 270, 276, 282, 288, 294, 300, 306, 312, 318, 324, 330, 336, 342, 348, 354, 393, 399, 405, 411, 417, 423, 429, 435, 441, 447, 453, 459, 522, 525, 528, 531, 534, 537, 540, and 543, (b) a CDR2 having an amino acid selected from the group consisting of SEQ TD NO: 205, 211, 217, 223, 229, 235, 241, 247, 253, 259, 265, 271, 277, 283, 289, 295, 301, 307, 313, 319, 325, 331, 337, 343, 349, 355, 394, 400, 406, 412, 418, 424, 430, 436, 442, 448, 454, 460, 523, 526, 529, 532, 535, 538, 541, and 544, and (c) a CDR3 having an amino acid selected from the group consisting of SEQ ID NO: 206, 212, 218, 224, 230, 236, 242, 248, 254, 260, 266, 272, 278, 284, 290, 296, 302, 308, 314, 320, 326, 332, 338, 344, 350, 356, 395, 401, 407, 413, 419, 425, 431, 437, 443, 449, 455, 461, 524, 527, 530, 533, 536, 539, 542, and 545; and a light chain variable region comprising (a) a CDR1 having an amino acid selected from the group consisting of SEQ ID NO: 207, 213, 219, 225, 231, 237, 243, 249, 255, 261, 267, 273, 279, 285, 291, 297, 303, 309, 315, 321, 327, 333, 339, 345, 351, 357, 396, 402, 408, 414, 420, 426, 432, 438, 444, 450, 456 and 462, (b) a CDR2 having an amino acid selected from the group consisting of SEQ ID NO: 208, 214, 220, 226, 232, 238, 244, 250, 256, 262, 268, 274, 280, 286, 292, 298, 304, 310, 316, 322, 328, 334, 340, 346, 352, 358, 397, 403, 409, 415, 421, 427, 433, 439, 445, 451, 457 and 463, and (c) a CDR3 having an amino acid selected from the group consisting of SEQ ID NO: 209, 215, 221, 227, 233, 239, 245, 251, 257, 263, 269, 275, 281, 287, 293, 299, 305, 311, 317, 323, 329, 335, 341, 347, 353, 359, 398, 404, 410, 416, 422, 428, 434, 440, 446, 452, 458 and 464.

In some embodiments, the GUCY2C antigen-binding domain is a nanobody and comprises (a) a VHH CDR1 having an amino acid selected from the group consisting of SEQ ID NO: 360, 363, 366, 369, 372, 375, 378, 381, 384, 387, and 390; (b) a VHH CDR2 having an amino acid selected from the group consisting of SEQ ID NO: 361, 364, 367, 370, 373, 376, 379, 382, 385, 388, and 391; and (c) a VHH CDR3 having an amino acid selected from the group consisting of SEQ ID NO: 362, 365, 368, 371, 374, 377, 380, 383, 386, 389, and 392.

Amino acid sequences of exemplary GUCY2C antigen-binding domain CDRs are provided in Table 6.

TABLE 6
Table of Sequences

| SEQ ID NO | Description | Amino Acid/Nucleic Acid |
|---|---|---|
| SEQ ID NO: 204 | YU653-F12 VH CDR1 | Amino Acid |
| SEQ ID NO: 205 | YU653-F12 VH CDR2 | Amino Acid |
| SEQ ID NO: 206 | YU653-F12 VH CDR3 | Amino Acid |
| SEQ ID NO: 207 | YU653-F12 VL CDR1 | Amino Acid |
| SEQ ID NO: 208 | YU653-F12 VL CDR2 | Amino Acid |
| SEQ ID NO: 209 | YU653-F12 VL CDR3 | Amino Acid |
| SEQ ID NO: 210 | YU652-B06 VH CDR1 | Amino Acid |
| SEQ ID NO: 211 | YU652-B06 VH CDR2 | Amino Acid |
| SEQ ID NO: 212 | YU652-B06 VH CDR3 | Amino Acid |
| SEQ ID NO: 213 | YU652-B06 VL CDR1 | Amino Acid |
| SEQ ID NO: 214 | YU652-B06 VL CDR2 | Amino Acid |
| SEQ ID NO: 215 | YU652-B06 VL CDR3 | Amino Acid |
| SEQ ID NO: 216 | YU653-D11 VH CDR1 | Amino Acid |
| SEQ ID NO: 217 | YU653-D11 VH CDR2 | Amino Acid |
| SEQ ID NO: 218 | YU653-D11 VH CDR3 | Amino Acid |
| SEQ ID NO: 219 | YU653-D11 VL CDR1 | Amino Acid |
| SEQ ID NO: 220 | YU653-D11 VL CDR2 | Amino Acid |
| SEQ ID NO: 221 | YU653-D11 VL CDR3 | Amino Acid |
| SEQ ID NO: 222 | YU652-C01 VH CDR1 | Amino Acid |
| SEQ ID NO: 223 | YU652-C01 VH CDR2 | Amino Acid |
| SEQ ID NO: 224 | YU652-C01 VH CDR3 | Amino Acid |
| SEQ ID NO: 225 | YU652-C01 VL CDR1 | Amino Acid |
| SEQ ID NO: 226 | YU652-C01 VL CDR2 | Amino Acid |
| SEQ ID NO: 227 | YU652-C01 VL CDR3 | Amino Acid |
| SEQ ID NO: 228 | YU652-H02 VH CDR1 | Amino Acid |
| SEQ ID NO: 229 | YU652-H02 VH CDR2 | Amino Acid |
| SEQ ID NO: 230 | YU652-H02 VH CDR3 | Amino Acid |
| SEQ ID NO: 231 | YU652-H02 VL CDR1 | Amino Acid |
| SEQ ID NO: 232 | YU652-H02 VL CDR2 | Amino Acid |
| SEQ ID NO: 233 | YU652-H02 VL CDR3 | Amino Acid |
| SEQ ID NO: 234 | YU654-E01 VH CDR1 | Amino Acid |
| SEQ ID NO: 235 | YU654-E01 VH CDR2 | Amino Acid |
| SEQ ID NO: 236 | YU654-E01 VH CDR3 | Amino Acid |
| SEQ ID NO: 237 | YU654-E01 VL CDR1 | Amino Acid |
| SEQ ID NO: 238 | YU654-E01 VL CDR2 | Amino Acid |
| SEQ ID NO: 239 | YU654-E01 VL CDR3 | Amino Acid |
| SEQ ID NO: 240 | YU653-B12 VH CDR 1 | Amino Acid |
| SEQ ID NO: 241 | YU653-B12 VH CDR 2 | Amino Acid |
| SEQ ID NO: 242 | YU653-B12 VH CDR 3 | Amino Acid |
| SEQ ID NO: 243 | YU653-B12 VL CDR 1 | Amino Acid |
| SEQ ID NO: 244 | YU653-B12 VL CDR 2 | Amino Acid |
| SEQ ID NO: 245 | YU653-B12 VL CDR 3 | Amino Acid |
| SEQ ID NO: 246 | YU652-F02 VH CDR 1 | Amino Acid |
| SEQ ID NO: 247 | YU652-F02 VH CDR 2 | Amino Acid |
| SEQ ID NO: 248 | YU652-F02 VH CDR 3 | Amino Acid |
| SEQ ID NO: 249 | YU652-F02 VL CDR 1 | Amino Acid |
| SEQ ID NO: 250 | YU652-F02 VL CDR 2 | Amino Acid |
| SEQ ID NO: 251 | YU652-F02 VL CDR 3 | Amino Acid |
| SEQ ID NO: 252 | YU667-C06 VH CDR 1 | Amino Acid |
| SEQ ID NO: 253 | YU667-C06 VH CDR 2 | Amino Acid |
| SEQ ID NO: 254 | YU667-C06 VH CDR 3 | Amino Acid |
| SEQ ID NO: 255 | YU667-C06 VL CDR 1 | Amino Acid |
| SEQ ID NO: 256 | YU667-C06 VL CDR 2 | Amino Acid |
| SEQ ID NO: 257 | YU667-C06 VL CDR 3 | Amino Acid |
| SEQ ID NO: 258 | YU667-E01 VH CDR 1 | Amino Acid |
| SEQ ID NO: 259 | YU667-E01 VH CDR 2 | Amino Acid |
| SEQ ID NO: 260 | YU667-E01 VH CDR 3 | Amino Acid |
| SEQ ID NO: 261 | YU667-E01 VL CDR 1 | Amino Acid |
| SEQ ID NO: 262 | YU667-E01 VL CDR 2 | Amino Acid |
| SEQ ID NO: 263 | YU667-E01 VL CDR 3 | Amino Acid |
| SEQ ID NO: 264 | YU667-B03 VH CDR 1 | Amino Acid |
| SEQ ID NO: 265 | YU667-B03 VH CDR 2 | Amino Acid |
| SEQ ID NO: 266 | YU667-B03 VH CDR 3 | Amino Acid |
| SEQ ID NO: 267 | YU667-B03 VL CDR 1 | Amino Acid |
| SEQ ID NO: 268 | YU667-B03 VL CDR 2 | Amino Acid |
| SEQ ID NO: 269 | YU667-B03 VL CDR 3 | Amino Acid |
| SEQ ID NO: 270 | YU667-F06 VH CDR 1 | Amino Acid |
| SEQ ID NO: 271 | YU667-F06 VH CDR 2 | Amino Acid |
| SEQ ID NO: 272 | YU667-F06 VH CDR 3 | Amino Acid |
| SEQ ID NO: 273 | YU667-F06 VL CDR 1 | Amino Acid |
| SEQ ID NO: 274 | YU667-F06 VL CDR 2 | Amino Acid |
| SEQ ID NO: 275 | YU667-F06 VL CDR 3 | Amino Acid |
| SEQ ID NO: 276 | YU667-C04 VH CDR 1 | Amino Acid |
| SEQ ID NO: 277 | YU667-C04 VH CDR 2 | Amino Acid |
| SEQ ID NO: 278 | YU667-C04 VH CDR 3 | Amino Acid |
| SEQ ID NO: 279 | YU667-C04 VL CDR 1 | Amino Acid |
| SEQ ID NO: 280 | YU667-C04 VL CDR 2 | Amino Acid |
| SEQ ID NO: 281 | YU667-C04 VL CDR 3 | Amino Acid |
| SEQ ID NO: 282 | YU667-G06 VH CDR 1 | Amino Acid |
| SEQ ID NO: 283 | YU667-G06 VH CDR 2 | Amino Acid |
| SEQ ID NO: 284 | YU667-G06 VH CDR 3 | Amino Acid |
| SEQ ID NO: 285 | YU667-G06 VL CDR 1 | Amino Acid |
| SEQ ID NO: 286 | YU667-G06 VL CDR 2 | Amino Acid |
| SEQ ID NO: 287 | YU667-G06 VL CDR 3 | Amino Acid |
| SEQ ID NO: 288 | YU667-D06 VH CDR 1 | Amino Acid |
| SEQ ID NO: 289 | YU667-D06 VH CDR 2 | Amino Acid |
| SEQ ID NO: 290 | YU667-D06 VH CDR 3 | Amino Acid |
| SEQ ID NO: 291 | YU667-D06 VL CDR 1 | Amino Acid |
| SEQ ID NO: 292 | YU667-D06 VL CDR 2 | Amino Acid |
| SEQ ID NO: 293 | YU667-D06 VL CDR 3 | Amino Acid |
| SEQ ID NO: 294 | YU667-G04 VH CDR 1 | Amino Acid |
| SEQ ID NO: 295 | YU667-G04 VH CDR 2 | Amino Acid |
| SEQ ID NO: 296 | YU667-G04 VH CDR 3 | Amino Acid |
| SEQ ID NO: 297 | YU667-G04 VL CDR 1 | Amino Acid |
| SEQ ID NO: 298 | YU667-G04 VL CDR 2 | Amino Acid |
| SEQ ID NO: 299 | YU667-G04 VL CDR 3 | Amino Acid |
| SEQ ID NO: 300 | YU667-H06 VH CDR 1 | Amino Acid |
| SEQ ID NO: 301 | YU667-H06 VH CDR 2 | Amino Acid |
| SEQ ID NO: 302 | YU667-H06 VH CDR 3 | Amino Acid |
| SEQ ID NO: 303 | YU667-H06 VL CDR 1 | Amino Acid |
| SEQ ID NO: 304 | YU667-H06 VL CDR 2 | Amino Acid |
| SEQ ID NO: 305 | YU667-H06 VL CDR 3 | Amino Acid |
| SEQ ID NO: 306 | YU667-E05 VH CDR 1 | Amino Acid |
| SEQ ID NO: 307 | YU667-E05 VH CDR 2 | Amino Acid |
| SEQ ID NO: 308 | YU667-E05 VH CDR 3 | Amino Acid |
| SEQ ID NO: 309 | YU667-E05 VL CDR 1 | Amino Acid |
| SEQ ID NO: 310 | YU667-E05 VL CDR 2 | Amino Acid |
| SEQ ID NO: 311 | YU667-E05 VL CDR 3 | Amino Acid |
| SEQ ID NO: 312 | YU667-A04 VH CDR 1 | Amino Acid |
| SEQ ID NO: 313 | YU667-A04 VH CDR 2 | Amino Acid |
| SEQ ID NO: 314 | YU667-A04 VH CDR 3 | Amino Acid |
| SEQ ID NO: 315 | YU667-A04 VL CDR 1 | Amino Acid |
| SEQ ID NO: 316 | YU667-A04 VL CDR 2 | Amino Acid |
| SEQ ID NO: 317 | YU667-A04 VL CDR 3 | Amino Acid |
| SEQ ID NO: 318 | YU667-G02 VH CDR 1 | Amino Acid |
| SEQ ID NO: 319 | YU667-G02 VH CDR 2 | Amino Acid |
| SEQ ID NO: 320 | YU667-G02 VH CDR 3 | Amino Acid |
| SEQ ID NO: 321 | YU667-G02 VL CDR 1 | Amino Acid |
| SEQ ID NO: 322 | YU667-G02 VL CDR 2 | Amino Acid |
| SEQ ID NO: 323 | YU667-G02 VL CDR 3 | Amino Acid |
| SEQ ID NO: 324 | YU667-A01 VH CDR 1 | Amino Acid |
| SEQ ID NO: 325 | YU667-A01 VH CDR 2 | Amino Acid |
| SEQ ID NO: 326 | YU667-A01 VH CDR 3 | Amino Acid |
| SEQ ID NO: 327 | YU667-A01 VL CDR 1 | Amino Acid |
| SEQ ID NO: 328 | YU667-A01 VL CDR 2 | Amino Acid |
| SEQ ID NO: 329 | YU667-A01 VL CDR 3 | Amino Acid |
| SEQ ID NO: 330 | YU667-F02 VH CDR 1 | Amino Acid |
| SEQ ID NO: 331 | YU667-F02 VH CDR 2 | Amino Acid |
| SEQ ID NO: 332 | YU667-F02 VH CDR 3 | Amino Acid |
| SEQ ID NO: 333 | YU667-F02 VL CDR 1 | Amino Acid |
| SEQ ID NO: 334 | YU667-F02 VL CDR 2 | Amino Acid |
| SEQ ID NO: 335 | YU667-F02 VL CDR 3 | Amino Acid |
| SEQ ID NO: 336 | YU667-B02 VH CDR 1 | Amino Acid |
| SEQ ID NO: 337 | YU667-B02 VH CDR 2 | Amino Acid |
| SEQ ID NO: 338 | YU667-B02 VH CDR 3 | Amino Acid |
| SEQ ID NO: 339 | YU667-B02 VL CDR 1 | Amino Acid |
| SEQ ID NO: 340 | YU667-B02 VL CDR 2 | Amino Acid |
| SEQ ID NO: 341 | YU667-B02 VL CDR 3 | Amino Acid |
| SEQ ID NO: 342 | YU667-B01 VH CDR 1 | Amino Acid |
| SEQ ID NO: 343 | YU667-B01 VH CDR 2 | Amino Acid |
| SEQ ID NO: 344 | YU667-B01 VH CDR 3 | Amino Acid |
| SEQ ID NO: 345 | YU667-B01 VL CDR 1 | Amino Acid |
| SEQ ID NO: 346 | YU667-B01 VL CDR 2 | Amino Acid |
| SEQ ID NO: 347 | YU667-B01 VL CDR 3 | Amino Acid |
| SEQ ID NO: 348 | YU667-A02 VH CDR 1 | Amino Acid |
| SEQ ID NO: 349 | YU667-A02 VH CDR 2 | Amino Acid |
| SEQ ID NO: 350 | YU667-A02 VH CDR 3 | Amino Acid |
| SEQ ID NO: 351 | YU667-A02 VL CDR 1 | Amino Acid |
| SEQ ID NO: 352 | YU667-A02 VL CDR 2 | Amino Acid |
| SEQ ID NO: 353 | YU667-A02 VL CDR 3 | Amino Acid |
| SEQ ID NO: 354 | YU667-D05 VH CDR 1 | Amino Acid |
| SEQ ID NO: 355 | YU667-D05 VH CDR 2 | Amino Acid |
| SEQ ID NO: 356 | YU667-D05 VH CDR 3 | Amino Acid |
| SEQ ID NO: 357 | YU667-D05 VL CDR 1 | Amino Acid |
| SEQ ID NO: 358 | YU667-D05 VL CDR 2 | Amino Acid |
| SEQ ID NO: 359 | YU667-D05 VL CDR 3 | Amino Acid |
| SEQ ID NO: 360 | TI001-V54 VHH CDR 1 | Amino Acid |
| SEQ ID NO: 361 | TI001-V54 VHH CDR 2 | Amino Acid |
| SEQ ID NO: 362 | TI001-V54 VHH CDR 3 | Amino Acid |
| SEQ ID NO: 363 | TI001-V65 VHH CDR 1 | Amino Acid |
| SEQ ID NO: 364 | TI001-V65 VHH CDR 2 | Amino Acid |
| SEQ ID NO: 365 | TI001-V65 VHH CDR 3 | Amino Acid |
| SEQ ID NO: 366 | TI001-V20 VHH CDR 1 | Amino Acid |
| SEQ ID NO: 367 | TI001-V20 VHH CDR 2 | Amino Acid |
| SEQ ID NO: 368 | TI001-V20 VHH CDR 3 | Amino Acid |
| SEQ ID NO: 369 | TI001-A23 VHH CDR 1 | Amino Acid |
| SEQ ID NO: 370 | TI001-A23 VHH CDR 2 | Amino Acid |
| SEQ ID NO: 371 | TI001-A23 VHH CDR 3 | Amino Acid |
| SEQ ID NO: 372 | TI001-A25 VHH CDR 1 | Amino Acid |
| SEQ ID NO: 373 | TI001-A25 VHH CDR 2 | Amino Acid |
| SEQ ID NO: 374 | TI001-A25 VHH CDR 3 | Amino Acid |
| SEQ ID NO: 375 | TI001-A28 VHH CDR 1 | Amino Acid |
| SEQ ID NO: 376 | TI001-A28 VHH CDR 2 | Amino Acid |
| SEQ ID NO: 377 | TI001-A28 VHH CDR 3 | Amino Acid |
| SEQ ID NO: 378 | TI001-A30 VHH CDR 1 | Amino Acid |
| SEQ ID NO: 379 | TI001-A30 VHH CDR 2 | Amino Acid |
| SEQ ID NO: 380 | TI001-A30 VHH CDR 3 | Amino Acid |
| SEQ ID NO: 381 | TI001-A48 VHH CDR 1 | Amino Acid |
| SEQ ID NO: 382 | TI001-A48 VHH CDR 2 | Amino Acid |
| SEQ ID NO: 383 | TI001-A48 VHH CDR 3 | Amino Acid |
| SEQ ID NO: 384 | TI001-V30 VHH CDR 1 | Amino Acid |
| SEQ ID NO: 385 | TI001-V30 VHH CDR 2 | Amino Acid |
| SEQ ID NO: 386 | TI001-V30 VHH CDR 3 | Amino Acid |
| SEQ ID NO: 387 | TI001-V67 VHH CDR 1 | Amino Acid |
| SEQ ID NO: 388 | TI001-V67 VHH CDR 2 | Amino Acid |
| SEQ ID NO: 389 | TI001-V67 VHH CDR 3 | Amino Acid |
| SEQ ID NO: 390 | TI001-V68 VHH CDR 1 | Amino Acid |
| SEQ ID NO: 391 | TI001-V68 VHH CDR 2 | Amino Acid |
| SEQ ID NO: 392 | TI001-V68 VHH CDR 3 | Amino Acid |
| SEQ ID NO: 393 | 6293-R4A-C2 VH CDR 1 | Amino Acid |
| SEQ ID NO: 394 | 6293-R4A-C2 VH CDR 2 | Amino Acid |
| SEQ ID NO: 395 | 6293-R4A-C2 VH CDR 3 | Amino Acid |
| SEQ ID NO: 396 | 6293-R4A-C2 VL CDR 1 | Amino Acid |
| SEQ ID NO: 397 | 6293-R4A-C2 VL CDR 2 | Amino Acid |
| SEQ ID NO: 398 | 6293-R4A-C2 VL CDR 3 | Amino Acid |
| SEQ ID NO: 399 | 6293-R4A-E3 VH CDR 1 | Amino Acid |
| SEQ ID NO: 400 | 6293-R4A-E3 VH CDR 2 | Amino Acid |
| SEQ ID NO: 401 | 6293-R4A-E3 VH CDR 3 | Amino Acid |
| SEQ ID NO: 402 | 6293-R4A-E3 VL CDR 1 | Amino Acid |
| SEQ ID NO: 403 | 6293-R4A-E3 VL CDR 2 | Amino Acid |
| SEQ ID NO: 404 | 6293-R4A-E3 VL CDR 3 | Amino Acid |
| SEQ ID NO: 405 | 6293-R4A-G4 VH CDR 1 | Amino Acid |
| SEQ ID NO: 406 | 6293-R4A-G4 VH CDR 2 | Amino Acid |
| SEQ ID NO: 407 | 6293-R4A-G4 VH CDR 3 | Amino Acid |
| SEQ ID NO: 408 | 6293-R4A-G4 VL CDR 1 | Amino Acid |
| SEQ ID NO: 409 | 6293-R4A-G4 VL CDR 2 | Amino Acid |
| SEQ ID NO: 410 | 6293-R4A-G4 VL CDR 3 | Amino Acid |
| SEQ ID NO: 411 | 6293-R5A-A5 VH CDR 1 | Amino Acid |
| SEQ ID NO: 412 | 6293-R5A-A5 VH CDR 2 | Amino Acid |
| SEQ ID NO: 413 | 6293-R5A-A5 VH CDR 3 | Amino Acid |
| SEQ ID NO: 414 | 6293-R5A-A5 VL CDR 1 | Amino Acid |
| SEQ ID NO: 415 | 6293-R5A-A5 VL CDR 2 | Amino Acid |
| SEQ ID NO: 416 | 6293-R5A-A5 VL CDR 3 | Amino Acid |
| SEQ ID NO: 417 | 6293-R5A-B6 VH CDR 1 | Amino Acid |
| SEQ ID NO: 418 | 6293-R5A-B6 VH CDR 2 | Amino Acid |
| SEQ ID NO: 419 | 6293-R5A-B6 VH CDR 3 | Amino Acid |
| SEQ ID NO: 420 | 6293-R5A-B6 VL CDR 1 | Amino Acid |
| SEQ ID NO: 421 | 6293-R5A-B6 VL CDR 2 | Amino Acid |
| SEQ ID NO: 422 | 6293-R5A-B6 VL CDR 3 | Amino Acid |
| SEQ ID NO: 423 | 6293-R5A-E7 VH CDR 1 | Amino Acid |
| SEQ ID NO: 424 | 6293-R5A-E7 VH CDR 2 | Amino Acid |
| SEQ ID NO: 425 | 6293-R5A-E7 VH CDR 3 | Amino Acid |
| SEQ ID NO: 426 | 6293-R5A-E7 VL CDR 1 | Amino Acid |
| SEQ ID NO: 427 | 6293-R5A-E7 VL CDR 2 | Amino Acid |
| SEQ ID NO: 428 | 6293-R5A-E7 VL CDR 3 | Amino Acid |
| SEQ ID NO: 429 | 6293-R5A-F6 VH CDR 1 | Amino Acid |
| SEQ ID NO: 430 | 6293-R5A-F6 VH CDR 2 | Amino Acid |
| SEQ ID NO: 431 | 6293-R5A-F6 VH CDR 3 | Amino Acid |
| SEQ ID NO: 432 | 6293-R5A-F6 VL CDR 1 | Amino Acid |
| SEQ ID NO: 433 | 6293-R5A-F6 VL CDR 2 | Amino Acid |
| SEQ ID NO: 434 | 6293-R5A-F6 VL CDR 3 | Amino Acid |
| SEQ ID NO: 435 | 6293-R6A-A10 VH CDR 1 | Amino Acid |
| SEQ ID NO: 436 | 6293-R6A-A10 VH CDR 2 | Amino Acid |
| SEQ ID NO: 437 | 6293-R6A-A10 VH CDR 3 | Amino Acid |
| SEQ ID NO: 438 | 6293-R6A-A10 VL CDR 1 | Amino Acid |
| SEQ ID NO: 439 | 6293-R6A-A10 VL CDR 2 | Amino Acid |
| SEQ ID NO: 440 | 6293-R6A-A10 VL CDR 3 | Amino Acid |
| SEQ ID NO: 441 | 6293-R6A-B11 VH CDR 1 | Amino Acid |
| SEQ ID NO: 442 | 6293-R6A-B11 VH CDR 2 | Amino Acid |
| SEQ ID NO: 443 | 6293-R6A-B11 VH CDR 3 | Amino Acid |
| SEQ ID NO: 444 | 6293-R6A-B11 VL CDR 1 | Amino Acid |
| SEQ ID NO: 445 | 6293-R6A-B11 VL CDR 2 | Amino Acid |
| SEQ ID NO: 446 | 6293-R6A-B11 VL CDR 3 | Amino Acid |
| SEQ ID NO: 447 | 6293-R6A-C9 VH CDR 1 | Amino Acid |
| SEQ ID NO: 448 | 6293-R6A-C9 VH CDR 2 | Amino Acid |
| SEQ ID NO: 449 | 6293-R6A-C9 VH CDR 3 | Amino Acid |
| SEQ ID NO: 450 | 6293-R6A-C9 VL CDR 1 | Amino Acid |
| SEQ ID NO: 451 | 6293-R6A-C9 VL CDR 2 | Amino Acid |
| SEQ ID NO: 452 | 6293-R6A-C9 VL CDR 3 | Amino Acid |
| SEQ ID NO: 453 | 6293-R6A-G9 VH CDR 1 | Amino Acid |
| SEQ ID NO: 454 | 6293-R6A-G9 VH CDR 2 | Amino Acid |
| SEQ ID NO: 455 | 6293-R6A-G9 VH CDR 3 | Amino Acid |
| SEQ ID NO: 456 | 6293-R6A-G9 VL CDR 1 | Amino Acid |
| SEQ ID NO: 457 | 6293-R6A-G9 VL CDR 2 | Amino Acid |
| SEQ ID NO: 458 | 6293-R6A-G9 VL CDR 3 | Amino Acid |
| SEQ ID NO: 459 | 6293-R6A-H11 VH CDR 1 | Amino Acid |
| SEQ ID NO: 460 | 6293-R6A-H11 VH CDR 2 | Amino Acid |
| SEQ ID NO: 461 | 6293-R6A-H11 VH CDR 3 | Amino Acid |
| SEQ ID NO: 462 | 6293-R6A-H11 VL CDR 1 | Amino Acid |
| SEQ ID NO: 463 | 6293-R6A-H11 VL CDR 2 | Amino Acid |
| SEQ ID NO: 464 | 6293-R6A-H11 VL CDR 3 | Amino Acid |
| SEQ ID NO: 522 | T1001-E8 CDR1 | Amino Acid |
| SEQ ID NO: 523 | T1001-E8 CDR2 | Amino Acid |
| SEQ ID NO: 524 | T1001-E8 CDR3 | Amino Acid |
| SEQ ID NO: 525 | T1001-E16 CDR1 | Amino Acid |
| SEQ ID NO: 526 | T1001-E16 CDR2 | Amino Acid |
| SEQ ID NO: 527 | T1001-E16 CDR3 | Amino Acid |
| SEQ ID NO: 528 | T1001-E6 CDR1 | Amino Acid |
| SEQ ID NO: 529 | T1001-E6 CDR2 | Amino Acid |
| SEQ ID NO: 530 | T1001-E6 CDR3 | Amino Acid |
| SEQ ID NO: 531 | T1001-E64 CDR1 | Amino Acid |
| SEQ ID NO: 532 | T1001-E64 CDR2 | Amino Acid |
| SEQ ID NO: 533 | T1001-E64 CDR3 | Amino Acid |
| SEQ ID NO: 534 | T1001-E55 CDR1 | Amino Acid |
| SEQ ID NO: 535 | T1001-E55 CDR2 | Amino Acid |
| SEQ ID NO: 536 | T1001-E55 CDR3 | Amino Acid |
| SEQ ID NO: 537 | T1001-E17 CDR1 | Amino Acid |
| SEQ ID NO: 538 | T1001-E17 CDR2 | Amino Acid |
| SEQ ID NO: 539 | T1001-E17 CDR3 | Amino Acid |
| SEQ ID NO: 540 | T1001-E24 CDR1 | Amino Acid |
| SEQ ID NO: 541 | T1001-E24 CDR2 | Amino Acid |
| SEQ ID NO: 542 | T1001-E24 CDR3 | Amino Acid |
| SEQ ID NO: 543 | T1001-E25 CDR1 | Amino Acid |
| SEQ ID NO: 544 | T1001-E25 CDR2 | Amino Acid |
| SEQ ID NO: 545 | T1001-E25 CDR3 | Amino Acid |

### Specific TACs

Disclosed herein, in certain embodiments, are GUYC2C TAC proteins comprising an amino acid sequence with the amino acid sequence of any one of SEQ TD NOs: 465-513, 546-590, or 686.

In some embodiments, the GUCY2C TAC protein comprises an amino acid sequence having at least 8000 sequence identity with the amino acid sequence of any one of SEQ TD NOs: 465-513, 546-590, or 686. In some embodiments, the GUCY2C TAC protein comprises an amino acid sequence having at least 850% sequence identity with the amino acid sequence of any one of SEQ TD NOs: 465-513, 546-590, or 686. In some embodiments, the GUCY2C TAC protein comprises an amino acid sequence having at least 900% sequence identity with the amino acid sequence of any one of SEQ ID NOs: 465-513, 546-590, or 686. In some embodiments, the GUCY2C TAC protein comprises an amino acid sequence having at least 9500 sequence identity with the amino acid sequence of any one of SEQ ID NOs: 465-513, 546-590, or 686. In some embodiments, the GUCY2C TAC protein comprises an amino acid sequence having at least 96% sequence identity with the amino acid sequence of any one of SEQ ID NOs: 465-513, 546-590, or 686. In some embodiments, the GUCY2C TAC protein comprises an amino acid sequence having at least 9700 sequence identity with the amino acid sequence of any one of SEQ ID NOs: 465-513, 546-590, or 686. In some embodiments, the GUCY2C TAC protein comprises an amino acid sequence having at least 98% sequence identity with the amino acid sequence of any one of SEQ ID NOs: 465-513, 546-590, or 686. In some embodiments, the GUCY2C TAC protein comprises an amino acid sequence having at least 99% sequence identity with the amino acid sequence of any one of SEQ ID NOs: 465-513, 546-590, or 686. In some embodiments, the GUCY2C TAC protein comprises an amino acid sequence of any one of SEQ ID NOs: 465-513, 546-590, or 686.

In some embodiments, the GUCY2C TAC protein comprises an amino acid sequence having at least 80% sequence identity with the amino acid sequence of SEQ ID NO: 569. In some embodiments, the GUCY2C TAC protein comprises an amino acid sequence having at least 85% sequence identity with the amino acid sequence of SEQ ID NO: 569. In some embodiments, the GUCY2C TAC protein comprises an amino acid sequence having at least 90% sequence identity with the amino acid sequence of SEQ ID NO: 569. In some embodiments, the GUCY2C TAC protein comprises an amino acid sequence having at least 95% sequence identity with the amino acid sequence of SEQ ID NO: 569. In some embodiments, the GUCY2C TAC protein comprises an amino acid sequence having at least 96% sequence identity with the amino acid sequence of SEQ ID NO: 569. In some embodiments, the GUCY2C TAC protein comprises an amino acid sequence having at least 97% sequence identity with the amino acid sequence of SEQ ID NO: 569. In some embodiments, the GUCY2C TAC protein comprises an amino acid sequence having at least 98% sequence identity with the amino acid sequence of SEQ ID NO: 569. In some embodiments, the GUCY2C TAC protein comprises an amino acid sequence having at least 99% sequence identity with the amino acid sequence of SEQ ID NO: 569. In some embodiments, the GUCY2C TAC protein comprises an amino acid sequence of SEQ ID NO: 569.

In some embodiments, the GUCY2C TAC protein comprises an amino acid sequence having at least 80% sequence identity with the amino acid sequence of SEQ ID NO: 570. In some embodiments, the GUCY2C TAC protein comprises an amino acid sequence having at least 85% sequence identity with the amino acid sequence of SEQ ID NO: 570. In some embodiments, the GUCY2C TAC protein comprises an amino acid sequence having at least 90% sequence identity with the amino acid sequence of SEQ ID NO: 570. In some embodiments, the GUCY2C TAC protein comprises an amino acid sequence having at least 95% sequence identity with the amino acid sequence of SEQ ID NO: 570. In some embodiments, the GUCY2C TAC protein comprises an amino acid sequence having at least 96% sequence identity with the amino acid sequence of SEQ ID NO: 570. In some embodiments, the GUCY2C TAC protein comprises an amino acid sequence having at least 97% sequence identity with the amino acid sequence of SEQ ID NO: 570. In some embodiments, the GUCY2C TAC protein comprises an amino acid sequence having at least 98% sequence identity with the amino acid sequence of SEQ ID NO: 570. In some embodiments, the GUCY2C TAC protein comprises an amino acid sequence having at least 99% sequence identity with the amino acid sequence of SEQ ID NO: 570. In some embodiments, the GUCY2C TAC protein comprises an amino acid sequence of SEQ ID NO: 570.

In some embodiments, the GUCY2C TAC protein comprises an amino acid sequence having at least 80% sequence identity with the amino acid sequence of SEQ ID NO: 580. In some embodiments, the GUCY2C TAC protein comprises an amino acid sequence having at least 85% sequence identity with the amino acid sequence of SEQ ID NO: 580. In some embodiments, the GUCY2C TAC protein comprises an amino acid sequence having at least 90% sequence identity with the amino acid sequence of SEQ ID NO: 580. In some embodiments, the GUCY2C TAC protein comprises an amino acid sequence having at least 95% sequence identity with the amino acid sequence of SEQ ID NO: 580. In some embodiments, the GUCY2C TAC protein comprises an amino acid sequence having at least 96% sequence identity with the amino acid sequence of SEQ ID NO: 580. In some embodiments, the GUCY2C TAC protein comprises an amino acid sequence having at least 97% sequence identity with the amino acid sequence of SEQ ID NO: 580. In some embodiments, the GUCY2C TAC protein comprises an amino acid sequence having at least 98% sequence identity with the amino acid sequence of SEQ ID NO: 580. In some embodiments, the GUCY2C TAC protein comprises an amino acid sequence having at least 99% sequence identity with the amino acid sequence of SEQ ID NO: 580. In some embodiments, the GUCY2C TAC protein comprises an amino acid sequence of SEQ ID NO: 580.

In some embodiments, the GUCY2C TAC protein comprises an amino acid sequence having at least 80% sequence identity with the amino acid sequence of any one of SEQ ID NOs: 465-513, 546-590, or 686, wherein the CDR sequences of the GUCY2C TAC protein sequence have 100% sequence identity to the CDR sequences of the sequence of the SEQ ID NO. In some embodiments, the GUCY2C TAC protein comprises an amino acid sequence having at least 85% sequence identity with the amino acid sequence of any one of SEQ ID NOs: 465-513, 546-590, or 686, wherein the CDR sequences of the GUCY2C TAC protein sequence have 100% sequence identity to the CDR sequences of the sequence of the SEQ ID NO. In some embodiments, the GUCY2C TAC protein comprises an amino acid sequence having at least 90% sequence identity with the amino acid sequence of any one of SEQ ID NOs: 465-513, 546-590, or 686, wherein the CDR sequences of the GUCY2C TAC protein sequence have 100% sequence identity to the CDR sequences of the sequence of the SEQ ID NO. In some embodiments, the GUCY2C TAC protein comprises an amino acid sequence having at least 95% sequence identity with the amino acid sequence of any one of SEQ ID NOs: 465-513, 546-590, or 686, wherein the CDR sequences of the GUCY2C TAC protein sequence have 100% sequence identity to the CDR sequences of the sequence of the SEQ ID NO. In some embodiments, the GUCY2C TAC protein comprises an amino acid sequence having at least 96% sequence identity with the amino acid sequence of any one of SEQ ID NOs: 465-513, 546-590, or 686, wherein the CDR sequences of the GUCY2C TAC protein sequence have 100% sequence identity to the CDR sequences of the sequence of the SEQ ID NO. In some embodiments, the GUCY2C TAC protein comprises an amino acid sequence having at least 97% sequence identity with the amino acid sequence of any one of SEQ ID NOs: 465-513, 546-590, or 686, wherein the CDR sequences of the GUCY2C TAC protein sequence have 100% sequence identity to the CDR sequences of the sequence of the SEQ ID NO. In some embodiments, the GUCY2C TAC protein comprises an amino acid sequence having at least 98% sequence identity with the amino acid sequence of any one of SEQ ID NOs: 465-513, 546-590, or 686, wherein the CDR sequences of the GUCY2C TAC protein sequence have 100% sequence identity to the CDR sequences of the sequence of the SEQ ID NO. In some embodiments, the GUCY2C TAC protein comprises an amino acid sequence having at least 99% sequence identity with the amino acid sequence of any one of SEQ ID NOs: 465-513, 546-590, or 686, wherein the CDR sequences of the GUCY2C TAC protein sequence have 100% sequence identity to the CDR sequences of the sequence of the SEQ ID NO.

In some embodiments, the GUCY2C TAC protein comprises an amino acid sequence having at least 80% sequence identity with the amino acid sequence of SEQ ID NO: 569, wherein the CDR sequences of the GUCY2C TAC protein sequence have 100% sequence identity to the CDR sequences of the sequence of SEQ ID NO: 569. In some embodiments, the GUCY2C TAC protein comprises an amino acid sequence having at least 85% sequence identity with the amino acid sequence of SEQ ID NO: 569, wherein the CDR sequences of the GUCY2C TAC protein sequence have 100% sequence identity to the CDR sequences of the sequence of SEQ ID NO: 569. In some embodiments, the GUCY2C TAC protein comprises an amino acid sequence having at least 90% sequence identity with the amino acid sequence of SEQ ID NO: 569, wherein the CDR sequences of the GUCY2C TAC protein sequence have 100% sequence identity to the CDR sequences of the sequence of SEQ ID NO: 569. In some embodiments, the GUCY2C TAC protein comprises an amino acid sequence having at least 95% sequence identity with the amino acid sequence of SEQ ID NO: 569, wherein the CDR sequences of the GUCY2C TAC protein sequence have 100% sequence identity to the CDR sequences of the sequence of SEQ ID NO: 569. In some embodiments, the GUCY2C TAC protein comprises an amino acid sequence having at least 96% sequence identity with the amino acid sequence of SEQ ID NO: 569, wherein the CDR sequences of the GUCY2C TAC protein sequence have 100% sequence identity to the CDR sequences of the sequence of SEQ ID NO: 569. In some embodiments, the GUCY2C TAC protein comprises an amino acid sequence having at least 97% sequence identity with the amino acid sequence of SEQ ID NO: 569, wherein the CDR sequences of the GUCY2C TAC protein sequence have 100% sequence identity to the CDR sequences of the sequence of SEQ ID NO: 569. In some embodiments, the GUCY2C TAC protein comprises an amino acid sequence having at least 98% sequence identity with the amino acid sequence of SEQ ID NO: 569, wherein the CDR sequences of the GUCY2C TAC protein sequence have 100% sequence identity to the CDR sequences of the sequence of SEQ ID NO: 569. In some embodiments, the GUCY2C TAC protein comprises an amino acid sequence having at least 99% sequence identity with the amino acid sequence of SEQ ID NO: 569, wherein the CDR sequences of the GUCY2C TAC protein sequence have 100% sequence identity to the CDR sequences of the sequence of SEQ ID NO: 569.

In some embodiments, the GUCY2C TAC protein comprises an amino acid sequence having at least 80% sequence identity with the amino acid sequence of SEQ ID NO: 570, wherein the CDR sequences of the GUCY2C TAC protein sequence have 100% sequence identity to the CDR sequences of the sequence of SEQ ID NO: 570. In some embodiments, the GUCY2C TAC protein comprises an amino acid sequence having at least 85% sequence identity with the amino acid sequence of SEQ ID NO: 570, wherein the CDR sequences of the GUCY2C TAC protein sequence have 100% sequence identity to the CDR sequences of the sequence of SEQ ID NO: 570. In some embodiments, the GUCY2C TAC protein comprises an amino acid sequence having at least 90% sequence identity with the amino acid sequence of SEQ ID NO: 570, wherein the CDR sequences of the GUCY2C TAC protein sequence have 100% sequence identity to the CDR sequences of the sequence of SEQ ID NO: 570. In some embodiments, the GUCY2C TAC protein comprises an amino acid sequence having at least 95% sequence identity with the amino acid sequence of SEQ ID NO: 570, wherein the CDR sequences of the GUCY2C TAC protein sequence have 100% sequence identity to the CDR sequences of the sequence of SEQ ID NO: 570. In some embodiments, the GUCY2C TAC protein comprises an amino acid sequence having at least 96% sequence identity with the amino acid sequence of SEQ ID NO: 570, wherein the CDR sequences of the GUCY2C TAC protein sequence have 100% sequence identity to the CDR sequences of the sequence of SEQ ID NO: 570. In some embodiments, the GUCY2C TAC protein comprises an amino acid sequence having at least 97% sequence identity with the amino acid sequence of SEQ ID NO: 570, wherein the CDR sequences of the GUCY2C TAC protein sequence have 100% sequence identity to the CDR sequences of the sequence of SEQ ID NO: 570. In some embodiments, the GUCY2C TAC protein comprises an amino acid sequence having at least 98% sequence identity with the amino acid sequence of SEQ ID NO: 570, wherein the CDR sequences of the GUCY2C TAC protein sequence have 100% sequence identity to the CDR sequences of the sequence of SEQ ID NO: 570. In some embodiments, the GUCY2C TAC protein comprises an amino acid sequence having at least 99% sequence identity with the amino acid sequence of SEQ ID NO: 570, wherein the CDR sequences of the GUCY2C TAC protein sequence have 100% sequence identity to the CDR sequences of the sequence of SEQ ID NO: 570.

In some embodiments, the GUCY2C TAC protein comprises an amino acid sequence having at least 80% sequence identity with the amino acid sequence of SEQ ID NO: 580, wherein the CDR sequences of the GUCY2C TAC protein sequence have 100% sequence identity to the CDR sequences of the sequence of SEQ ID NO: 580. In some embodiments, the GUCY2C TAC protein comprises an amino acid sequence having at least 85% sequence identity with the amino acid sequence of SEQ ID NO: 580, wherein the CDR sequences of the GUCY2C TAC protein sequence have 100% sequence identity to the CDR sequences of the sequence of SEQ ID NO: 580. In some embodiments, the GUCY2C TAC protein comprises an amino acid sequence having at least 90% sequence identity with the amino acid sequence of SEQ ID NO: 580, wherein the CDR sequences of the GUCY2C TAC protein sequence have 100% sequence identity to the CDR sequences of the sequence of SEQ ID NO: 580. In some embodiments, the GUCY2C TAC protein comprises an amino acid sequence having at least 95% sequence identity with the amino acid sequence of SEQ ID NO: 580, wherein the CDR sequences of the GUCY2C TAC protein sequence have 100% sequence identity to the CDR sequences of the sequence of SEQ ID NO: 580. In some embodiments, the GUCY2C TAC protein comprises an amino acid sequence having at least 96% sequence identity with the amino acid sequence of SEQ ID NO: 580, wherein the CDR sequences of the GUCY2C TAC protein sequence have 100% sequence identity to the CDR sequences of the sequence of SEQ ID NO: 580. In some embodiments, the GUCY2C TAC protein comprises an amino acid sequence having at least 97% sequence identity with the amino acid sequence of SEQ ID NO: 580, wherein the CDR sequences of the GUCY2C TAC protein sequence have 100% sequence identity to the CDR sequences of the sequence of SEQ ID NO: 580. In some embodiments, the GUCY2C TAC protein comprises an amino acid sequence having at least 98% sequence identity with the amino acid sequence of SEQ ID NO: 580, wherein the CDR sequences of the GUCY2C TAC protein sequence have 100% sequence identity to the CDR sequences of the sequence of SEQ ID NO: 580. In some embodiments, the GUCY2C TAC protein comprises an amino acid sequence having at least 99% sequence identity with the amino acid sequence of SEQ ID NO: 580, wherein the CDR sequences of the GUCY2C TAC protein sequence have 100% sequence identity to the CDR sequences of the sequence of SEQ ID NO: 580.

In some embodiments, the GUCY2C TAC protein is encoded by a nucleic acid sequence having at least 80% sequence identity with the nucleic acid sequence of any one of SEQ ID NOs: 591-685. In some embodiments, the GUCY2C TAC protein is encoded by a nucleic acid sequence having at least 85% sequence identity with the nucleic acid sequence of any one of SEQ ID NOs: 591-685. In some embodiments, the GUCY2C TAC protein is encoded by a nucleic acid sequence having at least 90% sequence identity with the nucleic acid sequence of any one of SEQ ID NOs: 591-685. In some embodiments, the GUCY2C TAC protein is encoded by a nucleic acid sequence having at least 95% sequence identity with the nucleic acid sequence of any one of SEQ ID NOs: 591-685. In some embodiments, the GUCY2C TAC protein is encoded by a nucleic acid sequence having at least 96% sequence identity with the nucleic acid sequence of any one of SEQ ID NOs: 591-685. In some embodiments, the GUCY2C TAC protein is encoded by a nucleic acid sequence having at least 97% sequence identity with the nucleic acid sequence of any one of SEQ ID NOs: 591-685. In some embodiments, the GUCY2C TAC protein is encoded by a nucleic acid sequence having at least 98% sequence identity with the nucleic acid sequence of any one of SEQ ID NOs: 591-685. In some embodiments, the GUCY2C TAC protein is encoded by a nucleic acid sequence having at least 99% sequence identity with the nucleic acid sequence of any one of SEQ ID NOs: 591-685. In some embodiments, the GUCY2C TAC protein is encoded by a nucleic acid sequence of any one of SEQ ID NOs: 591-685.

In some embodiments, the GUCY2C TAC protein is encoded by a nucleic acid sequence having at least 80% sequence identity with the nucleic acid sequence of SEQ ID NOs: 663. In some embodiments, the GUCY2C TAC protein is encoded by a nucleic acid sequence having at least 85% sequence identity with the nucleic acid sequence of SEQ ID NOs: 663. In some embodiments, the GUCY2C TAC protein is encoded by a nucleic acid sequence having at least 90% sequence identity with the nucleic acid sequence of SEQ ID NOs: 663. In some embodiments, the GUCY2C TAC protein is encoded by a nucleic acid sequence having at least 95% sequence identity with the nucleic acid sequence of SEQ ID NOs: 663. In some embodiments, the GUCY2C TAC protein is encoded by a nucleic acid sequence having at least 96% sequence identity with the nucleic acid sequence of SEQ ID NOs: 663. In some embodiments, the GUCY2C TAC protein is encoded by a nucleic acid sequence having at least 97% sequence identity with the nucleic acid sequence of SEQ ID NOs: 663. In some embodiments, the GUCY2C TAC protein is encoded by a nucleic acid sequence having at least 98% sequence identity with the nucleic acid sequence of SEQ ID NOs: 663. In some embodiments, the GUCY2C TAC protein is encoded by a nucleic acid sequence having at least 99% sequence identity with the nucleic acid sequence of SEQ ID NOs: 663. In some embodiments, the GUCY2C TAC protein is encoded by a nucleic acid sequence of SEQ ID NOs: 663.

In some embodiments, the GUCY2C TAC protein is encoded by a nucleic acid sequence having at least 80% sequence identity with the nucleic acid sequence of SEQ ID NOs: 664. In some embodiments, the GUCY2C TAC protein is encoded by a nucleic acid sequence having at least 85% sequence identity with the nucleic acid sequence of SEQ ID NOs: 664. In some embodiments, the GUCY2C TAC protein is encoded by a nucleic acid sequence having at least 90% sequence identity with the nucleic acid sequence of SEQ ID NOs: 664. In some embodiments, the GUCY2C TAC protein is encoded by a nucleic acid sequence having at least 95% sequence identity with the nucleic acid sequence of SEQ ID NOs: 664. In some embodiments, the GUCY2C TAC protein is encoded by a nucleic acid sequence having at least 96% sequence identity with the nucleic acid sequence of SEQ ID NOs: 664. In some embodiments, the GUCY2C TAC protein is encoded by a nucleic acid sequence having at least 97% sequence identity with the nucleic acid sequence of SEQ ID NOs: 664. In some embodiments, the GUCY2C TAC protein is encoded by a nucleic acid sequence having at least 98% sequence identity with the nucleic acid sequence of SEQ ID NOs: 664. In some embodiments, the GUCY2C TAC protein is encoded by a nucleic acid sequence having at least 99% sequence identity with the nucleic acid sequence of SEQ ID NOs: 664. In some embodiments, the GUCY2C TAC protein is encoded by a nucleic acid sequence of SEQ ID NOs: 664.

In some embodiments, the GUCY2C TAC protein is encoded by a nucleic acid sequence having at least 80% sequence identity with the nucleic acid sequence of SEQ ID NOs: 674. In some embodiments, the GUCY2C TAC protein is encoded by a nucleic acid sequence having at least 85% sequence identity with the nucleic acid sequence of SEQ ID NOs: 674. In some embodiments, the GUCY2C TAC protein is encoded by a nucleic acid sequence having at least 90% sequence identity with the nucleic acid sequence of SEQ ID NOs: 674. In some embodiments, the GUCY2C TAC protein is encoded by a nucleic acid sequence having at least 95% sequence identity with the nucleic acid sequence of SEQ ID NOs: 674. In some embodiments, the GUCY2C TAC protein is encoded by a nucleic acid sequence having at least 96% sequence identity with the nucleic acid sequence of SEQ ID NOs: 674. In some embodiments, the GUCY2C TAC protein is encoded by a nucleic acid sequence having at least 97% sequence identity with the nucleic acid sequence of SEQ ID NOs: 674. In some embodiments, the GUCY2C TAC protein is encoded by a nucleic acid sequence having at least 98% sequence identity with the nucleic acid sequence of SEQ ID NOs: 674. In some embodiments, the GUCY2C TAC protein is encoded by a nucleic acid sequence having at least 99% sequence identity with the nucleic acid sequence of SEQ ID NOs: 674. In some embodiments, the GUCY2C TAC protein is encoded by a nucleic acid sequence of SEQ ID NOs: 674.

Amino acid sequences and nucleic acid sequences of exemplary GUCY2C TACs are provided in Table 7.

TABLE 7
Table of Sequences

| SEQ ID NO. | Description | Amino Acid/Nucleic Acid |
|---|---|---|
| SEQ ID NO: 591 | YU652-B06-Whitlow-TAC | Nucleotide |
| SEQ ID NO: 465 | YU652-B06-Whitlow-TAC | Amino Acid |
| SEQ ID NO: 592 | YU652-C01-Whitlow-TAC | Nucleotide |
| SEQ ID NO: 466 | YU652-C01-Whitlow-TAC | Amino Acid |
| SEQ ID NO: 593 | YU652-F02-Whitlow-TAC | Nucleotide |
| SEQ ID NO: 467 | YU652-F02-Whitlow-TAC | Amino Acid |
| SEQ ID NO: 594 | YU652-H02-Whitlow-TAC | Nucleotide |
| SEQ ID NO: 468 | YU652-H02-Whitlow-TAC | Amino Acid |
| SEQ ID NO: 595 | YU653-B12-Whitlow-TAC | Nucleotide |
| SEQ ID NO: 469 | YU653-B12-Whitlow-TAC | Amino Acid |
| SEQ ID NO: 596 | YU653-D11-Whitlow-TAC | Nucleotide |
| SEQ ID NO: 470 | YU653-D11-Whitlow-TAC | Amino Acid |
| SEQ ID NO: 597 | YU653-F12-Whitlow-TAC | Nucleotide |
| SEQ ID NO: 471 | YU653-F12-Whitlow-TAC | Amino Acid |
| SEQ ID NO: 598 | YU654-E01-Whitlow-TAC | Nucleotide |
| SEQ ID NO: 472 | YU654-E01-Whitlow-TAC | Amino Acid |
| SEQ ID NO: 599 | YU667-A01-Whitlow-TAC | Nucleotide |
| SEQ ID NO: 473 | YU667-A01-Whitlow-TAC | Amino Acid |
| SEQ ID NO: 600 | YU667-A02-Whitlow-TAC | Nucleotide |
| SEQ ID NO: 474 | YU667-A02-Whitlow-TAC | Amino Acid |
| SEQ ID NO: 601 | YU667-A04-Whitlow-TAC | Nucleotide |
| SEQ ID NO: 475 | YU667-A04-Whitlow-TAC | Amino Acid |
| SEQ ID NO: 602 | YU667-B01-Whitlow-TAC | Nucleotide |
| SEQ ID NO: 476 | YU667-B01-Whitlow-TAC | Amino Acid |
| SEQ ID NO: 603 | YU667-B02-Whitlow-TAC | Nucleotide |
| SEQ ID NO: 477 | YU667-B02-Whitlow-TAC | Amino Acid |
| SEQ ID NO: 604 | YU667-B03-Whitlow-TAC | Nucleotide |
| SEQ ID NO: 478 | YU667-B03-Whitlow-TAC | Amino Acid |
| SEQ ID NO: 605 | YU667-C04-Whitlow-TAC | Nucleotide |
| SEQ ID NO: 479 | YU667-C04-Whitlow-TAC | Amino Acid |
| SEQ ID NO: 606 | YU667-C06-Whitlow-TAC | Nucleotide |
| SEQ ID NO: 480 | YU667-C06-Whitlow-TAC | Amino Acid |
| SEQ ID NO: 607 | YU667-D05-Whitlow-TAC | Nucleotide |
| SEQ ID NO: 481 | YU667-D05-Whitlow-TAC | Amino Acid |
| SEQ ID NO: 608 | YU667-D06-Whitlow-TAC | Nucleotide |
| SEQ ID NO: 482 | YU667-D06-Whitlow-TAC | Amino Acid |
| SEQ ID NO: 609 | YU667-E01-Whitlow-TAC | Nucleotide |
| SEQ ID NO: 483 | YU667-E01-Whitlow-TAC | Amino Acid |
| SEQ ID NO: 610 | YU667-E05-Whitlow-TAC | Nucleotide |
| SEQ ID NO: 484 | YU667-E05-Whitlow-TAC | Amino Acid |
| SEQ ID NO: 611 | YU667-F02-Whitlow-TAC | Nucleotide |
| SEQ ID NO: 485 | YU667-F02-Whitlow-TAC | Amino Acid |
| SEQ ID NO: 612 | YU667-F06-Whitlow-TAC | Nucleotide |
| SEQ ID NO: 486 | YU667-F06-Whitlow-TAC | Amino Acid |
| SEQ ID NO: 613 | YU667-G02-Whitlow-TAC | Nucleotide |
| SEQ ID NO: 487 | YU667-G02-Whitlow-TAC | Amino Acid |
| SEQ ID NO: 614 | YU667-G04-Whitlow-TAC | Nucleotide |
| SEQ ID NO: 488 | YU667-G04-Whitlow-TAC | Amino Acid |
| SEQ ID NO: 615 | YU667-G06-Whitlow-TAC | Nucleotide |
| SEQ ID NO: 489 | YU667-G06-Whitlow-TAC | Amino Acid |
| SEQ ID NO: 616 | YU667-H06-Whitlow-TAC | Nucleotide |
| SEQ ID NO: 490 | YU667-H06-Whitlow-TAC | Amino Acid |
| SEQ ID NO: 617 | 6293-R4A-C2-TAC | Nucleotide |
| SEQ ID NO: 491 | 6293-R4A-C2-TAC | Amino Acid |
| SEQ ID NO: 618 | 6293-R4A-E3-TAC | Nucleotide |
| SEQ ID NO: 492 | 6293-R4A-E3-TAC | Amino Acid |
| SEQ ID NO: 619 | 6293-R4A-G4-TAC | Nucleotide |
| SEQ ID NO: 493 | 6293-R4A-G4-TAC | Amino Acid |
| SEQ ID NO: 620 | 6293-R5A-A5-TAC | Nucleotide |
| SEQ ID NO: 494 | 6293-R5A-A5-TAC | Amino Acid |
| SEQ ID NO: 621 | 6293-R5A-B6-TAC | Nucleotide |
| SEQ ID NO: 495 | 6293-R5A-B6-TAC | Amino Acid |
| SEQ ID NO: 622 | 6293-R5A-E7-TAC | Nucleotide |
| SEQ ID NO: 496 | 6293-R5A-E7-TAC | Amino Acid |
| SEQ ID NO: 623 | 6293-R5A-F6-TAC | Nucleotide |
| SEQ ID NO: 497 | 6293-R5A-F6-TAC | Amino Acid |
| SEQ ID NO: 624 | 6293-R6A-A10-TAC | Nucleotide |
| SEQ ID NO: 498 | 6293-R6A-A10-TAC | Amino Acid |
| SEQ ID NO: 625 | 6293-R6A-B11-TAC | Nucleotide |
| SEQ ID NO: 499 | 6293-R6A-B11-TAC | Amino Acid |
| SEQ ID NO: 626 | 6293-R6A-C9-TAC | Nucleotide |
| SEQ ID NO: 500 | 6293-R6A-C9-TAC | Amino Acid |
| SEQ ID NO: 627 | 6293-R6A-G9-TAC | Nucleotide |
| SEQ ID NO: 501 | 6293-R6A-G9-TAC | Amino Acid |
| SEQ ID NO: 628 | 6293-R6A-H11-TAC | Nucleotide |
| SEQ ID NO: 502 | 6293-R6A-H11-TAC | Amino Acid |
| SEQ ID NO: 629 | TI001-A23-TAC | Nucleotide |
| SEQ ID NO: 503 | TI001-A23-TAC | Amino Acid |
| SEQ ID NO: 630 | TI001-A25-TAC | Nucleotide |
| SEQ ID NO: 504 | TI001-A25-TAC | Amino Acid |
| SEQ ID NO: 631 | TI001-A28-TAC | Nucleotide |
| SEQ ID NO: 505 | TI001-A28-TAC | Amino Acid |
| SEQ ID NO: 632 | TI001-A30-TAC | Nucleotide |
| SEQ ID NO: 506 | TI001-A30-TAC | Amino Acid |
| SEQ ID NO: 633 | TI001-A48-TAC | Nucleotide |
| SEQ ID NO: 507 | TI001-A48-TAC | Amino Acid |
| SEQ ID NO: 634 | TI001-V20-TAC | Nucleotide |
| SEQ ID NO: 508 | TI001-V20-TAC | Amino Acid |
| SEQ ID NO: 635 | TI001-V30-TAC | Nucleotide |
| SEQ ID NO: 509 | TI001-V30-TAC | Amino Acid |
| SEQ ID NO: 636 | TI001-V54-TAC | Nucleotide |
| SEQ ID NO: 510 | TI001-V54-TAC | Amino Acid |
| SEQ ID NO: 637 | TI001-V65-TAC | Nucleotide |
| SEQ ID NO: 511 | TI001-V65-TAC | Amino Acid |
| SEQ ID NO: 638 | TI001-V67-TAC | Nucleotide |
| SEQ ID NO: 512 | TI001-V67-TAC | Amino Acid |
| SEQ ID NO: 639 | TI001-V68-TAC | Nucleotide |
| SEQ ID NO: 513 | TI001-V68-TAC | Amino Acid |
| SEQ ID NO: 640 | T1001-E6_HC-TAC | Nucleotide |
| SEQ ID NO: 548 | T1001-E6 HC-TAC | Amino Acid |
| SEQ ID NO: 641 | T1001-E8 HC-TAC | Nucleotide |
| SEQ ID NO: 546 | T1001-E8 HC-TAC | Amino Acid |
| SEQ ID NO: 642 | T1001-E16 HC-TAC | Nucleotide |
| SEQ ID NO: 547 | T1001-E16 HC-TAC | Amino Acid |
| SEQ ID NO: 643 | T1001-E17 HC-TAC | Nucleotide |
| SEQ ID NO: 551 | T1001-E17 HC-TAC | Amino Acid |
| SEQ ID NO: 644 | T1001-E24 HC-TAC | Nucleotide |
| SEQ ID NO: 552 | T1001-E24 HC-TAC | Amino Acid |
| SEQ ID NO: 645 | T1001-E25_HC-TAC | Nucleotide |
| SEQ ID NO: 553 | T1001-E25 HC-TAC | Amino Acid |
| SEQ ID NO: 646 | T1001-E55 HC-TAC | Nucleotide |
| SEQ ID NO: 550 | T1001-E55 HC-TAC | Amino Acid |
| SEQ ID NO: 647 | T1001-E64 HC-TAC | Nucleotide |
| SEQ ID NO: 549 | T1001-E64 HC-TAC | Amino Acid |
| SEQ ID NO: 648 | GUCY2C-TAC G7 | Nucleotide |
| SEQ ID NO: 554 | GUCY2C-TAC G7 | Amino Acid |
| SEQ ID NO: 649 | GUCY2C-TAC G8 | Nucleotide |
| SEQ ID NO: 555 | GUCY2C-TAC G8 | Amino Acid |
| SEQ ID NO: 650 | GUCY2C-TAC G9 | Nucleotide |
| SEQ ID NO: 556 | GUCY2C-TAC G9 | Amino Acid |
| SEQ ID NO: 651 | GUCY2C-TAC G10 | Nucleotide |
| SEQ ID NO: 557 | GUCY2C-TAC G10 | Amino Acid |
| SEQ ID NO: 652 | GUCY2C-TAC G11 | Nucleotide |
| SEQ ID NO: 558 | GUCY2C-TAC G11 | Amino Acid |
| SEQ ID NO: 653 | GUCY2C-TAC G12 | Nucleotide |
| SEQ ID NO: 559 | GUCY2C-TAC G12 | Amino Acid |
| SEQ ID NO: 654 | GUCY2C-TAC G13 | Nucleotide |
| SEQ ID NO: 560 | GUCY2C-TAC G13 | Amino Acid |
| SEQ ID NO: 655 | GUCY2C-TAC G14 | Nucleotide |
| SEQ ID NO: 561 | GUCY2C-TAC G14 | Amino Acid |
| SEQ ID NO: 656 | GUCY2C-TAC G15 | Nucleotide |
| SEQ ID NO: 562 | GUCY2C-TAC G15 | Amino Acid |
| SEQ ID NO: 657 | GUCY2C-TAC G16 | Nucleotide |
| SEQ ID NO: 563 | GUCY2C-TAC G16 | Amino Acid |
| SEQ ID NO: 658 | GUCY2C-TAC G17 | Nucleotide |
| SEQ ID NO: 564 | GUCY2C-TAC G17 | Amino Acid |
| SEQ ID NO: 659 | GUCY2C-TAC G18 | Nucleotide |
| SEQ ID NO: 565 | GUCY2C-TAC G18 | Amino Acid |
| SEQ ID NO: 660 | GUCY2C-TAC G19 | Nucleotide |
| SEQ ID NO: 566 | GUCY2C-TAC G19 | Amino Acid |
| SEQ ID NO: 661 | GUCY2C-TAC G20 | Nucleotide |
| SEQ ID NO: 567 | GUCY2C-TAC G20 | Amino Acid |
| SEQ ID NO: 662 | GUCY2C-TAC G21 | Nucleotide |
| SEQ ID NO: 568 | GUCY2C-TAC G21 | Amino Acid |
| SEQ ID NO: 663 | GUCY2C-TAC G22 | Nucleotide |
| SEQ ID NO: 569 | GUCY2C-TAC G22 | Amino Acid |
| SEQ ID NO: 664 | GUCY2C-TAC G23 | Nucleotide |
| SEQ ID NO: 570 | GUCY2C-TAC G23 | Amino Acid |
| SEQ ID NO: 665 | GUCY2C-TAC G24 | Nucleotide |
| SEQ ID NO: 571 | GUCY2C-TAC G24 | Amino Acid |
| SEQ ID NO: 666 | GUCY2C-TAC G25 | Nucleotide |
| SEQ ID NO: 572 | GUCY2C-TAC G25 | Amino Acid |
| SEQ ID NO: 667 | GUCY2C-TAC G26 | Nucleotide |
| SEQ ID NO: 573 | GUCY2C-TAC G26 | Amino Acid |
| SEQ ID NO: 668 | GUCY2C-TAC G27 | Nucleotide |
| SEQ ID NO: 574 | GUCY2C-TAC G27 | Amino Acid |
| SEQ ID NO: 669 | GUCY2C-TAC G28 | Nucleotide |
| SEQ ID NO: 575 | GUCY2C-TAC G28 | Amino Acid |
| SEQ ID NO: 670 | GUCY2C-TAC G29 | Nucleotide |
| SEQ ID NO: 576 | GUCY2C-TAC G29 | Amino Acid |
| SEQ ID NO: 671 | GUCY2C-TAC G30 | Nucleotide |
| SEQ ID NO: 577 | GUCY2C-TAC G30 | Amino Acid |
| SEQ ID NO: 672 | GUCY2C-TAC G31 | Nucleotide |
| SEQ ID NO: 578 | GUCY2C-TAC G31 | Amino Acid |
| SEQ ID NO: 673 | GUCY2C-TAC G32 | Nucleotide |
| SEQ ID NO: 579 | GUCY2C-TAC G32 | Amino Acid |
| SEQ ID NO: 674 | GUCY2C-TAC G33 | Nucleotide |
| SEQ ID NO: 580 | GUCY2C-TAC G33 | Amino Acid |
| SEQ ID NO: 675 | GUCY2C-TAC G34 | Nucleotide |
| SEQ ID NO: 581 | GUCY2C-TAC G34 | Amino Acid |
| SEQ ID NO: 676 | GUCY2C-TAC G35 | Nucleotide |
| SEQ ID NO: 582 | GUCY2C-TAC G35 | Amino Acid |
| SEQ ID NO: 677 | GUCY2C-TAC G36 | Nucleotide |
| SEQ ID NO: 583 | GUCY2C-TAC G36 | Amino Acid |
| SEQ ID NO: 678 | GUCY2C-TAC G37 | Nucleotide |
| SEQ ID NO: 584 | GUCY2C-TAC G37 | Amino Acid |
| SEQ ID NO: 679 | GUCY2C-TAC G38 | Nucleotide |
| SEQ ID NO: 585 | GUCY2C-TAC G38 | Amino Acid |
| SEQ ID NO: 680 | GUCY2C-TAC G39 | Nucleotide |
| SEQ ID NO: 586 | GUCY2C-TAC G39 | Amino Acid |
| SEQ ID NO: 681 | GUCY2C-TAC G40 | Nucleotide |
| SEQ ID NO: 587 | GUCY2C-TAC G40 | Amino Acid |
| SEQ ID NO: 682 | GUCY2C-TAC G41 | Nucleotide |
| SEQ ID NO: 588 | GUCY2C-TAC G41 | Amino Acid |
| SEQ ID NO: 683 | GUCY2C-TAC G42 | Nucleotide |
| SEQ ID NO: 589 | GUCY2C-TAC G42 | Amino Acid |
| SEQ ID NO: 684 | GUCY2C-TAC G43 | Nucleotide |
| SEQ ID NO: 590 | GUCY2C-TAC G43 | Amino Acid |
| SEQ ID NO: 685 | GUCY2C-TAC G44 | Nucleotide |
| SEQ ID NO: 686 | GUCY2C-TAC G44 | Amino Acid |

### Vector Constructs

Disclosed herein, in certain embodiments, are vectors comprising a GUCY2C TAC nucleic acid sequence as disclosed herein. In some embodiments, the vectors further comprise a promoter. In some embodiments, the promoter is functional in a mammalian cell. Promoters, regions of DNA that initiate transcription of a particular nucleic acid sequence, are well known in the art. A “promoter functional in a mammalian cell” refers to a promoter that drives expression of the associated nucleic acid sequence in a mammalian cell. A promoter that drives expression of a nucleic acid sequence is referred to as being “operably connected” to the nucleic acid sequence.

A variety of delivery vectors and expression vehicles are employed to introduce nucleic acids described herein into a cell.

Disclosed herein, in certain embodiments, are vectors comprising:
(a) a first polynucleotide encoding an antigen-binding domain that binds GUCY2C;
(b) a second polynucleotide encoding an antigen-binding domain that binds a protein associated with a TCR complex;
(c) a third polynucleotide encoding a T cell receptor signaling domain polypeptide; and
(d) a promoter that is functional in a mammalian cell.

In some embodiments, the first polynucleotide and third polynucleotide are fused to the second polynucleotide and the coding sequence is operably connected to the promoter. In some embodiments, the second polynucleotide and third polynucleotide are fused to the first polynucleotide and the coding sequence is operably connected to the promoter. In some embodiments, the vector is designed for expression in mammalian cells. In some embodiments, the vector is a viral vector. In some embodiments, the viral vector is a retroviral vector.

In some embodiments, vectors that are useful comprise vectors derived from retroviruses, lentiviruses, Murine Stem Cell Viruses (MSCV), pox viruses, adenoviruses, and adeno-associated viruses. Other delivery vectors that are useful comprise vectors derived from herpes simplex viruses, transposons, vaccinia viruses, human papilloma virus, Simian immunodeficiency viruses, HTLV, human foamy virus and variants thereof. Further vectors that are useful comprise vectors derived from spumaviruses, mammalian type B retroviruses, mammalian type C retroviruses, avian type C retroviruses, mammalian type D retroviruses and HTLV/BLV type retroviruses. One example of a lentiviral vector useful in the disclosed compositions and methods is the pCCL4 vector.

### Pharmaceutical Compositions

Disclosed herein, in certain embodiments, are pharmaceutical compositions comprising an engineered T cell disclosed herein (transduced with and/or expressing a GUCY2C TAC polypeptide), and a pharmaceutically acceptable carrier. Pharmaceutically acceptable carriers include, but are not limited to, buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); or preservatives. In some embodiments, the engineered T cells are formulated for intravenous administration.

Pharmaceutical compositions are administered in a manner appropriate to the disease to be treated (or prevented). The quantity and frequency of administration is determined by such factors as the condition of the patient, and the type and severity of the patient's disease, although appropriate dosages are determined by clinical trials. When “an immunologically effective amount,” “an anti-tumor effective amount,” “a tumor-inhibiting effective amount,” or “therapeutic amount” is indicated, the precise amount of the compositions of the present invention to be administered is determined by a physician with consideration of individual differences in age, weight, tumor size, extent of infection or metastasis, and condition of the patient (subject).

In some embodiments, the engineered T cells and/or pharmaceutical compositions described herein are administered at a dosage of 10¹ to 10¹⁵ cells per kg body weight, 10⁴ to 10⁹ cells per kg body weight, optionally 10⁵ to 10⁸ cells per kg body weight, 10⁶ to 10⁷ cells per kg body weight or 10⁵ to 10⁶ cells per kg body weight, including all integer values within those ranges. In some embodiments, the modified T cells and/or pharmaceutical compositions described herein are administered at a dosage of greater than 10¹ cells per kg body weight. In some embodiments, the modified T cells and/or pharmaceutical compositions described herein are administered at a dosage of less than 10¹⁵ cells per kg body weight.

In some embodiments, the engineered T cells and/or pharmaceutical compositions described herein are administered at a dosage of 0.5×10⁶ cells, 2×10⁶ cells, 4×10⁶ cells, 5×10⁶ cells, 1.2×10⁷ cells, 2×10⁷ cells, 5×10⁷ cells, 2×10⁸ cells, 5×10⁸ cells, 2×10⁹ cells, 0.5-2000×10⁶ cells, 0.5-2×10⁶ cells, 0.5-2×10⁷ cells, 0.5-2×10⁸ cells, or 0.5-2×10⁹ cells, including all integer values within those ranges.

Also disclosed herein are pharmaceutical compositions comprising engineered/modified and unmodified T cells, or comprising different populations of engineered/modified T cells with or without unmodified T cells. One of ordinary skill in the art would understand that a therapeutic quantity of engineered/modified T cells need not be homogenous in nature.

In some embodiments, T cell compositions are administered multiple times at these dosages. In some embodiments, the dosage is administered a single time or multiple times, for example daily, weekly, biweekly, or monthly, hourly, or is administered upon recurrence, relapse or progression of the cancer being treated. The cells, in some embodiments, are administered by using infusion techniques that are commonly known in immunotherapy (see, e.g., Rosenberg et al., New Eng. J. of Med. 319:1676, 1988).

In some embodiments, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium a fungus, mycoplasma, IL-2, and IL-7.

In some embodiments, the modified/engineered T cells and/or pharmaceutical compositions are administered by methods including, but not limited to, aerosol inhalation, injection, infusion, ingestion, transfusion, implantation or transplantation. The modified T cells and/or pharmaceutical compositions may be administered to a subject transarterially, subcutaneously, intradermally, intratumorally, intranodally, intrameduliary, intramuscularly, by intravenous (i.v.) injection, by intravenous (i.v.) infusion, or intraperitoneally. The modified/engineered T cells and/or pharmaceutical compositions thereof may be administered to a patient by intradermal or subcutaneous injection. The modified/engineered T cells and/or pharmaceutical compositions thereof may be administered by i.v. injection. The modified/engineered T cells and/or pharmaceutical compositions thereof may be injected directly into a tumor, lymph node, or site of infection.

A pharmaceutical composition may be prepared by known methods for the preparation of pharmaceutically acceptable compositions that are administered to subjects, such that an effective quantity of the T cells is combined in a mixture with a pharmaceutically acceptable carrier. Suitable carriers are described, for example, in Remington's Pharmaceutical Sciences (Remington's Pharmaceutical Sciences, 20^{th} ed., Mack Publishing Company, Easton, Pa., USA, 2000). On this basis, the compositions may include, albeit not exclusively, solutions of the substances in association with one or more pharmaceutically acceptable carriers or diluents, and contained in buffered solutions with a suitable pH and iso-osmotic with the physiological fluids.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, N-(1 (2,3-dioleyloxy)propyl)N,N,N-trimethylammonium chloride (DOTMA), diolesylphosphotidyl-ethanolamine (DOPE), and liposomes. In some embodiments, such compositions contain a therapeutically effective amount of the compound, together with a suitable amount of carrier so as to provide the form for direct administration to the patient.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions.

A pharmaceutical composition disclosed herein may be formulated into a variety of forms and administered by a number of different means. A pharmaceutical formulation may be administered orally, rectally, or parenterally, in formulations containing conventionally acceptable carriers, adjuvants, and vehicles as desired. The term “parenteral” as used herein includes subcutaneous, intravenous, intramuscular, or intrasternal injection and infusion techniques. Administration includes injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration. In some exemplary embodiments, a route of administration is via an injection such as an intramuscular, intravenous, subcutaneous, or intraperitoneal injection.

Liquid formulations include an oral formulation, an intravenous formulation, an intranasal formulation, an ocular formulation, an otic formulation, an aerosol, and the like. In certain embodiments, a combination of various formulations is administered. In certain embodiments a composition is formulated for an extended release profile.

### Methods of Treatment and Use

Disclosed herein, in certain embodiments, are methods of using engineered T cells disclosed herein in the treatment of a GUCY2C-expressing cancer in an individual in need thereof.

In some embodiments, an antigen-binding domain that binds GUCY2C of a TAC polypeptide disclosed herein binds to GUCY2C on a tumor cell. In some embodiments, an antigen-binding domain that binds GUCY2C of a TAC polypeptide disclosed herein selectively binds to GUCY2C on a tumor cell.

Disclosed herein, in certain embodiments, are methods of treating a cancer expressing GUCY2C in an individual in need thereof, comprising administering to the individual an engineered T cell disclosed herein or a pharmaceutical composition comprising an engineered T cell disclosed herein.

Further disclosed herein is use of an engineered T cell disclosed herein in the preparation of a medicament to treat cancer expressing GUCY2C in an individual in need thereof. Additionally disclosed herein in certain embodiments is the use of an engineered T cell disclosed herein or a pharmaceutical composition disclosed herein to treat a cancer expressing GUCY2C in an individual in need thereof.

In some embodiments, the engineered T cells disclosed herein are part of a combination therapy. In some embodiments, effectiveness of a therapy disclosed herein is assessed multiple times. In some embodiments, patients are stratified based on a response to a treatment disclosed herein. In some embodiments, an effectiveness of treatment determines entrance into a trial.

In some embodiments, the engineered T cells disclosed herein are administered in combination with a lymphodepleting therapy, or are administered to a subject who has received a lymphodepleting therapy. Examples of lymphodepleting therapies include nonmyeloablative lymphodepleting chemotherapy, myeloablative lymphodepleting chemotherapy, fludarabine, cyclophosphamide, corticosteroids, alemtuzumab, total body irradiation (TBI), and any combination thereof.

Cancers that may be treated with engineered T cells disclosed herein include any form of neoplastic disease. In some embodiments, the cancer is a primary colorectal cancer, a primary gastric cancer, a primary gastroesophageal junction cancer, a primary esophageal cancer, or a primary pancreatic cancer. In some embodiments, the cancer is a metastatic colorectal cancer, a metastatic gastric cancer, a metastatic gastroesophageal junction cancer, a metastatic esophageal cancer, or a metastatic pancreatic cancer.

In some embodiments, the cancer that is to be treated is a primary colorectal cancer. Colorectal cancer affects both men and women, and is responsible for 9.2% of all cancer deaths. The lack of response to targeted therapy, such as anti-EGFR antibodies, has been correlated with mutations in the KRAS and BRAF oncogenes. In addition, immunotherapies, such as immune checkpoint inhibitors, have failed to show significant survival benefit in most patients with colorectal cancer, owing to low tumor mutational burden and reduced density of immune infiltration. Guanylyl Cyclase C (GUCY2C) is overexpressed in more than 90% of colorectal cancers across all stages.

In some embodiments, the cancer that is to be treated is a primary gastric cancer, for example a primary gastroesophageal junction cancer. Gastric cancers are the 6^{th} most common cancer in the world, and the second-leading cause of cancer-related deaths worldwide. In most of the world, stomach cancers form in the main part of the stomach (stomach body). In the United States, stomach cancer is more likely to affect the area where the esophagus meets the stomach, i.e., gastroesophageal junction cancer. Many gastric cancers evolve from intestinal metaplasia resulting in over 50% of gastric cancers and gastroesophageal junction cancers being characterized by ectopic over-expression of GUCY2C.

In some embodiments, the cancer that is to be treated is a primary pancreatic cancer. Pancreatic cancer has the highest mortality rate of all major cancers. For all stages combined, the 5-year relative survival rate is 10%. For people diagnosed with local disease, the 5-year survival is only 39%. Many pancreatic cancers evolve from intestinal metaplasia resulting in over 50% of pancreatic cancers being characterized by overexpression of GUCY2C.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Manufacturing of GUCY2C-TAC T Cells

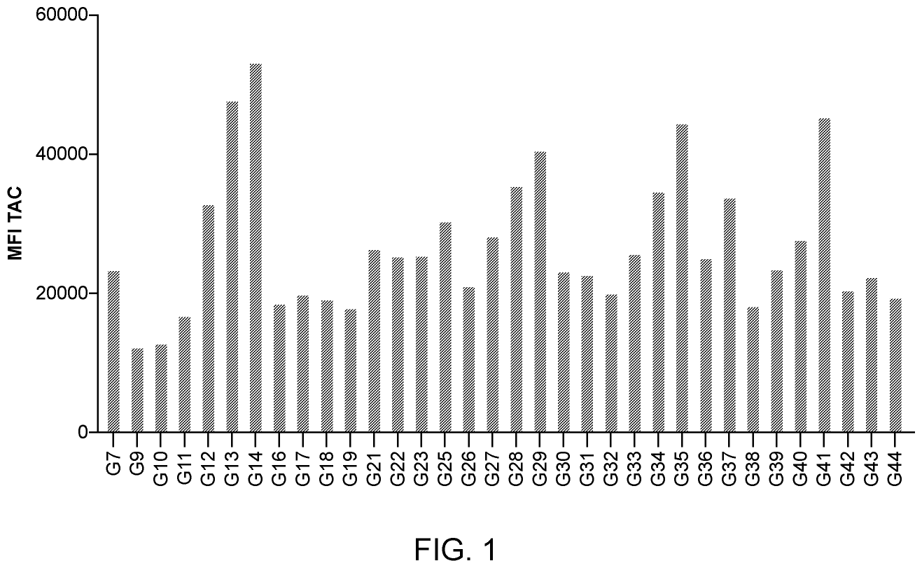

T cells were engineered with lentiviral vectors to express a variety of GUCY2C-TAC receptors listed in Table 8. Surface expression was analyzed via flow cytometry (FIG. 1). Results show that T cells expressed the 34 GUCY2C-TACs of Table 8.

TABLE 8

| TAC ID | Binding Domain | Sequence ID |
|---|---|---|
| G7 | muScFv | SEQ ID NO: 554 |
| G9 | muScFv | SEQ ID NO: 556 |
| G10 | muScFv | SEQ ID NO: 557 |
| G11 | muScFv | SEQ ID NO: 558 |
| G12 | muScFv | SEQ ID NO: 559 |
| G13 | muScFv | SEQ ID NO: 560 |
| G14 | muScFv | SEQ ID NO: 561 |
| G16 | Nanobody | SEQ ID NO: 563 |
| G17 | Nanobody | SEQ ID NO: 564 |
| G18 | Nanobody | SEQ ID NO: 565 |
| G19 | Nanobody | SEQ ID NO: 566 |
| G21 | Nanobody | SEQ ID NO: 568 |
| G22 | Nanobody | SEQ ID NO: 569 |
| G23 | Nanobody | SEQ ID NO: 570 |
| G25 | huScFv | SEQ ID NO: 572 |
| G26 | huScFv | SEQ ID NO: 573 |
| G27 | huScFv | SEQ ID NO: 574 |
| G28 | huScFv | SEQ ID NO: 575 |
| G29 | huScFv | SEQ ID NO: 576 |
| G30 | huScFv | SEQ ID NO: 577 |
| G31 | huScFv | SEQ ID NO: 578 |
| G32 | huScFv | SEQ ID NO: 579 |
| G33 | huScFv | SEQ ID NO: 580 |
| G34 | huScFv | SEQ ID NO: 581 |
| G35 | huScFv | SEQ ID NO: 582 |
| G36 | huScFv | SEQ ID NO: 583 |
| G37 | huScFv | SEQ ID NO: 584 |
| G38 | huScFv | SEQ ID NO: 585 |
| G39 | huScFv | SEQ ID NO: 586 |
| G40 | huScFv | SEQ ID NO: 587 |
| G41 | huScFv | SEQ ID NO: 588 |
| G42 | huScFv | SEQ ID NO: 589 |
| G43 | huScFv | SEQ ID NO: 590 |
| G44 | Nanobody | SEQ ID NO: 686 |

### Example 2: In Vitro Activation of GUCY2C-TAC T Cells

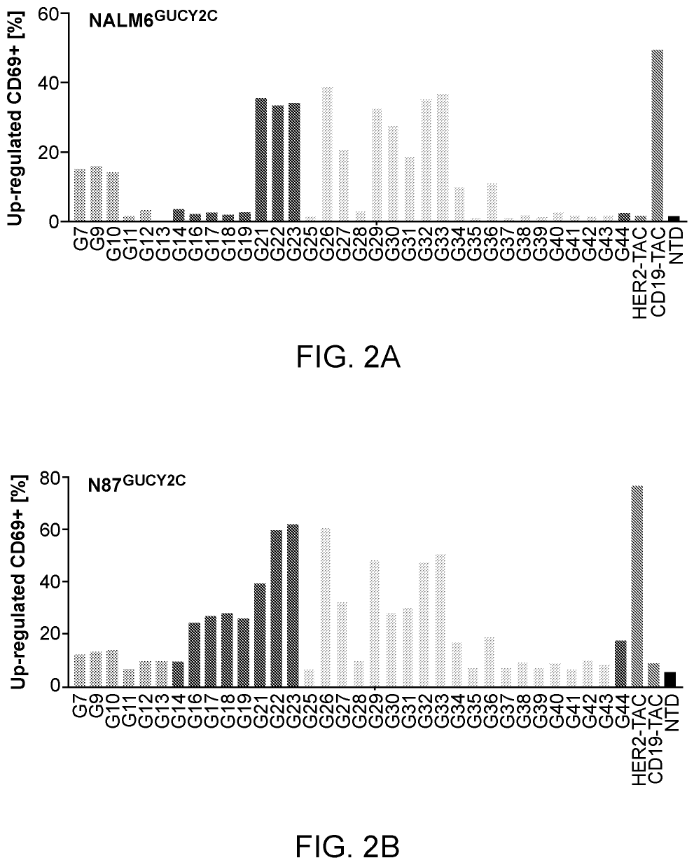

T cells were engineered to express the GUCY2C-TAC receptors listed in Table 8. T cell activation was measured as a function of the upregulation of the early T cell activation marker, CD69. GUCY2C-TAC T cells were co-cultured at a 1:1 E:T ratio with a variety of tumor cell lines expressing GUCY2C (N87^{GUCY2C}, NALM6^{GUCY2C}). Following a 4-hour co-culture, GUCY2C-TAC T cells were harvested and analyzed for CD69 surface expression by flow cytometry. As shown in FIGS. 2A-2B, GUCY2C-TAC T cells were activated when co-cultured with GUCY2C-positive target cells NALM6^{GUCY2C} (FIG. 2A) and N87^{GUCY2C} (FIG. 2B). Activation was not observed with GUCY2C negative control cells. The observed activation varied across all tested GUCY2C-TAC T cells. When stimulated with GUCY2C-positive target cells, GUCY2C-TAC T cells were able to induce the activation of non-transduced T cells in that same T cell populations.

### Example 3: In Vitro Expansion of GUCY2C-TAC T Cells

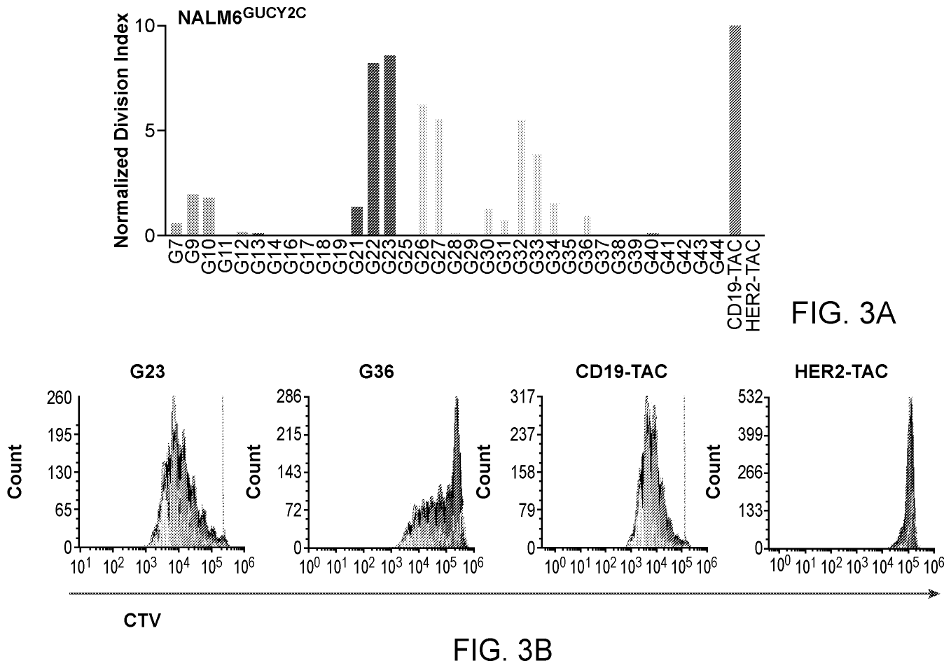

T cells were engineered to express a variety of GUCY2C-TAC receptors (Table 8). Proliferation of GUCY2C-TAC T cells co-cultured in a 1:3 E:T ratio for 4 days with NALM6^{GUCY2C} was evaluated. NALM6^{GUCY2C} is a leukemic cell line that was engineered to overexpress a truncated version of GUCY2C lacking the cytosolic domains. The parental NALM6 cell line lacking GUCY2C expression was used as negative control. GUCY2C-TAC T cells were evaluated via the CTV (cell trace violet) proliferation assay. Target cells were inactivated using mitomycin C, and T cells were loaded with CTV dye prior to co-culture with target cells at a 1: E:T ratio. After a 4-day co-culture, T cells were analyzed via flow cytometry. Results of the CTV proliferation assay were quantified (FIG. 3A), and representative examples are shown (FIG. 3B). The division index (DI), a measure of proliferation from all GUCY2C-TAC T cells was normalized to the division index of cells grown in the absence of target cells (FIG. 3A). The observed proliferation varied across all tested GUCY2C-TAC T cells. The majority of GUCY2C-TAC T cells showed proliferation, including several GUCY2C-TAC T cell candidates with high proliferative activity. No proliferation was observed against GUCY2C-negative control cells. GUCY2C-TAC G23 (SEQ ID NO: 570) and GUCY2C-TAC G36 (SEQ ID NO: 583) T cells showed various levels of proliferation relative to the positive control of CD19-TAC T cells. No proliferation was observed in the HER2-TAC negative control cells.

### Example 4: In Vitro Cytotoxicity of GUCY2C-TAC T Cells

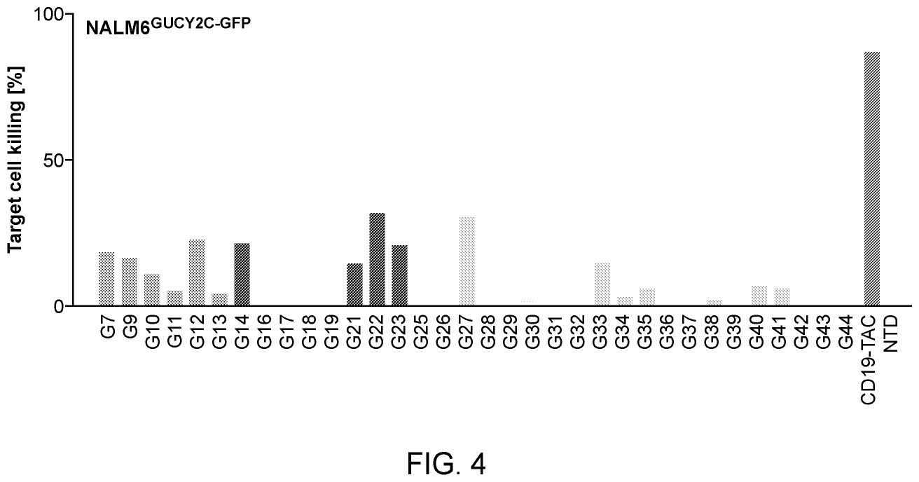

T cells were engineered to express GUCY2C-TAC receptors listed in Table 8. GUCY2C-TAC T cells were co-cultured at E:T ratios 1:10 and 1:20 with 1×10⁴ NALM6^{GUCY2C-GFPeLuc} target cells/well in a cell imaging reader. Photos were captured every 8 hours for 5 days. The area of GFP-expressing cells is calculated for each time point and E:T ratio. From these values, the area under the curve (AUC) for each of the GUCY2C-TAC T cells was calculated and plotted, representing target cell killing at each E:T ratio. Data shows percentage target cell killing of the GUCY2C-TAC T cells at E:T ratio of 1:10 (FIG. 4). The tested GUCY2C-TAC T cells showed varying levels of cytotoxicity. No cytotoxicity was observed against GUCY2C negative control cells. The positive control, CD19-TAC, showed nearly 100% killing of target cells at E:T of 1:10. NTD is shown as the negative control.

### Example 5: Antigen-Specific In Vitro Activation of GUCY2C-TAC T Cells

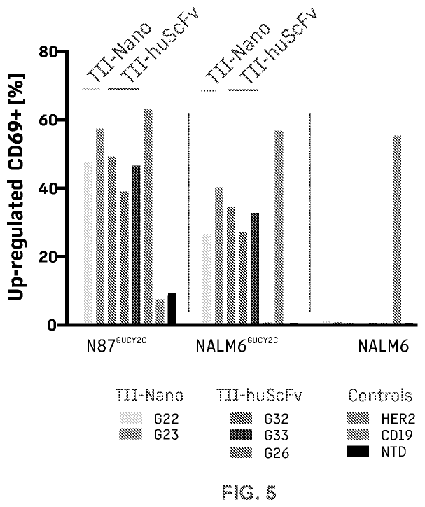

T cells were engineered to express GUCY2C-TAC receptors G22 (SEQ ID NO: 569), G23 (SEQ ID NO: 570), G26 (SEQ ID NO: 573), G32 (SEQ ID NO: 579), or G33 (SEQ ID NO: 580). T cell activation was measured as a function of the upregulation of the early T cell activation marker, CD69. T cells expressing the GUCY2C-TAC were co-cultured at a 1:1 E:T ratio with a variety of tumor cell lines expressing GUCY2C (NCI-N87^{GUCY2C}, NALM6^{GUCY2C}) Following a 4 hour co-culture, GUCY2C-TAC T cells were harvested and analyzed for CD69 surface expression by flow cytometry. As shown in FIG. 5, T cells expressing GUCY2C-TAC were activated when co-cultured with both GUCY2C positive target cells, N87^{GUCY2C} and NALM6^{GUCY2C}, but not with GUCY2C negative control cells, NALM6. All 5 tested GUCY2C-TAC T cell variants were activated in response to GUCY2C-expressing tumor cells, comparable to the relevant positive controls (i.e., HER2-TAC for N87^{GUCY2C}; CD19-TAC T for NALM6^{GUCY2C} target cells).

### Example 6: Antigen-Specific In Vitro Expansion of GUCY2C-TAC T Cells

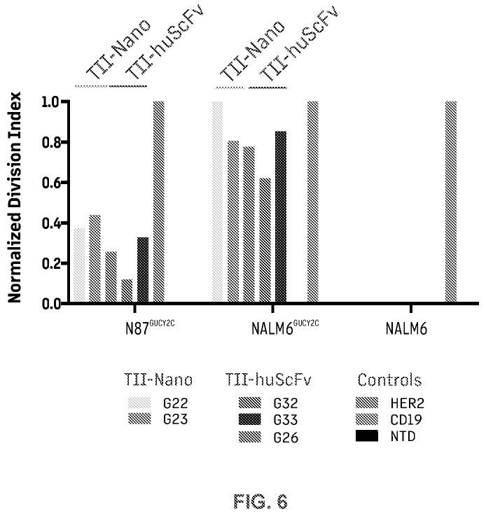

T cells were engineered to express GUCY2C-TAC receptors G22 (SEQ ID NO: 569), G23 (SEQ ID NO: 570), G26 (SEQ ID NO: 573), G32 (SEQ ID NO: 579), or G33 (SEQ ID NO: 580). GUCY2C-TAC T cells were evaluated via the CTV proliferation assay. Target cells (N87^{GUCY2}C, NALM6^{GUCY2C}) were inactivated using mitomycin, and T cells were loaded with cell tracing (CTV) dye prior to co-culture with target cells at a 1:3 E:T ratio. After a 4 day co-culture T cells were analyzed via flow cytometry. Results of the CTV proliferation assay were quantified (FIG. 6).

The division index (DI) was normalized to the respective positive controls (HER2-TAC for N87^{GUCY2C} and CD19-TAC for NALM6^{GUCY2C}). All 5 tested GUCY2C-TAC T cell products proliferated upon co-culture with GUCY2C-expressing target cells. No proliferation was observed against GUCY2C negative control cells.

### Example 7. Antigen-Specific In Vitro Cytotoxicity of GUCY2C-TAC T Cells

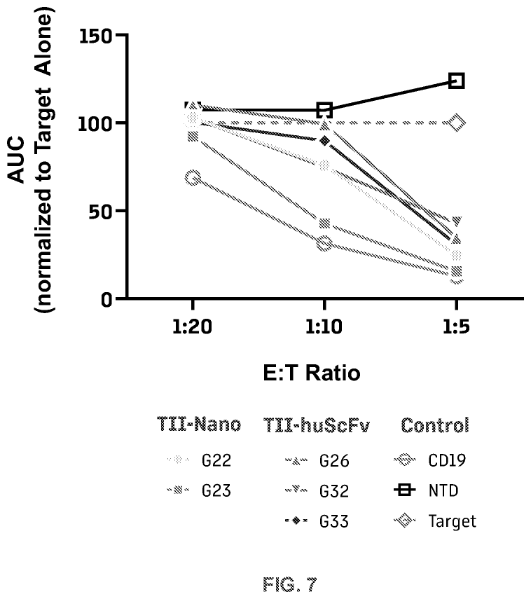

T cells were engineered to express GUCY2C-TAC receptors G22 (SEQ ID NO: 569), G23 (SEQ ID NO: 570), G26 (SEQ ID NO: 573), G32 (SEQ ID NO: 579), or G33 (SEQ ID NO: 580). GUCY2C-TAC T cells were co-cultured at E:T ratios 1:5, 1:10 and 1:20 with 1×10⁴ NALM6^{GUCY2C-GFPeLuc} target cells/well in a cell imaging reader. Photos were captured every 8 hours for 5 days. The area of GFP-expressing cells is calculated for each time point and E:T ratio. From these values, the area under the curve (AUC) for each of the GUCY2C-TAC T cells was calculated, normalized to target cells alone and plotted, representing target cell killing at each E:T ratio. FIG. 7 shows exemplary results of GUCY2C-TAC Nanobody 7 (closed circles), GUCY2C-TAC Nanobody 8 (closed squares), GUCY2C-TAC huScFV 2 (closed triangles), GUCY2C-TAC huScFV 8 (closed inverted triangles), and GUCY2C-TAC huScFV 9 (closed diamonds). Data shown demonstrate the different levels of target cell killing dependent on the E:T ratios used. NTD negative controls cells show no cytotoxicity. The graph demonstrated that all tested GUCY2C-TAC T cells show cytotoxicity, with some variants being close to the cytotoxicity observed by the positive control CD19-TAC T cells.

### Example 8: In Vitro Activity of GUCY2C-TAC T Cells Against Various Tumor Cell Types Endogenously Expressing GUCY2C

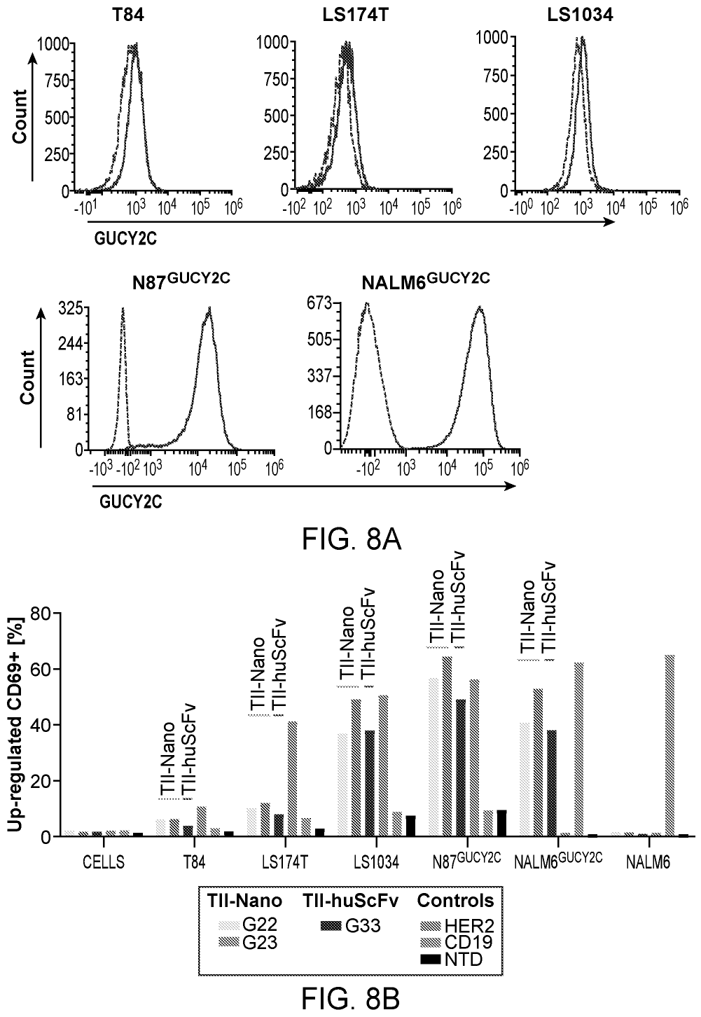

The natural surface expression levels of GUCY2C on T84, LS174T (colon carcinoma), LS1034 (colorectal carcinoma) cell lines were measured and activation of GUCY2C-TAC T cells against the cells was analyzed. Engineered cell lines N87^{GUCY2C} and NALM6^{GUCY2C} were used as positive control. Dotted lines represent secondary antibody only and were used as negative control. Natural cell lines showed surface expression levels that were above background, which indicates lower expression levels compared to the engineered positive controls (FIG. 8A).

T cells were engineered to express GUCY2C-TAC receptors G22 (SEQ ID NO: 569), G23 (SEQ ID NO: 570), or G33 (SEQ ID NO: 580). GUCY2C-TAC T cells were co-cultured at a 1:1 ratio with the GUCY2C-expressing cells (FIG. 8A), while GUCY2C-negative NALM6 cells were used as negative control. Following a 4-hour co-culture, GUCY2C-TAC T cells were harvested and analyzed for CD69 surface expression by flow cytometry. As shown in FIG. 8B, GUCY2C-TAC T cells were activated when co-cultured with the GUCY2C-positive target cells. The level of activation varied across cell lines with T84 cells inducing the lowest level of activation, while LS1034 cells induced the highest levels of T cell activation. With the exception of the LS174T cell line, GUCY2C-TAC T cell activation was comparable with the respective positive control TAC T cells. Neither NTD negative controls nor T cells alone showed activation.

### Example 9: In Vitro Screening of GUCY2C-TACs

T cells are engineered to express GUCY2C-TAC receptors (e.g., any one of SEQ ID NOs: 465-513, 546-590, or 686). T cell activation is measured as a function of the upregulation of the early T cell activation marker, CD69. Engineered T cells are co-cultured at a 1:1 E:T ratio with target cells expressing GUCY2C or negative control cells that do not express GUCY2C. Following a 4-hour co-culture, GUCY2C-TAC T cells are harvested and analyzed for CD69 surface expression by flow cytometry. Expansion of GUCY2C-TAC T cells is evaluated via the CTV proliferation assay. GUCY2C-positive target cells or GUCY2C-negative control cells are inactivated using mitomycin C, and T cells are loaded with CTV dye prior to co-culture with target or control cells at a 3:1 E:T ratio. After a 4-day co-culture, T cells are analyzed via flow cytometry. GFP/Luc-expressing GUCY2C-positive target cells or GUCY2C-negative control cells are used to assess cytotoxicity induced by TAC T cells in a cell imaging reader. Photos are captured every 8 hours for 5 days. The area of GFP-expressing cells is calculated for each time point and E:T ratio. From these values, the area under the curve (AUC) for each of the GUCY2C-TAC T cells is calculated and plotted, representing target cell killing at each E:T ratio. Results are analyzed to compare the relative effects of the GUCY2C-TAC T cells on GUCY2C-positive target cells or GUCY2C-negative control cells.

### Example 10: In Vivo Activity of GUCY2C-TAC T Cells in Mammalian Subjects

T cells are engineered to express GUCY2C-TAC receptors (e.g., any one of SEQ ID NOs: 465-513, 546-590, or 686). Mice are inoculated with 5×10⁵-1×10⁷ GUCY2C-expressing tumor cells. Four days after engraftment, mice are treated with a single intravenous dose of GUCY2C-TAC T cells. Non-treated (NT) mice and mice treated with non-transduced T cells (NTD) are used as negative controls. Mice are dosed with 4×10⁶ TAC T cells or an equivalent number of NTD cells that matches the total T cell dose used for TAC T cells. Total luminescence is measured weekly. The resulting total flux (photons/second) as the sum of the dorsal and ventral reads, overall survival, and relative change in body weight as a means to assess toxicity are measured. Results are analyzed to compare animals treated with GUCY2C-TAC T cells to NT and NTD animals.

### Example 11: Treatment of Human Subjects with GUCY2C-TAC T Cells

A human subject having a GUCY2C-expressing primary colorectal cancer presents. Autologous T cells are engineered to express a GUCY2C-TAC receptor (e.g., any one of SEQ ID NOs: 465-513, 546-590, or 686) and expression of the TAC is confirmed. The subject is administered lymphodepleting chemotherapy followed by administration of TAC-expressing T cells at an appropriate dose. The subject is monitored for toxicity and disease progression.

Sequence Listing

| SEQ | Nucleotide/ | |
|---|---|---|
| ID | Amino | |
| NO | Acid | Sequence |
| 1 | Nucleotide | ATGGATTTCCAGGTCCAGATTTTCTCCTTCCTGCT |
| | | GATTTCCGCAAGCGTCATT |
| 2 | Amino Acid | MDFQVQIFSFLLISASVI |
| 3 | Nucleotide | GAACAGAAACTGATTAGCGAAGAAGACCTG |
| 4 | Amino Acid | EQKLISEEDL |
| 5 | Nucleotide | ACTAGTGGCGGAGGAGGATCACTCGAG |
| 6 | Amino Acid | TSGGGGSLE |
| 7 | Nucleotide | AACCCCGGGGGAGGAGGAGGGAGCGGGGGAGGAGG |
| | | CAGCGGCGGGGGAGGCTCTGGAGGAGGAGGGAGCG |
| | | GATCC |
| 8 | Amino Acid | NPGGGGGSGGGGSGGGGSGGGGSGS |
| 9 | Nucleotide | AGCGGACAGGTGCTGCTGGAATCCAATATCAAAGT |
| | | CCTGCCCACTTGGTCTACCCCCGTGCAGCCT |
| 10 | Amino Acid | SGQVLLESNIKVLPTWSTPVQP |
| 11 | Nucleotide | GCCGAAGCAGCAGCAAAGGAGGCCGCAGCGAAGGA |
| | | AGCAGCTGCGAAGGCC |
| 12 | Amino Acid | AEAAAKEAAAKEAAAKA |
| 13 | Nucleotide | GCCGAGGCAGCTGCAAAGGAAGCTGCGGCGAAGGA |
| | | GGCCGCAGCGAAAGAAGCAGCGGCAAAAGAAGCAG |
| | | CCGCCAAAGCC |
| 14 | Amino Acid | AEAAAKEAAAKEAAAKEAAAKEAAAKA |
| 15 | Nucleotide | ATCGTAGTGTTGGCATTTCAAAAAGCGTCTAGCAT |
| | | CGTCTATAAGAAGGAAGGTGAACAAGTCGAGTTTT |
| | | CTTTCCCCCTTGCATTTACGGTGGAAAAGCTTACG |
| | | GGTAGCGGCGAGCTGTGGTGGCAAGCTGAACGGGC |
| | | TTCAAGCTCAAAATCTTGGATTACTTTTGACTTGA |
| | | AGAACAAAGAGGTGAGTGTCAAAAGAGTTACTCAG |
| | | GACCCAAAGCTTCAAATGGGGAAGAAACTTCCGCT |
| | | GCACCTGACGTTGCCTCAGGCCCTGCCTCAATATG |
| | | CCGGCTCAGGCAATCTGACCCTCGCGCTGGAAGCT |
| | | AAGACCGGAAAATTGCACCAGGAAGTCAATTTGGT |
| | | TGTGATGCGCGCCACTCAGCTCCAAAAAAATCTCA |
| | | CTTGCGAGGTATGGGGGCCTACGAGCCCAAAACTT |
| | | ATGCTGTCTTTGAAGCTTGAAAACAAGGAAGCGAA |
| | | AGTTTCTAAGCGCGAGAAAGCGGTATGGGTTTTGA |
| | | ATCCTGAGGCTGGAATGTGGCAATGCCTCCTGAGC |
| | | GATAGCGGGCAGGTGCTGTTGGAGAGCAACATCAA |
| | | GGTTTTGCCAGCAGCC |
| 16 | Amino Acid | IVVLAFQKASSIVYKKEGEQVEFSFPLAFTVEKLT |
| | | GSGELWWQAERASSSKSWITFDLKNKEVSVKRVTQ |
| | | DPKLQMGKKLPLHLTLPQALPQYAGSGNLTLALEA |
| | | KTGKLHQEVNLVVMRATQLQKNLTCEVWGPTSPKL |
| | | MLSLKLENKEAKVSKREKAVWVLNPEAGMWQCLLS |
| | | DSGQVLLESNIKVLPAA |
| 17 | Nucleotide | ATGGAGACCCCCGCCCAGCTGCTGTTCCTGCTGCT |
| | | GCTGTGGCTGCCCGACACCACCGGC |
| 18 | Amino Acid | METPAQLLFLLLLWLPDTTG |
| 19 | Nucleotide | ATGGCCCTGCCAGTGACCGCCCTGCTGCTGCCACT |
| | | GGCCCTGCTGCTGCACGCCGCCAGACCC |
| 20 | Amino Acid | MALPVTALLLPLALLLHAARP |
| 21 | Nucleotide | GGATCTACCAGCGGATCCGGCAAGCCTGGCAGCGG |
| | | AGAGGGATCCACAAAGGGA |
| 22 | Amino Acid | GSTSGSGKPGSGEGSTKG |
| 23 | Nucleotide | ggcggcggcggaagtggaggaggaggctcaggcgg |
| | | aggagggagc |
| 24 | Amino Acid | GGGGSGGGGSGGGGS |
| 25 | Nucleotide | ggaggaggagggagcgggggaggaggcagcggcgg |
| | | gggaggctctggaggaggagggagc |
| 26 | Amino Acid | GGGGSGGGGSGGGGSGGGGS |
| 27 | Nucleotide | GGAGGAGGAGGGAGC |
| 28 | Amino Acid | GGGGS |
| 29 | Nucleotide | ATGGCCCTGCCAGTGACCGCCCTGCTGCTGCCACT |
| | | GGCCCTGCTGCTGCACGCCGCCCGGCCT |
| 30 | Amino Acid | MALPVTALLLPLALLLHAARP |
| 31 | Nucleotide | ATGGACATCCAGATGACTCAGACCACAAGCTCCCT |
| | | GTCTGCAAGTCTGGGCGACCGGGTGACAATCTCCT |
| | | GCAGAGCCTCTCAGGATATTAGGAACTACCTGAAT |
| | | TGGTATCAGCAGAAACCTGATGGCACAGTCAAGCT |
| | | GCTGATCTACTATACCAGCCGGCTGCACTCAGGCG |
| | | TGCCAAGCAAATTCTCAGGAAGCGGCTCCGGGACT |
| | | GACTACTCCCTGACCATCTCTAACCTGGAGCAGGA |
| | | AGATATTGCTACCTATTTCTGCCAGCAGGGCAATA |
| | | CACTGCCCTGGACTTTTGCCGGAGGCACCAAACTG |
| | | GAGATCAAGGGGGGAGGCGGGAGTGGAGGCGGGGG |
| | | ATCAGGAGGAGGAGGCAGCGGAGGAGGAGGGTCCG |
| | | AGGTCCAGCTGCAGCAGAGCGGACCAGAACTGGTG |
| | | AAGCCCGGAGCAAGTATGAAAATCTCCTGTAAGGC |
| | | CTCAGGATACAGCTTCACCGGCTATACAATGAACT |
| | | GGGTGAAACAGTCCCATGGCAAGAACCTGGAATGG |
| | | ATGGGGCTGATTAATCCTTACAAAGGCGTCAGCAC |
| | | CTATAATCAGAAGTTTAAAGACAAGGCCACACTGA |
| | | CTGTGGATAAGTCTAGTTCAACCGCTTACATGGAG |
| | | CTGCTGTCCCTGACATCTGAAGACAGTGCCGTGTA |
| | | CTATTGTGCTCGGTCTGGCTACTATGGGGACAGTG |
| | | ATTGGTACTTCGATGTCTGGGGACAGGGCACTACC |
| | | CTGACCGTGTTTTCT |
| 32 | Amino Acid | MDIQMTQTTSSLSASLGDRVTISCRASQDIRNYLN |
| | | WYQQKPDGTVKLLIYYTSRLHSGVPSKFSGSGSGT |
| | | DYSLTISNLEQEDIATYFCQQGNTLPWTFAGGTKL |
| | | EIKGGGGSGGGGSGGGGSGGGGSEVQLQQSGPELV |
| | | KPGASMKISCKASGYSFTGYTMNWVKQSHGKNLEW |
| | | MGLINPYKGVSTYNQKFKDKATLTVDKSSSTAYME |
| | | LLSLTSEDSAVYYCARSGYYGDSDWYFDVWGQGTT |
| | | LTVFS |
| 33 | Nucleotide | ATGGCCGACATCGTGCTGACACAGAGCCCCGCCAT |
| | | CATGTCTGCCAGCCCTGGCGAGAAAGTGACCATGA |
| | | CCTGTAGCGCCAGCAGCAGCGTGTCCTACATGAAC |
| | | TGGTATCAGCAGAAGTCCGGCACCAGCCCCAAGCG |
| | | GTGGATCTACGACACAAGCAAGCTGGCCTCTGGCG |
| | | TGCCCGCCCACTTTAGAGGCTCTGGCAGCGGCACA |
| | | AGCTACAGCCTGACCATCAGCGGCATGGAAGCCGA |
| | | GGATGCCGCCACCTACTACTGCCAGCAGTGGTCCA |
| | | GCAACCCCTTCACCTTTGGCTCCGGCACAAAGCTG |
| | | GAAATCAACCGGGCCGACACCGCCCCTACAGGCGG |
| | | CGGAGGATCTGGCGGAGGCGGATCTGGGGGCGGAG |
| | | GAAGTGGGGGGGGAGGATCTATGGCTCAGGTGCAG |
| | | CTGCAGCAGTCTGGCGCCGAACTGGCTAGACCTGG |
| | | CGCCTCCGTGAAGATGAGCTGCAAGGCCAGCGGCT |
| | | ACACCTTCACCCGGTACACCATGCACTGGGTCAAG |
| | | CAGAGGCCTGGACAGGGCCTGGAATGGATCGGCTA |
| | | CATCAACCCCAGCCGGGGCTACACCAACTACAACC |
| | | AGAAGTTCAAGGACAAGGCCACCCTGACCACCGAC |
| | | AAGAGCAGCAGCACCGCCTACATGCAGCTGTCCTC |
| | | CCTGACCAGCGAGGACAGCGCCGTGTACTACTGCG |
| | | CCCGGTACTACGACGACCACTACTCCCTGGACTAC |
| | | TGGGGCCAGGGCACCACACTGACCGTGTCTAGTA |
| 34 | Amino Acid | MADIVLTQSPAIMSASPGEKVTMTCSASSSVSYMN |
| | | WYQQKSGTSPKRWIYDTSKLASGVPAHFRGSGSGT |
| | | SYSLTISGMEAEDAATYYCQQWSSNPFTFGSGTKL |
| | | EINRADTAPTGGGGSGGGGSGGGGSGGGGSMAQVQ |
| | | LQQSGAELARPGASVKMSCKASGYTFTRYTMHWVK |
| | | QRPGQGLEWIGYINPSRGYTNYNQKFKDKATLTTD |
| | | KSSSTAYMQLSSLTSEDSAVYYCARYYDDHYSLDY |
| | | WGQGTTLTVSS |
| 35 | Nucleotide | CAGACCGTGGTGACCCAGGAGCCCAGCCTGACCGT |
| | | GAGCCCCGGCGGCACCGTGACCCTGACCTGCGGCA |
| | | GCAGCACCGGCGCCGTGACCAGCGGCTACTACCCC |
| | | AACTGGGTGCAGCAGAAGCCCGGCCAGGCCCCCAG |
| | | GGGCCTGATCGGCGGCACCAAGTTCCTGGCCCCCG |
| | | GCACCCCCGCCAGGTTCAGCGGCAGCCTGCTGGGC |
| | | GGCAAGGCCGCCCTGACCCTGAGCGGCGTGCAGCC |
| | | CGAGGACGAGGCCGAGTACTACTGCGCCCTGTGGT |
| | | ACAGCAACAGGTGGGTGTTCGGCGGCGGCACCAAG |
| | | CTGACCGTGCTGGGCGGCGGCGGCAGCGGCGGCGG |
| | | CGGCAGCGGCGGCGGCGGCAGCGAGGTGCAGCTGC |
| | | TGGAGAGCGGCGGCGGCCTGGTGCAGCCCGGCGGC |
| | | AGCCTGAAGCTGAGCTGCGCCGCCAGCGGCTTCAC |
| | | CTTCAACATCTACGCCATGAACTGGGTGAGGCAGG |
| | | CCCCCGGCAAGGGCCTGGAGTGGGTGGCCAGGATC |
| | | AGGAGCAAGTACAACAACTACGCCACCTACTACGC |
| | | CGACAGCGTGAAGAGCAGGTTCACCATCAGCAGGG |
| | | ACGACAGCAAGAACACCGCCTACCTGCAGATGAAC |
| | | AACCTGAAGACCGAGGACACCGCCGTGTACTACTG |
| | | CGTGAGGCACGGCAACTTCGGCAACAGCTACGTGA |
| | | GCTTCTTCGCCTACTGGGGCCAGGGCACCCTGGTG |
| | | ACCGTGAGCAGC |
| 36 | Amino Acid | QTVVTQEPSLTVSPGGTVTLTCGSSTGAVTSGYYP |
| | | NWVQQKPGQAPRGLIGGTKFLAPGTPARFSGSLLG |
| | | GKAALTLSGVQPEDEAEYYCALWYSNRWVFGGGTK |
| | | LTVLGGGGSGGGGSGGGGSEVQLLESGGGLVQPGG |
| | | SLKLSCAASGFTFNIYAMNWVRQAPGKGLEWVARI |
| | | RSKYNNYATYYADSVKSRFTISRDDSKNTAYLQMN |
| | | NLKTEDTAVYYCVRHGNFGNSYVSFFAYWGQGTLV |
| | | TVSS |
| 37 | Nucleotide | GACATCCAGCTGACCCAGAGCCCCGCCATCATGAG |
| | | CGCCAGCCCCGGCGAGAAGGTGACCATGACCTGCA |
| | | GGGCCAGCAGCAGCGTGAGCTACATGAACTGGTAC |
| | | CAGCAGAAGAGCGGCACCAGCCCCAAGAGGTGGAT |
| | | CTACGACACCAGCAAGGTGGCCAGCGGCGTGCCCT |
| | | ACAGGTTCAGCGGCAGCGGCAGCGGCACCAGCTAC |
| | | AGCCTGACCATCAGCAGCATGGAGGCCGAGGACGC |
| | | CGCCACCTACTACTGCCAGCAGTGGAGCAGCAACC |
| | | CCCTGACCTTCGGCGCCGGCACCAAGCTGGAGCTG |
| | | AAGGGCGGCGGCGGCAGCGGCGGCGGCGGCAGCGG |
| | | CGGCGGCGGCAGCGACATCAAGCTGCAGCAGAGCG |
| | | GCGCCGAGCTGGCCAGGCCCGGCGCCAGCGTGAAG |
| | | ATGAGCTGCAAGACCAGCGGCTACACCTTCACCAG |
| | | GTACACCATGCACTGGGTGAAGCA |
| | | GAGGCCCGGCCAGGGCCTGGAGTGGATCGGCTACA |
| | | TCAACCCCAGCAGGGGCTACACCAACTACAACCAG |
| | | AAGTTCAAGGACAAGGCCACCCTGACCACCGACAA |
| | | GAGCAGCAGCACCGCCTACATGCAGCTGAGCAGCC |
| | | TGACCAGCGAGGACAGCGCCGTGTACTACTGCGCC |
| | | AGGTACTACGACGACCACTACTGCCTGGACTACTG |
| | | GGGCCAGGGCACCACCCTGACCGTGAGCAGC |
| 38 | Amino Acid | DIQLTQSPAIMSASPGEKVTMTCRASSSVSYMNWY |
| | | QQKSGTSPKRWIYDTSKVASGVPYRFSGSGSGTSY |
| | | SLTISSMEAEDAATYYCQQWSSNPLTFGAGTKLEL |
| | | KGGGGSGGGGSGGGGSDIKLQQSGAELARPGASVK |
| | | MSCKTSGYTFTRYTMHWVKQRPGQGLEWIGYINPS |
| | | RGYTNYNQKFKDKATLTTDKSSSTAYMQLSSLTSE |
| | | DSAVYYCARYYDDHYCLDYWGQGTTLTVSS |
| 39 | Nucleotide | ATGGATATCCAGATGACCCAGTCCCCGAGCTCCCT |
| | | GTCCGCCTCTGTGGGCGATAGGGTCACCATCACCT |
| | | GCCGTGCCAGTCAGGACATCCGTAATTATCTGAAC |
| | | TGGTATCAACAGAAACCAGGAAAAGCTCCGAAACT |
| | | ACTGATTTACTATACCTCCCGCCTGGAGTCTGGAG |
| | | TCCCTTCTCGCTTCTCTGGTTCTGGTTCTGGGACG |
| | | GATTACACTCTGACCATCAGCAGTCTGCAACCGGA |
| | | AGACTTCGCAACTTATTACTGTCAGCAAGGTAATA |
| | | CTCTGCCGTGGACGTTCGGACAGGGCACCAAGGTG |
| | | GAGATCAAAGGCGGCGGCGGAAGTGGAGGAGGAGG |
| | | CTCAGGCGGAGGAGGGAGCGAGGTTCAGCTGGTGG |
| | | AGTCTGGCGGTGGCCTGGTGCAGCCAGGGGGCTCA |
| | | CTCCGTTTGTCCTGTGCAGCTTCTGGCTACTCCTT |
| | | TACCGGCTACACTATGAACTGGGTGCGTCAGGCCC |
| | | CAGGTAAGGGCCTGGAATGGGTTGCACTGATTAAT |
| | | CCTTATAAAGGTGTTAGTACCTACAACCAGAAGTT |
| | | CAAGGACCGTTTCACTATAAGCGTAGATAAATCCA |
| | | AAAACACAGCCTACCTGCAAATGAACAGCCTGCGT |
| | | GCTGAGGACACTGCCGTCTATTATTGTGCTAGAAG |
| | | CGGATACTACGGCGATAGTGACTGGTATTTTGACG |
| | | TGTGGGGTCAAGGAACCCTGGTCACCGTCTCCTCG |
| 40 | Amino Acid | MDIQMTQSPSSLSASVGDRVTITCRASQDIRNYLN |
| | | WYQQKPGKAPKLLIYYTSRLESGVPSRFSGSGSGT |
| | | DYTLTISSLQPEDFATYYCQQGNTLPWTFGQGTKV |
| | | EIKGGGGSGGGGSGGGGSEVQLVESGGGLVQPGGS |
| | | LRLSCAASGYSFTGYTMNWVRQAPGKGLEWVALIN |
| | | PYKGVSTYNQKFKDRFTISVDKSKNTAYLQMNSLR |
| | | AEDTAVYYCARSGYYGDSDWYFDVWGQGTLVTVSS |
| 41 | Nucleotide | ATGGATATCCAGATGACCCAGTCCCCGAGCTCCCT |
| | | GTCCGCCTCTGTGGGCGATAGGGTCACCATCACCT |
| | | GCCGTGCCAGTCAGGACATCCGTAATTATCTGAAC |
| | | TGGTATCAACAGAAACCAGGAAAAGCTCCGAAACT |
| | | ACTGATTTACTATACCTCCCGCCTGGAGTCTGGAG |
| | | TCCCTTCTCGCTTCTCTGGTTCTGGTTCTGGGACG |
| | | GATTACACTCTGACCATCAGCAGTCTGCAACCGGA |
| | | AGACTTCGCAACTTATTACTGTCAGCAAGGTAATA |
| | | CTCTGCCGTGGACGTTCGGACAGGGCACCAAGGTG |
| | | GAGATCAAAGGCGGCGGCGGAAGTGGAGGAGGAGG |
| | | CTCAGGCGGAGGAGGGAGCGAGGTTCAGCTGGTGG |
| | | AGTCTGGCGGTGGCCTGGTGCAGCCAGGGGGCTCA |
| | | CTCCGTTTGTCCTGTGCAGCTTCTGGCTACTCCTT |
| | | TACCGGCTACACTATGAACTGGGTGCGTCAGGCCC |
| | | CAGGTAAGGGCCTGGAATGGGTTGCACTGATTAAT |
| | | CCTACCAAAGGTGTTAGTACCTACAACCAGAAGTT |
| | | CAAGGACCGTTTCACTATAAGCGTAGATAAATCCA |
| | | AAAACACAGCCTACCTGCAAATGAACAGCCTGCGT |
| | | GCTGAGGACACTGCCGTCTATTATTGTGCTAGAAG |
| | | CGGATACTACGGCGATAGTGACTGGTATTTTGACG |
| | | TGTGGGGTCAAGGAACCCTGGTCACCGTCTCCTCG |
| 42 | Amino Acid | MDIQMTQSPSSLSASVGDRVTITCRASQDIRNYLN |
| | | WYQQKPGKAPKLLIYYTSRLESGVPSRFSGSGSGT |
| | | DYTLTISSLQPEDFATYYCQQGNTLPWTFGQGTKV |
| | | EIKGGGGSGGGGSGGGGSEVQLVESGGGLVQPGGS |
| | | LRLSCAASGYSFTGYTMNWVRQAPGKGLEWVALIN |
| | | PTKGVSTYNQKFKDRFTISVDKSKNTAYLQMNSLR |
| | | AEDTAVYYCARSGYYGDSDWYFDVWGQGTLVTVSS |
| 43 | Nucleotide | ATGGACATCCAGATGACTCAGACCACAAGCTCCCT |
| | | GTCTGCAAGTCTGGGCGACCGGGTGACAATCTCCT |
| | | GCAGAGCCTCTCAGGATATTAGGAACTACCTGAAT |
| | | TGGTATCAGCAGAAACCTGATGGCACAGTCAAGCT |
| | | GCTGATCTACTATACCAGCCGGCTGCACTCAGGCG |
| | | TGCCAAGCAAATTCTCAGGAAGCGGCTCCGGGACT |
| | | GACTACTCCCTGACCATCTCTAACCTGGAGCAGGA |
| | | AGATATTGCTACCTATTTCTGCCAGCAGGGCAATA |
| | | CACTGCCCTGGACTTTTGCCGGAGGCACCAAACTG |
| | | GAGATCAAGGGGGGAGGCGGGAGTGGAGGCGGGGG |
| | | ATCAGGAGGAGGAGGCAGCGGAGGAGGAGGGTCCG |
| | | AGGTCCAGCTGCAGCAGAGCGGACCAGAACTGGTG |
| | | AAGCCCGGAGCAAGTATGAAAATCTCCTGTAAGGC |
| | | CTCAGGATACAGCTTCACCGGCTATACAATGAACT |
| | | GGGTGAAACAGTCCCATGGCAAGAACCTGGAATGG |
| | | ATGGGGCTGATTAATCCTACCAAAGGCGTCAGCAC |
| | | CTATAATCAGAAGTTTAAAGACAAGGCCACACTGA |
| | | CTGTGGATAAGTCTAGTTCAACCGCTTACATGGAG |
| | | CTGCTGTCCCTGACATCTGAAGACAGTGCCGTGTA |
| | | CTATTGTGCTCGGTCTGGCTACTATGGGGACAGTG |
| | | ATTGGTACTTCGATGTCTGGGGACAGGGCACTACC |
| | | CTGACCGTGTTTTCT |
| 44 | Amino Acid | MDIQMTQTTSSLSASLGDRVTISCRASQDIRNYLN |
| | | WYQQKPDGTVKLLIYYTSRLHSGVPSKFSGSGSGT |
| | | DYSLTISNLEQEDIATYFCQQGNTLPWTFAGGTKL |
| | | EIKGGGGSGGGGSGGGGSGGGG |
| | | SEVQLQQSGPELVKPGASMKISCKASGYSFTGYTM |
| | | NWVKQSHGKNLEWMGLINPTKGVSTYNQKFKDKAT |
| | | LTVDKSSSTAYMELLSLTSEDSAVYYCARSGYYGD |
| | | SDWYFDVWGQGTTLTVFS |
| 45 | Nucleotide | AGCGGACAGGTGCTGCTGGAATCCAATATCAAAGT |
| | | CCTGCCCACTTGGTCTACCCCCGTGCAGCCTATGG |
| | | CTCTGATTGTGCTGGGAGGAGTCGCAGGACTGCTG |
| | | CTGTTTATCGGGCTGGGAATTTTCTTTTGCGTGCG |
| | | CTGCCGGCACCGGAGAAGGCAGGCCGAGCGCATGA |
| | | GCCAGATCAAGCGACTGCTGAGCGAGAAGAAAACC |
| | | TGTCAGTGTCCCCATAGATTCCAGAAGACCTGTTC |
| | | ACCCATT |
| 46 | Amino Acid | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 47 | Nucleotide | CTCGAGCTGAGGCCCGAGGCTTCTAGACCTGCTGC |
| | | CGGCGGAGCCGTGCACACCAGAGGCCTGGACTTCG |
| | | CCAGCGACATCTACATCTGGGCCCCTCTGGCCGGC |
| | | ACCTGTGGCGTGCTGCTGCTGAGCCTGGTCATCAC |
| | | CCTGTACTGCAACCACCGGAACCGGCGGAGAGTGT |
| | | GCAAGTGCCCCAGACCCGTGGTCAAGAGCGGCGAC |
| | | AAGCCCAGCCTGAGCGCCAGATACGTG |
| 48 | Amino Acid | LELRPEASRPAAGGAVHTRGLDFASDIYIWAPLAG |
| | | TCGVLLLSLVITLYCNHRNRRRVCKCPRPVVKSGD |
| | | KPSLSARYV |
| 49 | Nucleotide | CTCGAGCTGAGGCCCGAGGCTTCTAGACCTGCTGC |
| | | CGGCGGAGCCGTGCACACCAGAGGCCTGGACTTCG |
| | | CCAGCGACATCTACATCTGGGCCCCTCTGGCCGGC |
| | | ACCTGTGGCGTGCTGCTGCTGAGCCTGGTCATCAC |
| | | CCTGTACCTGTGCTGCAGACGGCGGAGAGTGTGCA |
| | | AGTGCCCCAGACCCGTGGTCAAGAGCGGCGACAAG |
| | | CCCAGCCTGAGCGCCAGATACGTG |
| 50 | Amino Acid | LELRPEASRPAAGGAVHTRGLDFASDIYIWAPLAG |
| | | TCGVLLLSLVITLYLCCRRRRVCKCPRPVVKSGDK |
| | | PSLSARYV |
| 51 | Nucleotide | CTCGAGAAGAAGTCCACCCTGAAGAAACGGGTGTC |
| | | CCGGCTGCCCAGACCCGAGACACAGAAGGGCCCCC |
| | | TGAGCAGCCCTATCACCCTGGGACTGCTGGTGGCC |
| | | GGCGTGCTGGTGCTGCTGGTGTCTCTGGGAGTGGC |
| | | CATCCACCTGTGCTGCCGGCGGAGAAGGGCCTGCA |
| | | AGTGCCCCAGACTGCGGTTCATGAAGCAGTTCTAC |
| | | AAG |
| 52 | Amino Acid | LEKKSTLKKRVSRLPRPETQKGPLSSPITLGLLVA |
| | | GVLVLLVSLGVAIHLCCRRRRACKCPRLRFMKQFY |
| | | K |
| 53 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSSGGGSEGGGSEGGGSEGGG |
| | | QSVLTQPPSASGTPGQRVTISCSGGSSNIGSNYVY |
| | | WYQQLPGTAPKLLIYRNNQRPSGVPDRFSGSKSGT |
| | | SASLAISGLRSEDEADYYCAAWDDSLSGHVVFGGG |
| | | TKLTVL |
| 54 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSSGGGSEGGGSEGGGSEGGG |
| | | QSVLTQPPSASGTPGQRVTISCSGSSSNIGSNYVY |
| | | WYQQLPGTAPKLLIYRNNQRPSGVPDRLSGSKSGT |
| | | SASLAISGLRSEDEADYYCAAWDDSLSAVVFGGGT |
| | | KLTVL |
| 55 | Amino Acid | QVQLQESGGGVVQPGRSLRLSCAASGFTLSSYGMH |
| | | WVRQAPGKGLEWVAVISYDGSNKYYADSVKGRFTI |
| | | SRDNSKNTLYLQMNSLRAEDTAVYYCAKSGWRYYY |
| | | YYGMDVWGQGTLVTVSSGGGSEGGGSEGGGSEGGG |
| | | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGSVFGGG |
| | | TKLTVL |
| 56 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTMVTVSSGGGSEGGGSEGGGSEGGG |
| | | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGPSVVFGG |
| | | GTKLTVL |
| 57 | Amino Acid | QVQLVQSGAEVKKPGASVKVSCKASGYTFTSYGIS |
| | | WVRQAPGQGLEWMGWIGAYNGNTNYAQKLQGRVTM |
| | | TTDTSTSTAYMELRSLRSDDTAVYYCARDLRRYSS |
| | | SWDGPGYWGQGTLVTVSSGGGSEGGGSEGGGSEGG |
| | | GQSALTQPASVSGSPGQSITISCTGTSSDVGGYNY |
| | | VSWYQQHPGKAPKLMIYDVSNRPSGVSNRFSGSKS |
| | | GNTASLTISGLQAEDEADYYCSSYTSSSTRVFGTG |
| | | TKLTVL |
| 58 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSSGGGSEGGGSEGGGSEGGG |
| | | SYVLTQPPSASGTPGQRVTISCSGSSSNIGGNYVY |
| | | WYQQLPGTAPKLLIYRNNQRPSGVPDRFSGSKSGT |
| | | SASLAISGLRSEDEADYYCAAWDDSLSGWVFGGGT |
| | | KLTVL |
| 59 | Amino Acid | QVQLVQSGAEVKKPGSSVKVSCKASGGTFSSYAIS |
| | | WVRQAPGQGLEWMGGIIPIFGTANYAQKFQGRVTI |
| | | TADKSTSTAYMELSSLRSEDTAVYYCARDGGRYSY |
| | | GRSFDYWGQGTLVTVSSGGGSEGGGSEGGGSEGGG |
| | | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGPVVFGG |
| | | GTKLTVL |
| 60 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARPLRPLHY |
| | | YGMDVWGQGTLVTVSSGGGSE |
| | | GGGSEGGGSEGGGQSVLTQPPSASGTPGQRVTISC |
| | | SGSSSNIGSNYVYWYQQLPGTAPKLLIYRNNQRPS |
| | | GVPDRFSGSKSGTSASLAISGLRSEDEADYYCAAW |
| | | DDSLTEGVFGGGTKLTVL |
| 61 | Amino Acid | QVQLVESGGGVVQPGRSLRLSCAASGFTFSSYGMH |
| | | WVRRAPGKGLEWVAVISYDGSNKYYADSVKGRFTI |
| | | SRDNSKNTLYLQMNSLRAEDTAVYYCAKDGGDSGS |
| | | LDYWGQGTLVTVSSGGGSEGGGSEGGGSEGGGDIV |
| | | MTQSPDSLAVSLGERATINCKSSQSVLYSSNNKNY |
| | | LAWYQQKPGQPPKLLIYWASTRESGVPDRFSGSGS |
| | | GTDFTLTISSLQAEDVAVYYCQQYYSTPLTFGGGT |
| | | KLEIK |
| 62 | Amino Acid | EVQLLESGGGLVQPGGSLRLSCAASGFTFSSYWMS |
| | | WVRQAPGKGLEWVANIKQDGSEKYYVDSVKGRFTI |
| | | SRDNAKNSLYLQMNSLRAEDTAVYYCARPAGQLLY |
| | | GMDVWGQGTLVTVSSGGGSEGGGSEGGGSEGGGSY |
| | | VLTQPPSVSVSPGQTARITCSGDALPKQYAYWYQQ |
| | | KPGQAPVLVIYKDSERPSGIPERFSGSSSGTTVTL |
| | | TISGVQAEDEADYYCQSADSSGTWVFGGGTKLTVL |
| 63 | Amino Acid | EVQLLESGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCAKDAADMGA |
| | | FDYWGQGTLVTVSSGGGSEGGGSEGGGSEGGGDIQ |
| | | MTQSPDSLAVSLGERATMNCKSSQSVLYSSNNKNY |
| | | LAWYQQKPGQPPKLLIYWASARESGVPDRFSGSGS |
| | | GTDFTLTISSLQAEDVAVYYCQQYYSTPLTFGQGT |
| | | KVEIK |
| 64 | Amino Acid | EVQLLESGGGLVKPGGSLRLSCAASGFTFSSYSMN |
| | | WVRQAPGKGLEWVSAISGSGGSTYYADSVKGRFTI |
| | | SRDNSKNTLYLQMNSLRAEDTAVYYCARARGYSYG |
| | | SDAFDIWGQGTMVTVSSGGGSEGGGSEGGGSEGGG |
| | | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGVVFGGGT |
| | | KLTVL |
| 65 | Amino Acid | QVQLVQSGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCAKGVPRYYY |
| | | YYGMDVWGQGTMVTVSSGGGSEGGGSEGGGSEGGG |
| | | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGSVFGGG |
| | | TKLTVL |
| 66 | Amino Acid | EVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDRGRRYC |
| | | SGGSCPNAFDIWGQGTMVTVSSGGGSEGGGSEGGG |
| | | SEGGGEIVLTQSPATLSVSPGERATLSCRASQSVS |
| | | SNLAWYQQKPGQAPRLLIYGASTRATGIPARFSGS |
| | | GSGTEFTLTISSLQSEDFAVYYCQQYNNWPPALTF |
| | | GGGTKVEIK |
| 67 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARGGYVWGS |
| | | YRQNSNWFDPWGQGTLVTVSSGGGSEGGGSEGGGS |
| | | EGGGSYVLTQPPSASGTPGQRVTISCSGSSSNIGS |
| | | NYVYWYQQLPGTAPKLLIYRNNQRPSGVPDRFSGS |
| | | KSGTSASLAISGLRSEDEADYYCAAWDDSLSGWVF |
| | | GGGTKLTVL |
| 68 | Amino Acid | QVQLVESGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSSGGGSEGGGSEGGGSEGGG |
| | | QSVLTQPPSASGTPGRRVTISCSGSSSNIGSNYVY |
| | | WYQQLPGTAPKLLIYRNNQRPSGVPDRFSGSKSGT |
| | | SASLAISGLRSEDEADYYCAAWDDSLSGVVFGGGT |
| | | KLTVL |
| 59 | Amino Acid | QVQLVESGGGLVQPGGSLRLSCAASGFTFSSYWMS |
| | | WVRQAPGKGLEWVANIKQDGSEKYYVDSVKGRFTI |
| | | SRDNAKNSLYLQMNSLRAEDTAVYYCARWSHYYDS |
| | | SGLDYWGQGTLVTVSSGGGSEGGGSEGGGSEGGGS |
| | | YVLTQPPSVSVSPGQTARITCSGDALPKQYAYWYQ |
| | | QKPGQAPVLVIYKDSERPSGIPERFSGSSSGTTVT |
| | | LTISGVQAEDEADYYCQSADSSGTWVFGGGTKLTV |
| | | L |
| 70 | Amino Acid | QVQLVESGAEVKKPGSSVKVSCKASGGTFSSYAIS |
| | | WVRQAPGQGLEWMGGIIPIFGTANYAQKFQGRVTI |
| | | TADKSTSTAYMELSSLRSEDTAVYYCARTHLPYSY |
| | | GLGGFDYWGQGTLVTVSSGGGSEGGGSEGGGSEGG |
| | | GQSVLTQPPSVSGAPGQRVTISCTGSSSNIGAGYD |
| | | VHWYQQLPGTAPKLLIYGNSNRPSGVPDRFSGSKS |
| | | GTSASLAITGLQAEDEADYYCQSYDSSLSGPVVFG |
| | | GGTKVTVL |
| 71 | Amino Acid | EVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDPSRDYY |
| | | GSGRFSWFDPWGQGTLVTVSSGGGSEGGGSEGGGS |
| | | EGGGQSVLTQPPSASGTPGQRVTISCSGSSSNIGS |
| | | NYVYWYQQLPGTAPKLLIYRNNQRPSGVPDRFSGS |
| | | KSGTSASLAISGLRSEDEADYYCAAWDDSLSGRGV |
| | | FGGGTKLTVL |
| 72 | Amino Acid | QVQLVQSGAEVKKPGASVKVSCKASGYTFTSYGIS |
| | | WVRQAPGQGLEWMGWISAYNGNTNYAQKLQGRVTM |
| | | TTDTSTSTAYMELRSLRSDDTAVYYCARDIIRYCS |
| | | STSCYRGIDYWGQGTLVTVSSGGGSEGGGSEGGGS |
| | | EGGGQSALTQPASVSGSPGQSITISCTGTSSDVGG |
| | | YNYVSWYQQHPGKAPKLMIYDVSNRPSGVSNRFSG |
| | | SKSGNTASLTISGLQAEDEADYYCSSYTSSSPHVV |
| | | SGGGTKLTVL |
| 73 | Amino Acid | QVQLVESGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCARDGTDYRG |
| | | AFDIWGQGTMVTVSSGGGSEGGGSEGGGSEGGGDI |
| | | QLTQSPDSLAVPLGERATINCKSSQSVLYSSNNKN |
| | | YLAWYQQKPGQPPKLLIYWASTRESGVPDRFSGSG |
| | | SGTDFTLTISSLQAEDVAVYYCQQYYSTPLTFGQG |
| | | TKLEIK |
| 74 | Amino Acid | EVQLVQSGAEVKKPGSSVRVSCKASGGTFSSYAIS |
| | | WVRQAPGQGLEWMGGIIPIFGTANYAQKFQGRVTI |
| | | TADKSTSTAYMELSSLRSEDTAVYYCARGGGRWLH |
| | | SRLDVWGQGTTVTVSSGGGSEGGGSEGGGSEGGGQ |
| | | SVLTQPPSVSGAPGQRVTISCTGSSSNIGAGYDVH |
| | | WYQQFPGTAPKLLIFGNNNRPSGVPDRFSGSKSGT |
| | | SASLAITGLQAEDEANYYCQSYDRSLSGPVVFGGG |
| | | TKLTVL |
| 75 | Amino Acid | EVQLVESGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCAKDAGDLGA |
| | | FDIWGQGTMVTVSSGGGSEGGGSEGGGSEGGGDIV |
| | | MTQSPDSLAVSLGERATINCKSSQSVLYSSNNKNY |
| | | LAWYQQKPGQPPKLLIYWASTRESGVPDRFSGSGS |
| | | GTDFTLTISSLQAEDVAVYYCQQYYSTPLTFGPGT |
| | | KVEIK |
| 76 | Amino Acid | QVQLVQSGGGVVQPGRSLRLSCAASGFTFSSYAMH |
| | | WVRQAPGKGLEWVAVISYDGSNKYYADSVKGRFTI |
| | | SRDNSKNTLYLQMNSLRAEDTAVYYCARGRVYYYD |
| | | SSGYSYWGQGTLVTVSSGGGSEGGGSEGGGSEGGG |
| | | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGREVFGT |
| | | GTKLTVL |
| 77 | Amino Acid | QVQLVESGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARPLYAPRF |
| | | GYGMDVWGQGTLVTVSSGGGSEGGGSEGGGSEGGG |
| | | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGWVFGGGT |
| | | QLTVL |
| 78 | Amino Acid | EVQLLESGGGLVQPGGSLRLSCAASGFTFSSYAMS |
| | | WVRQAPGKGLEWVSAISGSGGSTYYADSVKGRFTI |
| | | SRDNSKNTLYLQMNSLRAEDTAVYYCAKDRGGQRG |
| | | FDYWGQGTLVTVSSGGGSEGGGSEGGGSEGGGQSV |
| | | LTQPPSASGTPGQRVTISCSGSSSNIGSNYVYWYQ |
| | | QLPGTAPKLLIYRNNQRPSGVPDRFSGSKSGTSAS |
| | | LAISGLRSEDEADYYCAAWDDSLSGLVFGGGTKLT |
| | | VL |
| 79 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSSGSTSGSGKPGSGEGSTKG |
| | | QSVLTQPPSASGTPGQRVTISCSGGSSNIGSNYVY |
| | | WYQQLPGTAPKLLIYRNNQRPSGVPDRFSGSKSGT |
| | | SASLAISGLRSEDEADYYCAAWDDSLSGHVVFGGG |
| | | TKLTVL |
| 80 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSSGSTSGSGKPGSGEGSTKG |
| | | QSVLTQPPSASGTPGQRVTISCSGSSSNIGSNYVY |
| | | WYQQLPGTAPKLLIYRNNQRPSGVPDRLSGSKSGT |
| | | SASLAISGLRSEDEADYYCAAWDDSLSAVVFGGGT |
| | | KLTVL |
| 81 | Amino Acid | QVQLQESGGGVVQPGRSLRLSCAASGFTLSSYGMH |
| | | WVRQAPGKGLEWVAVISYDGSNKYYADSVKGRFTI |
| | | SRDNSKNTLYLQMNSLRAEDTAVYYCAKSGWRYYY |
| | | YYGMDVWGQGTLVTVSSGSTSGSGKPGSGEGSTKG |
| | | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGSVFGGG |
| | | TKLTVL |
| 82 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTMVTVSSGSTSGSGKPGSGEGSTKG |
| | | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGPSVVFGG |
| | | GTKLTVL |
| 83 | Amino Acid | QVQLVQSGAEVKKPGASVKVSCKASGYTFTSYGIS |
| | | WVRQAPGQGLEWMGWIGAYNGNTNYAQKLQGRVTM |
| | | TTDTSTSTAYMELRSLRSDDTAVYYCARDLRRYSS |
| | | SWDGPGYWGQGTLVTVSSGSTSGSGKPGSGEGSTK |
| | | GQSALTQPASVSGSPGQSITISCTGTSSDVGGYNY |
| | | VSWYQQHPGKAPKLMIYDVSNRPSGVSNRFSGSKS |
| | | GNTASLTISGLQAEDEADYYCSSYTSSSTRVFGTG |
| | | TKLTVL |
| 84 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSSGSTSGSGKPGSGEGSTKG |
| | | SYVLTQPPSASGTPGQRVTISCSGSSSNIGGNYVY |
| | | WYQQLPGTAPKLLIYRNNQRPSGVPDRFSGSKSGT |
| | | SASLAISGLRSEDEADYYCAAWDDSLSGWVFGGGT |
| | | KLTVL |
| 85 | Amino Acid | QVQLVQSGAEVKKPGSSVKVSCKASGGTFSSYAIS |
| | | WVRQAPGQGLEWMGGIIPIFGTANYAQKFQGRVTI |
| | | TADKSTSTAYMELSSLRSEDTAVYYCARDGGRYSY |
| | | GRSFDYWGQGTLVTVSSGSTSGSGKPGSGEGSTKG |
| | | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGPVVFGG |
| | | GTKLTVL |
| 86 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARPLRPLHY |
| | | YGMDVWGQGTLVTVSSGSTSGSGKPGSGEGSTKGQ |
| | | SVLTQPPSASGTPGQRVTISCSGSSSNIGSNYVYW |
| | | YQQLPGTAPKLLIYRNNQRPSGVPDRFSGSKSGTS |
| | | ASLAISGLRSEDEADYYCAAWDDSLTEGVFGGGTK |
| | | LTVL |
| 87 | Amino Acid | QVQLVESGGGVVQPGRSLRLSCAASGFTFSSYGMH |
| | | WVRRAPGKGLEWVAVISYDGSNKYYADSVKGRFTI |
| | | SRDNSKNTLYLQMNSLRAEDTAVYYCAKDGGDSGS |
| | | LDYWGQGTLVTVSSGSTSGSGKPGSGEGSTKGDIV |
| | | MTQSPDSLAVSLGERATINCKSSQSVLYSSNNKNY |
| | | LAWYQQKPGQPPKLLIYWASTRESGVPDRFSGSGS |
| | | GTDFTLTISSLQAEDVAVYYCQQYYSTPLTFGGGT |
| | | KLEIK |
| 88 | Amino Acid | EVQLLESGGGLVQPGGSLRLSCAASGFTFSSYWMS |
| | | WVRQAPGKGLEWVANIKQDGSEKYYVDSVKGRFTI |
| | | SRDNAKNSLYLQMNSLRAEDTAVYYCARPAGQLLY |
| | | GMDVWGQGTLVTVSSGS |
| | | TSGSGKPGSGEGSTKGSYVLTQPPSVSVSPGQTAR |
| | | ITCSGDALPKQYAYWYQQKPGQAPVLVIYKDSERP |
| | | SGIPERFSGSSSGTTVTLTISGVQAEDEADYYCQS |
| | | ADSSGTWVFGGGTKLTVL |
| 89 | Amino Acid | EVQLLESGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCAKDAADMGA |
| | | FDYWGQGTLVTVSSGSTSGSGKPGSGEGSTKGDIQ |
| | | MTQSPDSLAVSLGERATMNCKSSQSVLYSSNNKNY |
| | | LAWYQQKPGQPPKLLIYWASARESGVPDRFSGSGS |
| | | GTDFTLTISSLQAEDVAVYYCQQYYSTPLTFGQGT |
| | | KVEIK |
| 90 | Amino Acid | EVQLLESGGGLVKPGGSLRLSCAASGFTFSSYSMN |
| | | WVRQAPGKGLEWVSAISGSGGSTYYADSVKGRFTI |
| | | SRDNSKNTLYLQMNSLRAEDTAVYYCARARGYSYG |
| | | SDAFDIWGQGTMVTVSSGSTSGSGKPGSGEGSTKG |
| | | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGVVFGGGT |
| | | KLTVL |
| 91 | Amino Acid | QVQLVQSGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCAKGVPRYYY |
| | | YYGMDVWGQGTMVTVSSGSTSGSGKPGSGEGSTKG |
| | | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGSVFGGG |
| | | TKLTVL |
| 92 | Amino Acid | EVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDRGRRYC |
| | | SGGSCPNAFDIWGQGTMVTVSSGSTSGSGKPGSGE |
| | | GSTKGEIVLTQSPATLSVSPGERATLSCRASQSVS |
| | | SNLAWYQQKPGQAPRLLIYGASTRATGIPARFSGS |
| | | GSGTEFTLTISSLQSEDFAVYYCQQYNNWPPALTF |
| | | GGGTKVEIK |
| 93 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARGGYVWGS |
| | | YRQNSNWFDPWGQGTLVTVSSGSTSGSGKPGSGEG |
| | | STKGSYVLTQPPSASGTPGQRVTISCSGSSSNIGS |
| | | NYVYWYQQLPGTAPKLLIYRNNQRPSGVPDRFSGS |
| | | KSGTSASLAISGLRSEDEADYYCAAWDDSLSGWVF |
| | | GGGTKLTVL |
| 94 | Amino Acid | QVQLVESGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSSGSTSGSGKPGSGEGSTKG |
| | | QSVLTQPPSASGTPGRRVTISCSGSSSNIGSNYVY |
| | | WYQQLPGTAPKLLIYRNNQRPSGVPDRFSGSKSGT |
| | | SASLAISGLRSEDEADYYCAAWDDSLSGVVFGGGT |
| | | KLTVL |
| 95 | Amino Acid | QVQLVESGGGLVQPGGSLRLSCAASGFTFSSYWMS |
| | | WVRQAPGKGLEWVANIKQDGSEKYYVDSVKGRFTI |
| | | SRDNAKNSLYLQMNSLRAEDTAVYYCARWSHYYDS |
| | | SGLDYWGQGTLVTVSSGSTSGSGKPGSGEGSTKGS |
| | | YVLTQPPSVSVSPGQTARITCSGDALPKQYAYWYQ |
| | | QKPGQAPVLVIYKDSERPSGIPERFSGSSSGTTVT |
| | | LTISGVQAEDEADYYCQSADSSGTWVFGGGTKLTV |
| | | L |
| 96 | Amino Acid | QVQLVESGAEVKKPGSSVKVSCKASGGTFSSYAIS |
| | | WVRQAPGQGLEWMGGIIPIFGTANYAQKFQGRVTI |
| | | TADKSTSTAYMELSSLRSEDTAVYYCARTHLPYSY |
| | | GLGGFDYWGQGTLVTVSSGSTSGSGKPGSGEGSTK |
| | | GQSVLTQPPSVSGAPGQRVTISCTGSSSNIGAGYD |
| | | VHWYQQLPGTAPKLLIYGNSNRPSGVPDRFSGSKS |
| | | GTSASLAITGLQAEDEADYYCQSYDSSLSGPVVFG |
| | | GGTKVTVL |
| 97 | Amino Acid | EVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDPSRDYY |
| | | GSGRFSWFDPWGQGTLVTVSSGSTSGSGKPGSGEG |
| | | STKGQSVLTQPPSASGTPGQRVTISCSGSSSNIGS |
| | | NYVYWYQQLPGTAPKLLIYRNNQRPSGVPDRFSGS |
| | | KSGTSASLAISGLRSEDEADYYCAAWDDSLSGRGV |
| | | FGGGTKLTVL |
| 98 | Amino Acid | QVQLVQSGAEVKKPGASVKVSCKASGYTFTSYGIS |
| | | WVRQAPGQGLEWMGWISAYNGNTNYAQKLQGRVTM |
| | | TTDTSTSTAYMELRSLRSDDTAVYYCARDIIRYCS |
| | | STSCYRGIDYWGQGTLVTVSSGSTSGSGKPGSGEG |
| | | STKGQSALTQPASVSGSPGQSITISCTGTSSDVGG |
| | | YNYVSWYQQHPGKAPKLMIYDVSNRPSGVSNRFSG |
| | | SKSGNTASLTISGLQAEDEADYYCSSYTSSSPHVV |
| | | SGGGTKLTVL |
| 99 | Amino Acid | QVQLVESGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCARDGTDYRG |
| | | AFDIWGQGTMVTVSSGSTSGSGKPGSGEGSTKGDI |
| | | QLTQSPDSLAVPLGERATINCKSSQSVLYSSNNKN |
| | | YLAWYQQKPGQPPKLLIYWASTRESGVPDRFSGSG |
| | | SGTDFTLTISSLQAEDVAVYYCQQYYSTPLTFGQG |
| | | TKLEIK |
| 100 | Amino Acid | EVQLVQSGAEVKKPGSSVRVSCKASGGTFSSYAIS |
| | | WVRQAPGQGLEWMGGIIPIFGTANYAQKFQGRVTI |
| | | TADKSTSTAYMELSSLRSEDTAVYYCARGGGRWLH |
| | | SRLDVWGQGTTVTVSSGSTSGSGKPGSGEGSTKGQ |
| | | SVLTQPPSVSGAPGQRVTISCTGSSSNIGAGYDVH |
| | | WYQQFPGTAPKLLIFGNNNRPSGVPDRFSGSKSGT |
| | | SASLAITGLQAEDEANYYCQSYDRSLSGPVVFGGG |
| | | TKLTVL |
| 101 | Amino Acid | EVQLVESGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCAKDAGDLGA |
| | | FDIWGQGTMVTVSSGSTSGSGKPGSGEGSTKGDIV |
| | | MTQSPDSLAVSLGERATINCKSSQSVLYSSNNKNY |
| | | LAWYQQKPGQPPKLLIYWASTRESGVPDRFSGSGS |
| | | GTDFTLTISSLQAEDVAVYYCQQYYSTPLTFGPGT |
| | | KVEIK |
| 102 | Amino Acid | QVQLVQSGGGVVQPGRSLRLSCAASGFTFSSYAMH |
| | | WVRQAPGKGLEWVAVISYDGSNKYYADSVKGRFTI |
| | | SRDNSKNTLYLQMNSLRAEDTAVYYCARGRVYYYD |
| | | SSGYSYWGQGTLVTVSSGSTSGSGKPGSGEGSTKG |
| | | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGREVFGT |
| | | GTKLTVL |
| 103 | Amino Acid | QVQLVESGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARPLYAPRF |
| | | GYGMDVWGQGTLVTVSSGSTSGSGKPGSGEGSTKG |
| | | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGWVFGGGT |
| | | QLTVL |
| 104 | Amino Acid | EVQLLESGGGLVQPGGSLRLSCAASGFTFSSYAMS |
| | | WVRQAPGKGLEWVSAISGSGGSTYYADSVKGRFTI |
| | | SRDNSKNTLYLQMNSLRAEDTAVYYCAKDRGGQRG |
| | | FDYWGQGTLVTVSSGSTSGSGKPGSGEGSTKGQSV |
| | | LTQPPSASGTPGQRVTISCSGSSSNIGSNYVYWYQ |
| | | QLPGTAPKLLIYRNNQRPSGVPDRFSGSKSGTSAS |
| | | LAISGLRSEDEADYYCAAWDDSLSGLVFGGGTKLT |
| | | VL |
| 105 | Amino Acid | EVKLVESGGGLVQPGGSLKLSCTTSGFTFSSYGLS |
| | | WIRQTPDKRLELVASINRNGDNTYYTDSVKGRFTI |
| | | SRHDAKNTLNLQMNSLKSEDTAMYYCTRGGFSYWG |
| | | QGTLVTVSAGSTSGSGKPGSGEGSTKGDIVLTQSP |
| | | AIMSSSPGEKVTMTCSASSSVSYMYWYQQRPGSSP |
| | | RLLIYDTSKLASGVPVRFSGSVSGTSYSLTISRME |
| | | SEDAATYYCQQWSGFPPITFGAGTKLELK |
| 106 | Amino Acid | EVKLVESGGGLVQPGGSLKLSCTTSGFTFSSYGLS |
| | | WIRQTPDKRLELVASINRNGDNTYYTDSVRGRFTI |
| | | SRDNAKNTLNLQMSSLKSEDTAMYYCTRGGFSYWG |
| | | QGTLVTVSAGSTSGSGKPGSGEGSTKGDIVLTQSP |
| | | ATMSASPGEKVTLTCSASPGVTYMYWYQQKPGSSP |
| | | RLLIYDTSNLASGVPLRFSGSGSGTSYSLTISRTE |
| | | AEDAATYYCQQWSGYPPITFGAGTKLELR |
| 107 | Amino Acid | EVKLVESGGGLVQPGGSLKLSCTTSGFTFSSYGLS |
| | | WIRQTPDKRLELVASINRNGDNAYYIDSVKGRFTI |
| | | SRDNAKNALNLQMSSLKSEDTAMYYCTRGGFSHWG |
| | | QGTLVTVAAGSTSGSGKPGSGEGSTKGQIVLTQSP |
| | | AIMSASPGEKVTMTCSASSSVSYMYWYQQKPGSSP |
| | | RLLIYDTSNLASGVPVRFSGSGSGTSYSLIISRME |
| | | AEDAATYYCQQWSGYPPITFGAGTKLELK |
| 108 | Amino Acid | EVKLVESGGGLVQPGGSLKLSCAASGFTFSSYGLS |
| | | WVRQTPDKGLELVASINRNGGNTYYTDSVKGRFTI |
| | | SRDNAKNTLNLQMSSLKSEDTAMYYCARGGFTYWG |
| | | QGTLVTVSAGSTSGSGKPGSGEGSTKGQIVLTQSP |
| | | AIMSAFPGEKVTMTCSASSSVGYMYWYQQKPGSSP |
| | | RLLIYDTSNLASGVPVRFSGSGSGTAYSLTISRME |
| | | AEDAATYYCQQWSGYPPITFGAGTKLELK |
| 109 | Amino Acid | QVQLKESGGGLVRPGGSLKLSCAASGFTFSNYGMS |
| | | WIRQTPDKNLELVASINTSGGNTYYPDSVKGRFTI |
| | | SRDNARATLNLQMSNLKSEDTAIYYCTRGGFTHWG |
| | | QGTLVAVSAGSTSGSGKPGSGEGSTKGQIVLTQSP |
| | | AIMSASPGEKVTMTCSASSSVSYMYWYQQKPGSSP |
| | | RLLIYDTSNLASGVPVRFSGSGSGTSYFLTISRME |
| | | AEDAATYYCQQWTGYPPITFGAGTKLELR |
| 110 | Amino Acid | QVQLKESGGGLVQPGGSLKLSCAASGFPFSSYGLS |
| | | WVRQTPDKRLELVATINRNGDSAYYPDSVKGRFTI |
| | | SRDDAKNTLYLQMSSLKSEDTAMYYCTRGGFAYWG |
| | | QGTLVTVSAGSTSGSGKPGSGEGSTKGQIVLTQSP |
| | | AIMSASPGEKVTMTCSGSSSVGYMYWYQQKPGFSP |
| | | RLLIYDTSNLASGVPVRFSGSGSGTSYSLTISRME |
| | | AEDAATYYCQQWSGYPPITFGAGTKLELK |
| 111 | Amino Acid | EVKLVESGGGLVRPGGSLKLSCAASGFTFSNYGMS |
| | | WIRQTPDKNLELVASINTSGGNTYYPDSVKGRFTI |
| | | SRDNARATLNLQMSNLKSEDTAIYYCTRGGFTHWG |
| | | QGTLVTVSAGSTSGSGKPGSGEGSTKGDIVLTQSP |
| | | AIMSASPGEKVTMTCSASSSVAFMYWYQQKPGSSP |
| | | RLLIYDTSKLASGVPVRFSGSGSGTSYSLTISRME |
| | | AEDAATYYCQQWSGYPPITFGAGTKLELK |
| 112 | Amino Acid | QVQLKESGGGLVQPGGSLKLSCTTSGFTFSSYGLS |
| | | WIRQTPDKRLELVASINRNGDNTYYTDSVRGRFTI |
| | | SRDNAKSTLNLQMSSLKSEDTAMYYCTRGGFSYWG |
| | | QGTLVTVSAGSTSGSGKPGSGEGSTKGQIVLTQSP |
| | | PLMSASPGEKVTMTCSASSSVGYMYWFQQRPGSSP |
| | | RLLIYDTYNLASGVPVRFSGSGSGTSYSLTISRLE |
| | | AEDAATYYCQQWSGYPPITFGAGTKLELR |
| 113 | Amino Acid | EVKLVESGGGLVQPGGSLKLSCAASGFTFSSYGLS |
| | | WVRQTPDKRLELVASVNRNGGNTYYTDSVKGRFTI |
| | | SRDNAKNTLNLQMSSLKSEDTAMYYCARGGFTYWG |
| | | QGTLVTVSAGSTSGSGKPGSGEGSTKGQIVLTQSP |
| | | AIMSASPGEKVTMTCSASSSVGYMYWYQQKPGSSP |
| | | RLLIYDTSHLASGVPVRFSGSGSGTSYSLTISRME |
| | | AEDAATYYCQQWSGYPPITFGAGTKLELK |
| 114 | Amino Acid | EVKLVESGGGLVQPGGSLKLSCAASGFPFSSYGLS |
| | | WVRQTPDKRLELVATINRNGDSAYYPDSVKGRFTI |
| | | SRDDAKNTLYLQMSSLKSEDTAMYYCTRGGFAYWG |
| | | QGTLVTVSAGSTSGSGKPGSGEGSTKGQIVLTQSP |
| | | VIMSASPGEKVTMTCSASSSVGYMYWYQQRPGSSP |
| | | RLLIYDTSNLASGAPVRFSGSGSGTSYSLTISRME |
| | | AEDAATYYCQQWSGYPPITFGAGTKLELK |
| 115 | Amino Acid | EVKLVESGGGLVQPGGSLKLSCTTSGFTFSSYGLS |
| | | WIRQTPDKRLELVASINRNGDNTYYTDSVKGRFTI |
| | | SRDNAKNTLNLQMSSLKSEDTAMYYCTRGGFSYWG |
| | | QGTLVTVSAGSTSGSGKPGSGEGSTKGQIVLTQSP |
| | | AIMSASPGEKVTMTCSASSSVSYMYWYQQKPGSSP |
| | | RLLIYDTSNLASGVPVRFSGSGSGTSYSLTISRME |
| | | AEDAATYYCQQWTGYPPITFGAGTKLELK |
| 116 | Amino Acid | EVKLVESGGVLVQPGGSLKLSCAASGFTFSSYGMS |
| | | WVRQTPDKRLELVASINKNGGSTYYPDSVKGRFTI |
| | | SRDNAKTTVYLQMSSLKSEDTAMYYCTRGGFAYWG |
| | | QGTLVTVSAGSTSGSGKPGSGEGSTKGQIVLTQSP |
| | | AIMSASPGEKVTLTCSASSSVGYMYWYQQRPGSSP |
| | | RLLIYDTSNLPSGVPVRFSGSGSGTSYSLTISRME |
| | | AEDAATYYCQQWSGYPPITFGAGTKLELK |
| 117 | Amino Acid | QLQLQESGGGLVQAGGSLRLSCAASGRTGSSYAMG |
| | | WFRQAPGKEREFVAAITWSGGITAYADSVKGRFTI |
| | | SRDNAKNTVYLQMNSLKPEDTAVYCCAAGVTGSPS |
| | | FDSWGQGTQVTVSS |
| 118 | Amino Acid | QVQLQESGGGLVQAGGSLRLSCAASGRTFSSYAMG |
| | | WFRQAPGKEREFVAAISGSGGSIYYGDSVKGRFTI |
| | | SRDNAKNTMYLQMNRLKPEDTAVYYCAAGPLGSPD |
| | | FDSWGQGTQVTVSS |
| 119 | Amino Acid | EVQVVESGGGLVQPGGSLRLSCVASGRTFSSYAMG |
| | | WFRQAPGKEREFVAAISGSGGSIYYGDSVKGRFTI |
| | | SRDNAKNTMYLQMNRLKPEDTAVYYCAAGPLGSPD |
| | | FDSWGQGTQVTVSS |
| 120 | Amino Acid | KVQLVESGGGLVQAGGSLRLSCAASGRTGSSYAMG |
| | | WFRQAPGKEREFVAAITWSGGITAYADSVKGRFTI |
| | | SRDNAKNTVYLQMNSLKPEDTAVYCCAAGVTGSPS |
| | | FDSWGQGTQVTVSS |
| 121 | Amino Acid | EVQLVESGGGLVQAGGSLRLSCAASGRTFSSYAMG |
| | | WFRQAPGKEREFVAAISGSGGVTFYAHSVKGRFTI |
| | | SRDNAKNTVYLQMNSLKPEDTAVYSCAGGAHGSPD |
| | | FGSWGQGTQVTVSS |
| 122 | Amino Acid | QVQLVESGGGLVQPGGSLRLSCAVSRNIASLYRVD |
| | | WYRQAPGKQRELVAGRTSGGTTTYLDAVEGRFTIS |
| | | RDNVKDTVYLQMNSLTPEDTAVYYCHAHDHWRDSW |
| | | GQGTQVTVSS |
| 123 | Amino Acid | QVQLVESGGDLVQPGGSLRLSCAASGSIGSIYAMG |
| | | WYRQAPGRQRELVATTTSGGTTNYADSVKGRFTIA |
| | | GDNAKNTVFLQMNSLRPEDTAVYYCKIQTHWYVYW |
| | | GQGTQVTVSS |
| 124 | Amino Acid | EVQLVESGGGLVQPGGSLRLSCAASRNIFSLYRVD |
| | | WYRQAPGKQRELVAGSTSGGTTTYADAVKGRFTIS |
| | | TDNVKDTVYLQMNSLTPEDTAVYYCHAHDHWRDSW |
| | | GQGTQVTVSS |
| 125 | Amino Acid | QVQLVESGGGWVHPGGSLRLSCAASRNIFSMYRVD |
| | | WYRQAPGKQRELVAGITSGGTTSYADAVKGRFTIS |
| | | TDNVKDTVYLQMNSVTPEDTAVYYCHAHDHWRDSW |
| | | GQGTQVTVSS |
| 126 | Amino Acid | EVQVQESGGDLVQPGGSLRLSCAASGSIGSIYRKG |
| | | WYRQAPGSQRELVATITSAGTTNYADSVKGRFTIS |
| | | RDNAKNTVYLQMNSLRPEDTAVYYCNFQTHWYVYW |
| | | GQGTQVTVSS |
| 127 | Amino Acid | EVRLVESGGGLVQPGGSLRLSCAVSKNIFSIYRVD |
| | | WYHQAPGKQRELVAGWTSGGSTSYADAVKGRFTIS |
| | | TDNVKDTVYLQMNSLTPEDTAVYYCHAHDHWRDYW |
| | | GQGTQVTVSS |
| 128 | Amino Acid | EVKLVESGGGLVQPGGSLKLSCTTSGFTFSSYGLS |
| | | WIRQTPDKRLELVASINRNGDNTYYTDSVKGRFTI |
| | | SRHDAKNTLNLQMNSLKSEDTAMYYCTRGGFSYWG |
| | | QGTLVTVSA |
| 129 | Amino Acid | DIVLTQSPAIMSSSPGEKVTMTCSASSSVSYMYWY |
| | | QQRPGSSPRLLIYDTSKLASGVPVRFSGSVSGTSY |
| | | SLTISRMESEDAATYYCQQWSGFPPITFGAGTKLE |
| | | LK |
| 130 | Amino Acid | EVKLVESGGGLVQPGGSLKLSCTTSGFTFSSYGLS |
| | | WIRQTPDKRLELVASINRNGDNTYYTDSVRGRFTI |
| | | SRDNAKNTLNLQMSSLKSEDTAMYYCTRGGFSYWG |
| | | QGTLVTVSA |
| 131 | Amino Acid | DIVLTQSPATMSASPGEKVTLTCSASPGVTYMYWY |
| | | QQKPGSSPRLLIYDTSNLASGVPLRFSGSGSGTSY |
| | | SLTISRTEAEDAATYYCQQWSGYPPITFGAGTKLE |
| | | LR |
| 132 | Amino Acid | EVKLVESGGGLVQPGGSLKLSCTTSGFTFSSYGLS |
| | | WIRQTPDKRLELVASINRNGDNAYYIDSVKGRFTI |
| | | SRDNAKNALNLQMSSLKSEDTAMYYCTRGGFSHWG |
| | | QGTLVTVAA |
| 133 | Amino Acid | QIVLTQSPAIMSASPGEKVTMTCSASSSVSYMYWY |
| | | QQKPGSSPRLLIYDTSNLASGVPVRFSGSGSGTSY |
| | | SLIISRMEAEDAATYYCQQWSGYPPITFGAGTKLE |
| | | LK |
| 134 | Amino Acid | EVKLVESGGGLVQPGGSLKLSCAASGFTFSSYGLS |
| | | WVRQTPDKGLELVASINRNGGNTYYTDSVKGRFTI |
| | | SRDNAKNTLNLQMSSLKSEDTAMYYCARGGFTYWG |
| | | QGTLVTVSA |
| 135 | Amino Acid | QIVLTQSPAIMSAFPGEKVTMTCSASSSVGYMYWY |
| | | QQKPGSSPRLLIYDTSNLASGVPVRFSGSGSGTAY |
| | | SLTISRMEAEDAATYYCQQWSGYPPITFGAGTKLE |
| | | LK |
| 136 | Amino Acid | QVQLKESGGGLVRPGGSLKLSCAASGFTFSNYGMS |
| | | WIRQTPDKNLELVASINTSGGNTYYPDSVKGRFTI |
| | | SRDNARATLNLQMSNLKSEDTAIYYCTRGGFTHWG |
| | | QGTLVAVSA |
| 137 | Amino Acid | QIVLTQSPAIMSASPGEKVTMTCSASSSVSYMYWY |
| | | QQKPGSSPRLLIYDTSNLASGVPVRFSGSGSGTSY |
| | | FLTISRMEAEDAATYYCQQWTGYPPITFGAGTKLE |
| | | LR |
| 138 | Amino Acid | QVQLKESGGGLVQPGGSLKLSCAASGFPFSSYGLS |
| | | WVRQTPDKRLELVATINRNGDSAYYPDSVKGRFTI |
| | | SRDDAKNTLYLQMSSLKSEDTAMYYCTRGGFAYWG |
| | | QGTLVTVSA |
| 139 | Amino Acid | QIVLTQSPAIMSASPGEKVTMTCSGSSSVGYMYWY |
| | | QQKPGFSPRLLIYDTSNLASGVPVRFSGSGSGTSY |
| | | SLTISRMEAEDAATYYCQQWSGYPPITFGAGTKLE |
| | | LK |
| 140 | Amino Acid | EVKLVESGGGLVRPGGSLKLSCAASGFTFSNYGMS |
| | | WIRQTPDKNLELVASINTSGGNTYYPDSVKGRFTI |
| | | SRDNARATLNLQMSNLKSEDTAIYYCTRGGFTHWG |
| | | QGTLVTVSA |
| 141 | Amino Acid | DIVLTQSPAIMSASPGEKVTMTCSASSSVAFMYWY |
| | | QQKPGSSPRLLIYDTSKLASGVPVRFSGSGSGTSY |
| | | SLTISRMEAEDAATYYCQQWSGYPPITFGAGTKLE |
| | | LK |
| 142 | Amino Acid | QVQLKESGGGLVQPGGSLKLSCTTSGFTFSSYGLS |
| | | WIRQTPDKRLELVASINRNGDNTYYTDSVRGRFTI |
| | | SRDNAKSTLNLQMSSLKSEDTAMYYCTRGGFSYWG |
| | | QGTLVTVSA |
| 143 | Amino Acid | QIVLTQSPPLMSASPGEKVTMTCSASSSVGYMYWF |
| | | QQRPGSSPRLLIYDTYNLASGVPVRFSGSGSGTSY |
| | | SLTISRLEAEDAATYYCQQWSGYPPITFGAGTKLE |
| | | LR |
| 144 | Amino Acid | EVKLVESGGGLVQPGGSLKLSCAASGFTFSSYGLS |
| | | WVRQTPDKRLELVASVNRNGGNTYYTDSVKGRFTI |
| | | SRDNAKNTLNLQMSSLKSEDTAMYYCARGGFTYWG |
| | | QGTLVTVSA |
| 145 | Amino Acid | QIVLTQSPAIMSASPGEKVTMTCSASSSVGYMYWY |
| | | QQKPGSSPRLLIYDTSHLASGVPVRFSGSGSGTSY |
| | | SLTISRMEAEDAATYYCQQWSGYPPITFGAGTKLE |
| | | LK |
| 146 | Amino Acid | EVKLVESGGGLVQPGGSLKLSCAASGFPFSSYGLS |
| | | WVRQTPDKRLELVATINRNGDSAYYPDSVKGRFTI |
| | | SRDDAKNTLYLQMSSLKSEDTAMYYCTRGGFAYWG |
| | | QGTLVTVSA |
| 147 | Amino Acid | QIVLTQSPVIMSASPGEKVTMTCSASSSVGYMYWY |
| | | QQRPGSSPRLLIYDTSNLASGAPVRFSGSGSGTSY |
| | | SLTISRMEAEDAATYYCQQWSGYPPITFGAGTKLE |
| | | LK |
| 148 | Amino Acid | EVKLVESGGGLVQPGGSLKLSCTTSGFTFSSYGLS |
| | | WIRQTPDKRLELVASINRNGDNTYYTDSVKGRFTI |
| | | SRDNAKNTLNLQMSSLKSEDTAMYYCTRGGFSYWG |
| | | QGTLVTVSA |
| 149 | Amino Acid | QIVLTQSPAIMSASPGEKVTMTCSASSSVSYMYWY |
| | | QQKPGSSPRLLIYDTSNLASGVPVRFSGSGSGTSY |
| | | SLTISRMEAEDAATYYCQQWTGYPPITFGAGTKLE |
| | | LK |
| 150 | Amino Acid | EVKLVESGGVLVQPGGSLKLSCAASGFTFSSYGMS |
| | | WVRQTPDKRLELVASINKNGGSTYYPDSVKGRFTI |
| | | SRDNAKTTVYLQMSSLKSEDTAMYYCTRGGFAYWG |
| | | QGTLVTVSA |
| 151 | Amino Acid | QIVLTQSPAIMSASPGEKVTLTCSASSSVGYMYWY |
| | | QQRPGSSPRLLIYDTSNLPSGVPVRFSGSGSGTSY |
| | | SLTISRMEAEDAATYYCQQWSGYPPITFGAGTKLE |
| | | LK |
| 152 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSS |
| 153 | Amino Acid | QSVLTQPPSASGTPGQRVTISCSGGSSNIGSNYVY |
| | | WYQQLPGTAPKLLIYRNNQRPSGVPDRFSGSKSGT |
| | | SASLAISGLRSEDEADYYCAAWDDSLSGHVVFGGG |
| | | TKLTVL |
| 154 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSS |
| 155 | Amino Acid | QSVLTQPPSASGTPGQRVTISCSGSSSNIGSNYVY |
| | | WYQQLPGTAPKLLIYRNNQRPSGVPDRLSGSKSGT |
| | | SASLAISGLRSEDEADYYCAAWDDSLSAVVFGGGT |
| | | KLTVL |
| 156 | Amino Acid | QVQLQESGGGVVQPGRSLRLSCAASGFTLSSYGMH |
| | | WVRQAPGKGLEWVAVISYDGSNKYYADSVKGRFTI |
| | | SRDNSKNTLYLQMNSLRAEDTAVYYCAKSGWRYYY |
| | | YYGMDVWGQGTLVTVSS |
| 157 | Amino Acid | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGSVFGGG |
| | | TKLTVL |
| 158 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTMVTVSS |
| 159 | Amino Acid | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGPSVVFGG |
| | | GTKLTVL |
| 160 | Amino Acid | QVQLVQSGAEVKKPGASVKVSCKASGYTFTSYGIS |
| | | WVRQAPGQGLEWMGWIGAYNGNTNYAQKLQGRVTM |
| | | TTDTSTSTAYMELRSLRSDDTAVYYCARDLRRYSS |
| | | SWDGPGYWGQGTLVTVSS |
| 161 | Amino Acid | QSALTQPASVSGSPGQSITISCTGTSSDVGGYNYV |
| | | SWYQQHPGKAPKLMIYDVSNRPSGVSNRFSGSKSG |
| | | NTASLTISGLQAEDEADYYCSSYTSSSTRVFGTGT |
| | | KLTVL |
| 162 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSS |
| 163 | Amino Acid | SYVLTQPPSASGTPGQRVTISCSGSSSNIGGNYVY |
| | | WYQQLPGTAPKLLIYRNNQRPSGVPDRFSGSKSGT |
| | | SASLAISGLRSEDEADYYCAAWDDSLSGWVFGGGT |
| | | KLTVL |
| 164 | Amino Acid | QVQLVQSGAEVKKPGSSVKVSCKASGGTFSSYAIS |
| | | WVRQAPGQGLEWMGGIIPIFGTANYAQKFQGRVTI |
| | | TADKSTSTAYMELSSLRSEDTAVYYCARDGGRYSY |
| | | GRSFDYWGQGTLVTVSS |
| 165 | Amino Acid | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGPVVFGG |
| | | GTKLTVL |
| 166 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARPLRPLHY |
| | | YGMDVWGQGTLVTVSS |
| 167 | Amino Acid | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLTEGVFGGGT |
| | | KLTVL |
| 168 | Amino Acid | QVQLVESGGGVVQPGRSLRLSCAASGFTFSSYGMH |
| | | WVRRAPGKGLEWVAVISYDGSNKYYADSVKGRFTI |
| | | SRDNSKNTLYLQMNSLRAEDTAVYYCAKDGGDSGS |
| | | LDYWGQGTLVTVSS |
| 169 | Amino Acid | DIVMTQSPDSLAVSLGERATINCKSSQSVLYSSNN |
| | | KNYLAWYQQKPGQPPKLLIYWASTRESGVPDRFSG |
| | | SGSGTDFTLTISSLQAEDVAVYYCQQYYSTPLTFG |
| | | GGTKLEIK |
| 170 | Amino Acid | EVQLLESGGGLVQPGGSLRLSCAASGFTFSSYWMS |
| | | WVRQAPGKGLEWVANIKQDGSEKYYVDSVKGRFTI |
| | | SRDNAKNSLYLQMNSLRAEDTAVYYCARPAGQLLY |
| | | GMDVWGQGTLVTVSS |
| 171 | Amino Acid | SYVLTQPPSVSVSPGQTARITCSGDALPKQYAYWY |
| | | QQKPGQAPVLVIYKDSERPSGIPERFSGSSSGTTV |
| | | TLTISGVQAEDEADYYCQSADSSGTWVFGGGTKLT |
| | | VL |
| 172 | Amino Acid | EVQLLESGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCAKDAADMGA |
| | | FDYWGQGTLVTVSS |
| 173 | Amino Acid | DIQMTQSPDSLAVSLGERATMNCKSSQSVLYSSNN |
| | | KNYLAWYQQKPGQPPKLLIYWASARESGVPDRFSG |
| | | SGSGTDFTLTISSLQAEDVAVYYCQQYYSTPLTFG |
| | | QGTKVEIK |
| 174 | Amino Acid | EVQLLESGGGLVKPGGSLRLSCAASGFTFSSYSMN |
| | | WVRQAPGKGLEWVSAISGSGGSTYYADSVKGRFTI |
| | | SRDNSKNTLYLQMNSLRAEDTAVYYCARARGYSYG |
| | | SDAFDIWGQGTMVTVSS |
| 175 | Amino Acid | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGVVFGGGT |
| | | KLTVL |
| 176 | Amino Acid | QVQLVQSGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCAKGVPRYYY |
| | | YYGMDVWGQGTMVTVSS |
| 177 | Amino Acid | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGSVFGGG |
| | | TKLTVL |
| 178 | Amino Acid | EVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDRGRRYC |
| | | SGGSCPNAFDIWGQGTMVTVSS |
| 179 | Amino Acid | EIVLTQSPATLSVSPGERATLSCRASQSVSSNLAW |
| | | YQQKPGQAPRLLIYGASTRATGIPARFSGSGSGTE |
| | | FTLTISSLQSEDFAVYYCQQYNNWPPALTFGGGTK |
| | | VEIK |
| 180 | Amino Acid | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARGGYVWGS |
| | | YRQNSNWFDPWGQGTLVTVSS |
| 181 | Amino Acid | SYVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGWVFGGGT |
| | | KLTVL |
| 182 | Amino Acid | QVQLVESGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDILRLSV |
| | | SSGMDVWGQGTLVTVSS |
| 183 | Amino Acid | QSVLTQPPSASGTPGRRVTISCSGSSSNIGSNYVY |
| | | WYQQLPGTAPKLLIYRNNQRPSGVPDRFSGSKSGT |
| | | SASLAISGLRSEDEADYYCAAWDDSLSGVVFGGGT |
| | | KLTVL |
| 184 | Amino Acid | QVQLVESGGGLVQPGGSLRLSCAASGFTFSSYWMS |
| | | WVRQAPGKGLEWVANIKQDGSEKYYVDSVKGRFTI |
| | | SRDNAKNSLYLQMNSLRAEDTAVYYCARWSHYYDS |
| | | SGLDYWGQGTLVTVSS |
| 185 | Amino Acid | SYVLTQPPSVSVSPGQTARITCSGDALPKQYAYWY |
| | | QQKPGQAPVLVIYKDSERPSGIPERFSGSSSGTTV |
| | | TLTISGVQAEDEADYYCQSADSSGTWVFGGGTKLT |
| | | VL |
| 186 | Amino Acid | QVQLVESGAEVKKPGSSVKVSCKASGGTFSSYAIS |
| | | WVRQAPGQGLEWMGGIIPIFGTANYAQKFQGRVTI |
| | | TADKSTSTAYMELSSLRSEDTAVYYCARTHLPYSY |
| | | GLGGFDYWGQGTLVTVSS |
| 187 | Amino Acid | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGPVVFGG |
| | | GTKVTVL |
| 188 | Amino Acid | EVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARDPSRDYY |
| | | GSGRFSWFDPWGQGTLVTVSS |
| 189 | Amino Acid | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGRGVFGGG |
| | | TKLTVL |
| 190 | Amino Acid | QVQLVQSGAEVKKPGASVKVSCKASGYTFTSYGIS |
| | | WVRQAPGQGLEWMGWISAYNGNTNYAQKLQGRVTM |
| | | TTDTSTSTAYMELRSLRSDDTAVYYCARDIIRYCS |
| | | STSCYRGIDYWGQGTLVTVSS |
| 191 | Amino Acid | QSALTQPASVSGSPGQSITISCTGTSSDVGGYNYV |
| | | SWYQQHPGKAPKLMIYDVSNRPSGVSNRFSGSKSG |
| | | NTASLTISGLQAEDEADYYCSSYTSSSPHVVSGGG |
| | | TKLTVL |
| 192 | Amino Acid | QVQLVESGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCARDGTDYRG |
| | | AFDIWGQGTMVTVSS |
| 193 | Amino Acid | DIQLTQSPDSLAVPLGERATINCKSSQSVLYSSNN |
| | | KNYLAWYQQKPGQPPKLLIYWASTRESGVPDRFSG |
| | | SGSGTDFTLTISSLQAEDVAVYYCQQYYSTPLTFG |
| | | QGTKLEIK |
| 194 | Amino Acid | EVQLVQSGAEVKKPGSSVRVSCKASGGTFSSYAIS |
| | | WVRQAPGQGLEWMGGIIPIFGTANYAQKFQGRVTI |
| | | TADKSTSTAYMELSSLRSEDTAVYYCARGGGRWLH |
| | | SRLDVWGQGTTVTVSS |
| 195 | Amino Acid | QSVLTQPPSVSGAPGQRVTISCTGSSSNIGAGYDV |
| | | HWYQQFPGTAPKLLIFGNNNRPSGVPDRFSGSKSG |
| | | TSASLAITGLQAEDEANYYCQSYDRSLSGPVVFGG |
| | | GTKLTVL |
| 196 | Amino Acid | EVQLVESGGGVVQPGRSLRLSCAASGFTFSSYGMH |
| | | WVRQAPGKGLEWVAVISYDGSNKYYADSVKGRFTI |
| | | SRDNSKNTLYLQMNSLRAEDTAVYYCAKDAGDLGA |
| | | FDIWGQGTMVTVSS |
| 197 | Amino Acid | DIVMTQSPDSLAVSLGERATINCKSSQSVLYSSNN |
| | | KNYLAWYQQKPGQPPKLLIYWASTRESGVPDRFSG |
| | | SGSGTDFTLTISSLQAEDVAVYYCQQYYSTPLTFG |
| | | PGTKVEIK |
| 198 | Amino Acid | QVQLVQSGGGVVQPGRSLRLSCAASGFTFSSYAMH |
| | | WVRQAPGKGLEWVAVISYDGSNKYYADSVKGRFTI |
| | | SRDNSKNTLYLQMNSLRAEDTAVYYCARGRVYYYD |
| | | SSGYSYWGQGTLVTVSS |
| 199 | Amino Acid | QSVLTQPPSVSGAPGQRVTISCTGSSSNIGAGYDV |
| | | HWYQQLPGTAPKLLIYGNSNRPSGVPDRFSGSKSG |
| | | TSASLAITGLQAEDEADYYCQSYDSSLSGREVFGT |
| | | GTKLTVL |
| 200 | Amino Acid | QVQLVESGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARPLYAPRF |
| | | GYGMDVWGQGTLVTVSS |
| 201 | Amino Acid | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGWVFGGGT |
| | | QLTVL |
| 202 | Amino Acid | EVQLLESGGGLVQPGGSLRLSCAASGFTFSSYAMS |
| | | WVRQAPGKGLEWVSAISGSGGSTYYADSVKGRFTI |
| | | SRDNSKNTLYLQMNSLRAEDTAVYYCAKDRGGQRG |
| | | FDYWGQGTLVTVSS |
| 203 | Amino Acid | QSVLTQPPSASGTPGQRVTISCSGSSSNIGSNYVY |
| | | WYQQLPGTAPKLLIYRNNQRPSGVPDRFSGSKSGT |
| | | SASLAISGLRSEDEADYYCAAWDDSLSGLVFGGGT |
| | | KLTVL |
| 204 | Amino Acid | SYAIS |
| 205 | Amino Acid | GIIPIFGTANYAQKFQG |
| 206 | Amino Acid | DGGRYSYGRSFDY |
| 207 | Amino Acid | TGSSSNIGAGYDVH |
| 208 | Amino Acid | GNSNRPS |
| 209 | Amino Acid | QSYDSSLSGPVV |
| 210 | Amino Acid | SYAIS |
| 211 | Amino Acid | GIIPIFGTANYAQKFQG |
| 212 | Amino Acid | DILRLSVSSGMDV |
| 213 | Amino Acid | SGGSSNIGSNYVY |
| 214 | Amino Acid | RNNQRPS |
| 215 | Amino Acid | AAWDDSLSGHVV |
| 216 | Amino Acid | SYAIS |
| 217 | Amino Acid | GIIPIFGTANYAQKFQG |
| 218 | Amino Acid | DILRLSVSSGMDV |
| 219 | Amino Acid | SGSSSNIGGNYVY |
| 220 | Amino Acid | RNNQRPS |
| 221 | Amino Acid | AAWDDSLSGWV |
| 222 | Amino Acid | SYAIS |
| 223 | Amino Acid | GIIPIFGTANYAQKFQG |
| 224 | Amino Acid | DILRLSVSSGMDV |
| 225 | Amino Acid | SGSSSNIGSNYVY |
| 226 | Amino Acid | RNNQRPS |
| 227 | Amino Acid | AAWDDSLSAVV |
| 228 | Amino Acid | SYAIS |
| 229 | Amino Acid | GIIPIFGTANYAQKFQG |
| 230 | Amino Acid | DILRLSVSSGMDV |
| 231 | Amino Acid | SGSSSNIGSNYVY |
| 232 | Amino Acid | RNNQRPS |
| 233 | Amino Acid | AAWDDSLSGPSVV |
| 234 | Amino Acid | SYAIS |
| 235 | Amino Acid | GIIPIFGTANYAQKFQG |
| 236 | Amino Acid | PLRPLHYYGMDV |
| 237 | Amino Acid | SGSSSNIGSNYVY |
| 238 | Amino Acid | RNNQRPS |
| 239 | Amino Acid | AAWDDSLTEGV |
| 240 | Amino Acid | SYGIS |
| 241 | Amino Acid | WIGAYNGNTNYAQKLQG |
| 242 | Amino Acid | DLRRYSSSWDGPGY |
| 243 | Amino Acid | TGTSSDVGGYNYVS |
| 244 | Amino Acid | DVSNRPS |
| 245 | Amino Acid | SSYTSSSTRV |
| 246 | Amino Acid | SYGMH |
| 247 | Amino Acid | VISYDGSNKYYADSVKG |
| 248 | Amino Acid | SGWRYYYYYGMDV |
| 249 | Amino Acid | TGSSSNIGAGYDVH |
| 250 | Amino Acid | GNSNRPS |
| 251 | Amino Acid | QSYDSSLSGSV |
| 252 | Amino Acid | SYAIS |
| 253 | Amino Acid | GIIPIFGTANYAQKFQG |
| 254 | Amino Acid | DILRLSVSSGMDV |
| 255 | Amino Acid | SGSSSNIGSNYVY |
| 256 | Amino Acid | RNNQRPS |
| 257 | Amino Acid | AAWDDSLSGVV |
| 258 | Amino Acid | SYAIS |
| 259 | Amino Acid | GIIPIFGTANYAQKFQG |
| 260 | Amino Acid | DPSRDYYGSGRFSWFDP |
| 261 | Amino Acid | SGSSSNIGSNYVY |
| 262 | Amino Acid | RNNQRPS |
| 263 | Amino Acid | AAWDDSLSGRGV |
| 264 | Amino Acid | SYAIS |
| 265 | Amino Acid | GIIPIFGTANYAQKFQG |
| 266 | Amino Acid | DRGRRYCSGGSCPNAFDI |
| 267 | Amino Acid | RASQSVSSNLA |
| 268 | Amino Acid | GASTRAT |
| 269 | Amino Acid | QQYNNWPPALT |
| 270 | Amino Acid | SYAIS |
| 271 | Amino Acid | GIIPIFGTANYAQKFQG |
| 272 | Amino Acid | GGGRWLHSRLDV |
| 273 | Amino Acid | TGSSSNIGAGYDVH |
| 274 | Amino Acid | GNNNRPS |
| 275 | Amino Acid | QSYDRSLSGPVV |
| 276 | Amino Acid | SYAIS |
| 277 | Amino Acid | GIIPIFGTANYAQKFQG |
| 278 | Amino Acid | GGYVWGSYRQNSNWFDP |
| 279 | Amino Acid | SGSSSNIGSNYVY |
| 280 | Amino Acid | RNNQRPS |
| 281 | Amino Acid | AAWDDSLSGWV |
| 282 | Amino Acid | SYAIS |
| 283 | Amino Acid | GIIPIFGTANYAQKFQG |
| 284 | Amino Acid | PLYAPRFGYGMDV |
| 285 | Amino Acid | SGSSSNIGSNYVY |
| 286 | Amino Acid | RNNQRPS |
| 287 | Amino Acid | AAWDDSLSGWV |
| 288 | Amino Acid | SYAIS |
| 289 | Amino Acid | GIIPIFGTANYAQKFQG |
| 290 | Amino Acid | THLPYSYGLGGFDY |
| 291 | Amino Acid | TGSSSNIGAGYDVH |
| 292 | Amino Acid | GNSNRPS |
| 293 | Amino Acid | QSYDSSLSGPVV |
| 294 | Amino Acid | SYAMH |
| 295 | Amino Acid | VISYDGSNKYYADSVKG |
| 296 | Amino Acid | GRVYYYDSSGYSY |
| 297 | Amino Acid | TGSSSNIGAGYDVH |
| 298 | Amino Acid | GNSNRPS |
| 299 | Amino Acid | QSYDSSLSGREV |
| 300 | Amino Acid | SYAMS |
| 301 | Amino Acid | AISGSGGSTYYADSVKG |
| 302 | Amino Acid | DRGGQRGFDY |
| 303 | Amino Acid | SGSSSNIGSNYVY |
| 304 | Amino Acid | RNNQRPS |
| 305 | Amino Acid | AAWDDSLSGLV |
| 306 | Amino Acid | SYGIS |
| 307 | Amino Acid | WISAYNGNTNYAQKLQG |
| 308 | Amino Acid | DIIRYCSSTSCYRGIDY |
| 309 | Amino Acid | TGTSSDVGGYNYVS |
| 310 | Amino Acid | DVSNRPS |
| 311 | Amino Acid | SSYTSSSPHVV |
| 312 | Amino Acid | SYGMH |
| 313 | Amino Acid | VISYDGSNKYYADSVKG |
| 314 | Amino Acid | DAADMGAFDY |
| 315 | Amino Acid | KSSQSVLYSSNNKNYLA |
| 316 | Amino Acid | WASARES |
| 317 | Amino Acid | QQYYSTPLT |
| 318 | Amino Acid | SYGMH |
| 319 | Amino Acid | VISYDGSNKYYADSVKG |
| 320 | Amino Acid | DAGDLGAFDI |
| 321 | Amino Acid | KSSQSVLYSSNNKNYLA |
| 322 | Amino Acid | WASTRES |
| 323 | Amino Acid | QQYYSTPLT |
| 324 | Amino Acid | SYGMH |
| 325 | Amino Acid | VISYDGSNKYYADSVKG |
| 326 | Amino Acid | DGGDSGSLDY |
| 327 | Amino Acid | KSSQSVLYSSNNKNYLA |
| 328 | Amino Acid | WASTRES |
| 329 | Amino Acid | QQYYSTPLT |
| 330 | Amino Acid | SYGMH |
| 331 | Amino Acid | VISYDGSNKYYADSVKG |
| 332 | Amino Acid | DGTDYRGAFDI |
| 333 | Amino Acid | KSSQSVLYSSNNKNYLA |
| 334 | Amino Acid | WASTRES |
| 335 | Amino Acid | QQYYSTPLT |
| 336 | Amino Acid | SYGMH |
| 337 | Amino Acid | VISYDGSNKYYADSVKG |
| 338 | Amino Acid | GVPRYYYYYGMDV |
| 339 | Amino Acid | TGSSSNIGAGYDVH |
| 340 | Amino Acid | GNSNRPS |
| 341 | Amino Acid | QSYDSSLSGSV |
| 342 | Amino Acid | SYSMN |
| 343 | Amino Acid | AISGSGGSTYYADSVKG |
| 344 | Amino Acid | ARGYSYGSDAFDI |
| 345 | Amino Acid | SGSSSNIGSNYVY |
| 346 | Amino Acid | RNNQRPS |
| 347 | Amino Acid | AAWDDSLSGVV |
| 348 | Amino Acid | SYWMS |
| 349 | Amino Acid | NIKQDGSEKYYVDSVKG |
| 350 | Amino Acid | PAGQLLYGMDV |
| 351 | Amino Acid | SGDALPKQYAY |
| 352 | Amino Acid | KDSERPS |
| 353 | Amino Acid | QSADSSGTWV |
| 354 | Amino Acid | SYWMS |
| 355 | Amino Acid | NIKQDGSEKYYVDSVKG |
| 356 | Amino Acid | WSHYYDSSGLDY |
| 357 | Amino Acid | SGDALPKQYAY |
| 358 | Amino Acid | KDSERPS |
| 359 | Amino Acid | QSADSSGTWV |
| 360 | Amino Acid | RNIFSLYRVD |
| 361 | Amino Acid | GSTSGGTTTYADA |
| 362 | Amino Acid | HAHDHWRDS |
| 363 | Amino Acid | RNIFSMYRVD |
| 364 | Amino Acid | GITSGGTTSYADA |
| 365 | Amino Acid | HAHDHWRDS |
| 366 | Amino Acid | RNIASLYRVD |
| 367 | Amino Acid | GRTSGGTTTYLDA |
| 368 | Amino Acid | HAHDHWRDS |
| 369 | Amino Acid | GRTGSSYAMG |
| 370 | Amino Acid | AITWSGGITAYADS |
| 371 | Amino Acid | AAGVTGSPSFDS |
| 372 | Amino Acid | GRTFSSYAMG |
| 373 | Amino Acid | AISGSGGSIYYGDS |
| 374 | Amino Acid | AAGPLGSPDFDS |
| 375 | Amino Acid | GRTFSSYAMG |
| 376 | Amino Acid | AISGSGGSIYYGDS |
| 377 | Amino Acid | AAGPLGSPDFDS |
| 378 | Amino Acid | GRTGSSYAMG |
| 379 | Amino Acid | AITWSGGITAYADS |
| 380 | Amino Acid | AAGVTGSPSFDS |
| 381 | Amino Acid | GRTFSSYAMG |
| 382 | Amino Acid | AISGSGGVTFYAHS |
| 383 | Amino Acid | AGGAHGSPDFGS |
| 384 | Amino Acid | GSIGSIYAMG |
| 385 | Amino Acid | TTTSGGTTNYADS |
| 386 | Amino Acid | KIQTHWYVY |
| 387 | Amino Acid | GSIGSIYRKG |
| 388 | Amino Acid | TITSAGTTNYADS |
| 389 | Amino Acid | NFQTHWYVY |
| 390 | Amino Acid | KNIFSIYRVD |
| 391 | Amino Acid | GWTSGGSTSYADA |
| 392 | Amino Acid | HAHDHWRDY |
| 393 | Amino Acid | SGFTFSSYGLSWI |
| 394 | Amino Acid | SINRNGDNTYYT |
| 395 | Amino Acid | TRGGFSY |
| 396 | Amino Acid | SASSSVSY |
| 397 | Amino Acid | DTSKLA |
| 398 | Amino Acid | QQWSGFPPITF |
| 399 | Amino Acid | SGFTFSSYGLSWI |
| 400 | Amino Acid | SINRNGDNTYYT |
| 401 | Amino Acid | TRGGFSY |
| 402 | Amino Acid | SASPGVTY |
| 403 | Amino Acid | DTSNLA |
| 404 | Amino Acid | QQWSGYPPITF |
| 405 | Amino Acid | SGFTFSSYGLSWI |
| 406 | Amino Acid | SINRNGDNAYYI |
| 407 | Amino Acid | TRGGFSH |
| 408 | Amino Acid | SASSSVSY |
| 409 | Amino Acid | DTSNLA |
| 410 | Amino Acid | QQWSGYPPITF |
| 411 | Amino Acid | SGFTFSSYGLSWV |
| 412 | Amino Acid | SINRNGGNTYYT |
| 413 | Amino Acid | ARGGFTY |
| 414 | Amino Acid | SASSSVGY |
| 415 | Amino Acid | DTSNLA |
| 416 | Amino Acid | QQWSGYPPITF |
| 417 | Amino Acid | SGFTFSNYGMSWI |
| 418 | Amino Acid | SINTSGGNTYYP |
| 419 | Amino Acid | TRGGFTH |
| 420 | Amino Acid | SASSSVSY |
| 421 | Amino Acid | DTSNLA |
| 422 | Amino Acid | QQWTGYPPITF |
| 423 | Amino Acid | SGFPFSSYGLSWV |
| 424 | Amino Acid | TINRNGDSAYYP |
| 425 | Amino Acid | TRGGFAY |
| 426 | Amino Acid | SGSSSVGY |
| 427 | Amino Acid | DTSNLA |
| 428 | Amino Acid | CQQWSGYPPITF |
| 429 | Amino Acid | SGFTFSNYGMSWI |
| 430 | Amino Acid | SINTSGGNTYYP |
| 431 | Amino Acid | TRGGFTH |
| 432 | Amino Acid | SASSSVAF |
| 433 | Amino Acid | DTSKLA |
| 434 | Amino Acid | CQQWSGYPPITF |
| 435 | Amino Acid | SGFTFSSYGLSWI |
| 436 | Amino Acid | SINRNGDNTYYT |
| 437 | Amino Acid | TRGGFSY |
| 438 | Amino Acid | SASSSVGY |
| 439 | Amino Acid | DTYNLA |
| 440 | Amino Acid | CQQWSGYPPITF |
| 441 | Amino Acid | SGFTFSSYGLSWV |
| 442 | Amino Acid | SVNRNGGNTYYT |
| 443 | Amino Acid | ARGGFTY |
| 444 | Amino Acid | SASSSVGY |
| 445 | Amino Acid | DTSHLA |
| 446 | Amino Acid | CQQWSGYPPITF |
| 447 | Amino Acid | SGFPFSSYGLSWV |
| 448 | Amino Acid | TINRNGDSAYYP |
| 449 | Amino Acid | TRGGFAY |
| 450 | Amino Acid | SASSSVGY |
| 451 | Amino Acid | DTSNLA |
| 452 | Amino Acid | CQQWSGYPPITF |
| 453 | Amino Acid | SGFTFSSYGLSWI |
| 454 | Amino Acid | SINRNGDNTYYT |
| 455 | Amino Acid | TRGGFSY |
| 456 | Amino Acid | SASSSVSY |
| 457 | Amino Acid | DTSNLA |
| 458 | Amino Acid | CQQWTGYPPITF |
| 459 | Amino Acid | SGFTFSSYGMSWV |
| 460 | Amino Acid | SINKNGGSTYYP |
| 461 | Amino Acid | TRGGFAY |
| 462 | Amino Acid | SASSSVGY |
| 463 | Amino Acid | DTSNLP |
| 464 | Amino Acid | CQQWSGYPPITF |
| 465 | Amino Acid | MALPVTALLLPLALLLHAARPQVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARDILRLSVSSGMDVWGQGTLVT |
| | | VSSGSTSGSGKPGSGEGSTKGQSVLTQPPSASGTP |
| | | GQRVTISCSGGSSNIGSNYVYWYQQLPGTAPKLLI |
| | | YRNNQRPSGVPDRFSGSKSGTSASLAISGLRSEDE |
| | | ADYYCAAWDDSLSGHVVFGGGTKLTVLEQKLISEE |
| | | DLNPGGGGGSGGGGSGGGGSGGGGSGSMDIQMTQS |
| | | PSSLSASVGDRVTITCRASQDIRNYLNWYQQKPGK |
| | | APKLLIYYTSRLESGVPSRFSGSGSGTDYTLTISS |
| | | LQPEDFATYYCQQGNTLPWTFGQGTKVEIKGGGGS |
| | | GGGGSGGGGSEVQLVESGGGLVQPGGSLRLSCAAS |
| | | GYSFTGYTMNWVRQAPGKGLEWVALINPTKGVSTY |
| | | NQKFKDRFTISVDKSKNTAYLQMNSLRAEDTAVYY |
| | | CARSGYYGDSDWYFDVWGQGTLVTVSSTSGGGGSL |
| | | ESGQVLLESNIKVLPTWSTPVQPMALIVLGGVAGL |
| | | LLFIGLGIFFCVRCRHRRRQAERMSQIKRLLSEKK |
| | | TCQCPHRFQKTCSPI |
| 466 | Amino Acid | MALPVTALLLPLALLLHAARPQVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARDILRLSVSSGMDVWGQGTLVT |
| | | VSSGSTSGSGKPGSGEGSTKGQSVLTQPPSASGTP |
| | | GQRVTISCSGSSSNIGSNYVYWYQQLPGTAPKLLI |
| | | YRNNQRPSGVPDRLSGSKSGTSASLAISGLRSEDE |
| | | ADYYCAAWDDSLSAVVFGGGTKLTVLEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPTKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 467 | Amino Acid | MALPVTALLLPLALLLHAARPQVQLQESGGGVVQP |
| | | GRSLRLSCAASGFTLSSYGMHWVRQAPGKGLEWVA |
| | | VISYDGSNKYYADSVKGRFTISRDNSKNTLYLQMN |
| | | SLRAEDTAVYYCAKSGWRYYYYYGMDVWGQGTLVT |
| | | VSSGSTSGSGKPGSGEGSTKGQSVLTQPPSVSGAP |
| | | GQRVTISCTGSSSNIGAGYDVHWYQQLPGTAPKLL |
| | | IYGNSNRPSGVPDRFSGSKSGTSASLAITGLQAED |
| | | EADYYCQSYDSSLSGSVFGGGTKLTVLEQKLISEE |
| | | DLNPGGGGGSGGGGSGGGGSGGGGSGSMDIQMTQS |
| | | PSSLSASVGDRVTITCRASQDIRNYLNWYQQKPGK |
| | | APKLLIYYTSRLESGVPSRFSGSGSGTDYTLTISS |
| | | LQPEDFATYYCQQGNTLPWTFGQGTKVEIKGGGGS |
| | | GGGGSGGGGSEVQLVESGGGLVQPGGSLRLSCAAS |
| | | GYSFTGYTMNWVRQAPGKGLEWVALINPTKGVSTY |
| | | NQKFKDRFTISVDKSKNTAYLQMNSLRAEDTAVYY |
| | | CARSGYYGDSDWYFDVWGQGTLVTVSSTSGGGGSL |
| | | ESGQVLLESNIKVLPTWSTPVQPMALIVLGGVAGL |
| | | LLFIGLGIFFCVRCRHRRRQAERMSQIKRLLSEKK |
| | | TCQCPHRFQKTCSPI |
| 468 | Amino Acid | MALPVTALLLPLALLLHAARPQVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARDILRLSVSSGMDVWGQGTMVT |
| | | VSSGSTSGSGKPGSGEGSTKGQSVLTQPPSASGTP |
| | | GQRVTISCSGSSSNIGSNYVYWYQQLPGTAPKLLI |
| | | YRNNQRPSGVPDRFSGSKSGTSASLAISGLRSEDE |
| | | ADYYCAAWDDSLSGPSVVFGGGTKLTVLEQKLISE |
| | | EDLNPGGGGGSGGGGSGGGGSGGGGSGSMDIQMTQ |
| | | SPSSLSASVGDRVTITCRASQDIRNYLNWYQQKPG |
| | | KAPKLLIYYTSRLESGVPSRFSGSGSGTDYTLTIS |
| | | SLQPEDFATYYCQQGNTLPWTFGQGTKVEIKGGGG |
| | | SGGGGSGGGGSEVQLVESGGGLVQPGGSLRLSCAA |
| | | SGYSFTGYTMNWVRQAPGKGLEWVALINPTKGVST |
| | | YNQKFKDRFTISVDKSKNTAYLQMNSLRAEDTAVY |
| | | YCARSGYYGDSDWYFDVWGQGTLVTVSSTSGGGGS |
| | | LESGQVLLESNIKVLPTWSTPVQPMALIVLGGVAG |
| | | LLLFIGLGIFFCVRCRHRRRQAERMSQIKRLLSEK |
| | | KTCQCPHRFQKTCSPI |
| 469 | Amino Acid | MALPVTALLLPLALLLHAARPQVQLVQSGAEVKKP |
| | | GASVKVSCKASGYTFTSYGISWVRQAPGQGLEWMG |
| | | WIGAYNGNTNYAQKLQGRVTMTTDTSTSTAYMELR |
| | | SLRSDDTAVYYCARDLRRYSSSWDGPGYWGQGTLV |
| | | TVSSGSTSGSGKPGSGEGSTKGQSALTQPASVSGS |
| | | PGQSITISCTGTSSDVGGYNYVSWYQQHPGKAPKL |
| | | MIYDVSNRPSGVSNRFSGSKSGNTASLTISGLQAE |
| | | DEADYYCSSYTSSSTRVFGTGTKLTVLEQKLISEE |
| | | DLNPGGGGGSGGGGSGGGGSGGGGSGSMDIQMTQS |
| | | PSSLSASVGDRVTITCRASQDIRNYLNWYQQKPGK |
| | | APKLLIYYTSRLESGVPSRFSGSGSGTDYTLTISS |
| | | LQPEDFATYYCQQGNTLPWTFGQGTKVEIKGGGGS |
| | | GGGGSGGGGSEVQLVESGGGLVQPGGSLRLSCAAS |
| | | GYSFTGYTMNWVRQAPGKGLEWVALINPTKGVSTY |
| | | NQKFKDRFTISVDKSKNTAYLQMNSLRAEDTAVYY |
| | | CARSGYYGDSDWYFDVWGQGTLVTVSSTSGGGGSL |
| | | ESGQVLLESNIKVLPTWSTPVQPMALIVLGGVAGL |
| | | LLFIGLGIFFCVRCRHRRRQAERMSQIKRLLSEKK |
| | | TCQCPHRFQKTCSPI |
| 470 | Amino Acid | MALPVTALLLPLALLLHAARPQVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARDILRLSVSSGMDVWGQGTLVT |
| | | VSSGSTSGSGKPGSGEGSTKGSYVLTQPPSASGTP |
| | | GQRVTISCSGSSSNIGGNYVYWYQQLPGTAPKLLI |
| | | YRNNQRPSGVPDRFSGSKSGTSASLAISGLRSEDE |
| | | ADYYCAAWDDSLSGWVFGGGTKLTVLEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPTKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 471 | Amino Acid | MALPVTALLLPLALLLHAARPQVQLVQSGAEVKKP |
| | | GSSVKVSCKASGGTFSSYAISWVRQAPGQGLEWMG |
| | | GIIPIFGTANYAQKFQGRVTITADKSTSTAYMELS |
| | | SLRSEDTAVYYCARDGGRYSYGRSFDYWGQGTLVT |
| | | VSSGSTSGSGKPGSGEGSTKGQSVLTQPPSVSGAP |
| | | GQRVTISCTGSSSNIGAGYDVHWYQQLPGTAPKLL |
| | | IYGNSNRPSGVPDRFSGSKSGTSASLAITGLQAED |
| | | EADYYCQSYDSSLSGPVVFGGGTKLTVLEQKLISE |
| | | EDLNPGGGGGSGGGGSGGGGSGGGGSGSMDIQMTQ |
| | | SPSSLSASVGDRVTITCRASQDIRNYLNWYQQKPG |
| | | KAPKLLIYYTSRLESGVPSRFSGSGSGTDYTLTIS |
| | | SLQPEDFATYYCQQGNTLPWTFGQGTKVEIKGGGG |
| | | SGGGGSGGGGSEVQLVESGGGLVQPGGSLRLSCAA |
| | | SGYSFTGYTMNWVRQAPGKGLEWVALINPTKGVST |
| | | YNQKFKDRFTISVDKSKNTAYLQMNSLRAEDTAVY |
| | | YCARSGYYGDSDWYFDVWGQGTLVTVSSTSGGGGS |
| | | LESGQVLLESNIKVLPTWSTPVQPMALIVLGGVAG |
| | | LLLFIGLGIFFCVRCRHRRRQAERMSQIKRLLSEK |
| | | KTCQCPHRFQKTCSPI |
| 472 | Amino Acid | MALPVTALLLPLALLLHAARPQVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARPLRPLHYYGMDVWGQGTLVTV |
| | | SSGSTSGSGKPGSGEGSTKGQSVLTQPPSASGTPG |
| | | QRVTISCSGSSSNIGSNYVYWYQQLPGTAPKLLIY |
| | | RNNQRPSGVPDRFSGSKSGTSASLAISGLRSEDEA |
| | | DYYCAAWDDSLTEGVFGGGTKLTVLEQKLISEEDL |
| | | NPGGGGGSGGGGSGGGGSGGGGSGSMDIQMTQSPS |
| | | SLSASVGDRVTITCRASQDIRNYLNWYQQKPGKAP |
| | | KLLIYYTSRLESGVPSRFSGSGSGTDYTLTISSLQ |
| | | PEDFATYYCQQGNTLPWTFGQGTKVEIKGGGGSGG |
| | | GGSGGGGSEVQLVESGGGLVQPGGSLRLSCAASGY |
| | | SFTGYTMNWVRQAPGKGLEWVALINPTKGVSTYNQ |
| | | KFKDRFTISVDKSKNTAYLQMNSLRAEDTAVYYCA |
| | | RSGYYGDSDWYFDVWGQGTLVTVSSTSGGGGSLES |
| | | GQVLLESNIKVLPTWSTPVQPMALIVLGGVAGLLL |
| | | FIGLGIFFCVRCRHRRRQAERMSQIKRLLSEKKTC |
| | | QCPHRFQKTCSPI |
| 473 | Amino Acid | MALPVTALLLPLALLLHAARPQVQLVESGGGVVQP |
| | | GRSLRLSCAASGFTFSSYGMHWVRRAPGKGLEWVA |
| | | VISYDGSNKYYADSVKGRFTISRDNSKNTLYLQMN |
| | | SLRAEDTAVYYCAKDGGDSGSLDYWGQGTLVTVSS |
| | | GSTSGSGKPGSGEGSTKGDIVMTQSPDSLAVSLGE |
| | | RATINCKSSQSVLYSSNNKNYLAWYQQKPGQPPKL |
| | | LIYWASTRESGVPDRFSGSGSGTDFTLTISSLQAE |
| | | DVAVYYCQQYYSTPLTFGGGTKLEIKEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPTKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 474 | Amino Acid | MALPVTALLLPLALLLHAARPEVQLLESGGGLVQP |
| | | GGSLRLSCAASGFTFSSYWMSWVRQAPGKGLEWVA |
| | | NIKQDGSEKYYVDSVKGRFTISRDNAKNSLYLQMN |
| | | SLRAEDTAVYYCARPAGQLLYGMDVWGQGTLVTVS |
| | | SGSTSGSGKPGSGEGSTKGSYVLTQPPSVSVSPGQ |
| | | TARITCSGDALPKQYAYWYQQKPGQAPVLVIYKDS |
| | | ERPSGIPERFSGSSSGTTVTLTISGVQAEDEADYY |
| | | CQSADSSGTWVFGGGTKLTVLEQKLISEEDLNPGG |
| | | GGGSGGGGSGGGGSGGGGSGSMDIQMTQSPSSLSA |
| | | SVGDRVTITCRASQDIRNYLNWYQQKPGKAPKLLI |
| | | YYTSRLESGVPSRFSGSGSGTDYTLTISSLQPEDF |
| | | ATYYCQQGNTLPWTFGQGTKVEIKGGGGSGGGGSG |
| | | GGGSEVQLVESGGGLVQPGGSLRLSCAASGYSFTG |
| | | YTMNWVRQAPGKGLEWVALINPTKGVSTYNQKFKD |
| | | RFTISVDKSKNTAYLQMNSLRAEDTAVYYCARSGY |
| | | YGDSDWYFDVWGQGTLVTVSSTSGGGGSLESGQVL |
| | | LESNIKVLPTWSTPVQPMALIVLGGVAGLLLFIGL |
| | | GIFFCVRCRHRRRQAERMSQIKRLLSEKKTCQCPH |
| | | RFQKTCSPI |
| 475 | Amino Acid | MALPVTALLLPLALLLHAARPEVQLLESGGGVVQP |
| | | GRSLRLSCAASGFTFSSYGMHWVRQAPGKGLEWVA |
| | | VISYDGSNKYYADSVKGRFTISRDNSKNTLYLQMN |
| | | SLRAEDTAVYYCAKDAADMGAFDYWGQGTLVTVSS |
| | | GSTSGSGKPGSGEGSTKGDIQMTQSPDSLAVSLGE |
| | | RATMNCKSSQSVLYSSNNKNYLAWYQQKPGQPPKL |
| | | LIYWASARESGVPDRFSGSGSGTDFTLTISSLQAE |
| | | DVAVYYCQQYYSTPLTFGQGTKVEIKEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGS |
| | | GTDYTLTISSLQPEDFATYYCQQGNTLPWTFGQGT |
| | | KVEIKGGGGSGGGGSGGGGSEVQLVESGGGLVQPG |
| | | GSLRLSCAASGYSFTGYTMNWVRQAPGKGLEWVAL |
| | | INPTKGVSTYNQKFKDRFTISVDKSKNTAYLQMNS |
| | | LRAEDTAVYYCARSGYYGDSDWYFDVWGQGTLVTV |
| | | SSTSGGGGSLESGQVLLESNIKVLPTWSTPVQPMA |
| | | LIVLGGVAGLLLFIGLGIFFCVRCRHRRRQAERMS |
| | | QIKRLLSEKKTCQCPHRFQKTCSPI |
| 476 | Amino Acid | MALPVTALLLPLALLLHAARPEVQLLESGGGLVKP |
| | | GGSLRLSCAASGFTFSSYSMNWVRQAPGKGLEWVS |
| | | AISGSGGSTYYADSVKGRFTISRDNSKNTLYLQMN |
| | | SLRAEDTAVYYCARARGYSYGSDAFDIWGQGTMVT |
| | | VSSGSTSGSGKPGSGEGSTKGQSVLTQPPSASGTP |
| | | GQRVTISCSGSSSNIGSNYVYWYQQLPGTAPKLLI |
| | | YRNNQRPSGVPDRFSGSKSGTSASLAISGLRSEDE |
| | | ADYYCAAWDDSLSGVVFGGGTKLTVLEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPTKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 477 | Amino Acid | MALPVTALLLPLALLLHAARPQVQLVQSGGGVVQP |
| | | GRSLRLSCAASGFTFSSYGMHWVRQAPGKGLEWVA |
| | | VISYDGSNKYYADSVKGRFTISRDNSKNTLYLQMN |
| | | SLRAEDTAVYYCAKGVPRYYYYYGMDVWGQGTMVT |
| | | VSSGSTSGSGKPGSGEGSTKGQSVLTQPPSVSGAP |
| | | GQRVTISCTGSSSNIGAGYDVHWYQQLPGTAPKLL |
| | | IYGNSNRPSGVPDRFSGSKSGTSASLAITGLQAED |
| | | EADYYCQSYDSSLSGSVFGGGTKLTVLEQKLISEE |
| | | DLNPGGGGGSGGGGSGGGGSGGGGSGSMDIQMTQS |
| | | PSSLSASVGDRVTITCRASQDIRNYLNWYQQKPGK |
| | | APKLLIYYTSRLESGVPSRFSGSGSGTDYTLTISS |
| | | LQPEDFATYYCQQGNTLPWTFGQGTKVEIKGGGGS |
| | | GGGGSGGGGSEVQLVESGGGLVQPGGSLRLSCAAS |
| | | GYSFTGYTMNWVRQAPGKGLEWVALINPTKGVSTY |
| | | NQKFKDRFTISVDKSKNTAYLQMNSLRAEDTAVYY |
| | | CARSGYYGDSDWYFDVWGQGTLVTVSSTSGGGGSL |
| | | ESGQVLLESNIKVLPTWSTPVQPMALIVLGGVAGL |
| | | LLFIGLGIFFCVRCRHRRRQAERMSQIKRLLSEKK |
| | | TCQCPHRFQKTCSPI |
| 478 | Amino Acid | MALPVTALLLPLALLLHAARPEVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARDRGRRYCSGGSCPNAFDIWGQ |
| | | GTMVTVSSGSTSGSGKPGSGEGSTKGEIVLTQSPA |
| | | TLSVSPGERATLSCRASQSVSSNLAWYQQKPGQAP |
| | | RLLIYGASTRATGIPARFSGSGSGTEFTLTISSLQ |
| | | SEDFAVYYCQQYNNWPPALTFGGGTKVEIKEQKLI |
| | | SEEDLNPGGGGGSGGGGSGGGGSGGGGSGSMDIQM |
| | | TQSPSSLSASVGDRVTITCRASQDIRNYLNWYQQK |
| | | PGKAPKLLIYYTSRLESGVPSRFSGSGSGTDYTLT |
| | | ISSLQPEDFATYYCQQGNTLPWTFGQGTKVEIKGG |
| | | GGSGGGGSGGGGSEVQLVESGGGLVQPGGSLRLSC |
| | | AASGYSFTGYTMNWVRQAPGKGLEWVALINPTKGV |
| | | STYNQKFKDRFTISVDKSKNTAYLQMNSLRAEDTA |
| | | VYYCARSGYYGDSDWYFDVWGQGTLVTVSSTSGGG |
| | | GSLESGQVLLESNIKVLPTWSTPVQPMALIVLGGV |
| | | AGLLLFIGLGIFFCVRCRHRRRQAERMSQIKRLLS |
| | | EKKTCQCPHRFQKTCSPI |
| 479 | Amino Acid | MALPVTALLLPLALLLHAARPQVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARGGYVWGSYRQNSNWFDPWGQG |
| | | TLVTVSSGSTSGSGKPGSGEGSTKGSYVLTQPPSA |
| | | SGTPGQRVTISCSGSSSNIGSNYVYWYQQLPGTAP |
| | | KLLIYRNNQRPSGVPDRFSGSKSGTSASLAISGLR |
| | | SEDEADYYCAAWDDSLSGWVFGGGTKLTVLEQKLI |
| | | SEEDLNPGGGGGSGGGGSGGGGSGGGGSGSMDIQM |
| | | TQSPSSLSASVGDRVTITCRASQDIRNYLNWYQQK |
| | | PGKAPKLLIYYTSRLESGVPSRFSGSGSGTDYTLT |
| | | ISSLQPEDFATYYCQQGNTLPWTFGQGTKVEIKGG |
| | | GGSGGGGSGGGGSEVQLVESGGGLVQPGGSLRLSC |
| | | AASGYSFTGYTMNWVRQAPGKGLEWVALINPTKGV |
| | | STYNQKFKDRFTISVDKSKNTAYLQMNSLRAEDTA |
| | | VYYCARSGYYGDSDWYFDVWGQGTLVTVSSTSGGG |
| | | GSLESGQVLLESNIKVLPTWSTPVQPMALIVLGGV |
| | | AGLLLFIGLGIFFCVRCRHRRRQAERMSQIKRLLS |
| | | EKKTCQCPHRFQKTCSPI |
| 480 | Amino Acid | MALPVTALLLPLALLLHAARPQVQLVESGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARDILRLSVSSGMDVWGQGTLVT |
| | | VSSGSTSGSGKPGSGEGSTKGQSVLTQPPSASGTP |
| | | GRRVTISCSGSSSNIGSNYVYWYQQLPGTAPKLLI |
| | | YRNNQRPSGVPDRFSGSKSGTSASLAISGLRSEDE |
| | | ADYYCAAWDDSLSGVVFGGGTKLTVLEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPTKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 481 | Amino Acid | MALPVTALLLPLALLLHAARPQVQLVESGGGLVQP |
| | | GGSLRLSCAASGFTFSSYWMSWVRQAPGKGLEWVA |
| | | NIKQDGSEKYYVDSVKGRFTISRDNAKNSLYLQMN |
| | | SLRAEDTAVYYCARWSHYYDSSGLDYWGQGTLVTV |
| | | SSGSTSGSGKPGSGEGSTKGSYVLTQPPSVSVSPG |
| | | QTARITCSGDALPKQYAYWYQQKPGQAPVLVIYKD |
| | | SERPSGIPERFSGSSSGTTVTLTISGVQAEDEADY |
| | | YCQSADSSGTWVFGGGTKLTVLEQKLISEEDLNPG |
| | | GGGGSGGGGSGGGGSGGGGSGSMDIQMTQSPSSLS |
| | | ASVGDRVTITCRASQDIRNYLNWYQQKPGKAPKLL |
| | | IYYTSRLESGVPSRFSGSGSGTDYTLTISSLQPED |
| | | FATYYCQQGNTLPWTFGQGTKVEIKGGGGSGGGGS |
| | | GGGGSEVQLVESGGGLVQPGGSLRLSCAASGYSFT |
| | | GYTMNWVRQAPGKGLEWVALINPTKGVSTYNQKFK |
| | | DRFTISVDKSKNTAYLQMNSLRAEDTAVYYCARSG |
| | | YYGDSDWYFDVWGQGTLVTVSSTSGGGGSLESGQV |
| | | LLESNIKVLPTWSTPVQPMALIVLGGVAGLLLFIG |
| | | LGIFFCVRCRHRRRQAERMSQIKRLLSEKKTCQCP |
| | | HRFQKTCSPI |
| 482 | Amino Acid | MALPVTALLLPLALLLHAARPQVQLVESGAEVKKP |
| | | GSSVKVSCKASGGTFSSYAISWVRQAPGQGLEWMG |
| | | GIIPIFGTANYAQKFQGRVTITADKSTSTAYMELS |
| | | SLRSEDTAVYYCARTHLPYSYGLGGFDYWGQGTLV |
| | | TVSSGSTSGSGKPGSGEGSTKGQSVLTQPPSVSGA |
| | | PGQRVTISCTGSSSNIGAGYDVHWYQQLPGTAPKL |
| | | LIYGNSNRPSGVPDRFSGSKSGTSASLAITGLQAE |
| | | DEADYYCQSYDSSLSGPVVFGGGTKVTVLEQKLIS |
| | | EEDLNPGGGGGSGGGGSGGGGSGGGGSGSMDIQMT |
| | | QSPSSLSASVGDRVTITCRASQDIRNYLNWYQQKP |
| | | GKAPKLLIYYTSRLESGVPSRFSGSGSGTDYTLTI |
| | | SSLQPEDFATYYCQQGNTLPWTFGQGTKVEIKGGG |
| | | GSGGGGSGGGGSEVQLVESGGGLVQPGGSLRLSCA |
| | | ASGYSFTGYTMNWVRQAPGKGLEWVALINPTKGVS |
| | | TYNQKFKDRFTISVDKSKNTAYLQMNSLRAEDTAV |
| | | YYCARSGYYGDSDWYFDVWGQGTLVTVSSTSGGGG |
| | | SLESGQVLLESNIKVLPTWSTPVQPMALIVLGGVA |
| | | GLLLFIGLGIFFCVRCRHRRRQAERMSQIKRLLSE |
| | | KKTCQCPHRFQKTCSPI |
| 483 | Amino Acid | MALPVTALLLPLALLLHAARPEVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARDPSRDYYGSGRFSWFDPWGQG |
| | | TLVTVSSGSTSGSGKPGSGEGSTKGQSVLTQPPSA |
| | | SGTPGQRVTISCSGSSSNIGSNYVYWYQQLPGTAP |
| | | KLLIYRNNQRPSGVPDRFSGSKSGTSASLAISGLR |
| | | SEDEADYYCAAWDDSLSGRGVFGGGTKLTVLEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 484 | Amino Acid | MALPVTALLLPLALLLHAARPQVQLVQSGAEVKKP |
| | | GASVKVSCKASGYTFTSYGISWVRQAPGQGLEWMG |
| | | WISAYNGNTNYAQKLQGRVTMTTDTSTSTAYMELR |
| | | SLRSDDTAVYYCARDIIRYCSSTSCYRGIDYWGQG |
| | | TLVTVSSGSTSGSGKPGSGEGSTKGQSALTQPASV |
| | | SGSPGQSITISCTGTSSDVGGYNYVSWYQQHPGKA |
| | | PKLMIYDVSNRPSGVSNRFSGSKSGNTASLTISGL |
| | | QAEDEADYYCSSYTSSSPHVVSGGGTKLTVLEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 485 | Amino Acid | MALPVTALLLPLALLLHAARPQVQLVESGGGVVQP |
| | | GRSLRLSCAASGFTFSSYGMHWVRQAPGKGLEWVA |
| | | VISYDGSNKYYADSVKGRFTISRDNSKNTLYLQMN |
| | | SLRAEDTAVYYCARDGTDYRGAFDIWGQGTMVTVS |
| | | SGSTSGSGKPGSGEGSTKGDIQLTQSPDSLAVPLG |
| | | ERATINCKSSQSVLYSSNNKNYLAWYQQKPGQPPK |
| | | LLIYWASTRESGVPDRFSGSGSGTDFTLTISSLQA |
| | | EDVAVYYCQQYYSTPLTFGQGTKLEIKEQKLISEE |
| | | DLNPGGGGGSGGGGSGGGGSGGGGSGSMDIQMTQS |
| | | PSSLSASVGDRVTITCRASQDIRNYLNWYQQKPGK |
| | | APKLLIYYTSRLESGVPSRFSGSGSGTDYTLTISS |
| | | LQPEDFATYYCQQGNTLPWTFGQGTKVEIKGGGGS |
| | | GGGGSGGGGSEVQLVESGGGLVQPGGSLRLSCAAS |
| | | GYSFTGYTMNWVRQAPGKGLEWVALINPTKGVSTY |
| | | NQKFKDRFTISVDKSKNTAYLQMNSLRAEDTAVYY |
| | | CARSGYYGDSDWYFDVWGQGTLVTVSSTSGGGGSL |
| | | ESGQVLLESNIKVLPTWSTPVQPMALIVLGGVAGL |
| | | LLFIGLGIFFCVRCRHRRRQAERMSQIKRLLSEKK |
| | | TCQCPHRFQKTCSPI |
| 486 | Amino Acid | MALPVTALLLPLALLLHAARPEVQLVQSGAEVKKP |
| | | GSSVRVSCKASGGTFSSYAISWVRQAPGQGLEWMG |
| | | GIIPIFGTANYAQKFQGRVTITADKSTSTAYMELS |
| | | SLRSEDTAVYYCARGGGRWLHSRLDVWGQGTTVTV |
| | | SSGSTSGSGKPGSGEGSTKGQSVLTQPPSVSGAPG |
| | | QRVTISCTGSSSNIGAGYDVHWYQQFPGTAPKLLI |
| | | FGNNNRPSGVPDRFSGSKSGTSASLAITGLQAEDE |
| | | ANYYCQSYDRSLSGPVVFGGGTKLTVLEQKLISEE |
| | | DLNPGGGGGSGGGGSGGGGSGGGGSGSMDIQMTQS |
| | | PSSLSASVGDRVTITCRASQDIRNYLNWYQQKPGK |
| | | APKLLIYYTSRLESGVPSRFSGSGSGTDYT |
| | | LTISSLQPEDFATYYCQQGNTLPWTFGQGTKVEIK |
| | | GGGGSGGGGSGGGGSEVQLVESGGGLVQPGGSLRL |
| | | SCAASGYSFTGYTMNWVRQAPGKGLEWVALINPTK |
| | | GVSTYNQKFKDRFTISVDKSKNTAYLQMNSLRAED |
| | | TAVYYCARSGYYGDSDWYFDVWGQGTLVTVSSTSG |
| | | GGGSLESGQVLLESNIKVLPTWSTPVQPMALIVLG |
| | | GVAGLLLFIGLGIFFCVRCRHRRRQAERMSQIKRL |
| | | LSEKKTCQCPHRFQKTCSPI |
| 487 | Amino Acid | MALPVTALLLPLALLLHAARPEVQLVESGGGVVQP |
| | | GRSLRLSCAASGFTFSSYGMHWVRQAPGKGLEWVA |
| | | VISYDGSNKYYADSVKGRFTISRDNSKNTLYLQMN |
| | | SLRAEDTAVYYCAKDAGDLGAFDIWGQGTMVTVSS |
| | | GSTSGSGKPGSGEGSTKGDIVMTQSPDSLAVSLGE |
| | | RATINCKSSQSVLYSSNNKNYLAWYQQKPGQPPKL |
| | | LIYWASTRESGVPDRFSGSGSGTDFTLTISSLQAE |
| | | DVAVYYCQQYYSTPLTFGPGTKVEIKEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPTKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 488 | Amino Acid | MALPVTALLLPLALLLHAARPQVQLVQSGGGVVQP |
| | | GRSLRLSCAASGFTFSSYAMHWVRQAPGKGLEWVA |
| | | VISYDGSNKYYADSVKGRFTISRDNSKNTLYLQMN |
| | | SLRAEDTAVYYCARGRVYYYDSSGYSYWGQGTLVT |
| | | VSSGSTSGSGKPGSGEGSTKGQSVLTQPPSVSGAP |
| | | GQRVTISCTGSSSNIGAGYDVHWYQQLPGTAPKLL |
| | | IYGNSNRPSGVPDRFSGSKSGTSASLAITGLQAED |
| | | EADYYCQSYDSSLSGREVFGTGTKLTVLEQKLISE |
| | | EDLNPGGGGGSGGGGSGGGGSGGGGSGSMDIQMTQ |
| | | SPSSLSASVGDRVTITCRASQDIRNYLNWYQQKPG |
| | | KAPKLLIYYTSRLESGVPSRFSGSGSGTDYTLTIS |
| | | SLQPEDFATYYCQQGNTLPWTFGQGTKVEIKGGGG |
| | | SGGGGSGGGGSEVQLVESGGGLVQPGGSLRLSCAA |
| | | SGYSFTGYTMNWVRQAPGKGLEWVALINPTKGVST |
| | | YNQKFKDRFTISVDKSKNTAYLQMNSLRAEDTAVY |
| | | YCARSGYYGDSDWYFDVWGQGTLVTVSSTSGGGGS |
| | | LESGQVLLESNIKVLPTWSTPVQPMALIVLGGVAG |
| | | LLLFIGLGIFFCVRCRHRRRQAERMSQIKRLLSEK |
| | | KTCQCPHRFQKTCSPI |
| 489 | Amino Acid | MALPVTALLLPLALLLHAARPQVQLVESGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARPLYAPRFGYGMDVWGQGTLVT |
| | | VSSGSTSGSGKPGSGEGSTKGQSVLTQPPSASGTP |
| | | GQRVTISCSGSSSNIGSNYVYWYQQLPGTAPKLLI |
| | | YRNNQRPSGVPDRFSGSKSGTSASLAISGLRSEDE |
| | | ADYYCAAWDDSLSGWVFGGGTQLTVLEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPTKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 490 | Amino Acid | MALPVTALLLPLALLLHAARPEVQLLESGGGLVQP |
| | | GGSLRLSCAASGFTFSSYAMSWVRQAPGKGLEWVS |
| | | AISGSGGSTYYADSVKGRFTISRDNSKNTLYLQMN |
| | | SLRAEDTAVYYCAKDRGGQRGFDYWGQGTLVTVSS |
| | | GSTSGSGKPGSGEGSTKGQSVLTQPPSASGTPGQR |
| | | VTISCSGSSSNIGSNYVYWYQQLPGTAPKLLIYRN |
| | | NQRPSGVPDRFSGSKSGTSASLAISGLRSEDEADY |
| | | YCAAWDDSLSGLVFGGGTKLTVLEQKLISEEDLNP |
| | | GGGGGSGGGGSGGGGSGGGGSGSMDIQMTQSPSSL |
| | | SASVGDRVTITCRASQDIRNYLNWYQQKPGKAPKL |
| | | LIYYTSRLESGVPSRFSGSGSGTDYTLTISSLQPE |
| | | DFATYYCQQGNTLPWTFGQGTKVEIKGGGGSGGGG |
| | | SGGGGSEVQLVESGGGLVQPGGSLRLSCAASGYSF |
| | | TGYTMNWVRQAPGKGLEWVALINPTKGVSTYNQKF |
| | | KDRFTISVDKSKNTAYLQMNSLRAEDTAVYYCARS |
| | | GYYGDSDWYFDVWGQGTLVTVSSTSGGGGSLESGQ |
| | | VLLESNIKVLPTWSTPVQPMALIVLGGVAGLLLFI |
| | | GLGIFFCVRCRHRRRQAERMSQIKRLLSEKKTCQC |
| | | PHRFQKTCSPI |
| 491 | Amino Acid | MALPVTALLLPLALLLHAARPEVKLVESGGGLVQP |
| | | GGSLKLSCTTSGFTFSSYGLSWIRQTPDKRLELVA |
| | | SINRNGDNTYYTDSVKGRFTISRHDAKNTLNLQMN |
| | | SLKSEDTAMYYCTRGGFSYWGQGTLVTVSAGSTSG |
| | | SGKPGSGEGSTKGDIVLTQSPAIMSSSPGEKVTMT |
| | | CSASSSVSYMYWYQQRPGSSPRLLIYDTSKLASGV |
| | | PVRFSGSVSGTSYSLTISRMESEDAATYYCQQWSG |
| | | FPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPTKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 492 | Amino Acid | MALPVTALLLPLALLLHAARPEVKLVESGGGLVQP |
| | | GGSLKLSCTTSGFTFSSYGLSWIRQTPDKRLELVA |
| | | SINRNGDNTYYTDSVRGRFTISRDNAKNTLNLQMS |
| | | SLKSEDTAMYYCTRGGFSYWGQGTLVTVSAGSTSG |
| | | SGKPGSGEGSTKGDIVLTQSPATMSASPGEKVTLT |
| | | CSASPGVTYMYWYQQKPGSSPRLLIYDTSNLASGV |
| | | PLRFSGSGSGTSYSLTISRTEAEDAATYYCQQWSG |
| | | YPPITFGAGTKLELREQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGGGGGSGGGGSEVQ |
| | | LVESGGGLVQPGGSLRLSCAASGYSFTGYTMNWVR |
| | | QAPGKGLEWVALINPTKGVSTYNQKFKDRFTISVD |
| | | KSKNTAYLQMNSLRAEDTAVYYCARSGYYGDSDWY |
| | | FDVWGQGTLVTVSSTSGGGGSLESGQVLLESNIKV |
| | | LPTWSTPVQPMALIVLGGVAGLLLFIGLGIFFCVR |
| | | CRHRRRQAERMSQIKRLLSEKKTCQCPHRFQKTCS |
| | | PI |
| 493 | Amino Acid | MALPVTALLLPLALLLHAARPEVKLVESGGGLVQP |
| | | GGSLKLSCTTSGFTFSSYGLSWIRQTPDKRLELVA |
| | | SINRNGDNAYYIDSVKGRFTISRDNAKNALNLQMS |
| | | SLKSEDTAMYYCTRGGFSHWGQGTLVTVAAGSTSG |
| | | SGKPGSGEGSTKGQIVLTQSPAIMSASPGEKVTMT |
| | | CSASSSVSYMYWYQQKPGSSPRLLIYDTSNLASGV |
| | | PVRFSGSGSGTSYSLIISRMEAEDAATYYCQQWSG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPTKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 494 | Amino Acid | MALPVTALLLPLALLLHAARPEVKLVESGGGLVQP |
| | | GGSLKLSCAASGFTFSSYGLSWVRQTPDKGLELVA |
| | | SINRNGGNTYYTDSVKGRFTISRDNAKNTLNLQMS |
| | | SLKSEDTAMYYCARGGFTYWGQGTLVTVSAGSTSG |
| | | SGKPGSGEGSTKGQIVLTQSPAIMSAFPGEKVTMT |
| | | CSASSSVGYMYWYQQKPGSSPRLLIYDTSNLASGV |
| | | PVRFSGSGSGTAYSLTISRMEAEDAATYYCQQWSG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPTKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 495 | Amino Acid | MALPVTALLLPLALLLHAARPQVQLKESGGGLVRP |
| | | GGSLKLSCAASGFTFSNYGMSWIRQTPDKNLELVA |
| | | SINTSGGNTYYPDSVKGRFTISRDNARATLNLQMS |
| | | NLKSEDTAIYYCTRGGFTHWGQGTLVAVSAGSTSG |
| | | SGKPGSGEGSTKGQIVLTQSPAIMSASPGEKVTMT |
| | | CSASSSVSYMYWYQQKPGSSPRLLIYDTSNLASGV |
| | | PVRFSGSGSGTSYFLTISRMEAEDAATYYCQQWTG |
| | | YPPITFGAGTKLELREQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPTKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 496 | Amino Acid | MALPVTALLLPLALLLHAARPQVQLKESGGGLVQP |
| | | GGSLKLSCAASGFPFSSYGLSWVRQTPDKRLELVA |
| | | TINRNGDSAYYPDSVKGRFTISRDDAKNTLYLQMS |
| | | SLKSEDTAMYYCTRGGFAYWGQGTLVTVSAGSTSG |
| | | SGKPGSGEGSTKGQIVLTQSPAIMSASPGEKVTMT |
| | | CSGSSSVGYMYWYQQKPGFSPRLLIYDTSNLASGV |
| | | PVRFSGSGSGTSYSLTISRMEAEDAATYYCQQWSG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGGGGGSEVQ |
| | | LVESGGGLVQPGGSLRLSCAASGYSFTGYTMNWVR |
| | | QAPGKGLEWVALINPTKGVSTYNQKFKDRFTISVD |
| | | KSKNTAYLQMNSLRAEDTAVYYCARSGYYGDSDWY |
| | | FDVWGQGTLVTVSSTSGGGGSLESGQVLLESNIKV |
| | | LPTWSTPVQPMALIVLGGVAGLLLFIGLGIFFCVR |
| | | CRHRRRQAERMSQIKRLLSEKKTCQCPHRFQKTCS |
| | | PI |
| 497 | Amino Acid | MALPVTALLLPLALLLHAARPEVKLVESGGGLVRP |
| | | GGSLKLSCAASGFTFSNYGMSWIRQTPDKNLELVA |
| | | SINTSGGNTYYPDSVKGRFTISRDNARATLNLQMS |
| | | NLKSEDTAIYYCTRGGFTHWGQGTLVTVSAGSTSG |
| | | SGKPGSGEGSTKGDIVLTQSPAIMSASPGEKVTMT |
| | | CSASSSVAFMYWYQQKPGSSPRLLIYDTSKLASGV |
| | | PVRFSGSGSGTSYSLTISRMEAEDAATYYCQQWSG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPTKGVSTYNQKFKDRFTISV |
| | | DKSKNTAYLQMNSLRAE |
| | | DTAVYYCARSGYYGDSDWYFDVWGQGTLVTVSSTS |
| | | GGGGSLESGQVLLESNIKVLPTWSTPVQPMALIVL |
| | | GGVAGLLLFIGLGIFFCVRCRHRRRQAERMSQIKR |
| | | LLSEKKTCQCPHRFQKTCSPI |
| 498 | Amino Acid | MALPVTALLLPLALLLHAARPQVQLKESGGGLVQP |
| | | GGSLKLSCTTSGFTFSSYGLSWIRQTPDKRLELVA |
| | | SINRNGDNTYYTDSVRGRFTISRDNAKSTLNLQMS |
| | | SLKSEDTAMYYCTRGGFSYWGQGTLVTVSAGSTSG |
| | | SGKPGSGEGSTKGQIVLTQSPPLMSASPGEKVTMT |
| | | CSASSSVGYMYWFQQRPGSSPRLLIYDTYNLASGV |
| | | PVRFSGSGSGTSYSLTISRLEAEDAATYYCQQWSG |
| | | YPPITFGAGTKLELREQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPTKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 499 | Amino Acid | MALPVTALLLPLALLLHAARPEVKLVESGGGLVQP |
| | | GGSLKLSCAASGFTFSSYGLSWVRQTPDKRLELVA |
| | | SVNRNGGNTYYTDSVKGRFTISRDNAKNTLNLQMS |
| | | SLKSEDTAMYYCARGGFTYWGQGTLVTVSAGSTSG |
| | | SGKPGSGEGSTKGQIVLTQSPAIMSASPGEKVTMT |
| | | CSASSSVGYMYWYQQKPGSSPRLLIYDTSHLASGV |
| | | PVRFSGSGSGTSYSLTISRMEAEDAATYYCQQWSG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGGGGGSGGGGSEVQ |
| | | LVESGGGLVQPGGSLRLSCAASGYSFTGYTMNWVR |
| | | QAPGKGLEWVALINPTKGVSTYNQKFKDRFTISVD |
| | | KSKNTAYLQMNSLRAEDTAVYYCARSGYYGDSDWY |
| | | FDVWGQGTLVTVSSTSGGGGSLESGQVLLESNIKV |
| | | LPTWSTPVQPMALIVLGGVAGLLLFIGLGIFFCVR |
| | | CRHRRRQAERMSQIKRLLSEKKTCQCPHRFQKTCS |
| | | PI |
| 500 | Amino Acid | MALPVTALLLPLALLLHAARPEVKLVESGGGLVQP |
| | | GGSLKLSCAASGFPFSSYGLSWVRQTPDKRLELVA |
| | | TINRNGDSAYYPDSVKGRFTISRDDAKNTLYLQMS |
| | | SLKSEDTAMYYCTRGGFAYWGQGTLVTVSAGSTSG |
| | | SGKPGSGEGSTKGQIVLTQSPVIMSASPGEKVTMT |
| | | CSASSSVGYMYWYQQRPGSSPRLLIYDTSNLASGA |
| | | PVRFSGSGSGTSYSLTISRMEAEDAATYYCQQWSG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPTKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 501 | Amino Acid | MALPVTALLLPLALLLHAARPEVKLVESGGGLVQP |
| | | GGSLKLSCTTSGFTFSSYGLSWIRQTPDKRLELVA |
| | | SINRNGDNTYYTDSVKGRFTISRDNAKNTLNLQMS |
| | | SLKSEDTAMYYCTRGGFSYWGQGTLVTVSAGSTSG |
| | | SGKPGSGEGSTKGQIVLTQSPAIMSASPGEKVTMT |
| | | CSASSSVSYMYWYQQKPGSSPRLLIYDTSNLASGV |
| | | PVRFSGSGSGTSYSLTISRMEAEDAATYYCQQWTG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPTKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 502 | Amino Acid | MALPVTALLLPLALLLHAARPEVKLVESGGVLVQP |
| | | GGSLKLSCAASGFTFSSYGMSWVRQTPDKRLELVA |
| | | SINKNGGSTYYPDSVKGRFTISRDNAKTTVYLQMS |
| | | SLKSEDTAMYYCTRGGFAYWGQGTLVTVSAGSTSG |
| | | SGKPGSGEGSTKGQIVLTQSPAIMSASPGEKVTLT |
| | | CSASSSVGYMYWYQQRPGSSPRLLIYDTSNLPSGV |
| | | PVRFSGSGSGTSYSLTISRMEAEDAATYYCQQWSG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPTKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 503 | Amino Acid | MALPVTALLLPLALLLHAARPQLQLQESGGGLVQA |
| | | GGSLRLSCAASGRTGSSYAMGWFRQAPGKEREFVA |
| | | AITWSGGITAYADSVKGRFTISRDNAKNTVYLQMN |
| | | SLKPEDTAVYCCAAGVTGSPSFDSWGQGTQVTVSS |
| | | EQKLISEEDLNPGGGGGSGGGGSGGGGSGGGGSGS |
| | | MDIQMTQSPSSLSASVGDRVTITCRASQDIRNYLN |
| | | WYQQKPGKAPKLLIYYTSRLESGVPSRFSGSGSGT |
| | | DYTLTISS |
| | | LQPEDFATYYCQQGNTLPWTFGQGTKVEIKGGGGS |
| | | GGGGSGGGGSEVQLVESGGGLVQPGGSLRLSCAAS |
| | | GYSFTGYTMNWVRQAPGKGLEWVALINPTKGVSTY |
| | | NQKFKDRFTISVDKSKNTAYLQMNSLRAEDTAVYY |
| | | CARSGYYGDSDWYFDVWGQGTLVTVSSTSGGGGSL |
| | | ESGQVLLESNIKVLPTWSTPVQPMALIVLGGVAGL |
| | | LLFIGLGIFFCVRCRHRRRQAERMSQIKRLLSEKK |
| | | TCQCPHRFQKTCSPI |
| 504 | Amino Acid | MALPVTALLLPLALLLHAARPQVQLQESGGGLVQA |
| | | GGSLRLSCAASGRTFSSYAMGWFRQAPGKEREFVA |
| | | AISGSGGSIYYGDSVKGRFTISRDNAKNTMYLQMN |
| | | RLKPEDTAVYYCAAGPLGSPDFDSWGQGTQVTVSS |
| | | EQKLISEEDLNPGGGGGSGGGGSGGGGSGGGGSGS |
| | | MDIQMTQSPSSLSASVGDRVTITCRASQDIRNYLN |
| | | WYQQKPGKAPKLLIYYTSRLESGVPSRFSGSGSGT |
| | | DYTLTISSLQPEDFATYYCQQGNTLPWTFGQGTKV |
| | | EIKGGGGSGGGGSGGGGSEVQLVESGGGLVQPGGS |
| | | LRLSCAASGYSFTGYTMNWVRQAPGKGLEWVALIN |
| | | PTKGVSTYNQKFKDRFTISVDKSKNTAYLQMNSLR |
| | | AEDTAVYYCARSGYYGDSDWYFDVWGQGTLVTVSS |
| | | TSGGGGSLESGQVLLESNIKVLPTWSTPVQPMALI |
| | | VLGGVAGLLLFIGLGIFFCVRCRHRRRQAERMSQI |
| | | KRLLSEKKTCQCPHRFQKTCSPI |
| 505 | Amino Acid | MALPVTALLLPLALLLHAARPEVQVVESGGGLVQP |
| | | GGSLRLSCVASGRTFSSYAMGWFRQAPGKEREFVA |
| | | AISGSGGSIYYGDSVKGRFTISRDNAKNTMYLQMN |
| | | RLKPEDTAVYYCAAGPLGSPDFDSWGQGTQVTVSS |
| | | EQKLISEEDLNPGGGGGSGGGGSGGGGSGGGGSGS |
| | | MDIQMTQSPSSLSASVGDRVTITCRASQDIRNYLN |
| | | WYQQKPGKAPKLLIYYTSRLESGVPSRFSGSGSGT |
| | | DYTLTISSLQPEDFATYYCQQGNTLPWTFGQGTKV |
| | | EIKGGGGSGGGGSGGGGSEVQLVESGGGLVQPGGS |
| | | LRLSCAASGYSFTGYTMNWVRQAPGKGLEWVALIN |
| | | PTKGVSTYNQKFKDRFTISVDKSKNTAYLQMNSLR |
| | | AEDTAVYYCARSGYYGDSDWYFDVWGQGTLVTVSS |
| | | TSGGGGSLESGQVLLESNIKVLPTWSTPVQPMALI |
| | | VLGGVAGLLLFIGLGIFFCVRCRHRRRQAERMSQI |
| | | KRLLSEKKTCQCPHRFQKTCSPI |
| 506 | Amino Acid | MALPVTALLLPLALLLHAARPKVQLVESGGGLVQA |
| | | GGSLRLSCAASGRTGSSYAMGWFRQAPGKEREFVA |
| | | AITWSGGITAYADSVKGRFTISRDNAKNTVYLQMN |
| | | SLKPEDTAVYCCAAGVTGSPSFDSWGQGTQVTVSS |
| | | EQKLISEEDLNPGGGGGSGGGGSGGGGSGGGGSGS |
| | | MDIQMTQSPSSLSASVGDRVTITCRASQDIRNYLN |
| | | WYQQKPGKAPKLLIYYTSRLESGVPSRFSGSGSGT |
| | | DYTLTISSLQPEDFATYYCQQGNTLPWTFGQGTKV |
| | | EIKGGGGSGGGGSGGGGSEVQLVESGGGLVQPGGS |
| | | LRLSCAASGYSFTGYTMNWVRQAPGKGLEWVALIN |
| | | PTKGVSTYNQKFKDRFTISVDKSKNTAYLQMNSLR |
| | | AEDTAVYYCARSGYYGDSDWYFDVWGQGTLVTVSS |
| | | TSGGGGSLESGQVLLESNIKVLPTWSTPVQPMALI |
| | | VLGGVAGLLLFIGLGIFFCVRCRHRRRQAERMSQI |
| | | KRLLSEKKTCQCPHRFQKTCSPI |
| 507 | Amino Acid | MALPVTALLLPLALLLHAARPEVQLVESGGGLVQA |
| | | GGSLRLSCAASGRTFSSYAMGWFRQAPGKEREFVA |
| | | AISGSGGVTFYAHSVKGRFTISRDNAKNTVYLQMN |
| | | SLKPEDTAVYSCAGGAHGSPDFGSWGQGTQVTVSS |
| | | EQKLISEEDLNPGGGGGSGGGGSGGGGSGGGGSGS |
| | | MDIQMTQSPSSLSASVGDRVTITCRASQDIRNYLN |
| | | WYQQKPGKAPKLLIYYTSRLESGVPSRFSGSGSGT |
| | | DYTLTISSLQPEDFATYYCQQGNTLPWTFGQGTKV |
| | | EIKGGGGSGGGGSGGGGSEVQLVESGGGLVQPGGS |
| | | LRLSCAASGYSFTGYTMNWVRQAPGKGLEWVALIN |
| | | PTKGVSTYNQKFKDRFTISVDKSKNTAYLQMNSLR |
| | | AEDTAVYYCARSGYYGDSDWYFDVWGQGTLVTVSS |
| | | TSGGGGSLESGQVLLESNIKVLPTWSTPVQPMALI |
| | | VLGGVAGLLLFIGLGIFFCVRCRHRRRQAERMSQI |
| | | KRLLSEKKTCQCPHRFQKTCSPI |
| 508 | Amino Acid | MALPVTALLLPLALLLHAARPQVQLVESGGGLVQP |
| | | GGSLRLSCAVSRNIASLYRVDWYRQAPGKQRELVA |
| | | GRTSGGTTTYLDAVEGRFTISRDNVKDTVYLQMNS |
| | | LTPEDTAVYYCHAHDHWRDSWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 509 | Amino Acid | MALPVTALLLPLALLLHAARPQVQLVESGGDLVQP |
| | | GGSLRLSCAASGSIGSIYAMGWYRQAPGRQRELVA |
| | | TTTSGGTTNYADSVKGRFTIAGDNAKNTVFLQMNS |
| | | LRPEDTAVYYCKIQTHWYVYWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 510 | Amino Acid | MALPVTALLLPLALLLHAARPEVQLVESGGGLVQP |
| | | GGSLRLSCAASRNIFSLYRVDWYRQAPGKQRELVA |
| | | GSTSGGTTTYADAVKGRFTISTDNVKDTVYLQMNS |
| | | LTPEDTAVYYCHAHDHWRDSWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 511 | Amino Acid | MALPVTALLLPLALLLHAARPQVQLVESGGGWVHP |
| | | GGSLRLSCAASRNIFSMYRVDWYRQAPGKQRELVA |
| | | GITSGGTTSYADAVKGRFTISTDNVKDTVYLQMNS |
| | | VTPEDTAVYYCHAHDHWRDSWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 512 | Amino Acid | MALPVTALLLPLALLLHAARPEVQVQESGGDLVQP |
| | | GGSLRLSCAASGSIGSIYRKGWYRQAPGSQRELVA |
| | | TITSAGTTNYADSVKGRFTISRDNAKNTVYLQMNS |
| | | LRPEDTAVYYCNFQTHWYVYWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 513 | Amino Acid | MALPVTALLLPLALLLHAARPEVRLVESGGGLVQP |
| | | GGSLRLSCAVSKNIFSIYRVDWYHQAPGKQRELVA |
| | | GWTSGGSTSYADAVKGRFTISTDNVKDTVYLQMNS |
| | | LTPEDTAVYYCHAHDHWRDYWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 514 | Amino Acid | QVQLQESGGGLVQPGGSLRLSCAASGIIFSINTMA |
| | | WYRQGPGKQRDLVALISSGGNTSYADSVNGRFTIS |
| | | RDNAKNTVYLQMNGLKPEDTAVYYCNSAGRSYSGS |
| | | YGAYWGQGTQVTVSS |
| 515 | Amino Acid | EVQLVESGGGLVQPGGSLRLSCAASRSIFVIAFMD |
| | | WYRQAPGKQREFVAGISSSGATSYASSVKGRFTVS |
| | | RDTAKNTMYLQMNNLLPEDSAVYYCKADTATTTDW |
| | | GQGTQVTVSS |
| 516 | Amino Acid | QVQLQESGGGLVQPGGSLRLSCAASRSIFGIAFMD |
| | | WYRQAPGKQREFVAGISHSGATSYADSVKGRFTVS |
| | | RDNAKNTMYLQMNNLLPEDSAVYYCKADTATNTDW |
| | | GQGTQVTVSS |
| 517 | Amino Acid | QLQLVESGGGLVQAGGSLRLSCAVSGNIWVFTVMA |
| | | WYRQVPGKQRELVAASTNGGSTNYADSVKGRFTIS |
| | | RDNVKNTVYLQMNSLKPEDTAVYYCNRQRTVIGMN |
| | | PLAPWGQGTQVTVSS |
| 518 | Amino Acid | QVQLVESGGGLVQPGGSLRLSCATSGIIFHIYTMG |
| | | WYRGAPGKQRELVARITSGGDTNYADSVKGRFTIS |
| | | RDNVKNTMYLQMNSLKPEDTAVYYCNRFPGATFSW |
| | | GQGTQVTVSS |
| 519 | Amino Acid | EVQLVESGGGLVQPGGSLRLSCAASGIIFSINTMA |
| | | WYRQGPGKERDLVAVIKGDGSTSYADSVNGRFTIS |
| | | RDNAKNTVYLRMNGLKPEDTAVYYCNSAGRSYSGV |
| | | YGAYWGQGTQVTVSS |
| 520 | Amino Acid | EVQLQESGGGLVQPGGSLKLSCAASGSIFSIGAMR |
| | | WYRQVPGNERELVAGITNGGNTNYADSVKARFTIS |
| | | RDNAKNTVYLQMNSLKPEDTTVYFCNADVQNSGYV |
| | | WGNYWGQGTQVTVSS |
| 521 | Amino Acid | EVQLVESGGGLVQPGGSLRLSCAASRSIFVIAFMD |
| | | WYRQAPGKQREFVAGISSSGATSYADSVKGRFTVS |
| | | RDTAKNTMYLQMNNLLPEDSAVYYCKADTATNTDW |
| | | GQGTQVTVSS |
| 522 | Amino Acid | GIIFSINTMA |
| 523 | Amino Acid | LISSGGNTSYADS |
| 524 | Amino Acid | NSAGRSYSGSYGAY |
| 525 | Amino Acid | RSIFVIAFMD |
| 526 | Amino Acid | GISSSGATSYASS |
| 527 | Amino Acid | KADTATTTD |
| 528 | Amino Acid | RSIFGIAFMD |
| 529 | Amino Acid | GISHSGATSYADS |
| 530 | Amino Acid | KADTATNTD |
| 531 | Amino Acid | GNIWVFTVMA |
| 532 | Amino Acid | ASTNGGSTNYADS |
| 533 | Amino Acid | NRQRTVIGMNPLAP |
| 534 | Amino Acid | GIIFHIYTMG |
| 535 | Amino Acid | RITSGGDTNYADS |
| 536 | Amino Acid | NRFPGATFS |
| 537 | Amino Acid | GIIFSINTMA |
| 538 | Amino Acid | VIKGDGSTSYADS |
| 539 | Amino Acid | NSAGRSYSGVYGAY |
| 540 | Amino Acid | GSIFSIGAMR |
| 541 | Amino Acid | GITNGGNTNYADS |
| 542 | Amino Acid | NADVQNSGYVWGNY |
| 543 | Amino Acid | RSIFVIAFMD |
| 544 | Amino Acid | GISSSGATSYADS |
| 545 | Amino Acid | KADTATNTD |
| 546 | Amino Acid | MALPVTALLLPLALLLHAARPQVQLQESGGGLVQP |
| | | GGSLRLSCAASGIIFSINTMAWYRQGPGKQRDLVA |
| | | LISSGGNTSYADSVNGRFTISRDNAKNTVYLQMNG |
| | | LKPEDTAVYYCNSAGRSYSGSYGAYWGQGTQVTVS |
| | | SEQKLISEEDLNPGGGGGSGGGGSGGGGSGGGGSG |
| | | SMDIQMTQSPSSLSASVGDRVTITCRASQDIRNYL |
| | | NWYQQKPGKAPKLLIYYTSRLESGVPSRFSGSGSG |
| | | TDYTLTISSLQPEDFATYYCQQGNTLPWTFGQGTK |
| | | VEIKGGGGSGGGGSGGGGSEVQLVESGGGLVQPGG |
| | | SLRLSCAASGYSFTGYTMNWVRQAPGKGLEWVALI |
| | | NPTKGVSTYNQKFKDRFTISVDKSKNTAYLQMNSL |
| | | RAEDTAVYYCARSGYYGDSDWYFDVWGQGTLVTVS |
| | | STSGGGGSLESGQVLLESNIKVLPTWSTPVQPMAL |
| | | IVLGGVAGLLLFIGLGIFFCVRCRHRRRQAERMSQ |
| | | IKRLLSEKKTCQCPHRFQKTCSPI |
| 547 | Amino Acid | MALPVTALLLPLALLLHAARPEVQLVESGGGLVQP |
| | | GGSLRLSCAASRSIFVIAFMDWYRQAPGKQREFVA |
| | | GISSSGATSYASSVKGRFTVSRDTAKNTMYLQMNN |
| | | LLPEDSAVYYCKADTATTTDWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 548 | Amino Acid | MALPVTALLLPLALLLHAARPQVQLQESGGGLVQP |
| | | GGSLRLSCAASRSIFGIAFMDWYRQAPGKQREFVA |
| | | GISHSGATSYADSVKGRFTVSRDNAKNTMYLQMNN |
| | | LLPEDSAVYYCKADTATNTDWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 549 | Amino Acid | MALPVTALLLPLALLLHAARPQLQLVESGGGLVQA |
| | | GGSLRLSCAVSGNIWVFTVMAWYRQVPGKQRELVA |
| | | ASTNGGSTNYADSVKGRFTISRDNVKNTVYLQMNS |
| | | LKPEDTAVYYCNRQRTVIGMNPLAPWGQGTQVTVS |
| | | SEQKLISEEDLNPGGGGGSGGGGSGGGGSGGGGSG |
| | | SMDIQMTQSPSSLSASVGDRVTITCRASQDIRNYL |
| | | NWYQQKPGKAPKLLIYYTSRLESGVPSRFSGSGSG |
| | | TDYTLTISSLQPEDFATYYCQQGNTLPWTFGQGTK |
| | | VEIKGGGGSGGGGSGGGGSEVQLVESGGGLVQPGG |
| | | SLRLSCAASGYSFTGYTMNWVRQAPGKGLEWVALI |
| | | NPTKGVSTYNQKFKDRFTISVDKSKNTAYLQMNSL |
| | | RAEDTAVYYCARSGYYGDSDWYFDVWGQGTLVTVS |
| | | STSGGGGSLESGQVLLESNIKVLPTWSTPVQPMAL |
| | | IVLGGVAGLLLFIGLGIFFCVRCRHRRRQAERMSQ |
| | | IKRLLSEKKTCQCPHRFQKTCSPI |
| 550 | Amino Acid | MALPVTALLLPLALLLHAARPQVQLVESGGGLVQP |
| | | GGSLRLSCATSGIIFHIYTMGWYRGAPGKQRELVA |
| | | RITSGGDTNYADSVKGRFTISRDNVKNTMYLQMNS |
| | | LKPEDTAVYYCNRFPGATFSWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 551 | Amino Acid | MALPVTALLLPLALLLHAARPEVQLVESGGGLVQP |
| | | GGSLRLSCAASGIIFSINTMAWYRQGPGKERDLVA |
| | | VIKGDGSTSYADSVNGRFTISRDNAKNTVYLRMNG |
| | | LKPEDTAVYYCNSAGRSYSGVYGAYWGQGTQVTVS |
| | | SEQKLISEEDLNPGGGGGSGGGGSGGGGSGGGGSG |
| | | SMDIQMTQSPSSLSASVGDRVTITCRASQDIRNYL |
| | | NWYQQKPGKAPKLLIYYTSRLESGVPSRFSGSGSG |
| | | TDYTLTISSLQPEDFATYYCQQGNTLPWTFGQGTK |
| | | VEIKGGGGSGGGGSGGGGSEVQLVESGGGLVQPGG |
| | | SLRLSCAASGYSFTGYTMNWVRQAPGKGLEWVALI |
| | | NPTKGVSTYNQKFKDRFTISVDKSKNTAYLQMNSL |
| | | RAEDTAVYYCARSGYYGDSDWYFDVWGQGTLVTVS |
| | | STSGGGGSLESGQVLLESNIKVLPTWSTPVQPMAL |
| | | IVLGGVAGLLLFIGLGIFFCVRCRHRRRQAERMSQ |
| | | IKRLLSEKKTCQCPHRFQKTCSPI |
| 552 | Amino Acid | MALPVTALLLPLALLLHAARPEVQLQESGGGLVQP |
| | | GGSLKLSCAASGSIFSIGAMRWYRQVPGNERELVA |
| | | GITNGGNTNYADSVKARFTISRDNAKNTVYLQMNS |
| | | LKPEDTTVYFCNADVQNSGYVWGNYWGQGTQVTVS |
| | | SEQKLISEEDLNPGGGGGSGGGGSGGGGSGGGGSG |
| | | SMDIQMTQSPSSLSASVGDRVTITCRASQDIRNYL |
| | | NWYQQKPGKAPKLLIYYTSRLESGVPSRFSGSGSG |
| | | TDYTLTISSLQPEDFATYYCQQGNTLPWTFGQGTK |
| | | VEIKGGGGSGGGGSGGGGSEVQLVESGGGLVQPGG |
| | | SLRLSCAASGYSFTGYTMNWVRQAPGKGLEWVALI |
| | | NPTKGVSTYNQKFKDRFTISVDKSKNTAYLQMNSL |
| | | RAEDTAVYYCARSGYYGDSDWYFDVWGQGTLVTVS |
| | | STSGGGGSLESGQVLLESNIKVLPTWSTPVQPMAL |
| | | IVLGGVAGLLLFIGLGIFFCVRCRHRRRQAERMSQ |
| | | IKRLLSEKKTCQCPHRFQKTCSPI |
| 553 | Amino Acid | MALPVTALLLPLALLLHAARPEVQLVESGGGLVQP |
| | | GGSLRLSCAASRSIFVIAFMDWYRQAPGKQREFVA |
| | | GISSSGATSYADSVKGRFTVSRDTAKNTMYLQMNN |
| | | LLPEDSAVYYCKADTATNTDWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPTKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 554 | Amino Acid | MALPVTALLLPLALLLHAARPEVKLVESGGGLVQP |
| | | GGSLKLSCAASGFTFSSYGLSWVRQTPDKGLELVA |
| | | SINRNGGNTYYTDSVKGRFTISRDNAKNTLNLQMS |
| | | SLKSEDTAMYYCARGGFTYWGQGTLVTVSAGSTSG |
| | | SGKPGSGEGSTKGQIVLTQSPAIMSAFPGEKVTMT |
| | | CSASSSVGYMYWYQQKPGSSPRLLIYDTSNLASGV |
| | | PVRFSGSGSGTAYSLTISRMEAEDAATYYCQQWSG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPYKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 555 | Amino Acid | MALPVTALLLPLALLLHAARPQIVLTQSPAIMSAF |
| | | PGEKVTMTCSASSSVGYMYWYQQKPGSSPRLLIYD |
| | | TSNLASGVPVRFSGSGSGTAYSLTISRMEAEDAAT |
| | | YYCQQWSGYPPITFGAGTKLELKGSTSGSGKPGSG |
| | | EGSTKGEVKLVESGGGLVQPGGSLKLSCAASGFTF |
| | | SSYGLSWVRQTPDKGLELVASINRNGGNTYYTDSV |
| | | KGRFTISRDNAKNTLNLQMSSLKSEDTAMYYCARG |
| | | GFTYWGQGTLVTVSAEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPYKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 556 | Amino Acid | MALPVTALLLPLALLLHAARPQVQLKESGGGLVQP |
| | | GGSLKLSCTTSGFTFSSYGLSWIRQTPDKRLELVA |
| | | SINRNGDNTYYTDSVRGRFTISRDNAKSTLNLQMS |
| | | SLKSEDTAMYYCTRGGFSYWGQGTLVTVSAGSTSG |
| | | SGKPGSGEGSTKGQIVLTQSPPLMSASPGEKVTMT |
| | | CSASSSVGYMYWFQQRPGSSPRLLIYDTYNLASGV |
| | | PVRFSGSGSGTSYSLTISRLEAEDAATYYCQQWSG |
| | | YPPITFGAGTKLELREQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPYKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 557 | Amino Acid | MALPVTALLLPLALLLHAARPQIVLTQSPPLMSAS |
| | | PGEKVTMTCSASSSVGYMYWFQQRPGSSPRLLIYD |
| | | TYNLASGVPVRFSGSGSGTSYSLTISRLEAEDAAT |
| | | YYCQQWSGYPPITFGAGTKLELRGSTSGSGKPGSG |
| | | EGSTKGQVQLKESGGGLVQPGGSLKLSCTTSGFTF |
| | | SSYGLSWIRQTPDKRLELVASINRNGDNTYYTDSV |
| | | RGRFTISRDNAKSTLNLQMSSLKSEDTAMYYCTRG |
| | | GFSYWGQGTLVTVSAEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPYKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 558 | Amino Acid | MALPVTALLLPLALLLHAARPQVQLKESGGGLVRP |
| | | GGSLKLSCAASGFTFSNYGMSWIRQTPDKNLELVA |
| | | SINTSGGNTYYPDSVKGRFTISRDNARATLNLQMS |
| | | NLKSEDTAIYYCTRGGFTHWGQGTLVAVSAGSTSG |
| | | SGKPGSGEGSTKGQIVLTQSPAIMSASPGEKVTMT |
| | | CSASSSVSYMYWYQQKPGSSPRLLIYDTSNLASGV |
| | | PVRFSGSGSGTSYFLTISRMEAEDAATYYCQQWTG |
| | | YPPITFGAGTKLELREQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPYKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 559 | Amino Acid | MALPVTALLLPLALLLHAARPQIVLTQSPAIMSAS |
| | | PGEKVTMTCSASSSVSYMYWYQQKPGSSPRLLIYD |
| | | TSNLASGVPVRFSGSGSGTSYFLTISRMEAEDAAT |
| | | YYCQQWTGYPPITFGAGTKLELRGSTSGSGKPGSG |
| | | EGSTKGQVQLKESGGGLVRPGGSLKLSCAASGFTF |
| | | SNYGMSWIRQTPDKNLELVASINTSGGNTYYPDSV |
| | | KGRFTISRDNARATLNLQMSNLKSEDTAIYYCTRG |
| | | GFTHWGQGTLVAVSAEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPYKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 560 | Amino Acid | MALPVTALLLPLALLLHAARPQVQLKESGGGLVQP |
| | | GGSLKLSCAASGFPFSSYGLSWVRQTPDKRLELVA |
| | | TINRNGDSAYYPDSVKGRFTISRDDAKNTLYLQMS |
| | | SLKSEDTAMYYCTRGGFAYWGQGTLVTVSAGSTSG |
| | | SGKPGSGEGSTKGQIVLTQSPAIMSASPGEKVTMT |
| | | CSGSSSVGYMYWYQQKPGFSPRLLIYDTSNLASGV |
| | | PVRFSGSGSGTSYSLTISRMEAEDAATYYCQQWSG |
| | | YPPITFGAGTKLELKEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPYKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWSTPVQPMALIVLGGVAGLLLFIGLGIFFCV |
| | | RCRHRRRQAERMSQIKRLLSEKKTCQCPHRFQKTC |
| | | SPI |
| 561 | Amino Acid | MALPVTALLLPLALLLHAARPQIVLTQSPAIMSAS |
| | | PGEKVTMTCSGSSSVGYMYWYQQKPGFSPRLLIYD |
| | | TSNLASGVPVRFSGSGSGTSYSLTISRMEAEDAAT |
| | | YYCQQWSGYPPITFGAGTKLELKGSTSGSGKPGSG |
| | | EGSTKGQVQLKESGGGLVQPGGSLKLSCAASGFPF |
| | | SSYGLSWVRQTPDKRLELVATINRNGDSAYYPDSV |
| | | KGRFTISRDDAKNTLYLQMSSLKSEDTAMYYCTRG |
| | | GFAYWGQGTLVTVSAEQKLISEEDLNPGGGGGSGG |
| | | GGSGGGGSGGGGSGSMDIQMTQSPSSLSASVGDRV |
| | | TITCRASQDIRNYLNWYQQKPGKAPKLLIYYTSRL |
| | | ESGVPSRFSGSGSGTDYTLTISSLQPEDFATYYCQ |
| | | QGNTLPWTFGQGTKVEIKGGGGSGGGGSGGGGSEV |
| | | QLVESGGGLVQPGGSLRLSCAASGYSFTGYTMNWV |
| | | RQAPGKGLEWVALINPYKGVSTYNQKFKDRFTISV |
| | | DKSKNTAYLQMNSLRAEDTAVYYCARSGYYGDSDW |
| | | YFDVWGQGTLVTVSSTSGGGGSLESGQVLLESNIK |
| | | VLPTWST |
| | | PVQPMALIVLGGVAGLLLFIGLGIFFCVRCRHRRR |
| | | QAERMSQIKRLLSEKKTCQCPHRFQKTCSPI |
| 562 | Amino Acid | MALPVTALLLPLALLLHAARPQVQLQESGGGLVQP |
| | | GGSLRLSCAASRSIFGIAFMDWYRQAPGKQREFVA |
| | | GISHSGATSYADSVKGRFTVSRDNAKNTMYLQMNN |
| | | LLPEDSAVYYCKADTATNTDWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGGGGGSGGGGSEVQLVESGGGLVQPGGSLRLSC |
| | | AASGYSFTGYTMNWVRQAPGKGLEWVALINPYKGV |
| | | STYNQKFKDRFTISVDKSKNTAYLQMNSLRAEDTA |
| | | VYYCARSGYYGDSDWYFDVWGQGTLVTVSSTSGGG |
| | | GSLESGQVLLESNIKVLPTWSTPVQPMALIVLGGV |
| | | AGLLLFIGLGIFFCVRCRHRRRQAERMSQIKRLLS |
| | | EKKTCQCPHRFQKTCSPI |
| 563 | Amino Acid | MALPVTALLLPLALLLHAARPEVQLVESGGGLVQP |
| | | GGSLRLSCAASRSIFVIAFMDWYRQAPGKQREFVA |
| | | GISSSGATSYASSVKGRFTVSRDTAKNTMYLQMNN |
| | | LLPEDSAVYYCKADTATTTDWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPYKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 564 | Amino Acid | MALPVTALLLPLALLLHAARPEVQLVESGGGLVQP |
| | | GGSLRLSCAASGIIFSINTMAWYRQGPGKERDLVA |
| | | VIKGDGSTSYADSVNGRFTISRDNAKNTVYLRMNG |
| | | LKPEDTAVYYCNSAGRSYSGVYGAYWGQGTQVTVS |
| | | SEQKLISEEDLNPGGGGGSGGGGSGGGGSGGGGSG |
| | | SMDIQMTQSPSSLSASVGDRVTITCRASQDIRNYL |
| | | NWYQQKPGKAPKLLIYYTSRLESGVPSRFSGSGSG |
| | | TDYTLTISSLQPEDFATYYCQQGNTLPWTFGQGTK |
| | | VEIKGGGGSGGGGSGGGGSEVQLVESGGGLVQPGG |
| | | SLRLSCAASGYSFTGYTMNWVRQAPGKGLEWVALI |
| | | NPYKGVSTYNQKFKDRFTISVDKSKNTAYLQMNSL |
| | | RAEDTAVYYCARSGYYGDSDWYFDVWGQGTLVTVS |
| | | STSGGGGSLESGQVLLESNIKVLPTWSTPVQPMAL |
| | | IVLGGVAGLLLFIGLGIFFCVRCRHRRRQAERMSQ |
| | | IKRLLSEKKTCQCPHRFQKTCSPI |
| 565 | Amino Acid | MALPVTALLLPLALLLHAARPEVQLQESGGGLVQP |
| | | GGSLKLSCAASGSIFSIGAMRWYRQVPGNERELVA |
| | | GITNGGNTNYADSVKARFTISRDNAKNTVYLQMNS |
| | | LKPEDTTVYFCNADVQNSGYVWGNYWGQGTQVTVS |
| | | SEQKLISEEDLNPGGGGGSGGGGSGGGGSGGGGSG |
| | | SMDIQMTQSPSSLSASVGDRVTITCRASQDIRNYL |
| | | NWYQQKPGKAPKLLIYYTSRLESGVPSRFSGSGSG |
| | | TDYTLTISSLQPEDFATYYCQQGNTLPWTFGQGTK |
| | | VEIKGGGGSGGGGSGGGGSEVQLVESGGGLVQPGG |
| | | SLRLSCAASGYSFTGYTMNWVRQAPGKGLEWVALI |
| | | NPYKGVSTYNQKFKDRFTISVDKSKNTAYLQMNSL |
| | | RAEDTAVYYCARSGYYGDSDWYFDVWGQGTLVTVS |
| | | STSGGGGSLESGQVLLESNIKVLPTWSTPVQPMAL |
| | | IVLGGVAGLLLFIGLGIFFCVRCRHRRRQAERMSQ |
| | | IKRLLSEKKTCQCPHRFQKTCSPI |
| 566 | Amino Acid | MALPVTALLLPLALLLHAARPEVQLVESGGGLVQP |
| | | GGSLRLSCAASRSIFVIAFMDWYRQAPGKQREFVA |
| | | GISSSGATSYADSVKGRFTVSRDTAKNTMYLQMNN |
| | | LLPEDSAVYYCKADTATNTDWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPYKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 567 | Amino Acid | MALPVTALLLPLALLLHAARPQVQLVESGGGLVQP |
| | | GGSLRLSCATSGIIFHIYTMGWYRGAPGKQRELVA |
| | | RITSGGDTNYADSVKGRFTISRDNVKNTMYLQMNS |
| | | LKPEDTAVYYCNRFPGATFSWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPYKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 568 | Amino Acid | MALPVTALLLPLALLLHAARPQLQLVESGGGLVQA |
| | | GGSLRLSCAVSGNIWVFTVMAWYRQVPGKQRELVA |
| | | ASTNGGSTNYADSVKGRFTISRDNVKNTVYLQMNS |
| | | LKPEDTAVYYCNRQRTVIGMNPLAPWGQGTQVTVS |
| | | SEQKLISEEDLNPGGGGGSGGGGSGGGGSGGGGSG |
| | | SMDIQMTQSPSSLSASVGDRVTITCRASQDIRNYL |
| | | NWYQQKPGKAPKLLIYYTSRLESGVPSRFSGSGSG |
| | | TDYTLTISSLQPEDFATYYCQQGNTLPWTFGQGTK |
| | | VEIKGGGGSGGGGSGGGGSEVQLVESGGGLVQPGG |
| | | SLRLSCAASGYSFTGYTMNWVRQAPGKGLEWVALI |
| | | NPYKGVSTYNQKFKDRFTISVDKSKNTAYLQMNSL |
| | | RAEDTAVYYCARSGYYGDSDWYFDVWGQGTLVTVS |
| | | STSGGGGSLESGQVLLESNIKVLPTWSTPVQPMAL |
| | | IVLGGVAGLLLFIGLGIFFCVRCRHRRRQAERMSQ |
| | | IKRLLSEKKTCQCPHRFQKTCSPI |
| 569 | Amino Acid | MALPVTALLLPLALLLHAARPQVQLVESGGGLVQP |
| | | GGSLRLSCAVSRNIASLYRVDWYRQAPGKQRELVA |
| | | GRTSGGTTTYLDAVEGRFTISRDNVKDTVYLQMNS |
| | | LTPEDTAVYYCHAHDHWRDSWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPYKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 570 | Amino Acid | MALPVTALLLPLALLLHAARPEVQLVESGGGLVQP |
| | | GGSLRLSCAASRNIFSLYRVDWYRQAPGKQRELVA |
| | | GSTSGGTTTYADAVKGRFTISTDNVKDTVYLQMNS |
| | | LTPEDTAVYYCHAHDHWRDSWGQGTQVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPYKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 571 | Amino Acid | MALPVTALLLPLALLLHAARPQVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARDILRLSVSSGMDVWGQGTLVT |
| | | VSSGSTSGSGKPGSGEGSTKGQSVLTQPPSASGTP |
| | | GQRVTISCSGSSSNIGSNYVYWYQQLPGTAPKLLI |
| | | YRNNQRPSGVPDRLSGSKSGTSASLAISGLRSEDE |
| | | ADYYCAAWDDSLSAVVFGGGTKLTVLEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPYKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 572 | Amino Acid | MALPVTALLLPLALLLHAARPQSVLTQPPSASGTP |
| | | GQRVTISCSGSSSNIGSNYVYWYQQLPGTAPKLLI |
| | | YRNNQRPSGVPDRLSGSKSGTSASLAISGLRSEDE |
| | | ADYYCAAWDDSLSAVVFGGGTKLTVLGSTSGSGKP |
| | | GSGEGSTKGQVQLVQSGAEVKKPGSSVKVSCKASG |
| | | GTFSSYAISWVRQAPGQGLEWMGGIIPIFGTANYA |
| | | QKFQGRVTITADESTSTAYMELSSLRSEDTAVYYC |
| | | ARDILRLSVSSGMDVWGQGTLVTVSSEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPYKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 573 | Amino Acid | MALPVTALLLPLALLLHAARPQVQLQESGGGVVQP |
| | | GRSLRLSCAASGFTLSSYGMHWVRQAPGKGLEWVA |
| | | VISYDGSNKYYADSVKGRFTISRDNSKNTLYLQMN |
| | | SLRAEDTAVYYCAKSGWRYYYYYGMDVWGQGTLVT |
| | | VSSGSTSGSGKPGSGEGSTKGQSVLTQPPSVSGAP |
| | | GQRVTISCTGSSSNIGAGYDVHWYQQLPGTAPKLL |
| | | IYGNSNRPSGVPDRFSGSKSGTSASLAITGLQAED |
| | | EADYYCQSYDSSLSGSVFGGGTKLTVLEQKLISEE |
| | | DLNPGGGGGSGGGGSGGGGSGGGGSGSMDIQMTQS |
| | | PSSLSASVGDRVTITCRASQDIRNYLNWYQQKPGK |
| | | APKLLIYYTSRLESGVPSRFSGSGSGTDYTLTISS |
| | | LQPEDFATYYCQQGNTLPWTFGQGTKVEIKGGGGG |
| | | GGGSGGGGSEVQLVESGGGLVQPGGSLRLSCAASG |
| | | YSFTGYTMNWVRQAPGKGLEWVALINPYKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 574 | Amino Acid | MALPVTALLLPLALLLHAARPQSVLTQPPSVSGAP |
| | | GQRVTISCTGSSSNIGAGYDVHWYQQLPGTAPKLL |
| | | IYGNSNRPSGVPDRFSGSKSGTSASLAITGLQAED |
| | | EADYYCQSYDSSLSGSVFGGGTKLTVLGSTSGSGK |
| | | PGSGEGSTKGQVQLQESGGGVVQPGRSLRLSCAAS |
| | | GFTLSSYGMHWVRQAPGKGLEWVAVISYDGSNKYY |
| | | ADSVKGRFTISRDNSKNTLYLQMNSLRAEDTAVYY |
| | | CAKSGWRYYYYYGMDVWGQGTLVTVSSEQKLISEE |
| | | DLNPGGGGGSGGGGSGGGGSGGGGSGSMDIQMTQS |
| | | PSSLSASVGDRVTITCRASQDIRNYLNWYQQKPGK |
| | | APKLLIYYTSRLESGVPSRFSGSGSGTDYTLTISS |
| | | LQPEDFATYYCQQGNTLPWTFGQGTKVEIKGGGGS |
| | | GGGGSGGGGSEVQLVESGGGLVQPGGSLRLSCAAS |
| | | GYSFTGYTMNWVRQAPGKGLEWVALINPYKGVSTY |
| | | NQKFKDRFTISVDKSKNTAYLQMNSLRAEDTAVYY |
| | | CARSGYYGDSDWYFDVWGQGTLVTVSSTSGGGGSL |
| | | ESGQVLLESNIKVLPTWSTPVQPMALIVLGGVAGL |
| | | LLFIGLGIFFCVRCRHRRRQAERMSQIKRLLSEKK |
| | | TCQCPHRFQKTCSPI |
| 575 | Amino Acid | MALPVTALLLPLALLLHAARPEVQLLESGGGLVQP |
| | | GGSLRLSCAASGFTFSSYWMSWVRQAPGKGLEWVA |
| | | NIKQDGSEKYYVDSVKGRFTISRDNAKNSLYLQMN |
| | | SLRAEDTAVYYCARPAGQLLYGMDVWGQGTLVTVS |
| | | SGSTSGSGKPGSGEGSTKGSYVLTQPPSVSVSPGQ |
| | | TARITCSGDALPKQYAYWYQQKPGQAPVLVIYKDS |
| | | ERPSGIPERFSGSSSGTTVTLTISGVQAEDEADYY |
| | | CQSADSSGTWVFGGGTKLTVLEQKLISEEDLNPGG |
| | | GGGSGGGGSGGGGSGGGGSGSMDIQMTQSPSSLSA |
| | | SVGDRVTITCRASQDIRNYLNWYQQKPGKAPKLLI |
| | | YYTSRLESGVPSRFSGSGSGTDYTLTISSLQPEDF |
| | | ATYYCQQGNTLPWTFGQGTKVEIKGGGGSGGGGSG |
| | | GGGSEVQLVESGGGLVQPGGSLRLSCAASGYSFTG |
| | | YTMNWVRQAPGKGLEWVALINPYKGVSTYNQKFKD |
| | | RFTISVDKSKNTAYLQMNSLRAEDTAVYYCARSGY |
| | | YGDSDWYFDVWGQGTLVTVSSTSGGGGSLESGQVL |
| | | LESNIKVLPTWSTPVQPMALIVLGGVAGLLLFIGL |
| | | GIFFCVRCRHRRRQAERMSQIKRLLSEKKTCQCPH |
| | | RFQKTCSPI |
| 576 | Amino Acid | MALPVTALLLPLALLLHAARPSYVLTQPPSVSVSP |
| | | GQTARITCSGDALPKQYAYWYQQKPGQAPVLVIYK |
| | | DSERPSGIPERFSGSSSGTTVTLTISGVQAEDEAD |
| | | YYCQSADSSGTWVFGGGTKLTVLGSTSGSGKPGSG |
| | | EGSTKGEVQLLESGGGLVQPGGSLRLSCAASGFTF |
| | | SSYWMSWVRQAPGKGLEWVANIKQDGSEKYYVDSV |
| | | KGRFTISRDNAKNSLYLQMNSLRAEDTAVYYCARP |
| | | AGQLLYGMDVWGQGTLVTVSSEQKLISEEDLNPGG |
| | | GGGSGGGGSGGGGSGGGGSGSMDIQMTQSPSSLSA |
| | | SVGDRVTITCRASQDIRNYLNWYQQKPGKAPKLLI |
| | | YYTSRLESGVPSRFSGSGSGTDYTLTISSLQPEDF |
| | | ATYYCQQGNTLPWTFGQGTKVEIKGGGGSGGGGGG |
| | | GGSEVQLVESGGGLVQPGGSLRLSCAASGYSFTGY |
| | | TMNWVRQAPGKGLEWVALINPYKGVSTYNQKFKDR |
| | | FTISVDKSKNTAYLQMNSLRAEDTAVYYCARSGYY |
| | | GDSDWYFDVWGQGTLVTVSSTSGGGGSLESGQVLL |
| | | ESNIKVLPTWSTPVQPMALIVLGGVAGLLLFIGLG |
| | | IFFCVRCRHRRRQAERMSQIKRLLSEKKTCQCPHR |
| | | FQKTCSPI |
| 577 | Amino Acid | MALPVTALLLPLALLLHAARPEVQLLESGGGVVQP |
| | | GRSLRLSCAASGFTFSSYGMHWVRQAPGKGLEWVA |
| | | VISYDGSNKYYADSVKGRFTISRDNSKNTLYLQMN |
| | | SLRAEDTAVYYCAKDAADMGAFDYWGQGTLVTVSS |
| | | GSTSGSGKPGSGEGSTKGDIQMTQSPDSLAVSLGE |
| | | RATMNCKSSQSVLYSSNNKNYLAWYQQKPGQPPKL |
| | | LIYWASARESGVPDRFSGSGSGTDFTLTISSLQAE |
| | | DVAVYYCQQYYSTPLTFGQGTKVEIKEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPYKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 578 | Amino Acid | MALPVTALLLPLALLLHAARPDIQMTQSPDSLAVS |
| | | LGERATMNCKSSQSVLYSSNNKNYLAWYQQKPGQP |
| | | PKLLIYWASARESGVPDRFSGSGSGTDFTLTISSL |
| | | QAEDVAVYYCQQYYSTPLTFGQGTKVEIKGSTSGS |
| | | GKPGSGEGSTKGEVQLLESGGGVVQPGRSLRLSCA |
| | | ASGFTFSSYGMHWVRQAPGKGLEWVAVISYDGSNK |
| | | YYADSVKGRFTISRDNSKNTLYLQMNSLRAEDTAV |
| | | YYCAKDAADMGAFDYWGQGTLVTVSSEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGGGGGSEVQLVESGGGLVQPGGSLRLSCAASGY |
| | | SFTGYTMNWVRQAPGKGLEWVALINPYKGVSTYNQ |
| | | KFKDRFTISVDKSKNTAYLQMNSLRAEDTAVYYCA |
| | | RSGYYGDSDWYFDVWGQGTLVTVSSTSGGGGSLES |
| | | GQVLLESNIKVLPTWSTPVQPMALIVLGGVAGLLL |
| | | FIGLGIFFCVRCRHRRRQAERMSQIKRLLSEKKTC |
| | | QCPHRFQKTCSPI |
| 579 | Amino Acid | MALPVTALLLPLALLLHAARPEVQLLESGGGLVKP |
| | | GGSLRLSCAASGFTFSSYSMNWVRQAPGKGLEWVS |
| | | AISGSGGSTYYADSVKGRFTISRDNSKNTLYLQMN |
| | | SLRAEDTAVYYCARARGYSYGSDAFDIWGQGTMVT |
| | | VSSGSTSGSGKPGSGEGSTKGQSVLTQPPSASGTP |
| | | GQRVTISCSGSSSNIGSNYVYWYQQLPGTAPKLLI |
| | | YRNNQRPSGVPDRFSGSKSGTSASLAISGLRSEDE |
| | | ADYYCAAWDDSLSGVVFGGGTKLTVLEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPYKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 580 | Amino Acid | MALPVTALLLPLALLLHAARPQSVLTQPPSASGTP |
| | | GQRVTISCSGSSSNIGSNYVYWYQQLPGTAPKLLI |
| | | YRNNQRPSGVPDRFSGSKSGTSASLAISGLRSEDE |
| | | ADYYCAAWDDSLSGVVFGGGTKLTVLGSTSGSGKP |
| | | GSGEGSTKGEVQLLESGGGLVKPGGSLRLSCAASG |
| | | FTFSSYSMNWVRQAPGKGLEWVSAISGSGGSTYYA |
| | | DSVKGRFTISRDNSKNTLYLQMNSLRAEDTAVYYC |
| | | ARARGYSYGSDAFDIWGQGTMVTVSSEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGSGGGGSEVQLVESGGGLVQPGGSLRLSCAASG |
| | | YSFTGYTMNWVRQAPGKGLEWVALINPYKGVSTYN |
| | | QKFKDRFTISVDKSKNTAYLQMNSLRAEDTAVYYC |
| | | ARSGYYGDSDWYFDVWGQGTLVTVSSTSGGGGSLE |
| | | SGQVLLESNIKVLPTWSTPVQPMALIVLGGVAGLL |
| | | LFIGLGIFFCVRCRHRRRQAERMSQIKRLLSEKKT |
| | | CQCPHRFQKTCSPI |
| 581 | Amino Acid | MALPVTALLLPLALLLHAARPEVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARDRGRRYCSGGSCPNAFDIWGQ |
| | | GTMVTVSSGSTSGSGKPGSGEGSTKGEIVLTQSPA |
| | | TLSVSPGERATLSCRASQSVSSNLAWYQQKPGQAP |
| | | RLLIYGASTRATGIPARFSGSGSGTEFTLTISSLQ |
| | | SEDFAVYYCQQYNNWPPALTFGGGTKVEIKEQKLI |
| | | SEEDLNPGGGGGSGGGGSGGGGSGGGGSGSMDIQM |
| | | TQSPSSLSASVGDRVTITCRASQDIRNYLNWYQQK |
| | | PGKAPKLLIYYTSRLESGVPSRFSGSGSGTDYTLT |
| | | ISSLQPEDFATYYCQQGNTLPWTFGQGTKVEIKGG |
| | | GGGGGGSGGGGSEVQLVESGGGLVQPGGSLRLSCA |
| | | ASGYSFTGYTMNWVRQAPGKGLEWVALINPYKGVS |
| | | TYNQKFKDRFTISVDKSKNTAYLQMNSLRAEDTAV |
| | | YYCARSGYYGDSDWYFDVWGQGTLVTVSSTSGGGG |
| | | SLESGQVLLESNIKVLPTWSTPVQPMALIVLGGVA |
| | | GLLLFIGLGIFFCVRCRHRRRQAERMSQIKRLLSE |
| | | KKTCQCPHRFQKTCSPI |
| 582 | Amino Acid | MALPVTALLLPLALLLHAARPEIVLTQSPATLSVS |
| | | PGERATLSCRASQSVSSNLAWYQQKPGQAPRLLIY |
| | | GASTRATGIPARFSGSGSGTEFTLTISSLQSEDFA |
| | | VYYCQQYNNWPPALTFGGGTKVEIKGSTSGSGKPG |
| | | SGEGSTKGEVQLVQSGAEVKKPGSSVKVSCKASGG |
| | | TFSSYAISWVRQAPGQGLEWMGGIIPIFGTANYAQ |
| | | KFQGRVTITADESTSTAYMELSSLRSEDTAVYYCA |
| | | RDRGRRYCSGGSCPNAFDIWGQGTMVTVSSEQKLI |
| | | SEEDLNPGGGGGSGGGGSGGGGSGGGGSGSMDIQM |
| | | TQSPSSLSASVGDRVTITCRASQDIRNYLNWYQQK |
| | | PGKAPKLLIYYTSRLESGVPSRFSGSGSGTDYTLT |
| | | ISSLQPEDFATYYCQQGNTLPWTFGQGTKVEIKGG |
| | | GGSGGGGGGGGSEVQLVESGGGLVQPGGSLRLSCA |
| | | ASGYSFTGYTMNWVRQAPGKGLEWVALINPYKGVS |
| | | TYNQKFKDRFTISVDKSKNTAYLQMNSLRAEDTAV |
| | | YYCARSGYYGDSDWYFDVWGQGTLVTVSSTSGGGG |
| | | SLESGQVLLESNIKVLPTWSTPVQPMALIVLGGVA |
| | | GLLLFIGLGIFFCVRCRHRRRQAERMSQIKRLLSE |
| | | KKTCQCPHRFQKTCSPI |
| 583 | Amino Acid | MALPVTALLLPLALLLHAARPQVQLVQSGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARGGYVWGSYRQNSNWFDPWGQG |
| | | TLVTVSSGSTSGSGKPGSGEGSTKGSYVLTQPPSA |
| | | SGTPGQRVTISCSGSSSNIGSNYVYWYQQLPGTAP |
| | | KLLIYRNNQRPSGVPDRFSGSKSGTSASLAISGLR |
| | | SEDEADYYCAAWDDSLSGWVFGGGTKLTVLEQKLI |
| | | SEEDLNPGGGGGSGGGGSGGGGSGGGGSGSMDIQM |
| | | TQSPSSLSASVGDRVTITCRASQDIRNYLNWYQQK |
| | | PGKAPKLLIYYTSRLESGVPSRFSGSGSGTDYTLT |
| | | ISSLQPEDFATYYCQQGNTLPWTFGQGTKVEIKGG |
| | | GGSGGGGSGGGGSEVQLVESGGGLVQPGGSLRLSC |
| | | AASGYSFTGYTMNWVRQAPGKGLEWVALINPYKGV |
| | | STYNQKFKDRFTISVDKSKNTAYLQMNSLRAEDTA |
| | | VYYCARSGYYGDSDWYFDVWGQGTLVTVSSTSGGG |
| | | GSLESGQVLLESNIKVLPTWSTPVQPMALIVLGGV |
| | | AGLLLFIGLGIFFCVRCRHRRRQAERMSQIKRLLS |
| | | EKKTCQCPHRFQKTCSPI |
| 584 | Amino Acid | MALPVTALLLPLALLLHAARPSYVLTQPPSASGTP |
| | | GQRVTISCSGSSSNIGSNYVYWYQQLPGTAPKLLI |
| | | YRNNQRPSGVPDRFSGSKSGTSASLAISGLRSEDE |
| | | ADYYCAAWDDSLSGWVFGGGTKLTVLGSTSGSGKP |
| | | GSGEGSTKGQVQLVQSGAEVKKPGSSVKVSCKASG |
| | | GTFSSYAISWVRQAPGQGLEWMGGIIPIFGTANYA |
| | | QKFQGRVTITADESTSTAYMELSSLRSEDTAVYYC |
| | | ARGGYVWGSYRQNSNWFDPWGQGTLVTVSSEQKLI |
| | | SEEDLNPGGGGGSGGGGSGGGGSGGGGSGSMDIQM |
| | | TQSPSSLSASVGDRVTITCRASQDIRNYLNWYQQK |
| | | PGKAPKLLIYYTSRLESGVPSRFSGSGSGTDYTLT |
| | | ISSLQPEDFATYYCQQGNTLPWTFGQGTKVEIKGG |
| | | GGSGGGGSGGGGSEVQLVESGGGLVQPGGSLRLSC |
| | | AASGYSFTGYTMNWVRQAPGKGLEWVALINPYKGV |
| | | STYNQKFKDRFTISVDKSKNTAYLQMNSLRAEDTA |
| | | VYYCARSGYYGDSDWYFDVWGQGTLVTVSSTSGGG |
| | | GSLESGQVLLESNIKVLPTWSTPVQPMALIVLGGV |
| | | AGLLLFIGLGIFFCVRCRHRRRQAERMSQIKRLLS |
| | | EKKTCQCPHRFQKTCSPI |
| 585 | Amino Acid | MALPVTALLLPLALLLHAARPQVQLVESGAEVKKP |
| | | GSSVKVSCKASGGTFSSYAISWVRQAPGQGLEWMG |
| | | GIIPIFGTANYAQKFQGRVTITADKSTSTAYMELS |
| | | SLRSEDTAVYYCARTHLPYSYGLGGFDYWGQGTLV |
| | | TVSSGSTSGSGKPGSGEGSTKGQSVLTQPPSVSGA |
| | | PGQRVTISCTGSSSNIGAGYDVHWYQQLPGTAPKL |
| | | LIYGNSNRPSGVPDRFSGSKSGTSASLAITGLQAE |
| | | DEADYYCQSYDSSLSGPVVFGGGTKVTVLEQKLIS |
| | | EEDLNPGGGGGSGGGGSGGGGSGGGGSGSMDIQMT |
| | | QSPSSLSASVGDRVTITCRASQDIRNYLNWYQQKP |
| | | GKAPKLLIYYTSRLESGVPSRFSGSGSGTDYTLTI |
| | | SSLQPEDFATYYCQQGNTLPWTFGQGTKVEIKGGG |
| | | GSGGGGSGGGGSEVQLVESGGGLVQPGGSLRLSCA |
| | | ASGYSFTGYTMNWVRQAPGKGLEWVALINPYKGVS |
| | | TYNQKFKDRFTISVDKSKNTAYLQMNSLRAEDTAV |
| | | YYCARSGYYGDSDWYFDVWGQGTLVTVSSTSGGGG |
| | | SLESGQVLLESNIKVLPTWSTPVQPMALIVLGGVA |
| | | GLLLFIGLGIFFCVRCRHRRRQAERMSQIKRLLSE |
| | | KKTCQCPHRFQKTCSPI |
| 586 | Amino Acid | MALPVTALLLPLALLLHAARPQSVLTQPPSVSGAP |
| | | GQRVTISCTGSSSNIGAGYDVHWYQQLPGTAPKLL |
| | | IYGNSNRPSGVPDRFSGSKSGTSASLAITGLQAED |
| | | EADYYCQSYDSSLSGPVVFGGGTKVTVLGSTSGSG |
| | | KPGSGEGSTKGQVQLVESGAEVKKPGSSVKVSCKA |
| | | SGGTFSSYAISWVRQAPGQGLEWMGGIIPIFGTAN |
| | | YAQKFQGRVTITADKSTSTAYMELSSLRSEDTAVY |
| | | YCARTHLPYSYGLGGFDYWGQGTLVTVSSEQKLIS |
| | | EEDLNPGGGGGSGGGGSGGGGSGGGGSGSMDIQMT |
| | | QSPSSLSASVGDRVTITCRASQDIRNYLNWYQQKP |
| | | GKAPKLLIYYTSRLESGVPSRFSGSGSGTDYTLTI |
| | | SSLQPEDFATYYCQQGNTLPWTFGQGTKVEIKGGG |
| | | GSGGGGSGGGGSEVQLVESGGGLVQPGGSLRLSCA |
| | | ASGYSFTGYTMNWVRQAPGKGLEWVALINPYKGVS |
| | | TYNQKFKDRFTISVDKSKNTAYLQMNSLRAEDTAV |
| | | YYCARSGYYGDSDWYFDVWGQGTLVTVSSTSGGGG |
| | | SLESGQVLLESNIKVLPTWSTPVQPMALIVLGGVA |
| | | GLLLFIGLGIFFCVRCRHRRRQAERMSQIKRLLSE |
| | | KKTCQCPHRFQKTCSPI |
| 587 | Amino Acid | MALPVTALLLPLALLLHAARPQVQLVQSGAEVKKP |
| | | GASVKVSCKASGYTFTSYGISWVRQAPGQGLEWMG |
| | | WISAYNGNTNYAQKLQGRVTMTTDTSTSTAYMELR |
| | | SLRSDDTAVYYCARDIIRYCSSTSCYRGIDYWGQG |
| | | TLVTVSSGSTSGSGKPGSGEGSTKGQSALTQPASV |
| | | SGSPGQSITISCTGTSSDVGGYNYVSWYQQHPGKA |
| | | PKLMIYDVSNRPSGVSNRFSGSKSGNTASLTISGL |
| | | QAEDEADYYCSSYTSSSPHVVSGGGTKLTVLEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPYKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 588 | Amino Acid | MALPVTALLLPLALLLHAARPQSALTQPASVSGSP |
| | | GQSITISCTGTSSDVGGYNYVSWYQQHPGKAPKLM |
| | | IYDVSNRPSGVSNRFSGSKSGNTASLTISGLQAED |
| | | EADYYCSSYTSSSPHVVSGGGTKLTVLGSTSGSGK |
| | | PGSGEGSTKGQVQLVQSGAEVKKPGASVKVSCKAS |
| | | GYTFTSYGISWVRQAPGQGLEWMGWISAYNGNTNY |
| | | AQKLQGRVTMTTDTSTSTAYMELRSLRSDDTAVYY |
| | | CARDIIRYCSSTSCYRGIDYWGQGTLVTVSSEQKL |
| | | ISEEDLNPGGGGGSGGGGSGGGGSGGGGSGSMDIQ |
| | | MTQSPSSLSASVGDRVTITCRASQDIRNYLNWYQQ |
| | | KPGKAPKLLIYYTSRLESGVPSRFSGSGSGTDYTL |
| | | TISSLQPEDFATYYCQQGNTLPWTFGQGTKVEIKG |
| | | GGGSGGGGSGGGGSEVQLVESGGGLVQPGGSLRLS |
| | | CAASGYSFTGYTMNWVRQAPGKGLEWVALINPYKG |
| | | VSTYNQKFKDRFTISVDKSKNTAYLQMNSLRAEDT |
| | | AVYYCARSGYYGDSDWYFDVWGQGTLVTVSSTSGG |
| | | GGSLESGQVLLESNIKVLPTWSTPVQPMALIVLGG |
| | | VAGLLLFIGLGIFFCVRCRHRRRQAERMSQIKRLL |
| | | SEKKTCQCPHRFQKTCSPI |
| 589 | Amino Acid | MALPVTALLLPLALLLHAARPQVQLVESGAEVKKP |
| | | GSSVKVSCKASGGTFSSYAISWVRQAPGQGLEWMG |
| | | GIIPIFGTANYAQKFQGRVTITADESTSTAYMELS |
| | | SLRSEDTAVYYCARPLYAPRFGYGMDVWGQGTLVT |
| | | VSSGSTSGSGKPGSGEGSTKGQSVLTQPPSASGTP |
| | | GQRVTISCSGSSSNIGSNYVYWYQQLPGTAPKLLI |
| | | YRNNQRPSGVPDRFSGSKSGTSASLAISGLRSEDE |
| | | ADYYCAAWDDSLSGWVFGGGTQLTVLEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKGGGGSG |
| | | GGGGGGGSEVQLVESGGGLVQPGGSLRLSCAASGY |
| | | SFTGYTMNWVRQAPGKGLEWVALINPYKGVSTYNQ |
| | | KFKDRFTISVDKSKNTAYLQMNSLRAEDTAVYYCA |
| | | RSGYYGDSDWYFDVWGQGTLVTVSSTSGGGGSLES |
| | | GQVLLESNIKVLPTWSTPVQPMALIVLGGVAGLLL |
| | | FIGLGIFFCVRCRHRRRQAERMSQIKRLLSEKKTC |
| | | QCPHRFQKTCSPI |
| 590 | Amino Acid | MALPVTALLLPLALLLHAARPQSVLTQPPSASGTP |
| | | GQRVTISCSGSSSNIGSNYVYWYQQLPGTAPKLLI |
| | | YRNNQRPSGVPDRFSGSKSGTSASLAISGLRSEDE |
| | | ADYYCAAWDDSLSGWVFGGGTQLTVLGSTSGSGKP |
| | | GSGEGSTKGQVQLVESGAEVKKPGSSVKVSCKASG |
| | | GTFSSYAISWVRQAPGQGLEWMGGIIPIFGTANYA |
| | | QKFQGRVTITADESTSTAYMELSSLRSEDTAVYYC |
| | | ARPLYAPRFGYGMDVWGQGTLVTVSSEQKLISEED |
| | | LNPGGGGGSGGGGSGGGGSGGGGSGSMDIQMTQSP |
| | | SSLSASVGDRVTITCRASQDIRNYLNWYQQKPGKA |
| | | PKLLIYYTSRLESGVPSRFSGSGSGTDYTLTISSL |
| | | QPEDFATYYCQQGNTLPWTFGQGTKVEIKG |
| | | GGGSGGGGSGGGGSEVQLVESGGGLVQP |
| | | GGSLRLSCAASGYSFTGYTMNWVRQAPGKGLEWVA |
| | | LINPYKGVSTYNQKFKDRFTISVDKSK |
| | | NTAYLQMNSLRAEDTAVYYCARSGYYGDSDWYFDV |
| | | WGQGTLVTVSSTSGGGGSLESGQVLL |
| | | ESNIKVLPTWSTPVQPMALIVLGGVAGLLLFIGLG |
| | | IFFCVRCRHRRRQAERMSQIKRLLSEKKTC |
| | | QCPHRFQKTCSPI |
| 591 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGACATATTACGATTGAGCGTGTCCTCGG |
| | | GTATGGACGTCTGGGGCCAAGGGACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGTTGACGCAGCCGCCCTCAGCGTCTGGGACCCCC |
| | | GGGCAGAGGGTCACCATCTCTTGTTCTGGAGGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGTCATGTGGTATTCGGCGGAGGGACCAAGC |
| | | TGACCGTCCTAGAGCAGAAACTGATCAGCGAGGAA |
| | | GATCTGAATCCTGGCGGAGGCGGAGGAAGTGGTGG |
| | | CGGAGGTTCTGGTGGCGGTGGATCAGGCGGTGGCG |
| | | GATCTGGATCTATGGATATCCAGATGACCCAGTCC |
| | | CCGAGCTCCCTGTCCGCCTCTGTGGGCGATAGGGT |
| | | CACCATCACCTGCCGTGCCAGTCAGGACATCCGTA |
| | | ATTATCTGAACTGGTATCAACAGAAACCAGGAAAA |
| | | GCTCCGAAACTACTGATTTACTATACCTCCCGCCT |
| | | GGAGTCTGGAGTCCCTTCTCGCTTCTCTGGTTCTG |
| | | GTTCTGGGACGGATTACACTCTGACCATCAGCAGT |
| | | CTGCAACCGGAAGACTTCGCAACTTATTACTGTCA |
| | | GCAAGGTAATACTCTGCCGTGGACGTTCGGACAGG |
| | | GCACCAAGGTGGAGATCAAAGGCGGCGGCGGAAGT |
| | | GGAGGAGGAGGCTCAGGCGGAGGAGGGAGCGAGGT |
| | | TCAGCTGGTGGAGTCTGGCGGTGGCCTGGTGCAGC |
| | | CAGGGGGCTCACTCCGTTTGTCCTGTGCAGCTTCT |
| | | GGCTACTCCTTTACCGGCTACACTATGAACTGGGT |
| | | GCGTCAGGCCCCAGGTAAGGGCCTGGAATGGGTTG |
| | | CACTGATTAATCCTACCAAAGGTGTTAGTACCTAC |
| | | AACCAGAAGTTCAAGGACCGTTTCACTATAAGCGT |
| | | AGATAAATCCAAAAACACAGCCTACCTGCAAATGA |
| | | ACAGCCTGCGTGCTGAGGACACTGCCGTCTATTAT |
| | | TGTGCTAGAAGCGGATACTACGGCGATAGTGACTG |
| | | GTATTTTGACGTGTGGGGTCAAGGAACCCTGGTCA |
| | | CCGTCTCCTCGACATCTGGCGGCGGAGGATCTCTG |
| | | GAATCTGGACAGGTGCTGCTGGAAAGCAACATCAA |
| | | GGTGCTGCCCACCTGGTCTACCCCAGTTCAGCCTA |
| | | TGGCTCTGATTGTGCTTGGCGGAGTTGCCGGCCTG |
| | | CTGCTCTTTATCGGCCTGGGCATCTTCTTTTGCGT |
| | | GCGGTGCAGACATCGGCGGAGACAGGCTGAGAGAA |
| | | TGAGCCAGATCAAGCGGCTGCTGAGCGAGAAGAAA |
| | | ACCTGTCAGTGCCCTCACCGGTTCCAGAAAACATG |
| | | CAGCCCCATC |
| 592 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGACATATTACGATTGAGCGTGTCCTCGG |
| | | GTATGGACGTCTGGGGCCAAGGGACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGCTGACTCAGCCACCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGACTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGCTGTGGTATTCGGCGGAGGGACCAAGCTGA |
| | | CCGTCCTAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTACCAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 593 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGCAGGAGTCGGGGGGAGGCGTGGTCCAGCCT |
| | | GGGAGGTCCCTGAGACTCTCCTGTGCAGCCTCTGG |
| | | ATTCACCCTCAGTAGCTATGGCATGCACTGGGTCC |
| | | GCCAGGCTCCAGGCAAGGGGCTGGAGTGGGTGGCA |
| | | GTTATATCATATGATGGAAGTAATAAATACTATGC |
| | | AGACTCCGTGAAGGGCCGATTCACCATCTCCAGAG |
| | | ACAATTCCAAGAACACGCTGTATCTGCAAATGAAC |
| | | AGCCTGAGAGCTGAGGACACGGCTGTGTATTACTG |
| | | TGCGAAGAGCGGCTGGAGATATTACTACTACTACG |
| | | GTATGGACGTCTGGGGCCAAGGGACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGTTGACGCAGCCGCCCTCAGTGTCTGGGGCCCCA |
| | | GGGCAGAGGGTCACCATCTCCTGCACTGGGAGCAG |
| | | CTCCAACATCGGGGCAGGTTATGATGTACACTGGT |
| | | ACCAGCAGCTTCCAGGAACAGCCCCCAAACTCCTC |
| | | ATCTATGGTAACAGCAATCGGCCCTCAGGGGTCCC |
| | | TGACCGATTCTCTGGCTCCAAGTCTGGCACCTCAG |
| | | CCTCCCTGGCCATCACTGGGCTCCAGGCTGAGGAT |
| | | GAGGCTGATTATTACTGCCAGTCCTATGACAGCAG |
| | | CCTGAGTGGTTCGGTATTCGGCGGAGGGACCAAGC |
| | | TGACCGTCCTAGAGCAGAAACTGATCAGCGAGGAA |
| | | GATCTGAATCCTGGCGGAGGCGGAGGAAGTGGTGG |
| | | CGGAGGTTCTGGTGGCGGTGGATCAGGCGGTGGCG |
| | | GATCTGGATCTATGGATATCCAGATGACCCAGTCC |
| | | CCGAGCTCCCTGTCCGCCTCTGTGGGCGATAGGGT |
| | | CACCATCACCTGCCGTGCCAGTCAGGACATCCGTA |
| | | ATTATCTGAACTGGTATCAACAGAAACCAGGAAAA |
| | | GCTCCGAAACTACTGATTTACTATACCTCCCGCCT |
| | | GGAGTCTGGAGTCCCTTCTCGCTTCTCTGGTTCTG |
| | | GTTCTGGGACGGATTACACTCTGACCATCAGCAGT |
| | | CTGCAACCGGAAGACTTCGCAACTTATTACTGTCA |
| | | GCAAGGTAATACTCTGCCGTGGACGTTCGGACAGG |
| | | GCACCAAGGTGGAGATCAAAGGCGGCGGCGGAAGT |
| | | GGAGGAGGAGGCTCAGGCGGAGGAGGGAGCGAGGT |
| | | TCAGCTGGTGGAGTCTGGCGGTGGCCTGGTGCAGC |
| | | CAGGGGGCTCACTCCGTTTGTCCTGTGCAGCTTCT |
| | | GGCTACTCCTTTACCGGCTACACTATGAACTGGGT |
| | | GCGTCAGGCCCCAGGTAAGGGCCTGGAATGGGTTG |
| | | CACTGATTAATCCTACCAAAGGTGTTAGTACCTAC |
| | | AACCAGAAGTTCAAGGACCGTTTCACTATAAGCGT |
| | | AGATAAATCCAAAAACACAGCCTACCTGCAAATGA |
| | | ACAGCCTGCGTGCTGAGGACACTGCCGTCTATTAT |
| | | TGTGCTAGAAGCGGATACTACGGCGATAGTGACTG |
| | | GTATTTTGACGTGTGGGGTCAAGGAACCCTGGTCA |
| | | CCGTCTCCTCGACATCTGGCGGCGGAGGATCTCTG |
| | | GAATCTGGACAGGTGCTGCTGGAAAGCAACATCAA |
| | | GGTGCTGCCCACCTGGTCTACCCCAGTTCAGCCTA |
| | | TGGCTCTGATTGTGCTTGGCGGAGTTGCCGGCCTG |
| | | CTGCTCTTTATCGGCCTGGGCATCTTCTTTTGCGT |
| | | GCGGTGCAGACATCGGCGGAGACAGGCTGAGAGAA |
| | | TGAGCCAGATCAAGCGGCTGCTGAGCGAGAAGAAA |
| | | ACCTGTCAGTGCCCTCACCGGTTCCAGAAAACATG |
| | | CAGCCCCATC |
| 594 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGACATATTACGATTGAGCGTGTCCTCGG |
| | | GTATGGACGTCTGGGGCCAAGGGACAATGGTCACC |
| | | GTGTCTTCAGGGTCAACCTCTGGTAGCGGTAAGCC |
| | | TGGCTCCGGCGAAGGCTCCACAAAGGGTCAGTCTG |
| | | TGCTGACTCAGCCACCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGCCCCTCCGTGGTATTCGGCGGAGGGACCA |
| | | AGCTGACCGTCCTAGAGCAGAAACTGATCAGCGAG |
| | | GAAGATCTGAATCCTGGCGGAGGCGGAGGAAGTGG |
| | | TGGCGGAGGTTCTGGTGGCGGTGGATCAGGCGGTG |
| | | GCGGATCTGGATCTATGGATATCCAGATGACCCAG |
| | | TCCCCGAGCTCCCTGTCCGCCTCTGTGGGCGATAG |
| | | GGTCACCATCACCTGCCGTGCCAGTCAGGACATCC |
| | | GTAATTATCTGAACTGGTATCAACAGAAACCAGGA |
| | | AAAGCTCCGAAACTACTGATTTACTATACCTCCCG |
| | | CCTGGAGTATGGCTCTGCCTGTGACAGCTCTGTTG |
| | | CTGCCTCTGGCTCTGCTGCTGCATGCTGCTAGACC |
| | | TCAGGTGCAGCTGCAGGAGTCGGGGGGAGGCGTGG |
| | | TCCAGCCTGGGAGGTCCCTGAGACTCTCCTGTGCA |
| | | GCCTCTGGATTCACCCTCAGTAGCTATGGCATGCA |
| | | CTGGGTCCGCCAGGCTCCAGGCAAGGGGCTGGAGT |
| | | GGGTGGCAGTTATATCATATGATGGAAGTAATAAA |
| | | TACTATGCAGACTCCGTGAAGGGCCGATTCACCAT |
| | | CTCCAGAGACAATTCCAAGAACACGCTGTATCTGC |
| | | AAATGAACAGCCTGAGAGCTGAGGACACGGCTGTG |
| | | TATTACTGTGCGAAGAGCGGCTGGAGATATTACTA |
| | | CTACTACGGTATGGACGTCTGGGGCCAAGGGACCC |
| | | TGGTCACCGTGTCCTCAGGGTCAACCTCTGGTAGC |
| | | GGTAAGCCTGGCTCCGGCGAAGGCTCCACAAAGGG |
| | | TCAGTCTGTGTTGACGCAGCCGCCCTCAGTGTCTG |
| | | GGGCCCCAGGGCAGAGGGTCACCATCTCCTGCACT |
| | | GGGAGCAGCTCCAACATCGGGGCAGGTTATGATGT |
| | | ACACTGGTACCAGCAGCTTCCAGGAACAGCCCCCA |
| | | AACTCCTCATCTATGGTAACAGCAATCGGCCCTCA |
| | | GGGGTCCCTGACCGATTCTCTGGCTCCAAGTCTGG |
| | | CACCTCAGCCTCCCTGGCCATCACTGGGCTCCAGG |
| | | CTGAGGATGAGGCTGATTATTACTGCCAGTCCTAT |
| | | GACAGCAGCCTGAGTGGTTCGGTATTCGGCGGAGG |
| | | GACCAAGCTGACCGTCCTAGAGCAGAAACTGATCA |
| | | GCGAGGAAGATCTGAATCCTGGCGGAGGCGGAGGA |
| | | AGTGGTGGCGGAGGTTCTGGTGGCGGTGGATCAGG |
| | | CGGTGGCGGATCTGGATCTATGGATATCCAGATGA |
| | | CCCAGTCCCCGAGCTCCCTGTCCGCCTCTGTGGGC |
| | | GATAGGGTCACCATCACCTGCCGTGCCAGTCAGGA |
| | | CATCCGTAATTATCTGAACTGGTATCAACAGAAAC |
| | | CAGGAAAAGCTCCGAAACTACTGATTTACTATACC |
| | | TCCCGCCTGGAGTCTGGAGTCCCTTCTCGCTTCTC |
| | | TGGTTCTGGTTCTGGGACGGATTACACTCTGACCA |
| | | TCAGCAGTCTGCAACCGGAAGACTTCGCAACTTAT |
| | | TACTGTCAGCAAGGTAATACTCTGCCGTGGACGTT |
| | | CGGACAGGGCACCAAGGTGGAGATCAAAGGCGGCG |
| | | GCGGAAGTGGAGGAGGAGGCTCAGGCGGAGGAGGG |
| | | AGCGAGGTTCAGCTGGTGGAGTCTGGCGGTGGCCT |
| | | GGTGCAGCCAGGGGGCTCACTCCGTTTGTCCTGTG |
| | | CAGCTTCTGGCTACTCCTTTACCGGCTACACTATG |
| | | AACTGGGTGCGTCAGGCCCCAGGTAAGGGCCTGGA |
| | | ATGGGTTGCACTGATTAATCCTACCAAAGGTGTTA |
| | | GTACCTACAACCAGAAGTTCAAGGACCGTTTCACT |
| | | ATAAGCGTAGATAAATCCAAAAACACAGCCTACCT |
| | | GCAAATGAACAGCCTGCGTGCTGAGGACACTGCCG |
| | | TCTATTATTGTGCTAGAAGCGGATACTACGGCGAT |
| | | AGTGACTGGTATTTTGACGTGTGGGGTCAAGGAAC |
| | | CCTGGTCACCGTCTCCTCGACATCTGGCGGCGGAG |
| | | GATCTCTGGAATCTGGACAGGTGCTGCTGGAAAGC |
| | | AACATCAAGGTGCTGCCCACCTGGTCTACCCCAGT |
| | | TCAGCCTATGGCTCTGATTGTGCTTGGCGGAGTTG |
| | | CCGGCCTGCTGCTCTTTATCGGCCTGGGCATCTTC |
| | | TTTTGCGTGCGGTGCAGACATCGGCGGAGACAGGC |
| | | TGAGAGAATGAGCCAGATCAAGCGGCTGCTGAGCG |
| | | AGAAGAAAACCTGTCAGTGCCCTCACCGGTTCCAG |
| | | AAAACATGCAGCCCCATC |
| 595 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGCAGTCTGGGGCTGAGGTGAAGAAGCCT |
| | | GGGGCCTCAGTGAAGGTCTCCTGCAAGGCTTCTGG |
| | | TTACACCTTTACCAGCTATGGTATCAGCTGGGTGC |
| | | GACAGGCCCCTGGACAAGGGCTTGAGTGGATGGGA |
| | | TGGATCGGCGCTTACAATGGTAACACAAACTATGC |
| | | ACAGAAGCTCCAGGGCAGAGTCACCATGACCACAG |
| | | ACACATCCACGAGCACAGCCTACATGGAGCTGAGG |
| | | AGCCTGAGGAGCGACGACACGGCCGTGTATTACTG |
| | | TGCGAGGGACCTGAGGCGGTATAGCAGCAGCTGGG |
| | | ACGGCCCCGGGTACTGGGGCCAGGGAACCCTGGTC |
| | | ACCGTGTCCTCAGGGTCAACCTCTGGTAGCGGTAA |
| | | GCCTGGCTCCGGCGAAGGCTCCACAAAGGGTCAGT |
| | | CTGCCCTGACTCAGCCTGCCTCCGTGTCTGGGTCT |
| | | CCTGGACAGTCGATCACCATCTCCTGCACTGGAAC |
| | | CAGCAGTGACGTTGGTGGTTATAACTATGTCTCCT |
| | | GGTACCAACAACACCCAGGCAAAGCCCCCAAACTC |
| | | ATGATTTATGATGTCAGTAATCGGCCCTCAGGGGT |
| | | TTCTAATCGCTTCTCTGGCTCCAAGTCTGGCAACA |
| | | CGGCCTCCCTGACCATCTCTGGGCTCCAGGCTGAG |
| | | GACGAGGCTGATTATTACTGCAGCTCATATACAAG |
| | | CAGCAGCACTCGGGTCTTCGGAACTGGGACCAAGC |
| | | TGACCGTCCTAGAGCAGAAACTGATCAGCGAGGAA |
| | | GATCTGAATCCTGGCGGAGGCGGAGGAAGTGGTGG |
| | | CGGAGGTTCTGGTGGCGGTGGATCAGGCGGTGGCG |
| | | GATCTGGATCTATGGATATCCAGATGACCCAGTCC |
| | | CCGAGCTCCCTGTCCGCCTCTGTGGGCGATAGGGT |
| | | CACCATCACCTGCCGTGCCAGTCAGGACATCCGTA |
| | | ATTATCTGAACTGGTATCAACAGAAACCAGGAAAA |
| | | GCTCCGAAACTACTGATTTACTATACCTCCCGCCT |
| | | GGAGTCTGGAGTCCCTTCTCGCTTCTCTGGTTCTG |
| | | GTTCTGGGACGGATTACACTCTGACCATCAGCAGT |
| | | CTGCAACCGGAAGACTTCGCAACTTATTACTGTCA |
| | | GCAAGGTAATACTCTGCCGTGGACGTTCGGACAGG |
| | | GCACCAAGGTGGAGATCAAAGGCGGCGGCGGAAGT |
| | | GGAGGAGGAGGCTCAGGCGGAGGAGGGAGCGAGGT |
| | | TCAGCTGGTGGAGTCTGGCGGTGGCCTGGTGCAGC |
| | | CAGGGGGCTCACTCCGTTTGTCCTGTGCAGCTTCT |
| | | GGCTACTCCTTTACCGGCTACACTATGAACTGGGT |
| | | GCGTCAGGCCCCAGGTAAGGGCCTGGAATGGGTTG |
| | | CACTGATTAATCCTACCAAAGGTGTTAGTACCTAC |
| | | AACCAGAAGTTCAAGGACCGTTTCACTATAAGCGT |
| | | AGATAAATCCAAAAACACAGCCTACCTGCAAATGA |
| | | ACAGCCTGCGTGCTGAGGACACTGCCGTCTATTAT |
| | | TGTGCTAGAAGCGGATACTACGGCGATAGTGACTG |
| | | GTATTTTGACGTGTGGGGTCAAGGAACCCTGGTCA |
| | | CCGTCTCCTCGACATCTGGCGGCGGAGGATCTCTG |
| | | GAATCTGGACAGGTGCTGCTGGAAAGCAACATCAA |
| | | GGTGCTGCCCACCTGGTCTACCCCAGTTCAGCCTA |
| | | TGGCTCTGATTGTGCTTGGCGGAGTTGCCGGCCTG |
| | | CTGCTCTTTATCGGCCTGGGCATCTTCTTTTGCGT |
| | | GCGGTGCAGACATCGGCGGAGACAGGCTGAGAGAA |
| | | TGAGCCAGATCAAGCGGCTGCTGAGCGAGAAGAAA |
| | | ACCTGTCAGTGCCCTCACCGGTTCCAGAAAACATG |
| | | CAGCCCCATC |
| 596 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGACATATTACGATTGAGCGTGTCCTCGG |
| | | GTATGGACGTCTGGGGCCAAGGGACCCTGGTCACC |
| | | GTGTCCTCAGGGTCAACCTCTGGTAGCGGTAAGCC |
| | | TGGCTCCGGCGAAGGCTCCACAAAGGGTTCCTATG |
| | | TGCTGACTCAGCCACCCTCAGCGTCTGGGACCCCC |
| | | GGGCAGAGGGTCACCATCTCTTGTTCTGGAAGCAG |
| | | CTCCAACATCGGAGGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGTTGGGTGTTCGGCGGAGGGACCAAGCTGA |
| | | CCGTCCTAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTACCAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 597 | Nucleotide | ATGGCTCTGCCTGTGACAGCTCTGTTGCTGCCTCT |
| | | GGCTCTGCTGCTGCATGCTGCTAGACCTCAGGTTC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACAAATCCACGAGCACAGCCTACATGGAGCTGAGC |
| | | AGCCTGAGGAGCGAGGACACGGCCGTGTATTACTG |
| | | TGCGAGAGATGGGGGTCGATACAGCTATGGGAGGT |
| | | CCTTTGACTACTGGGGCCAGGGAACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGTTGACGCAGCCGCCCTCAGTGTCTGGGGCCCCA |
| | | GGGCAGAGGGTCACCATCTCCTGCACTGGGAGCAG |
| | | CTCCAACATCGGGGCAGGTTATGATGTACACTGGT |
| | | ACCAGCAGCTTCCAGGAACAGCCCCCAAACTCCTC |
| | | ATCTATGGTAACAGCAATCGGCCCTCAGGGGTCCC |
| | | TGACCGATTCTCCGGCTCCAAGTCTGGCACCTCAG |
| | | CCTCCCTGGCCATCACTGGGCTCCAGGCTGAGGAT |
| | | GAGGCTGATTATTACTGCCAGTCCTATGACAGCAG |
| | | CCTGAGTGGTCCCGTGGTATTCGGCGGAGGGACCA |
| | | AGCTGACCGTCCTAGAGCAGAAACTGATCAGCGAG |
| | | GAAGATCTGAATCCTGGCGGAGGCGGAGGAAGTGG |
| | | TGGCGGAGGTTCTGGTGGCGGTGGATCAGGCGGTG |
| | | GCGGATCTGGATCTATGGATATCCAGATGACCCAG |
| | | TCCCCGAGCTCCCTGTCCGCCTCTGTGGGCGATAG |
| | | GGTCACCATCACCTGCCGTGCCAGTCAGGACATCC |
| | | GTAATTATCTGAACTGGTATCAACAGAAACCAGGA |
| | | AAAGCTCCGAAACTACTGATTTACTATACCTCCCG |
| | | CCTGGAGTCTGGAGTCCCTTCTCGCTTCTCTGGTT |
| | | CTGGTTCTGGGACGGATTACACTCTGACCATCAGC |
| | | AGTCTGCAACCGGAAGACTTCGCAACTTATTACTG |
| | | TCAGCAAGGTAATACTCTGCCGTGGACGTTCGGAC |
| | | AGGGCACCAAGGTGGAGATCAAAGGCGGCGGCGGA |
| | | AGTGGAGGAGGAGGCTCAGGCGGAGGAGGGAGCGA |
| | | GGTTCAGCTGGTGGAGTCTGGCGGTGGCCTGGTGC |
| | | AGCCAGGGGGCTCACTCCGTTTGTCCTGTGCAGCT |
| | | TCTGGCTACTCCTTTACCGGCTACACTATGAACTG |
| | | GGTGCGTCAGGCCCCAGGTAAGGGCCTGGAATGGG |
| | | TTGCACTGATTAATCCTACCAAAGGTGTTAGTACC |
| | | TACAACCAGAAGTTCAAGGACCGTTTCACTATAAG |
| | | CGTAGATAAATCCAAAAACACAGCCTACCTGCAAA |
| | | TGAACAGCCTGCGTGCTGAGGACACTGCCGTCTAT |
| | | TATTGTGCTAGAAGCGGATACTACGGCGATAGTGA |
| | | CTGGTATTTTGACGTGTGGGGTCAAGGAACCCTGG |
| | | TCACCGTCTCCTCGACATCTGGCGGCGGAGGATCT |
| | | CTGGAATCTGGACAGGTGCTGCTGGAAAGCAACAT |
| | | CAAGGTGCTGCCCACCTGGTCTACCCCAGTTCAGC |
| | | CTATGGCTCTGATTGTGCTTGGCGGAGTTGCCGGC |
| | | CTGCTGCTCTTTATCGGCCTGGGCATCTTCTTTTG |
| | | CGTGCGGTGCAGACATCGGCGGAGACAGGCTGAGA |
| | | GAATGAGCCAGATCAAGCGGCTGCTGAGCGAGAAG |
| | | AAAACCTGTCAGTGCCCTCACCGGTTCCAGAAAAC |
| | | ATGCAGCCCCATC |
| 598 | Nucleotide | ATGGCTCTGCCTGTGACAGCTCTGTTGCTGCCTCT |
| | | GGCTCTGCTGCTGCATGCTGCTAGACCTCAGGTTC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGACCATTACGGCCCCTACACTACTACGGTA |
| | | TGGACGTCTGGGGCCAAGGGACCCTGGTCACCGTG |
| | | TCCTCAGGGTCAACCTCTGGTAGCGGTAAGCCTGG |
| | | CTCCGGCGAAGGCTCCACAAAGGGTCAGTCTGTGC |
| | | TGACGCAGCCGCCCTCAGCGTCTGGGACCCCCGGG |
| | | CAGAGGGTCACCATCTCTTGTTCTGGAAGCAGCTC |
| | | CAACATCGGAAGTAATTATGTATACTGGTACCAGC |
| | | AGCTCCCAGGAACGGCCCCCAAACTCCTCATCTAT |
| | | AGGAATAATCAGCGGCCCTCAGGGGTCCCTGACCG |
| | | ATTCTCTGGCTCCAAGTCTGGCACCTCAGCCTCCC |
| | | TGGCCATCAGTGGGCTCCGGTCCGAGGATGAGGCT |
| | | GATTATTACTGTGCAGCATGGGATGACAGCCTGAC |
| | | CGAGGGGGTGTTCGGCGGAGGGACCAAGCTGACCG |
| | | TCCTAGAGCAGAAACTGATCAGCGAGGAAGATCTG |
| | | AATCCTGGCGGAGGCGGAGGAAGTGGTGGCGGAGG |
| | | TTCTGGTGGCGGTGGATCAGGCGGTGGCGGATCTG |
| | | GATCTATGGATATCCAGATGACCCAGTCCCCGAGC |
| | | TCCCTGTCCGCCTCTGTGGGCGATAGGGTCACCAT |
| | | CACCTGCCGTGCCAGTCAGGACATCCGTAATTATC |
| | | TGAACTGGTATCAACAGAAACCAGGAAAAGCTCCG |
| | | AAACTACTGATTTACTATACCTCCCGCCTGGAGTC |
| | | TGGAGTCCCTTCTCGCTTCTCTGGTTCTGGTTCTG |
| | | GGACGGATTACACTCTGACCATCAGCAGTCTGCAA |
| | | CCGGAAGACTTCGCAACTTATTACTGTCAGCAAGG |
| | | TAATACTCTGCCGTGGACGTTCGGACAGGGCACCA |
| | | AGGTGGAGATCAAAGGCGGCGGCGGAAGTGGAGGA |
| | | GGAGGCTCAGGCGGAGGAGGGAGCGAGGTTCAGCT |
| | | GGTGGAGTCTGGCGGTGGCCTGGTGCAGCCAGGGG |
| | | GCTCACTCCGTTTGTCCTGTGCAGCTTCTGGCTAC |
| | | TCCTTTACCGGCTACACTATGAACTGGGTGCGTCA |
| | | GGCCCCAGGTAAGGGCCTGGAATGGGTTGCACTGA |
| | | TTAATCCTACCAAAGGTGTTAGTACCTACAACCAG |
| | | AAGTTCAAGGACCGTTTCACTATAAGCGTAGATAA |
| | | ATCCAAAAACACAGCCTACCTGCAAATGAACAGCC |
| | | TGCGTGCTGAGGACACTGCCGTCTATTATTGTGCT |
| | | AGAAGCGGATACTACGGCGATAGTGACTGGTATTT |
| | | TGACGTGTGGGGTCAAGGAACCCTGGTCACCGTCT |
| | | CCTCGACATCTGGCGGCGGAGGATCTCTGGAATCT |
| | | GGACAGGTGCTGCTGGAAAGCAACATCAAGGTGCT |
| | | GCCCACCTGGTCTACCCCAGTTCAGCCTATGGCTC |
| | | TGATTGTGCTTGGCGGAGTTGCCGGCCTGCTGCTC |
| | | TTTATCGGCCTGGGCATCTTCTTTTGCGTGCGGTG |
| | | CAGACATCGGCGGAGACAGGCTGAGAGAATGAGCC |
| | | AGATCAAGCGGCTGCTGAGCGAGAAGAAAACCTGT |
| | | CAGTGCCCTCACCGGTTCCAGAAAACATGCAGCCC |
| | | CATC |
| 599 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGTCTGGGGGAGGCGTGGTCCAGCCT |
| | | GGGAGGTCCCTGAGACTCTCCTGTGCAGCGTCTGG |
| | | ATTCACCTTCAGTAGCTATGGCATGCACTGGGTCC |
| | | GCCGGGCTCCAGGCAAGGGGCTGGAGTGGGTGGCA |
| | | GTTATATCATATGATGGAAGTAATAAATACTATGC |
| | | AGACTCCGTGAAGGGCCGATTCACCATCTCCAGAG |
| | | ACAATTCCAAGAACACGCTGTATCTGCAAATGAAC |
| | | AGCCTGAGAGCTGAGGACACGGCTGTGTATTACTG |
| | | TGCGAAAGATGGGGGCGACAGTGGGAGTCTTGACT |
| | | ACTGGGGCCAGGGAACCCTGGTCACCGTGTCCTCA |
| | | GGGTCAACCTCTGGTAGCGGTAAGCCTGGCTCCGG |
| | | CGAAGGCTCCACAAAGGGTGACATCGTGATGACCC |
| | | AGTCTCCAGACTCCCTGGCTGTGTCTCTGGGCGAG |
| | | AGGGCCACCATCAACTGCAAGTCCAGCCAGAGTGT |
| | | TTTATACAGCTCCAACAATAAGAACTACTTAGCTT |
| | | GGTACCAGCAGAAACCAGGACAGCCTCCTAAGCTG |
| | | CTCATTTACTGGGCATCTACCCGGGAATCCGGGGT |
| | | CCCTGACCGATTCAGTGGCAGCGGGTCTGGGACAG |
| | | ATTTCACTCTCACCATCAGCAGCCTGCAGGCTGAA |
| | | GATGTGGCAGTTTATTACTGTCAGCAATATTATAG |
| | | TACTCCGCTCACTTTCGGCGGAGGGACCAAGCTGG |
| | | AGATCAAAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTACCAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 600 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGTTGGAGTCTGGGGGAGGCTTGGTCCAGCCT |
| | | GGGGGGTCCCTGAGACTCTCCTGTGCAGCCTCTGG |
| | | ATTCACCTTTAGTAGCTATTGGATGAGCTGGGTCC |
| | | GCCAGGCTCCAGGGAAGGGGCTGGAGTGGGTGGCC |
| | | AACATAAAGCAAGATGGAAGTGAGAAATACTATGT |
| | | GGACTCTGTGAAGGGCCGATTCACCATCTCCAGAG |
| | | ACAACGCCAAGAACTCACTGTATCTGCAAATGAAC |
| | | AGCCTGAGAGCCGAGGACACGGCTGTGTATTACTG |
| | | TGCGAGACCGGCGGGCCAGCTGCTCTACGGTATGG |
| | | ACGTCTGGGGCCAAGGGACCCTGGTCACCGTGTCC |
| | | TCAGGGTCAACCTCTGGTAGCGGTAAGCCTGGCTC |
| | | CGGCGAAGGCTCCACAAAGGGTTCCTATGTGCTGA |
| | | CTCAGCCACCCTCGGTGTCAGTGTCCCCAGGACAG |
| | | ACGGCCAGGATCACCTGCTCTGGAGATGCATTGCC |
| | | AAAGCAATATGCTTATTGGTACCAGCAGAAGCCAG |
| | | GCCAGGCCCCTGTGCTGGTGATATATAAAGACAGT |
| | | GAGAGGCCCTCAGGGATCCCTGAGCGATTCTCTGG |
| | | CTCCAGCTCAGGGACAACAGTCACGTTGACCATCA |
| | | GTGGAGTCCAGGCAGAAGACGAGGCTGACTATTAC |
| | | TGTCAATCAGCAGACAGCAGTGGTACTTGGGTGTT |
| | | CGGCGGAGGCACCAAGCTGACCGTCCTCGAGCAGA |
| | | AACTGATCAGCGAGGAAGATCTGAATCCTGGCGGA |
| | | GGCGGAGGAAGTGGTGGCGGAGGTTCTGGTGGCGG |
| | | TGGATCAGGCGGTGGCGGATCTGGATCTATGGATA |
| | | TCCAGATGACCCAGTCCCCGAGCTCCCTGTCCGCC |
| | | TCTGTGGGCGATAGGGTCACCATCACCTGCCGTGC |
| | | CAGTCAGGACATCCGTAATTATCTGAACTGGTATC |
| | | AACAGAAACCAGGAAAAGCTCCGAAACTACTGATT |
| | | TACTATACCTCCCGCCTGGAGTCTGGAGTCCCTTC |
| | | TCGCTTCTCTGGTTCTGGTTCTGGGACGGATTACA |
| | | CTCTGACCATCAGCAGTCTGCAACCGGAAGACTTC |
| | | GCAACTTATTACTGTCAGCAAGGTAATACTCTGCC |
| | | GTGGACGTTCGGACAGGGCACCAAGGTGGAGATCA |
| | | AAGGCGGCGGCGGAAGTGGAGGAGGAGGCTCAGGC |
| | | GGAGGAGGGAGCGAGGTTCAGCTGGTGGAGTCTGG |
| | | CGGTGGCCTGGTGCAGCCAGGGGGCTCACTCCGTT |
| | | TGTCCTGTGCAGCTTCTGGCTACTCCTTTACCGGC |
| | | TACACTATGAACTGGGTGCGTCAGGCCCCAGGTAA |
| | | GGGCCTGGAATGGGTTGCACTGATTAATCCTACCA |
| | | AAGGTGTTAGTACCTACAACCAGAAGTTCAAGGAC |
| | | CGTTTCACTATAAGCGTAGATAAATCCAAAAACAC |
| | | AGCCTACCTGCAAATGAACAGCCTGCGTGCTGAGG |
| | | ACACTGCCGTCTATTATTGTGCTAGAAGCGGATAC |
| | | TACGGCGATAGTGACTGGTATTTTGACGTGTGGGG |
| | | TCAAGGAACCCTGGTCACCGTCTCCTCGACATCTG |
| | | GCGGCGGAGGATCTCTGGAATCTGGACAGGTGCTG |
| | | CTGGAAAGCAACATCAAGGTGCTGCCCACCTGGTC |
| | | TACCCCAGTTCAGCCTATGGCTCTGATTGTGCTTG |
| | | GCGGAGTTGCCGGCCTGCTGCTCTTTATCGGCCTG |
| | | GGCATCTTCTTTTGCGTGCGGTGCAGACATCGGCG |
| | | GAGACAGGCTGAGAGAATGAGCCAGATCAAGCGGC |
| | | TGCTGAGCGAGAAGAAAACCTGTCAGTGCCCTCAC |
| | | CGGTTCCAGAAAACATGCAGCCCCATC |
| 601 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGTTGGAGTCTGGGGGAGGCGTGGTCCAGCCT |
| | | GGGAGGTCCCTGAGACTCTCCTGTGCAGCCTCTGG |
| | | ATTCACCTTCAGTAGCTATGGCATGCACTGGGTCC |
| | | GCCAGGCTCCAGGCAAGGGGCTGGAGTGGGTGGCA |
| | | GTTATATCATATGATGGAAGTAATAAATACTATGC |
| | | AGACTCCGTGAAGGGCCGATTCACCATCTCCAGAG |
| | | ACAATTCCAAGAACACGCTGTATCTGCAAATGAAC |
| | | AGCCTGAGAGCTGAGGACACGGCTGTGTATTACTG |
| | | TGCGAAAGATGCCGCGGATATGGGGGCCTTTGACT |
| | | ACTGGGGCCAGGGAACCCTGGTCACCGTGTCCTCA |
| | | GGGTCAACCTCTGGTAGCGGTAAGCCTGGCTCCGG |
| | | CGAAGGCTCCACAAAGGGTGACATCCAGATGACCC |
| | | AGTCTCCAGACTCCCTGGCTGTGTCTCTGGGCGAG |
| | | AGGGCCACCATGAACTGCAAGTCCAGCCAGAGTGT |
| | | TTTATACAGCTCCAACAATAAGAACTACTTAGCTT |
| | | GGTACCAGCAGAAACCAGGACAGCCTCCTAAGCTG |
| | | CTCATTTACTGGGCATCTGCCCGGGAATCTGGGGT |
| | | CCCTGACCGATTCAGTGGCAGCGGGTCTGGGACAG |
| | | ATTTCACTCTCACCATCAGCAGCCTGCAGGCTGAA |
| | | GATGTGGCAGTTTATTACTGTCAGCAATATTATAG |
| | | TACTCCTCTCACTTTTGGCCAGGGGACCAAGGTGG |
| | | AAATCAAAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTACCAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 602 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGTTGGAGTCTGGGGGAGGCCTGGTCAAGCCT |
| | | GGGGGGTCCCTGAGACTCTCCTGTGCAGCCTCTGG |
| | | ATTCACCTTCAGTAGCTATAGCATGAACTGGGTCC |
| | | GCCAGGCTCCAGGGAAGGGGCTGGAGTGGGTCTCA |
| | | GCTATTAGTGGTAGTGGTGGTAGCACATACTACGC |
| | | AGACTCCGTGAAGGGCCGGTTCACCATCTCCAGAG |
| | | ACAATTCCAAGAACACGCTGTATCTGCAAATGAAC |
| | | AGCCTGAGAGCCGAGGACACGGCTGTGTATTACTG |
| | | TGCGAGAGCACGTGGATACAGCTATGGCTCTGATG |
| | | CTTTTGATATCTGGGGCCAAGGGACAATGGTCACC |
| | | GTGTCTTCAGGGTCAACCTCTGGTAGCGGTAAGCC |
| | | TGGCTCCGGCGAAGGCTCCACAAAGGGTCAGTCTG |
| | | TGCTGACTCAGCCACCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGCGTGGTATTCGGCGGAGGGACCAAGCTGA |
| | | CCGTCCTAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTACCAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 603 | Nucleotide | ATGGCTCTGCCTGTGACAGCTCTGTTGCTGCCTCT |
| | | GGCTCTGCTGCTGCATGCTGCTAGACCTCAGGTTC |
| | | AGCTGGTGCAGTCTGGGGGAGGCGTGGTCCAGCCT |
| | | GGGAGGTCCCTGAGACTCTCCTGTGCAGCCTCTGG |
| | | ATTCACCTTCAGTAGCTATGGCATGCACTGGGTCC |
| | | GCCAGGCTCCAGGCAAGGGGCTGGAGTGGGTGGCA |
| | | GTTATATCATATGATGGAAGTAATAAATACTATGC |
| | | AGACTCCGTGAAGGGCCGATTCACCATCTCCAGAG |
| | | ACAATTCCAAGAACACGCTGTATCTGCAAATGAAC |
| | | AGCCTGAGAGCTGAGGACACGGCTGTGTATTACTG |
| | | TGCGAAAGGTGTCCCCCGGTACTACTACTACTACG |
| | | GTATGGACGTCTGGGGCCAAGGGACAATGGTCACC |
| | | GTGTCTTCAGGGTCAACCTCTGGTAGCGGTAAGCC |
| | | TGGCTCCGGCGAAGGCTCCACAAAGGGTCAGTCTG |
| | | TGTTGACGCAGCCGCCCTCAGTGTCTGGGGCCCCA |
| | | GGGCAGAGGGTCACCATCTCCTGCACTGGGAGCAG |
| | | CTCCAACATCGGGGCAGGTTATGATGTACACTGGT |
| | | ACCAGCAGCTTCCAGGAACAGCCCCCAAACTCCTC |
| | | ATCTATGGTAACAGCAATCGGCCCTCAGGGGTCCC |
| | | TGACCGATTCTCTGGCTCCAAGTCTGGCACCTCAG |
| | | CCTCCCTGGCCATCACTGGGCTCCAGGCTGAGGAT |
| | | GAGGCTGATTATTACTGCCAGTCCTATGACAGCAG |
| | | CCTGAGTGGTTCGGTGTTCGGCGGAGGGACCAAGC |
| | | TGACCGTCCTAGAGCAGAAACTGATCAGCGAGGAA |
| | | GATCTGAATCCTGGCGGAGGCGGAGGAAGTGGTGG |
| | | CGGAGGTTCTGGTGGCGGTGGATCAGGCGGTGGCG |
| | | GATCTGGATCTATGGATATCCAGATGACCCAGTCC |
| | | CCGAGCTCCCTGTCCGCCTCTGTGGGCGATAGGGT |
| | | CACCATCACCTGCCGTGCCAGTCAGGACATCCGTA |
| | | ATTATCTGAACTGGTATCAACAGAAACCAGGAAAA |
| | | GCTCCGAAACTACTGATTTACTATACCTCCCGCCT |
| | | GGAGTCTGGAGTCCCTTCTCGCTTCTCTGGTTCTG |
| | | GTTCTGGGACGGATTACACTCTGACCATCAGCAGT |
| | | CTGCAACCGGAAGACTTCGCAACTTATTACTGTCA |
| | | GCAAGGTAATACTCTGCCGTGGACGTTCGGACAGG |
| | | GCACCAAGGTGGAGATCAAAGGCGGCGGCGGAAGT |
| | | GGAGGAGGAGGCTCAGGCGGAGGAGGGAGCGAGGT |
| | | TCAGCTGGTGGAGTCTGGCGGTGGCCTGGTGCAGC |
| | | CAGGGGGCTCACTCCGTTTGTCCTGTGCAGCTTCT |
| | | GGCTACTCCTTTACCGGCTACACTATGAACTGGGT |
| | | GCGTCAGGCCCCAGGTAAGGGCCTGGAATGGGTTG |
| | | CACTGATTAATCCTACCAAAGGTGTTAGTACCTAC |
| | | AACCAGAAGTTCAAGGACCGTTTCACTATAAGCGT |
| | | AGATAAATCCAAAAACACAGCCTACCTGCAAATGA |
| | | ACAGCCTGCGTGCTGAGGACACTGCCGTCTATTAT |
| | | TGTGCTAGAAGCGGATACTACGGCGATAGTGACTG |
| | | GTATTTTGACGTGTGGGGTCAAGGAACCCTGGTCA |
| | | CCGTCTCCTCGACATCTGGCGGCGGAGGATCTCTG |
| | | GAATCTGGACAGGTGCTGCTGGAAAGCAACATCAA |
| | | GGTGCTGCCCACCTGGTCTACCCCAGTTCAGCCTA |
| | | TGGCTCTGATTGTGCTTGGCGGAGTTGCCGGCCTG |
| | | CTGCTCTTTATCGGCCTGGGCATCTTCTTTTGCGT |
| | | GCGGTGCAGACATCGGCGGAGACAGGCTGAGAGAA |
| | | TGAGCCAGATCAAGCGGCTGCTGAGCGAGAAGAAA |
| | | ACCTGTCAGTGCCCTCACCGGTTCCAGAAAACATG |
| | | CAGCCCCATC |
| 604 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGCAGTCTGGGGCTGAGGTGAAGAAGCCT |
| | | GGGTCCTCGGTGAAGGTCTCCTGCAAGGCTTCCGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGATCGGGGGAGAAGATATTGTAGTGGTG |
| | | GTAGCTGCCCTAATGCTTTTGATATCTGGGGCCAA |
| | | GGGACAATGGTCACCGTGTCTTCAGGGTCAACCTC |
| | | TGGTAGCGGTAAGCCTGGCTCCGGCGAAGGCTCCA |
| | | CAAAGGGTGAAATTGTGTTGACACAGTCTCCAGCC |
| | | ACCCTGTCTGTGTCTCCAGGGGAAAGAGCCACCCT |
| | | CTCCTGCAGGGCCAGTCAGAGTGTTAGCAGCAACT |
| | | TAGCCTGGTACCAGCAGAAACCTGGCCAGGCTCCC |
| | | AGGCTCCTCATCTATGGTGCATCCACCAGGGCCAC |
| | | TGGTATCCCAGCCAGGTTCAGTGGCAGTGGGTCTG |
| | | GGACAGAGTTCACTCTCACCATCAGCAGCCTGCAG |
| | | TCTGAAGATTTTGCAGTTTATTACTGTCAGCAGTA |
| | | TAATAACTGGCCCCCGGCCCTCACTTTCGGCGGAG |
| | | GGACCAAGGTGGAAATCAAAGAGCAGAAACTGATC |
| | | AGCGAGGAAGATCTGAATCCTGGCGGAGGCGGAGG |
| | | AAGTGGTGGCGGAGGTTCTGGTGGCGGTGGATCAG |
| | | GCGGTGGCGGATCTGGATCTATGGATATCCAGATG |
| | | ACCCAGTCCCCGAGCTCCCTGTCCGCCTCTGTGGG |
| | | CGATAGGGTCACCATCACCTGCCGTGCCAGTCAGG |
| | | ACATCCGTAATTATCTGAACTGGTATCAACAGAAA |
| | | CCAGGAAAAGCTCCGAAACTACTGATTTACTATAC |
| | | CTCCCGCCTGGAGTCTGGAGTCCCTTCTCGCTTCT |
| | | CTGGTTCTGGTTCTGGGACGGATTACACTCTGACC |
| | | ATCAGCAGTCTGCAACCGGAAGACTTCGCAACTTA |
| | | TTACTGTCAGCAAGGTAATACTCTGCCGTGGACGT |
| | | TCGGACAGGGCACCAAGGTGGAGATCAAAGGCGGC |
| | | GGCGGAAGTGGAGGAGGAGGCTCAGGCGGAGGAGG |
| | | GAGCGAGGTTCAGCTGGTGGAGTCTGGCGGTGGCC |
| | | TGGTGCAGCCAGGGGGCTCACTCCGTTTGTCCTGT |
| | | GCAGCTTCTGGCTACTCCTTTACCGGCTACACTAT |
| | | GAACTGGGTGCGTCAGGCCCCAGGTAAGGGCCTGG |
| | | AATGGGTTGCACTGATTAATCCTACCAAAGGTGTT |
| | | AGTACCTACAACCAGAAGTTCAAGGACCGTTTCAC |
| | | TATAAGCGTAGATAAATCCAAAAACACAGCCTACC |
| | | TGCAAATGAACAGCCTGCGTGCTGAGGACACTGCC |
| | | GTCTATTATTGTGCTAGAAGCGGATACTACGGCGA |
| | | TAGTGACTGGTATTTTGACGTGTGGGGTCAAGGAA |
| | | CCCTGGTCACCGTCTCCTCGACATCTGGCGGCGGA |
| | | GGATCTCTGGAATCTGGACAGGTGCTGCTGGAAAG |
| | | CAACATCAAGGTGCTGCCCACCTGGTCTACCCCAG |
| | | TTCAGCCTATGGCTCTGATTGTGCTTGGCGGAGTT |
| | | GCCGGCCTGCTGCTCTTTATCGGCCTGGGCATCTT |
| | | CTTTTGCGTGCGGTGCAGACATCGGCGGAGACAGG |
| | | CTGAGAGAATGAGCCAGATCAAGCGGCTGCTGAGC |
| | | GAGAAGAAAACCTGTCAGTGCCCTCACCGGTTCCA |
| | | GAAAACATGCAGCCCCATC |
| 605 | Nucleotide | ATGGCTCTGCCTGTGACAGCTCTGTTGCTGCCTCT |
| | | GGCTCTGCTGCTGCATGCTGCTAGACCTCAGGTTC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGGGGGTTACGTTTGGGGGAGTTATCGTC |
| | | AGAACTCCAACTGGTTCGACCCCTGGGGCCAGGGA |
| | | ACCCTGGTCACCGTGTCCTCAGGGTCAACCTCTGG |
| | | TAGCGGTAAGCCTGGCTCCGGCGAAGGCTCCACAA |
| | | AGGGTTCCTATGTGCTGACTCAGCCACCCTCAGCG |
| | | TCTGGGACCCCCGGGCAGAGGGTCACCATCTCTTG |
| | | TTCTGGAAGCAGCTCCAACATCGGAAGTAATTATG |
| | | TATACTGGTACCAGCAGCTCCCAGGAACGGCCCCC |
| | | AAACTCCTCATCTATAGGAATAATCAGCGGCCCTC |
| | | AGGGGTCCCTGACCGATTCTCTGGCTCCAAGTCTG |
| | | GCACCTCAGCCTCCCTGGCCATCAGTGGGCTCCGG |
| | | TCCGAGGATGAGGCTGATTATTACTGTGCAGCATG |
| | | GGATGACAGCCTGAGTGGTTGGGTGTTCGGCGGAG |
| | | GGACCAAGCTGACCGTCCTAGAGCAGAAACTGATC |
| | | AGCGAGGAAGATCTGAATCCTGGCGGAGGCGGAGG |
| | | AAGTGGTGGCGGAGGTTCTGGTGGCGGTGGATCAG |
| | | GCGGTGGCGGATCTGGATCTATGGATATCCAGATG |
| | | ACCCAGTCCCCGAGCTCCCTGTCCGCCTCTGTGGG |
| | | CGATAGGGTCACCATCACCTGCCGTGCCAGTCAGG |
| | | ACATCCGTAATTATCTGAACTGGTATCAACAGAAA |
| | | CCAGGAAAAGCTCCGAAACTACTGATTTACTATAC |
| | | CTCCCGCCTGGAGTCTGGAGTCCCTTCTCGCTTCT |
| | | CTGGTTCTGGTTCTGGGACGGATTACACTCTGACC |
| | | ATCAGCAGTCTGCAACCGGAAGACTTCGCAACTTA |
| | | TTACTGTCAGCAAGGTAATACTCTGCCGTGGACGT |
| | | TCGGACAGGGCACCAAGGTGGAGATCAAAGGCGGC |
| | | GGCGGAAGTGGAGGAGGAGGCTCAGGCGGAGGAGG |
| | | GAGCGAGGTTCAGCTGGTGGAGTCTGGCGGTGGCC |
| | | TGGTGCAGCCAGGGGGCTCACTCCGTTTGTCCTGT |
| | | GCAGCTTCTGGCTACTCCTTTACCGGCTACACTAT |
| | | GAACTGGGTGCGTCAGGCCCCAGGTAAGGGCCTGG |
| | | AATGGGTTGCACTGATTAATCCTACCAAAGGTGTT |
| | | AGTACCTACAACCAGAAGTTCAAGGACCGTTTCAC |
| | | TATAAGCGTAGATAAATCCAAAAACACAGCCTACC |
| | | TGCAAATGAACAGCCTGCGTGCTGAGGACACTGCC |
| | | GTCTATTATTGTGCTAGAAGCGGATACTACGGCGA |
| | | TAGTGACTGGTATTTTGACGTGTGGGGTCAAGGAA |
| | | CCCTGGTCACCGTCTCCTCGACATCTGGCGGCGGA |
| | | GGATCTCTGGAATCTGGACAGGTGCTGCTGGAAAG |
| | | CAACATCAAGGTGCTGCCCACCTGGTCTACCCCAG |
| | | TTCAGCCTATGGCTCTGATTGTGCTTGGCGGAGTT |
| | | GCCGGCCTGCTGCTCTTTATCGGCCTGGGCATCTT |
| | | CTTTTGCGTGCGGTGCAGACATCGGCGGAGACAGG |
| | | CTGAGAGAATGAGCCAGATCAAGCGGCTGCTGAGC |
| | | GAGAAGAAAACCTGTCAGTGCCCTCACCGGTTCCA |
| | | GAAAACATGCAGCCCCATC |
| 606 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGTCTGGGGCTGAGGTGAAGAAGCCT |
| | | GGGTCCTCGGTGAAGGTCTCCTGCAAGGCTTCGGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGACATATTACGATTGAGCGTGTCCTCGG |
| | | GTATGGACGTCTGGGGCCAAGGGACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGCTGACTCAGCCACCCTCAGCGTCTGGGACCCCC |
| | | GGGCGGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGTGTGGTATTCGGCGGAGGGACCAAGCTGA |
| | | CCGTCCTAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTACCAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 607 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGTCTGGGGGAGGCTTGGTCCAGCCT |
| | | GGGGGGTCCCTGAGACTCTCCTGTGCAGCCTCTGG |
| | | ATTCACCTTTAGTAGCTATTGGATGAGCTGGGTCC |
| | | GCCAGGCTCCAGGGAAGGGGCTGGAGTGGGTGGCC |
| | | AACATAAAGCAAGATGGAAGTGAGAAATACTATGT |
| | | GGACTCTGTGAAGGGCCGATTCACCATCTCCAGAG |
| | | ACAACGCCAAGAACTCACTGTATCTGCAAATGAAC |
| | | AGCCTGAGAGCCGAGGACACGGCTGTGTATTACTG |
| | | TGCGAGATGGAGTCATTACTATGATAGTAGTGGTC |
| | | TTGACTACTGGGGCCAGGGAACCCTGGTCACCGTG |
| | | TCCTCAGGGTCAACCTCTGGTAGCGGTAAGCCTGG |
| | | CTCCGGCGAAGGCTCCACAAAGGGTTCCTATGTGC |
| | | TGACTCAGCCACCCTCGGTGTCAGTGTCCCCAGGA |
| | | CAGACGGCCAGGATCACCTGCTCTGGAGATGCATT |
| | | GCCAAAGCAATATGCTTATTGGTACCAGCAGAAGC |
| | | CAGGCCAGGCCCCTGTGCTGGTGATATATAAAGAC |
| | | AGTGAGAGGCCCTCAGGGATCCCTGAGCGATTCTC |
| | | TGGCTCCAGCTCAGGGACAACAGTCACGTTGACCA |
| | | TCAGTGGAGTCCAGGCAGAAGACGAGGCTGACTAT |
| | | TACTGTCAATCAGCAGACAGCAGTGGTACTTGGGT |
| | | GTTCGGCGGAGGGACCAAGCTGACCGTCCTAGAGC |
| | | AGAAACTGATCAGCGAGGAAGATCTGAATCCTGGC |
| | | GGAGGCGGAGGAAGTGGTGGCGGAGGTTCTGGTGG |
| | | CGGTGGATCAGGCGGTGGCGGATCTGGATCTATGG |
| | | ATATCCAGATGACCCAGTCCCCGAGCTCCCTGTCC |
| | | GCCTCTGTGGGCGATAGGGTCACCATCACCTGCCG |
| | | TGCCAGTCAGGACATCCGTAATTATCTGAACTGGT |
| | | ATCAACAGAAACCAGGAAAAGCTCCGAAACTACTG |
| | | ATTTACTATACCTCCCGCCTGGAGTCTGGAGTCCC |
| | | TTCTCGCTTCTCTGGTTCTGGTTCTGGGACGGATT |
| | | ACACTCTGACCATCAGCAGTCTGCAACCGGAAGAC |
| | | TTCGCAACTTATTACTGTCAGCAAGGTAATACTCT |
| | | GCCGTGGACGTTCGGACAGGGCACCAAGGTGGAGA |
| | | TCAAAGGCGGCGGCGGAAGTGGAGGAGGAGGCTCA |
| | | GGCGGAGGAGGGAGCGAGGTTCAGCTGGTGGAGTC |
| | | TGGCGGTGGCCTGGTGCAGCCAGGGGGCTCACTCC |
| | | GTTTGTCCTGTGCAGCTTCTGGCTACTCCTTTACC |
| | | GGCTACACTATGAACTGGGTGCGTCAGGCCCCAGG |
| | | TAAGGGCCTGGAATGGGTTGCACTGATTAATCCTA |
| | | CCAAAGGTGTTAGTACCTACAACCAGAAGTTCAAG |
| | | GACCGTTTCACTATAAGCGTAGATAAATCCAAAAA |
| | | CACAGCCTACCTGCAAATGAACAGCCTGCGTGCTG |
| | | AGGACACTGCCGTCTATTATTGTGCTAGAAGCGGA |
| | | TACTACGGCGATAGTGACTGGTATTTTGACGTGTG |
| | | GGGTCAAGGAACCCTGGTCACCGTCTCCTCGACAT |
| | | CTGGCGGCGGAGGATCTCTGGAATCTGGACAGGTG |
| | | CTGCTGGAAAGCAACATCAAGGTGCTGCCCACCTG |
| | | GTCTACCCCAGTTCAGCCTATGGCTCTGATTGTGC |
| | | TTGGCGGAGTTGCCGGCCTGCTGCTCTTTATCGGC |
| | | CTGGGCATCTTCTTTTGCGTGCGGTGCAGACATCG |
| | | GCGGAGACAGGCTGAGAGAATGAGCCAGATCAAGC |
| | | GGCTGCTGAGCGAGAAGAAAACCTGTCAGTGCCCT |
| | | CACCGGTTCCAGAAAACATGCAGCCCCATC |
| 608 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACAAATCCACGAGCACAGCCTACATGGAGCTGAGC |
| | | AGCCTGAGGAGCGAGGACACGGCCGTGTATTACTG |
| | | TGCGAGGACCCATCTACCATACAGCTATGGTTTAG |
| | | GCGGGTTTGACTACTGGGGCCAGGGAACCCTGGTC |
| | | ACCGTGTCCTCAGGGTCAACCTCTGGTAGCGGTAA |
| | | GCCTGGCTCCGGCGAAGGCTCCACAAAGGGTCAGT |
| | | CTGTGCTGACGCAGCCGCCCTCAGTGTCTGGGGCC |
| | | CCAGGGCAGAGGGTCACCATCTCCTGCACTGGGAG |
| | | CAGCTCCAACATCGGGGCAGGTTATGATGTACACT |
| | | GGTACCAGCAGCTTCCAGGAACAGCCCCCAAACTC |
| | | CTCATCTATGGTAACAGCAATCGGCCCTCAGGGGT |
| | | CCCTGACCGATTCTCTGGCTCCAAGTCTGGCACCT |
| | | CAGCCTCCCTGGCCATCACTGGGCTCCAGGCTGAG |
| | | GATGAGGCTGATTATTACTGCCAGTCCTATGACAG |
| | | CAGCCTGAGTGGCCCGGTGGTATTCGGCGGAGGGA |
| | | CCAAGGTCACCGTCCTAGAGCAGAAACTGATCAGC |
| | | GAGGAAGATCTGAATCCTGGCGGAGGCGGAGGAAG |
| | | TGGTGGCGGAGGTTCTGGTGGCGGTGGATCAGGCG |
| | | GTGGCGGATCTGGATCTATGGATATCCAGATGACC |
| | | CAGTCCCCGAGCTCCCTGTCCGCCTCTGTGGGCGA |
| | | TAGGGTCACCATCACCTGCCGTGCCAGTCAGGACA |
| | | TCCGTAATTATCTGAACTGGTATCAACAGAAACCA |
| | | GGAAAAGCTCCGAAACTACTGATTTACTATACCTC |
| | | CCGCCTGGAGTCTGGAGTCCCTTCTCGCTTCTCTG |
| | | GTTCTGGTTCTGGGACGGATTACACTCTGACCATC |
| | | AGCAGTCTGCAACCGGAAGACTTCGCAACTTATTA |
| | | CTGTCAGCAAGGTAATACTCTGCCGTGGACGTTCG |
| | | GACAGGGCACCAAGGTGGAGATCAAAGGCGGCGGC |
| | | GGAAGTGGAGGAGGAGGCTCAGGCGGAGGAGGGAG |
| | | CGAGGTTCAGCTGGTGGAGTCTGGCGGTGGCCTGG |
| | | TGCAGCCAGGGGGCTCACTCCGTTTGTCCTGTGCA |
| | | GCTTCTGGCTACTCCTTTACCGGCTACACTATGAA |
| | | CTGGGTGCGTCAGGCCCCAGGTAAGGGCCTGGAAT |
| | | GGGTTGCACTGATTAATCCTACCAAAGGTGTTAGT |
| | | ACCTACAACCAGAAGTTCAAGGACCGTTTCACTAT |
| | | AAGCGTAGATAAATCCAAAAACACAGCCTACCTGC |
| | | AAATGAACAGCCTGCGTGCTGAGGACACTGCCGTC |
| | | TATTATTGTGCTAGAAGCGGATACTACGGCGATAG |
| | | TGACTGGTATTTTGACGTGTGGGGTCAAGGAACCC |
| | | TGGTCACCGTCTCCTCGACATCTGGCGGCGGAGGA |
| | | TCTCTGGAATCTGGACAGGTGCTGCTGGAAAGCAA |
| | | CATCAAGGTGCTGCCCACCTGGTCTACCCCAGTTC |
| | | AGCCTATGGCTCTGATTGTGCTTGGCGGAGTTGCC |
| | | GGCCTGCTGCTCTTTATCGGCCTGGGCATCTTCTT |
| | | TTGCGTGCGGTGCAGACATCGGCGGAGACAGGCTG |
| | | AGAGAATGAGCCAGATCAAGCGGCTGCTGAGCGAG |
| | | AAGAAAACCTGTCAGTGCCCTCACCGGTTCCAGAA |
| | | AACATGCAGCCCCATC |
| 609 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGATCCAAGTCGGGATTACTATGGTTCGG |
| | | GGAGATTCTCATGGTTCGACCCCTGGGGCCAGGGA |
| | | ACCCTGGTCACCGTGTCCTCAGGGTCAACCTCTGG |
| | | TAGCGGTAAGCCTGGCTCCGGCGAAGGCTCCACAA |
| | | AGGGTCAGTCTGTGCTGACTCAGCCACCCTCAGCG |
| | | TCTGGGACCCCCGGGCAGAGGGTCACCATCTCTTG |
| | | TTCTGGAAGCAGCTCCAACATCGGAAGTAATTATG |
| | | TATACTGGTACCAGCAGCTCCCAGGAACGGCCCCC |
| | | AAACTCCTCATCTATAGGAATAATCAGCGGCCCTC |
| | | AGGGGTCCCTGACCGATTCTCTGGCTCCAAGTCTG |
| | | GCACCTCAGCCTCCCTGGCCATCAGTGGGCTCCGG |
| | | TCCGAGGATGAGGCTGATTATTACTGTGCAGCATG |
| | | GGATGACAGCCTGAGTGGTCGGGGGGTATTCGGCG |
| | | GAGGGACCAAGCTGACCGTCCTAGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 610 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGCAGTCTGGGGCTGAGGTGAAGAAGCCT |
| | | GGGGCCTCAGTGAAGGTCTCCTGCAAGGCTTCTGG |
| | | TTACACCTTTACCAGCTATGGTATCAGCTGGGTGC |
| | | GACAGGCCCCTGGACAAGGGCTTGAGTGGATGGGA |
| | | TGGATCAGCGCTTACAATGGTAACACAAACTATGC |
| | | ACAGAAGCTCCAGGGCAGAGTCACCATGACCACAG |
| | | ACACATCCACGAGCACAGCCTACATGGAGCTGAGG |
| | | AGCCTGAGGAGCGACGACACGGCCGTGTATTACTG |
| | | TGCGAGAGATATAATACGATATTGTAGTAGTACCA |
| | | GCTGCTATAGAGGGATTGACTACTGGGGCCAGGGA |
| | | ACCCTGGTCACCGTGTCCTCAGGGTCAACCTCTGG |
| | | TAGCGGTAAGCCTGGCTCCGGCGAAGGCTCCACAA |
| | | AGGGTCAGTCTGCCCTGACTCAGCCTGCCTCCGTG |
| | | TCTGGGTCTCCTGGACAGTCGATCACCATCTCCTG |
| | | CACTGGAACCAGCAGTGACGTTGGTGGTTATAACT |
| | | ATGTCTCCTGGTACCAACAGCACCCAGGCAAAGCC |
| | | CCCAAACTCATGATTTATGATGTCAGTAATCGGCC |
| | | CTCAGGGGTTTCTAATCGCTTCTCTGGCTCCAAGT |
| | | CTGGCAACACGGCCTCCCTGACCATCTCTGGGCTC |
| | | CAGGCTGAGGACGAGGCTGATTATTACTGCAGCTC |
| | | ATATACAAGCAGCAGCCCCCATGTGGTATCCGGCG |
| | | GAGGGACCAAGCTGACGGTCCTAGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 611 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGTCTGGGGGAGGCGTGGTCCAGCCT |
| | | GGGAGGTCCCTGAGACTCTCCTGTGCAGCCTCTGG |
| | | ATTCACCTTCAGTAGCTATGGCATGCACTGGGTCC |
| | | GCCAGGCTCCAGGCAAGGGGCTGGAGTGGGTGGCA |
| | | GTTATATCATATGATGGAAGTAATAAATACTATGC |
| | | AGACTCCGTGAAGGGCCGATTCACCATCTCCAGAG |
| | | ACAATTCCAAGAACACGCTGTATCTGCAAATGAAC |
| | | AGCCTGAGAGCTGAGGACACGGCTGTGTATTACTG |
| | | TGCGAGAGATGGAACGGACTACCGAGGTGCTTTTG |
| | | ATATCTGGGGCCAAGGGACAATGGTCACCGTGTCT |
| | | TCAGGGTCAACCTCTGGTAGCGGTAAGCCTGGCTC |
| | | CGGCGAAGGCTCCACAAAGGGTGACATCCAGTTGA |
| | | CCCAGTCTCCAGACTCCCTGGCTGTGCCTCTGGGC |
| | | GAGAGGGCCACCATCAACTGCAAGTCCAGCCAGAG |
| | | TGTTTTATACAGCTCCAACAATAAGAACTACTTAG |
| | | CTTGGTACCAGCAGAAACCAGGACAGCCTCCTAAG |
| | | CTGCTCATTTACTGGGCATCTACCCGGGAATCCGG |
| | | GGTCCCTGACCGATTCAGTGGCAGCGGGTCTGGGA |
| | | CAGATTTCACTCTCACCATCAGCAGCCTGCAGGCT |
| | | GAAGATGTGGCAGTTTATTACTGTCAGCAATATTA |
| | | TAGTACTCCTCTGACGTTCGGCCAAGGGACCAAGC |
| | | TGGAGATCAAAGAGCAGAAACTGATCAGCGAGGAA |
| | | GATCTGAATCCTGGCGGAGGCGGAGGAAGTGGTGG |
| | | CGGAGGTTCTGGTGGCGGTGGATCAGGCGGTGGCG |
| | | GATCTGGATCTATGGATATCCAGATGACCCAGTCC |
| | | CCGAGCTCCCTGTCCGCCTCTGTGGGCGATAGGGT |
| | | CACCATCACCTGCCGTGCCAGTCAGGACATCCGTA |
| | | ATTATCTGAACTGGTATCAACAGAAACCAGGAAAA |
| | | GCTCCGAAACTACTGATTTACTATACCTCCCGCCT |
| | | GGAGTCTGGAGTCCCTTCTCGCTTCTCTGGTTCTG |
| | | GTTCTGGGACGGATTACACTCTGACCATCAGCAGT |
| | | CTGCAACCGGAAGACTTCGCAACTTATTACTGTCA |
| | | GCAAGGTAATACTCTGCCGTGGACGTTCGGACAGG |
| | | GCACCAAGGTGGAGATCAAAGGCGGCGGCGGAAGT |
| | | GGAGGAGGAGGCTCAGGCGGAGGAGGGAGCGAGGT |
| | | TCAGCTGGTGGAGTCTGGCGGTGGCCTGGTGCAGC |
| | | CAGGGGGCTCACTCCGTTTGTCCTGTGCAGCTTCT |
| | | GGCTACTCCTTTACCGGCTACACTATGAACTGGGT |
| | | GCGTCAGGCCCCAGGTAAGGGCCTGGAATGGGTTG |
| | | CACTGATTAATCCTACCAAAGGTGTTAGTACCTAC |
| | | AACCAGAAGTTCAAGGACCGTTTCACTATAAGCGT |
| | | AGATAAATCCAAAAACACAGCCTACCTGCAAATGA |
| | | ACAGCCTGCGTGCTGAGGACACTGCCGTCTATTAT |
| | | TGTGCTAGAAGCGGATACTACGGCGATAGTGACTG |
| | | GTATTTTGACGTGTGGGGTCAAGGAACCCTGGTCA |
| | | CCGTCTCCTCGACATCTGGCGGCGGAGGATCTCTG |
| | | GAATCTGGACAGGTGCTGCTGGAAAGCAACATCAA |
| | | GGTGCTGCCCACCTGGTCTACCCCAGTTCAGCCTA |
| | | TGGCTCTGATTGTGCTTGGCGGAGTTGCCGGCCTG |
| | | CTGCTCTTTATCGGCCTGGGCATCTTCTTTTGCGT |
| | | GCGGTGCAGACATCGGCGGAGACAGGCTGAGAGAA |
| | | TGAGCCAGATCAAGCGGCTGCTGAGCGAGAAGAAA |
| | | ACCTGTCAGTGCCCTCACCGGTTCCAGAAAACATG |
| | | CAGCCCCATC |
| 612 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGCAGTCTGGGGCTGAGGTGAAGAAGCCT |
| | | GGGTCCTCGGTGAGGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACAAATCCACGAGCACAGCCTACATGGAGCTGAGC |
| | | AGCCTGAGGAGCGAGGACACGGCCGTGTATTACTG |
| | | TGCGAGAGGGGGCGGAAGGTGGCTACACTCCCGTC |
| | | TGGACGTCTGGGGCCAAGGGACCACGGTCACCGTG |
| | | TCCTCAGGGTCAACCTCTGGTAGCGGTAAGCCTGG |
| | | CTCCGGCGAAGGCTCCACAAAGGGTCAGTCTGTGT |
| | | TGACGCAGCCGCCCTCAGTGTCTGGGGCCCCAGGG |
| | | CAGAGGGTCACCATCTCCTGCACTGGGAGCAGCTC |
| | | CAACATCGGGGCAGGTTATGATGTACACTGGTATC |
| | | AGCAGTTTCCAGGAACAGCCCCCAAACTCCTCATC |
| | | TTTGGTAACAACAATCGGCCCTCAGGGGTCCCTGA |
| | | CCGATTCTCTGGCTCCAAGTCTGGCACCTCAGCCT |
| | | CCCTGGCCATCACTGGGCTCCAGGCTGAGGATGAG |
| | | GCTAATTATTACTGCCAGTCCTATGACAGGAGCCT |
| | | GAGTGGTCCCGTGGTCTTCGGCGGAGGGACCAAGC |
| | | TGACCGTCCTAGAGCAGAAACTGATCAGCGAGGAA |
| | | GATCTGAATCCTGGCGGAGGCGGAGGAAGTGGTGG |
| | | CGGAGGTTCTGGTGGCGGTGGATCAGGCGGTGGCG |
| | | GATCTGGATCTATGGATATCCAGATGACCCAGTCC |
| | | CCGAGCTCCCTGTCCGCCTCTGTGGGCGATAGGGT |
| | | CACCATCACCTGCCGTGCCAGTCAGGACATCCGTA |
| | | ATTATCTGAACTGGTATCAACAGAAACCAGGAAAA |
| | | GCTCCGAAACTACTGATTTACTATACCTCCCGCCT |
| | | GGAGTCTGGAGTCCCTTCTCGCTTCTCTGGTTCTG |
| | | GTTCTGGGACGGATTACACTCTGACCATCAGCAGT |
| | | CTGCAACCGGAAGACTTCGCAACTTATTACTGTCA |
| | | GCAAGGTAATACTCTGCCGTGGACGTTCGGACAGG |
| | | GCACCAAGGTGGAGATCAAAGGCGGCGGCGGAAGT |
| | | GGAGGAGGAGGCTCAGGCGGAGGAGGGAGCGAGGT |
| | | TCAGCTGGTGGAGTCTGGCGGTGGCCTGGTGCAGC |
| | | CAGGGGGCTCACTCCGTTTGTCCTGTGCAGCTTCT |
| | | GGCTACTCCTTTACCGGCTACACTATGAACTGGGT |
| | | GCGTCAGGCCCCAGGTAAGGGCCTGGAATGGGTTG |
| | | CACTGATTAATCCTACCAAAGGTGTTAGTACCTAC |
| | | AACCAGAAGTTCAAGGACCGTTTCACTATAAGCGT |
| | | AGATAAATCCAAAAACACAGCCTACCTGCAAATGA |
| | | ACAGCCTGCGTGCTGAGGACACTGCCGTCTATTAT |
| | | TGTGCTAGAAGCGGATACTACGGCGATAGTGACTG |
| | | GTATTTTGACGTGTGGGGTCAAGGAACCCTGGTCA |
| | | CCGTCTCCTCGACATCTGGCGGCGGAGGATCTCTG |
| | | GAATCTGGACAGGTGCTGCTGGAAAGCAACATCAA |
| | | GGTGCTGCCCACCTGGTCTACCCCAGTTCAGCCTA |
| | | TGGCTCTGATTGTGCTTGGCGGAGTTGCCGGCCTG |
| | | CTGCTCTTTATCGGCCTGGGCATCTTCTTTTGCGT |
| | | GCGGTGCAGACATCGGCGGAGACAGGCTGAGAGAA |
| | | TGAGCCAGATCAAGCGGCTGCTGAGCGAGAAGAAA |
| | | ACCTGTCAGTGCCCTCACCGGTTCCAGAAAACATG |
| | | CAGCCCCATC |
| 613 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGTCTGGGGGAGGCGTGGTCCAGCCT |
| | | GGGAGGTCCCTGAGACTCTCCTGTGCAGCCTCTGG |
| | | ATTCACCTTCAGTAGCTATGGCATGCACTGGGTCC |
| | | GCCAGGCTCCAGGCAAGGGGCTGGAGTGGGTGGCA |
| | | GTTATATCATATGATGGAAGTAATAAATACTATGC |
| | | AGACTCCGTGAAGGGCCGATTCACCATCTCCAGAG |
| | | ACAATTCCAAGAACACGCTGTATCTGCAAATGAAC |
| | | AGCCTGAGAGCTGAGGACACGGCTGTGTATTACTG |
| | | TGCGAAAGATGCTGGGGACCTGGGTGCTTTTGATA |
| | | TCTGGGGCCAAGGGACAATGGTCACCGTGTCTTCA |
| | | GGGTCAACCTCTGGTAGCGGTAAGCCTGGCTCCGG |
| | | CGAAGGCTCCACAAAGGGTGACATCGTGATGACCC |
| | | AGTCTCCAGACTCCCTGGCTGTGTCTCTGGGCGAG |
| | | AGGGCCACCATCAACTGCAAGTCCAGCCAGAGTGT |
| | | TTTATACAGCTCCAACAATAAGAACTACTTAGCTT |
| | | GGTACCAGCAGAAACCAGGACAGCCTCCTAAGCTG |
| | | CTCATTTACTGGGCATCTACCCGGGAATCCGGGGT |
| | | CCCTGACCGATTCAGTGGCAGCGGGTCTGGGACAG |
| | | ATTTCACTCTCACCATCAGCAGCCTGCAGGCTGAA |
| | | GATGTGGCAGTTTATTACTGTCAGCAATATTATAG |
| | | TACTCCTCTCACTTTCGGCCCTGGGACCAAGGTGG |
| | | AAATCAAAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTACCAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 614 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGCAGTCTGGGGGAGGCGTGGTCCAGCCT |
| | | GGGAGGTCCCTGAGACTCTCCTGTGCAGCCTCTGG |
| | | ATTCACCTTCAGTAGCTATGCTATGCACTGGGTCC |
| | | GCCAGGCTCCAGGCAAGGGGCTGGAGTGGGTGGCA |
| | | GTTATATCATATGATGGAAGTAATAAATACTACGC |
| | | AGACTCCGTGAAGGGCCGATTCACCATCTCCAGAG |
| | | ACAATTCCAAGAACACGCTGTATCTGCAAATGAAC |
| | | AGCCTGAGAGCTGAGGACACGGCTGTGTATTACTG |
| | | TGCGAGAGGGCGGGTGTATTACTATGATAGTAGTG |
| | | GTTATAGCTACTGGGGCCAGGGAACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGTTGACGCAGCCGCCCTCAGTGTCTGGGGCCCCA |
| | | GGGCAGAGGGTCACCATCTCCTGCACTGGGAGCAG |
| | | CTCCAACATCGGGGCAGGTTATGATGTACACTGGT |
| | | ACCAGCAGCTTCCAGGAACAGCCCCCAAACTCCTC |
| | | ATCTATGGTAACAGCAATCGGCCCTCAGGGGTCCC |
| | | TGACCGATTCTCTGGCTCCAAGTCTGGCACCTCAG |
| | | CCTCCCTGGCCATCACTGGGCTCCAGGCTGAGGAT |
| | | GAGGCTGATTATTACTGCCAGTCCTATGACAGCAG |
| | | CCTGAGTGGCCGGGAGGTCTTCGGAACTGGGACCA |
| | | AGCTGACCGTCCTAGAGCAGAAACTGATCAGCGAG |
| | | GAAGATCTGAATCCTGGCGGAGGCGGAGGAAGTGG |
| | | TGGCGGAGGTTCTGGTGGCGGTGGATCAGGCGGTG |
| | | GCGGATCTGGATCTATGGATATCCAGATGACCCAG |
| | | TCCCCGAGCTCCCTGTCCGCCTCTGTGGGCGATAG |
| | | GGTCACCATCACCTGCCGTGCCAGTCAGGACATCC |
| | | GTAATTATCTGAACTGGTATCAACAGAAACCAGGA |
| | | AAAGCTCCGAAACTACTGATTTACTATACCTCCCG |
| | | CCTGGAGTCTGGAGTCCCTTCTCGCTTCTCTGGTT |
| | | CTGGTTCTGGGACGGATTACACTCTGACCATCAGC |
| | | AGTCTGCAACCGGAAGACTTCGCAACTTATTACTG |
| | | TCAGCAAGGTAATACTCTGCCGTGGACGTTCGGAC |
| | | AGGGCACCAAGGTGGAGATCAAAGGCGGCGGCGGA |
| | | AGTGGAGGAGGAGGCTCAGGCGGAGGAGGGAGCGA |
| | | GGTTCAGCTGGTGGAGTCTGGCGGTGGCCTGGTGC |
| | | AGCCAGGGGGCTCACTCCGTTTGTCCTGTGCAGCT |
| | | TCTGGCTACTCCTTTACCGGCTACACTATGAACTG |
| | | GGTGCGTCAGGCCCCAGGTAAGGGCCTGGAATGGG |
| | | TTGCACTGATTAATCCTACCAAAGGTGTTAGTACC |
| | | TACAACCAGAAGTTCAAGGACCGTTTCACTATAAG |
| | | CGTAGATAAATCCAAAAACACAGCCTACCTGCAAA |
| | | TGAACAGCCTGCGTGCTGAGGACACTGCCGTCTAT |
| | | TATTGTGCTAGAAGCGGATACTACGGCGATAGTGA |
| | | CTGGTATTTTGACGTGTGGGGTCAAGGAACCCTGG |
| | | TCACCGTCTCCTCGACATCTGGCGGCGGAGGATCT |
| | | CTGGAATCTGGACAGGTGCTGCTGGAAAGCAACAT |
| | | CAAGGTGCTGCCCACCTGGTCTACCCCAGTTCAGC |
| | | CTATGGCTCTGATTGTGCTTGGCGGAGTTGCCGGC |
| | | CTGCTGCTCTTTATCGGCCTGGGCATCTTCTTTTG |
| | | CGTGCGGTGCAGACATCGGCGGAGACAGGCTGAGA |
| | | GAATGAGCCAGATCAAGCGGCTGCTGAGCGAGAAG |
| | | AAAACCTGTCAGTGCCCTCACCGGTTCCAGAAAAC |
| | | ATGCAGCCCCATC |
| 615 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGCGCCCCCTGTATGCCCCTCGCTTCGGATACG |
| | | GTATGGACGTCTGGGGCCAAGGGACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGTTGACGCAGCCGCCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGTTGGGTGTTCGGCGGAGGCACCCAGCTGA |
| | | CCGTCCTCGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTACCAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 616 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGTTGGAGTCTGGGGGAGGCTTGGTACAGCCT |
| | | GGGGGGTCCCTGAGACTCTCCTGTGCAGCCTCTGG |
| | | ATTCACCTTTAGCAGCTATGCCATGAGCTGGGTCC |
| | | GCCAGGCTCCAGGGAAGGGGCTGGAGTGGGTCTCA |
| | | GCTATTAGTGGTAGTGGTGGTAGCACATACTACGC |
| | | AGACTCCGTGAAGGGCCGGTTCACCATCTCCAGAG |
| | | ACAATTCCAAGAACACGCTGTATCTGCAAATGAAC |
| | | AGCCTGAGAGCCGAGGACACGGCCGTATATTACTG |
| | | TGCGAAAGATCGAGGGGGGCAGCGGGGTTTTGACT |
| | | ACTGGGGCCAGGGAACCCTGGTCACCGTGTCCTCA |
| | | GGGTCAACCTCTGGTAGCGGTAAGCCTGGCTCCGG |
| | | CGAAGGCTCCACAAAGGGTCAGTCTGTGCTGACTC |
| | | AGCCACCCTCAGCGTCTGGGACCCCCGGGCAGAGG |
| | | GTCACCATCTCTTGTTCTGGAAGCAGCTCCAACAT |
| | | CGGAAGTAATTATGTATACTGGTACCAGCAGCTCC |
| | | CAGGAACGGCCCCCAAACTCCTCATCTATAGGAAT |
| | | AATCAGCGGCCCTCAGGGGTCCCTGACCGATTCTC |
| | | TGGCTCCAAGTCTGGCACCTCAGCCTCCCTGGCCA |
| | | TCAGTGGGCTCCGGTCCGAGGATGAGGCTGATTAT |
| | | TACTGTGCAGCATGGGATGACAGCCTGAGTGGTCT |
| | | GGTGTTCGGCGGAGGGACCAAGCTGACCGTCCTAG |
| | | AGCAGAAACTGATCAGCGAGGAAGATCTGAATCCT |
| | | GGCGGAGGCGGAGGAAGTGGTGGCGGAGGTTCTGG |
| | | TGGCGGTGGATCAGGCGGTGGCGGATCTGGATCTA |
| | | TGGATATCCAGATGACCCAGTCCCCGAGCTCCCTG |
| | | TCCGCCTCTGTGGGCGATAGGGTCACCATCACCTG |
| | | CCGTGCCAGTCAGGACATCCGTAATTATCTGAACT |
| | | GGTATCAACAGAAACCAGGAAAAGCTCCGAAACTA |
| | | CTGATTTACTATACCTCCCGCCTGGAGTCTGGAGT |
| | | CCCTTCTCGCTTCTCTGGTTCTGGTTCTGGGACGG |
| | | ATTACACTCTGACCATCAGCAGTCTGCAACCGGAA |
| | | GACTTCGCAACTTATTACTGTCAGCAAGGTAATAC |
| | | TCTGCCGTGGACGTTCGGACAGGGCACCAAGGTGG |
| | | AGATCAAAGGCGGCGGCGGAAGTGGAGGAGGAGGC |
| | | TCAGGCGGAGGAGGGAGCGAGGTTCAGCTGGTGGA |
| | | GTCTGGCGGTGGCCTGGTGCAGCCAGGGGGCTCAC |
| | | TCCGTTTGTCCTGTGCAGCTTCTGGCTACTCCTTT |
| | | ACCGGCTACACTATGAACTGGGTGCGTCAGGCCCC |
| | | AGGTAAGGGCCTGGAATGGGTTGCACTGATTAATC |
| | | CTACCAAAGGTGTTAGTACCTACAACCAGAAGTTC |
| | | AAGGACCGTTTCACTATAAGCGTAGATAAATCCAA |
| | | AAACACAGCCTACCTGCAAATGAACAGCCTGCGTG |
| | | CTGAGGACACTGCCGTCTATTATTGTGCTAGAAGC |
| | | GGATACTACGGCGATAGTGACTGGTATTTTGACGT |
| | | GTGGGGTCAAGGAACCCTGGTCACCGTCTCCTCGA |
| | | CATCTGGCGGCGGAGGATCTCTGGAATCTGGACAG |
| | | GTGCTGCTGGAAAGCAACATCAAGGTGCTGCCCAC |
| | | CTGGTCTACCCCAGTTCAGCCTATGGCTCTGATTG |
| | | TGCTTGGCGGAGTTGCCGGCCTGCTGCTCTTTATC |
| | | GGCCTGGGCATCTTCTTTTGCGTGCGGTGCAGACA |
| | | TCGGCGGAGACAGGCTGAGAGAATGAGCCAGATCA |
| | | AGCGGCTGCTGAGCGAGAAGAAAACCTGTCAGTGC |
| | | CCTCACCGGTTCCAGAAAACATGCAGCCCCATC |
| 617 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGGCCTGGTGCAGCCC |
| | | GGCGGCAGCCTGAAGCTGAGCTGCACCACCAGCGG |
| | | CTTCACCTTCAGCAGCTACGGCCTGAGCTGGATCA |
| | | GGCAGACCCCCGACAAGAGGCTGGAGCTGGTGGCC |
| | | AGCATCAACAGGAACGGCGACAACACCTACTACAC |
| | | CGACAGCGTGAAGGGCAGGTTCACCATCAGCAGGC |
| | | ACGACGCCAAGAACACCCTGAACCTGCAGATGAAC |
| | | AGCCTGAAGAGCGAGGACACCGCCATGTACTACTG |
| | | CACCAGGGGCGGCTTCAGCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCGACATCGTGCTGACCCAGAGCCCCGCCATCA |
| | | TGAGCAGCAGCCCCGGCGAGAAGGTGACCATGACC |
| | | TGCAGCGCCAGCAGCAGCGTGAGCTACATGTACTG |
| | | GTACCAGCAGAGGCCCGGCAGCAGCCCCAGGCTGC |
| | | TGATCTACGACACCAGCAAGCTGGCCAGCGGCGTG |
| | | CCCGTGAGGTTCAGCGGCAGCGTGAGCGGCACCAG |
| | | CTACAGCCTGACCATCAGCAGGATGGAGAGCGAGG |
| | | ACGCCGCCACCTACTACTGCCAGCAGTGGAGCGGC |
| | | TTCCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 618 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGGCCTGGTGCAGCCC |
| | | GGCGGCAGCCTGAAGCTGAGCTGCACCACCAGCGG |
| | | CTTCACCTTCAGCAGCTACGGCCTGAGCTGGATCA |
| | | GGCAGACCCCCGACAAGAGGCTGGAGCTGGTGGCC |
| | | AGCATCAACAGGAACGGCGACAACACCTACTACAC |
| | | CGACAGCGTGAGGGGCAGGTTCACCATCAGCAGGG |
| | | ACAACGCCAAGAACACCCTGAACCTGCAGATGAGC |
| | | AGCCTGAAGAGCGAGGACACCGCCATGTACTACTG |
| | | CACCAGGGGCGGCTTCAGCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCGACATCGTGCTGACCCAGAGCCCCGCCACCA |
| | | TGAGCGCCAGCCCCGGCGAGAAGGTGACCCTGACC |
| | | TGCAGCGCCAGCCCCGGCGTGACCTACATGTACTG |
| | | GTACCAGCAGAAGCCCGGCAGCAGCCCCAGGCTGC |
| | | TGATCTACGACACCAGCAACCTGGCCAGCGGCGTG |
| | | CCCCTGAGGTTCAGCGGCAGCGGCAGCGGCACCAG |
| | | CTACAGCCTGACCATCAGCAGGACCGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAGGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 619 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGGCCTGGTGCAGCCC |
| | | GGCGGCAGCCTGAAGCTGAGCTGCACCACCAGCGG |
| | | CTTCACCTTCAGCAGCTACGGCCTGAGCTGGATCA |
| | | GGCAGACCCCCGACAAGAGGCTGGAGCTGGTGGCC |
| | | AGCATCAACAGGAACGGCGACAACGCCTACTACAT |
| | | CGACAGCGTGAAGGGCAGGTTCACCATCAGCAGGG |
| | | ACAACGCCAAGAACGCCCTGAACCTGCAGATGAGC |
| | | AGCCTGAAGAGCGAGGACACCGCCATGTACTACTG |
| | | CACCAGGGGCGGCTTCAGCCACTGGGGCCAGGGCA |
| | | CCCTGGTGACCGTGGCCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCAGCCCCGGCGAGAAGGTGACCATGACC |
| | | TGCAGCGCCAGCAGCAGCGTGAGCTACATGTACTG |
| | | GTACCAGCAGAAGCCCGGCAGCAGCCCCAGGCTGC |
| | | TGATCTACGACACCAGCAACCTGGCCAGCGGCGTG |
| | | CCCGTGAGGTTCAGCGGCAGCGGCAGCGGCACCAG |
| | | CTACAGCCTGATCATCAGCAGGATGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 620 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGGCCTGGTGCAGCCC |
| | | GGCGGCAGCCTGAAGCTGAGCTGCGCCGCCAGCGG |
| | | CTTCACCTTCAGCAGCTACGGCCTGAGCTGGGTGA |
| | | GGCAGACCCCCGACAAGGGCCTGGAGCTGGTGGCC |
| | | AGCATCAACAGGAACGGCGGCAACACCTACTACAC |
| | | CGACAGCGTGAAGGGCAGGTTCACCATCAGCAGGG |
| | | ACAACGCCAAGAACACCCTGAACCTGCAGATGAGC |
| | | AGCCTGAAGAGCGAGGACACCGCCATGTACTACTG |
| | | CGCCAGGGGCGGCTTCACCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCTTCCCCGGCGAGAAGGTGACCATGACC |
| | | TGCAGCGCCAGCAGCAGCGTGGGCTACATGTACTG |
| | | GTACCAGCAGAAGCCCGGCAGCAGCCCCAGGCTGC |
| | | TGATCTACGACACCAGCAACCTGGCCAGCGGCGTG |
| | | CCCGTGAGGTTCAGCGGCAGCGGCAGCGGCACCGC |
| | | CTACAGCCTGACCATCAGCAGGATGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 621 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGAAGGAGAGCGGCGGCGGCCTGGTGAGGCCC |
| | | GGCGGCAGCCTGAAGCTGAGCTGCGCCGCCAGCGG |
| | | CTTCACCTTCAGCAACTACGGCATGAGCTGGATCA |
| | | GGCAGACCCCCGACAAGAACCTGGAGCTGGTGGCC |
| | | AGCATCAACACCAGCGGCGGCAACACCTACTACCC |
| | | CGACAGCGTGAAGGGCAGGTTCACCATCAGCAGGG |
| | | ACAACGCCAGGGCCACCCTGAACCTGCAGATGAGC |
| | | AACCTGAAGAGCGAGGACACCGCCATCTACTACTG |
| | | CACCAGGGGCGGCTTCACCCACTGGGGCCAGGGCA |
| | | CCCTGGTGGCCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCAGCCCCGGCGAGAAGGTGACCATGACC |
| | | TGCAGCGCCAGCAGCAGCGTGAGCTACATGTACTG |
| | | GTACCAGCAGAAGCCCGGCAGCAGCCCCAGGCTGC |
| | | TGATCTACGACACCAGCAACCTGGCCAGCGGCGTG |
| | | CCCGTGAGGTTCAGCGGCAGCGGCAGCGGCACCAG |
| | | CTACTTCCTGACCATCAGCAGGATGGAGGCCGAGG |
| | | ACGCCGCCACCTACTACTGCCAGCAGTGGACCGGC |
| | | TACCCCCCCATCACCTTCGGCGCCGGCACCAAGCT |
| | | GGAGCTGAGGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 622 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGAAGGAGAGCGGCGGCGGCCTGGTGCAGCCC |
| | | GGCGGCAGCCTGAAGCTGAGCTGCGCCGCCAGCGG |
| | | CTTCCCCTTCAGCAGCTACGGCCTGAGCTGGGTGA |
| | | GGCAGACCCCCGACAAGAGGCTGGAGCTGGTGGCC |
| | | ACCATCAACAGGAACGGCGACAGCGCCTACTACCC |
| | | CGACAGCGTGAAGGGCAGGTTCACCATCAGCAGGG |
| | | ACGACGCCAAGAACACCCTGTACCTGCAGATGAGC |
| | | AGCCTGAAGAGCGAGGACACCGCCATGTACTACTG |
| | | CACCAGGGGCGGCTTCGCCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCAGCCCCGGCGAGAAGGTGACCATGACC |
| | | TGCAGCGGCAGCAGCAGCGTGGGCTACATGTACTG |
| | | GTACCAGCAGAAGCCCGGCTTCAGCCCCAGGCTGC |
| | | TGATCTACGACACCAGCAACCTGGCCAGCGGCGTG |
| | | CCCGTGAGGTTCAGCGGCAGCGGCAGCGGCACCAG |
| | | CTACAGCCTGACCATCAGCAGGATGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 623 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGGCCTGGTGAGGCCC |
| | | GGCGGCAGCCTGAAGCTGAGCTGCGCCGCCAGCGG |
| | | CTTCACCTTCAGCAACTACGGCATGAGCTGGATCA |
| | | GGCAGACCCCCGACAAGAACCTGGAGCTGGTGGCC |
| | | AGCATCAACACCAGCGGCGGCAACACCTACTACCC |
| | | CGACAGCGTGAAGGGCAGGTTCACCATCAGCAGGG |
| | | ACAACGCCAGGGCCACCCTGAACCTGCAGATGAGC |
| | | AACCTGAAGAGCGAGGACACCGCCATCTACTACTG |
| | | CACCAGGGGCGGCTTCACCCACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCGACATCGTGCTGACCCAGAGCCCCGCCATCA |
| | | TGAGCGCCAGCCCCGGCGAGAAGGTGACCATGACC |
| | | TGCAGCGCCAGCAGCAGCGTGGCCTTCATGTACTG |
| | | GTACCAGCAGAAGCCCGGCAGCAGCCCCAGGCTGC |
| | | TGATCTACGACACCAGCAAGCTGGCCAGCGGCGTG |
| | | CCCGTGAGGTTCAGCGGCAGCGGCAGCGGCACCAG |
| | | CTACAGCCTGACCATCAGCAGGATGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 624 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGAAGGAGAGCGGCGGCGGCCTGGTGCAGCCC |
| | | GGCGGCAGCCTGAAGCTGAGCTGCACCACCAGCGG |
| | | CTTCACCTTCAGCAGCTACGGCCTGAGCTGGATCA |
| | | GGCAGACCCCCGACAAGAGGCTGGAGCTGGTGGCC |
| | | AGCATCAACAGGAACGGCGACAACACCTACTACAC |
| | | CGACAGCGTGAGGGGCAGGTTCACCATCAGCAGGG |
| | | ACAACGCCAAGAGCACCCTGAACCTGCAGATGAGC |
| | | AGCCTGAAGAGCGAGGACACCGCCATGTACTACTG |
| | | CACCAGGGGCGGCTTCAGCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCCCCCTGA |
| | | TGAGCGCCAGCCCCGGCGAGAAGGTGACCATGACC |
| | | TGCAGCGCCAGCAGCAGCGTGGGCTACATGTACTG |
| | | GTTCCAGCAGAGGCCCGGCAGCAGCCCCAGGCTGC |
| | | TGATCTACGACACCTACAACCTGGCCAGCGGCGTG |
| | | CCCGTGAGGTTCAGCGGCAGCGGCAGCGGCACCAG |
| | | CTACAGCCTGACCATCAGCAGGCTGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAGGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 625 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGGCCTGGTGCAGCCC |
| | | GGCGGCAGCCTGAAGCTGAGCTGCGCCGCCAGCGG |
| | | CTTCACCTTCAGCAGCTACGGCCTGAGCTGGGTGA |
| | | GGCAGACCCCCGACAAGAGGCTGGAGCTGGTGGCC |
| | | AGCGTGAACAGGAACGGCGGCAACACCTACTACAC |
| | | CGACAGCGTGAAGGGCAGGTTCACCATCAGCAGGG |
| | | ACAACGCCAAGAACACCCTGAACCTGCAGATGAGC |
| | | AGCCTGAAGAGCGAGGACACCGCCATGTACTACTG |
| | | CGCCAGGGGCGGCTTCACCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCAGCCCCGGCGAGAAGGTGACCATGACC |
| | | TGCAGCGCCAGCAGCAGCGTGGGCTACATGTACTG |
| | | GTACCAGCAGAAGCCCGGCAGCAGCCCCAGGCTGC |
| | | TGATCTACGACACCAGCCACCTGGCCAGCGGCGTG |
| | | CCCGTGAGGTTCAGCGGCAGCGGCAGCGGCACCAG |
| | | CTACAGCCTGACCATCAGCAGGATGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 626 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGGCCTGGTGCAGCCC |
| | | GGCGGCAGCCTGAAGCTGAGCTGCGCCGCCAGCGG |
| | | CTTCCCCTTCAGCAGCTACGGCCTGAGCTGGGTGA |
| | | GGCAGACCCCCGACAAGAGGCTGGAGCTGGTGGCC |
| | | ACCATCAACAGGAACGGCGACAGCGCCTACTACCC |
| | | CGACAGCGTGAAGGGCAGGTTCACCATCAGCAGGG |
| | | ACGACGCCAAGAACACCCTGTACCTGCAGATGAGC |
| | | AGCCTGAAGAGCGAGGACACCGCCATGTACTACTG |
| | | CACCAGGGGCGGCTTCGCCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGTGATCA |
| | | TGAGCGCCAGCCCCGGCGAGAAGGTGACCATGACC |
| | | TGCAGCGCCAGCAGCAGCGTGGGCTACATGTACTG |
| | | GTACCAGCAGAGGCCCGGCAGCAGCCCCAGGCTGC |
| | | TGATCTACGACACCAGCAACCTGGCCAGCGGCGCC |
| | | CCCGTGAGGTTCAGCGGCAGCGGCAGCGGCACCAG |
| | | CTACAGCCTGACCATCAGCAGGATGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 627 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGGCCTGGTGCAGCCC |
| | | GGCGGCAGCCTGAAGCTGAGCTGCACCACCAGCGG |
| | | CTTCACCTTCAGCAGCTACGGCCTGAGCTGGATCA |
| | | GGCAGACCCCCGACAAGAGGCTGGAGCTGGTGGCC |
| | | AGCATCAACAGGAACGGCGACAACACCTACTACAC |
| | | CGACAGCGTGAAGGGCAGGTTCACCATCAGCAGGG |
| | | ACAACGCCAAGAACACCCTGAACCTGCAGATGAGC |
| | | AGCCTGAAGAGCGAGGACACCGCCATGTACTACTG |
| | | CACCAGGGGCGGCTTCAGCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCAGCCCCGGCGAGAAGGTGACCATGACC |
| | | TGCAGCGCCAGCAGCAGCGTGAGCTACATGTACTG |
| | | GTACCAGCAGAAGCCCGGCAGCAGCCCCAGGCTGC |
| | | TGATCTACGACACCAGCAACCTGGCCAGCGGCGTG |
| | | CCCGTGAGGTTCAGCGGCAGCGGCAGCGGCACCAG |
| | | CTACAGCCTGACCATCAGCAGGATGGAGGCCGAGG |
| | | ACGCCGCCACCTACTACTGCCAGCAGTGGACCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 628 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGTGCTGGTGCAGCCC |
| | | GGCGGCAGCCTGAAGCTGAGCTGCGCCGCCAGCGG |
| | | CTTCACCTTCAGCAGCTACGGCATGAGCTGGGTGA |
| | | GGCAGACCCCCGACAAGAGGCTGGAGCTGGTGGCC |
| | | AGCATCAACAAGAACGGCGGCAGCACCTACTACCC |
| | | CGACAGCGTGAAGGGCAGGTTCACCATCAGCAGGG |
| | | ACAACGCCAAGACCACCGTGTACCTGCAGATGAGC |
| | | AGCCTGAAGAGCGAGGACACCGCCATGTACTACTG |
| | | CACCAGGGGCGGCTTCGCCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCAGCCCCGGCGAGAAGGTGACCCTGACC |
| | | TGCAGCGCCAGCAGCAGCGTGGGCTACATGTACTG |
| | | GTACCAGCAGAGGCCCGGCAGCAGCCCCAGGCTGC |
| | | TGATCTACGACACCAGCAACCTGCCCAGCGGCGTG |
| | | CCCGTGAGGTTCAGCGGCAGCGGCAGCGGCACCAG |
| | | CTACAGCCTGACCATCAGCAGGATGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTACCAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 629 | Nucleotide | ATGGCTCTGCCTGTGACAGCTCTGTTGCTGCCTCT |
| | | GGCTCTGCTGCTGCATGCTGCTAGACCTCAGCTGC |
| | | AGCTGCAGGAGAGCGGCGGCGGCCTGGTGCAGGCC |
| | | GGCGGCAGCCTGAGGCTGAGCTGCGCCGCCAGCGG |
| | | CAGGACCGGCAGCAGCTACGCCATGGGCTGGTTCA |
| | | GGCAGGCCCCCGGCAAGGAGAGGGAGTTCGTGGCC |
| | | GCCATCACCTGGAGCGGCGGCATCACCGCCTACGC |
| | | CGACAGCGTGAAGGGCAGGTTCACCATCAGCAGGG |
| | | ACAACGCCAAGAACACCGTGTACCTGCAGATGAAC |
| | | AGCCTGAAGCCCGAGGACACCGCCGTGTACTGCTG |
| | | CGCCGCCGGCGTGACCGGCAGCCCCAGCTTCGACA |
| | | GCTGGGGCCAGGGCACCCAGGTGACCGTGAGCAGC |
| | | GAGCAGAAACTGATCAGCGAGGAAGATCTGAATCC |
| | | TGGCGGAGGCGGAGGAAGTGGTGGCGGAGGTTCTG |
| | | GTGGCGGTGGATCAGGCGGTGGCGGATCTGGATCT |
| | | ATGGATATCCAGATGACCCAGTCCCCGAGCTCCCT |
| | | GTCCGCCTCTGTGGGCGATAGGGTCACCATCACCT |
| | | GCCGTGCCAGTCAGGACATCCGTAATTATCTGAAC |
| | | TGGTATCAACAGAAACCAGGAAAAGCTCCGAAACT |
| | | ACTGATTTACTATACCTCCCGCCTGGAGTCTGGAG |
| | | TCCCTTCTCGCTTCTCTGGTTCTGGTTCTGGGACG |
| | | GATTACACTCTGACCATCAGCAGTCTGCAACCGGA |
| | | AGACTTCGCAACTTATTACTGTCAGCAAGGTAATA |
| | | CTCTGCCGTGGACGTTCGGACAGGGCACCAAGGTG |
| | | GAGATCAAAGGCGGCGGCGGAAGTGGAGGAGGAGG |
| | | CTCAGGCGGAGGAGGGAGCGAGGTTCAGCTGGTGG |
| | | AGTCTGGCGGTGGCCTGGTGCAGCCAGGGGGCTCA |
| | | CTCCGTTTGTCCTGTGCAGCTTCTGGCTACTCCTT |
| | | TACCGGCTACACTATGAACTGGGTGCGTCAGGCCC |
| | | CAGGTAAGGGCCTGGAATGGGTTGCACTGATTAAT |
| | | CCTACCAAAGGTGTTAGTACCTACAACCAGAAGTT |
| | | CAAGGACCGTTTCACTATAAGCGTAGATAAATCCA |
| | | AAAACACAGCCTACCTGCAAATGAACAGCCTGCGT |
| | | GCTGAGGACACTGCCGTCTATTATTGTGCTAGAAG |
| | | CGGATACTACGGCGATAGTGACTGGTATTTTGACG |
| | | TGTGGGGTCAAGGAACCCTGGTCACCGTCTCCTCG |
| | | ACATCTGGCGGCGGAGGATCTCTGGAATCTGGACA |
| | | GGTGCTGCTGGAAAGCAACATCAAGGTGCTGCCCA |
| | | CCTGGTCTACCCCAGTTCAGCCTATGGCTCTGATT |
| | | GTGCTTGGCGGAGTTGCCGGCCTGCTGCTCTTTAT |
| | | CGGCCTGGGCATCTTCTTTTGCGTGCGGTGCAGAC |
| | | ATCGGCGGAGACAGGCTGAGAGAATGAGCCAGATC |
| | | AAGCGGCTGCTGAGCGAGAAGAAAACCTGTCAGTG |
| | | CCCTCACCGGTTCCAGAAAACATGCAGCCCCATC |
| 630 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGCAGGAGAGCGGCGGCGGCCTGGTGCAGGCC |
| | | GGCGGCAGCCTGAGGCTGAGCTGCGCCGCCAGCGG |
| | | CAGGACCTTCAGCAGCTACGCCATGGGCTGGTTCA |
| | | GGCAGGCCCCCGGCAAGGAGAGGGAGTTCGTGGCC |
| | | GCCATCAGCGGCAGCGGCGGCAGCATCTACTACGG |
| | | CGACAGCGTGAAGGGCAGGTTCACCATCAGCAGGG |
| | | ACAACGCCAAGAACACCATGTACCTGCAGATGAAC |
| | | AGGCTGAAGCCCGAGGACACCGCCGTGTACTACTG |
| | | CGCCGCCGGCCCCCTGGGCAGCCCCGACTTCGACA |
| | | GCTGGGGCCAGGGCACCCAGGTGACCGTGAGCAGC |
| | | GAGCAGAAACTGATCAGCGAGGAAGATCTGAATCC |
| | | TGGCGGAGGCGGAGGAAGTGGTGGCGGAGGTTCTG |
| | | GTGGCGGTGGATCAGGCGGTGGCGGATCTGGATCT |
| | | ATGGATATCCAGATGACCCAGTCCCCGAGCTCCCT |
| | | GTCCGCCTCTGTGGGCGATAGGGTCACCATCACCT |
| | | GCCGTGCCAGTCAGGACATCCGTAATTATCTGAAC |
| | | TGGTATCAACAGAAACCAGGAAAAGCTCCGAAACT |
| | | ACTGATTTACTATACCTCCCGCCTGGAGTCTGGAG |
| | | TCCCTTCTCGCTTCTCTGGTTCTGGTTCTGGGACG |
| | | GATTACACTCTGACCATCAGCAGTCTGCAACCGGA |
| | | AGACTTCGCAACTTATTACTGTCAGCAAGGTAATA |
| | | CTCTGCCGTGGACGTTCGGACAGGGCACCAAGGTG |
| | | GAGATCAAAGGCGGCGGCGGAAGTGGAGGAGGAGG |
| | | CTCAGGCGGAGGAGGGAGCGAGGTTCAGCTGGTGG |
| | | AGTCTGGCGGTGGCCTGGTGCAGCCAGGGGGCTCA |
| | | CTCCGTTTGTCCTGTGCAGCTTCTGGCTACTCCTT |
| | | TACCGGCTACACTATGAACTGGGTGCGTCAGGCCC |
| | | CAGGTAAGGGCCTGGAATGGGTTGCACTGATTAAT |
| | | CCTACCAAAGGTGTTAGTACCTACAACCAGAAGTT |
| | | CAAGGACCGTTTCACTATAAGCGTAGATAAATCCA |
| | | AAAACACAGCCTACCTGCAAATGAACAGCCTGCGT |
| | | GCTGAGGACACTGCCGTCTATTATTGTGCTAGAAG |
| | | CGGATACTACGGCGATAGTGACTGGTATTTTGACG |
| | | TGTGGGGTCAAGGAACCCTGGTCACCGTCTCCTCG |
| | | ACATCTGGCGGCGGAGGATCTCTGGAATCTGGACA |
| | | GGTGCTGCTGGAAAGCAACATCAAGGTGCTGCCCA |
| | | CCTGGTCTACCCCAGTTCAGCCTATGGCTCTGATT |
| | | GTGCTTGGCGGAGTTGCCGGCCTGCTGCTCTTTAT |
| | | CGGCCTGGGCATCTTCTTTTGCGTGCGGTGCAGAC |
| | | ATCGGCGGAGACAGGCTGAGAGAATGAGCCAGATC |
| | | AAGCGGCTGCTGAGCGAGAAGAAAACCTGTCAGTG |
| | | CCCTCACCGGTTCCAGAAAACATGCAGCCCCATC |
| 631 | Nucleotide | ATGGCTCTGCCTGTGACAGCTCTGTTGCTGCCTCT |
| | | GGCTCTGCTGCTGCATGCTGCTAGACCTGAGGTGC |
| | | AGGTGGTGGAGAGCGGCGGCGGCCTGGTGCAGCCC |
| | | GGCGGCAGCCTGAGGCTGAGCTGCGTGGCCAGCGG |
| | | CAGGACCTTCAGCAGCTACGCCATGGGCTGGTTCA |
| | | GGCAGGCCCCCGGCAAGGAGAGGGAGTTCGTGGCC |
| | | GCCATCAGCGGCAGCGGCGGCAGCATCTACTACGG |
| | | CGACAGCGTGAAGGGCAGGTTCACCATCAGCAGGG |
| | | ACAACGCCAAGAACACCATGTACCTGCAGATGAAC |
| | | AGGCTGAAGCCCGAGGACACCGCCGTGTACTACTG |
| | | CGCCGCCGGCCCCCTGGGCAGCCCCGACTTCGACA |
| | | GCTGGGGCCAGGGCACCCAGGTGACCGTGAGCAGC |
| | | GAGCAGAAACTGATCAGCGAGGAAGATCTGAATCC |
| | | TGGCGGAGGCGGAGGAAGTGGTGGCGGAGGTTCTG |
| | | GTGGCGGTGGATCAGGCGGTGGCGGATCTGGATCT |
| | | ATGGATATCCAGATGACCCAGTCCCCGAGCTCCCT |
| | | GTCCGCCTCTGTGGGCGATAGGGTCACCATCACCT |
| | | GCCGTGCCAGTCAGGACATCCGTAATTATCTGAAC |
| | | TGGTATCAACAGAAACCAGGAAAAGCTCCGAAACT |
| | | ACTGATTTACTATACCTCCCGCCTGGAGTCTGGAG |
| | | TCCCTTCTCGCTTCTCTGGTTCTGGTTCTGGGACG |
| | | GATTACACTCTGACCATCAGCAGTCTGCAACCGGA |
| | | AGACTTCGCAACTTATTACTGTCAGCAAGGTAATA |
| | | CTCTGCCGTGGACGTTCGGACAGGGCACCAAGGTG |
| | | GAGATCAAAGGCGGCGGCGGAAGTGGAGGAGGAGG |
| | | CTCAGGCGGAGGAGGGAGCGAGGTTCAGCTGGTGG |
| | | AGTCTGGCGGTGGCCTGGTGCAGCCAGGGGGCTCA |
| | | CTCCGTTTGTCCTGTGCAGCTTCTGGCTACTCCTT |
| | | TACCGGCTACACTATGAACTGGGTGCGTCAGGCCC |
| | | CAGGTAAGGGCCTGGAATGGGTTGCACTGATTAAT |
| | | CCTACCAAAGGTGTTAGTACCTACAACCAGAAGTT |
| | | CAAGGACCGTTTCACTATAAGCGTAGATAAATCCA |
| | | AAAACACAGCCTACCTGCAAATGAACAGCCTGCGT |
| | | GCTGAGGACACTGCCGTCTATTATTGTGCTAGAAG |
| | | CGGATACTACGGCGATAGTGACTGGTATTTTGACG |
| | | TGTGGGGTCAAGGAACCCTGGTCACCGTCTCCTCG |
| | | ACATCTGGCGGCGGAGGATCTCTGGAATCTGGACA |
| | | GGTGCTGCTGGAAAGCAACATCAAGGTGCTGCCCA |
| | | CCTGGTCTACCCCAGTTCAGCCTATGGCTCTGATT |
| | | GTGCTTGGCGGAGTTGCCGGCCTGCTGCTCTTTAT |
| | | CGGCCTGGGCATCTTCTTTTGCGTGCGGTGCAGAC |
| | | ATCGGCGGAGACAGGCTGAGAGAATGAGCCAGATC |
| | | AAGCGGCTGCTGAGCGAGAAGAAAACCTGTCAGTG |
| | | CCCTCACCGGTTCCAGAAAACATGCAGCCCCATC |
| 632 | Nucleotide | ATGGCTCTGCCTGTGACAGCTCTGTTGCTGCCTCT |
| | | GGCTCTGCTGCTGCATGCTGCTAGACCTAAGGTGC |
| | | AGCTGGTGGAGAGCGGCGGCGGCCTGGTGCAGGCC |
| | | GGCGGCAGCCTGAGGCTGAGCTGCGCCGCCAGCGG |
| | | CAGGACCGGCAGCAGCTACGCCATGGGCTGGTTCA |
| | | GGCAGGCCCCCGGCAAGGAGAGGGAGTTCGTGGCC |
| | | GCCATCACCTGGAGCGGCGGCATCACCGCCTACGC |
| | | CGACAGCGTGAAGGGCAGGTTCACCATCAGCAGGG |
| | | ACAACGCCAAGAACACCGTGTACCTGCAGATGAAC |
| | | AGCCTGAAGCCCGAGGACACCGCCGTGTACTGCTG |
| | | CGCCGCCGGCGTGACCGGCAGCCCCAGCTTCGACA |
| | | GCTGGGGCCAGGGCACCCAGGTGACCGTGAGCAGC |
| | | GAGCAGAAACTGATCAGCGAGGAAGATCTGAATCC |
| | | TGGCGGAGGCGGAGGAAGTGGTGGCGGAGGTTCTG |
| | | GTGGCGGTGGATCAGGCGGTGGCGGATCTGGATCT |
| | | ATGGATATCCAGATGACCCAGTCCCCGAGCTCCCT |
| | | GTCCGCCTCTGTGGGCGATAGGGTCACCATCACCT |
| | | GCCGTGCCAGTCAGGACATCCGTAATTATCTGAAC |
| | | TGGTATCAACAGAAACCAGGAAAAGCTCCGAAACT |
| | | ACTGATTTACTATACCTCCCGCCTGGAGTCTGGAG |
| | | TCCCTTCTCGCTTCTCTGGTTCTGGTTCTGGGACG |
| | | GATTACACTCTGACCATCAGCAGTCTGCAACCGGA |
| | | AGACTTCGCAACTTATTACTGTCAGCAAGGTAATA |
| | | CTCTGCCGTGGACGTTCGGACAGGGCACCAAGGTG |
| | | GAGATCAAAGGCGGCGGCGGAAGTGGAGGAGGAGG |
| | | CTCAGGCGGAGGAGGGAGCGAGGTTCAGCTGGTGG |
| | | AGTCTGGCGGTGGCCTGGTGCAGCCAGGGGGCTCA |
| | | CTCCGTTTGTCCTGTGCAGCTTCTGGCTACTCCTT |
| | | TACCGGCTACACTATGAACTGGGTGCGTCAGGCCC |
| | | CAGGTAAGGGCCTGGAATGGGTTGCACTGATTAAT |
| | | CCTACCAAAGGTGTTAGTACCTACAACCAGAAGTT |
| | | CAAGGACCGTTTCACTATAAGCGTAGATAAATCCA |
| | | AAAACACAGCCTACCTGCAAATGAACAGCCTGCGT |
| | | GCTGAGGACACTGCCGTCTATTATTGTGCTAGAAG |
| | | CGGATACTACGGCGATAGTGACTGGTATTTTGACG |
| | | TGTGGGGTCAAGGAACCCTGGTCACCGTCTCCTCG |
| | | ACATCTGGCGGCGGAGGATCTCTGGAATCTGGACA |
| | | GGTGCTGCTGGAAAGCAACATCAAGGTGCTGCCCA |
| | | CCTGGTCTACCCCAGTTCAGCCTATGGCTCTGATT |
| | | GTGCTTGGCGGAGTTGCCGGCCTGCTGCTCTTTAT |
| | | CGGCCTGGGCATCTTCTTTTGCGTGCGGTGCAGAC |
| | | ATCGGCGGAGACAGGCTGAGAGAATGAGCCAGATC |
| | | AAGCGGCTGCTGAGCGAGAAGAAAACCTGTCAGTG |
| | | CCCTCACCGGTTCCAGAAAACATGCAGCCCCATC |
| 633 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGAGCGGCGGCGGCCTGGTGCAGGCC |
| | | GGCGGCAGCCTGAGGCTGAGCTGCGCCGCCAGCGG |
| | | CAGGACCTTCAGCAGCTACGCCATGGGCTGGTTCA |
| | | GGCAGGCCCCCGGCAAGGAGAGGGAGTTCGTGGCC |
| | | GCCATCAGCGGCAGCGGCGGCGTGACCTTCTACGC |
| | | CCACAGCGTGAAGGGCAGGTTCACCATCAGCAGGG |
| | | ACAACGCCAAGAACACCGTGTACCTGCAGATGAAC |
| | | AGCCTGAAGCCCGAGGACACCGCCGTGTACAGCTG |
| | | CGCCGGCGGCGCCCACGGCAGCCCCGACTTCGGCA |
| | | GCTGGGGCCAGGGCACCCAGGTGACCGTGAGCAGC |
| | | GAGCAGAAACTGATCAGCGAGGAAGATCTGAATCC |
| | | TGGCGGAGGCGGAGGAAGTGGTGGCGGAGGTTCTG |
| | | GTGGCGGTGGATCAGGCGGTGGCGGATCTGGATCT |
| | | ATGGATATCCAGATGACCCAGTCCCCGAGCTCCCT |
| | | GTCCGCCTCTGTGGGCGATAGGGTCACCATCACCT |
| | | GCCGTGCCAGTCAGGACATCCGTAATTATCTGAAC |
| | | TGGTATCAACAGAAACCAGGAAAAGCTCCGAAACT |
| | | ACTGATTTACTATACCTCCCGCCTGGAGTCTGGAG |
| | | TCCCTTCTCGCTTCTCTGGTTCTGGTTCTGGGACG |
| | | GATTACACTCTGACCATCAGCAGTCTGCAACCGGA |
| | | AGACTTCGCAACTTATTACTGTCAGCAAGGTAATA |
| | | CTCTGCCGTGGACGTTCGGACAGGGCACCAAGGTG |
| | | GAGATCAAAGGCGGCGGCGGAAGTGGAGGAGGAGG |
| | | CTCAGGCGGAGGAGGGAGCGAGGTTCAGCTGGTGG |
| | | AGTCTGGCGGTGGCCTGGTGCAGCCAGGGGGCTCA |
| | | CTCCGTTTGTCCTGTGCAGCTTCTGGCTACTCCTT |
| | | TACCGGCTACACTATGAACTGGGTGCGTCAGGCCC |
| | | CAGGTAAGGGCCTGGAATGGGTTGCACTGATTAAT |
| | | CCTACCAAAGGTGTTAGTACCTACAACCAGAAGTT |
| | | CAAGGACCGTTTCACTATAAGCGTAGATAAATCCA |
| | | AAAACACAGCCTACCTGCAAATGAACAGCCTGCGT |
| | | GCTGAGGACACTGCCGTCTATTATTGTGCTAGAAG |
| | | CGGATACTACGGCGATAGTGACTGGTATTTTGACG |
| | | TGTGGGGTCAAGGAACCCTGGTCACCGTCTCCTCG |
| | | ACATCTGGCGGCGGAGGATCTCTGGAATCTGGACA |
| | | GGTGCTGCTGGAAAGCAACATCAAGGTGCTGCCCA |
| | | CCTGGTCTACCCCAGTTCAGCCTATGGCTCTGATT |
| | | GTGCTTGGCGGAGTTGCCGGCCTGCTGCTCTTTAT |
| | | CGGCCTGGGCATCTTCTTTTGCGTGCGGTGCAGAC |
| | | ATCGGCGGAGACAGGCTGAGAGAATGAGCCAGATC |
| | | AAGCGGCTGCTGAGCGAGAAGAAAACCTGTCAGTG |
| | | CCCTCACCGGTTCCAGAAAACATGCAGCCCCATC |
| 634 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGAGCGGCGGCGGCCTGGTGCAGCCC |
| | | GGCGGCAGCCTGAGGCTGAGCTGCGCCGTGAGCAG |
| | | GAACATCGCCAGCCTGTACAGGGTGGACTGGTACA |
| | | GGCAGGCCCCCGGCAAGCAGAGGGAGCTGGTGGCC |
| | | GGCAGGACCAGCGGCGGCACCACCACCTACCTGGA |
| | | CGCCGTGGAGGGCAGGTTCACCATCAGCAGGGACA |
| | | ACGTGAAGGACACCGTGTACCTGCAGATGAACAGC |
| | | CTGACCCCCGAGGACACCGCCGTGTACTACTGCCA |
| | | CGCCCACGACCACTGGAGGGACAGCTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 635 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGAGCGGCGGCGACCTGGTGCAGCCC |
| | | GGCGGCAGCCTGAGGCTGAGCTGCGCCGCCAGCGG |
| | | CAGCATCGGCAGCATCTACGCCATGGGCTGGTACA |
| | | GGCAGGCCCCCGGCAGGCAGAGGGAGCTGGTGGCC |
| | | ACCACCACCAGCGGCGGCACCACCAACTACGCCGA |
| | | CAGCGTGAAGGGCAGGTTCACCATCGCCGGCGACA |
| | | ACGCCAAGAACACCGTGTTCCTGCAGATGAACAGC |
| | | CTGAGGCCCGAGGACACCGCCGTGTACTACTGCAA |
| | | GATCCAGACCCACTGGTACGTGTACTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 636 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGAGCGGCGGCGGCCTGGTGCAGCCC |
| | | GGCGGCAGCCTGAGGCTGAGCTGCGCCGCCAGCAG |
| | | GAACATCTTCAGCCTGTACAGGGTGGACTGGTACA |
| | | GGCAGGCCCCCGGCAAGCAGAGGGAGCTGGTGGCC |
| | | GGCAGCACCAGCGGCGGCACCACCACCTACGCCGA |
| | | CGCCGTGAAGGGCAGGTTCACCATCAGCACCGACA |
| | | ACGTGAAGGACACCGTGTACCTGCAGATGAACAGC |
| | | CTGACCCCCGAGGACACCGCCGTGTACTACTGCCA |
| | | CGCCCACGACCACTGGAGGGACAGCTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 637 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGAGCGGCGGCGGCTGGGTGCACCCC |
| | | GGCGGCAGCCTGAGGCTGAGCTGCGCCGCCAGCAG |
| | | GAACATCTTCAGCATGTACAGGGTGGACTGGTACA |
| | | GGCAGGCCCCCGGCAAGCAGAGGGAGCTGGTGGCC |
| | | GGCATCACCAGCGGCGGCACCACCAGCTACGCCGA |
| | | CGCCGTGAAGGGCAGGTTCACCATCAGCACCGACA |
| | | ACGTGAAGGACACCGTGTACCTGCAGATGAACAGC |
| | | GTGACCCCCGAGGACACCGCCGTGTACTACTGCCA |
| | | CGCCCACGACCACTGGAGGGACAGCTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 638 | Nucleotide | ATGGCTCTGCCTGTGACAGCTCTGTTGCTGCCTCT |
| | | GGCTCTGCTGCTGCATGCTGCTAGACCTGAGGTGC |
| | | AGGTGCAGGAGAGCGGCGGCGACCTGGTGCAGCCC |
| | | GGCGGCAGCCTGAGGCTGAGCTGCGCCGCCAGCGG |
| | | CAGCATCGGCAGCATCTACAGGAAGGGCTGGTACA |
| | | GGCAGGCCCCCGGCAGCCAGAGGGAGCTGGTGGCC |
| | | ACCATCACCAGCGCCGGCACCACCAACTACGCCGA |
| | | CAGCGTGAAGGGCAGGTTCACCATCAGCAGGGACA |
| | | ACGCCAAGAACACCGTGTACCTGCAGATGAACAGC |
| | | CTGAGGCCCGAGGACACCGCCGTGTACTACTGCAA |
| | | CTTCCAGACCCACTGGTACGTGTACTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 639 | Nucleotide | ATGGCTCTGCCTGTGACAGCTCTGTTGCTGCCTCT |
| | | GGCTCTGCTGCTGCATGCTGCTAGACCTGAGGTGA |
| | | GGCTGGTGGAGAGCGGCGGCGGCCTGGTGCAGCCC |
| | | GGCGGCAGCCTGAGGCTGAGCTGCGCCGTGAGCAA |
| | | GAACATCTTCAGCATCTACAGGGTGGACTGGTACC |
| | | ACCAGGCCCCCGGCAAGCAGAGGGAGCTGGTGGCC |
| | | GGCTGGACCAGCGGCGGCAGCACCAGCTACGCCGA |
| | | CGCCGTGAAGGGCAGGTTCACCATCAGCACCGACA |
| | | ACGTGAAGGACACCGTGTACCTGCAGATGAACAGC |
| | | CTGACCCCCGAGGACACCGCCGTGTACTACTGCCA |
| | | CGCCCACGACCACTGGAGGGACTACTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 640 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGCAGGAGAGCGGCGGCGGCCTGGTGCAGCCC |
| | | GGCGGCAGCCTGAGGCTGAGCTGCGCCGCCAGCAG |
| | | GAGCATCTTCGGCATCGCCTTCATGGACTGGTACA |
| | | GGCAGGCCCCCGGCAAGCAGAGGGAGTTCGTGGCC |
| | | GGCATCAGCCACAGCGGCGCCACCAGCTACGCCGA |
| | | CAGCGTGAAGGGCAGGTTCACCGTGAGCAGGGACA |
| | | ACGCCAAGAACACCATGTACCTGCAGATGAACAAC |
| | | CTGCTGCCCGAGGACAGCGCCGTGTACTACTGCAA |
| | | GGCCGACACCGCCACCAACACCGACTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 641 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGCAGGAGAGCGGCGGCGGCCTGGTGCAGCCC |
| | | GGCGGCAGCCTGAGGCTGAGCTGCGCCGCCAGCGG |
| | | CATCATCTTCAGCATCAACACCATGGCCTGGTACA |
| | | GGCAGGGCCCCGGCAAGCAGAGGGACCTGGTGGCC |
| | | CTGATCAGCAGCGGCGGCAACACCAGCTACGCCGA |
| | | CAGCGTGAACGGCAGGTTCACCATCAGCAGGGACA |
| | | ACGCCAAGAACACCGTGTACCTGCAGATGAACGGC |
| | | CTGAAGCCCGAGGACACCGCCGTGTACTACTGCAA |
| | | CAGCGCCGGCAGGAGCTACAGCGGCAGCTACGGCG |
| | | CCTACTGGGGCCAGGGCACCCAGGTGACCGTGAGC |
| | | AGCGAGCAGAAACTGATCAGCGAGGAAGATCTGAA |
| | | TCCTGGCGGAGGCGGAGGAAGTGGTGGCGGAGGTT |
| | | CTGGTGGCGGTGGATCAGGCGGTGGCGGATCTGGA |
| | | TCTATGGATATCCAGATGACCCAGTCCCCGAGCTC |
| | | CCTGTCCGCCTCTGTGGGCGATAGGGTCACCATCA |
| | | CCTGCCGTGCCAGTCAGGACATCCGTAATTATCTG |
| | | AACTGGTATCAACAGAAACCAGGAAAAGCTCCGAA |
| | | ACTACTGATTTACTATACCTCCCGCCTGGAGTCTG |
| | | GAGTCCCTTCTCGCTTCTCTGGTTCTGGTTCTGGG |
| | | ACGGATTACACTCTGACCATCAGCAGTCTGCAACC |
| | | GGAAGACTTCGCAACTTATTACTGTCAGCAAGGTA |
| | | ATACTCTGCCGTGGACGTTCGGACAGGGCACCAAG |
| | | GTGGAGATCAAAGGCGGCGGCGGAAGTGGAGGAGG |
| | | AGGCTCAGGCGGAGGAGGGAGCGAGGTTCAGCTGG |
| | | TGGAGTCTGGCGGTGGCCTGGTGCAGCCAGGGGGC |
| | | TCACTCCGTTTGTCCTGTGCAGCTTCTGGCTACTC |
| | | CTTTACCGGCTACACTATGAACTGGGTGCGTCAGG |
| | | CCCCAGGTAAGGGCCTGGAATGGGTTGCACTGATT |
| | | AATCCTACCAAAGGTGTTAGTACCTACAACCAGAA |
| | | GTTCAAGGACCGTTTCACTATAAGCGTAGATAAAT |
| | | CCAAAAACACAGCCTACCTGCAAATGAACAGCCTG |
| | | CGTGCTGAGGACACTGCCGTCTATTATTGTGCTAG |
| | | AAGCGGATACTACGGCGATAGTGACTGGTATTTTG |
| | | ACGTGTGGGGTCAAGGAACCCTGGTCACCGTCTCC |
| | | TCGACATCTGGCGGCGGAGGATCTCTGGAATCTGG |
| | | ACAGGTGCTGCTGGAAAGCAACATCAAGGTGCTGC |
| | | CCACCTGGTCTACCCCAGTTCAGCCTATGGCTCTG |
| | | ATTGTGCTTGGCGGAGTTGCCGGCCTGCTGCTCTT |
| | | TATCGGCCTGGGCATCTTCTTTTGCGTGCGGTGCA |
| | | GACATCGGCGGAGACAGGCTGAGAGAATGAGCCAG |
| | | ATCAAGCGGCTGCTGAGCGAGAAGAAAACCTGTCA |
| | | GTGCCCTCACCGGTTCCAGAAAACATGCAGCCCCA |
| | | TC |
| 642 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGAGCGGCGGCGGCCTGGTGCAGCCC |
| | | GGCGGCAGCCTGAGGCTGAGCTGCGCCGCCAGCAG |
| | | GAGCATCTTCGTGATCGCCTTCATGGACTGGTACA |
| | | GGCAGGCCCCCGGCAAGCAGAGGGAGTTCGTGGCC |
| | | GGCATCAGCAGCAGCGGCGCCACCAGCTACGCCAG |
| | | CAGCGTGAAGGGCAGGTTCACCGTGAGCAGGGACA |
| | | CCGCCAAGAACACCATGTACCTGCAGATGAACAAC |
| | | CTGCTGCCCGAGGACAGCGCCGTGTACTACTGCAA |
| | | GGCCGACACCGCCACCACCACCGACTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 643 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGAGCGGCGGCGGCCTGGTGCAGCCC |
| | | GGCGGCAGCCTGAGGCTGAGCTGCGCCGCCAGCGG |
| | | CATCATCTTCAGCATCAACACCATGGCCTGGTACA |
| | | GGCAGGGCCCCGGCAAGGAGAGGGACCTGGTGGCC |
| | | GTGATCAAGGGCGACGGCAGCACCAGCTACGCCGA |
| | | CAGCGTGAACGGCAGGTTCACCATCAGCAGGGACA |
| | | ACGCCAAGAACACCGTGTACCTGAGGATGAACGGC |
| | | CTGAAGCCCGAGGACACCGCCGTGTACTACTGCAA |
| | | CAGCGCCGGCAGGAGCTACAGCGGCGTGTACGGCG |
| | | CCTACTGGGGCCAGGGCACCCAGGTGACCGTGAGC |
| | | AGCGAGCAGAAACTGATCAGCGAGGAAGATCTGAA |
| | | TCCTGGCGGAGGCGGAGGAAGTGGTGGCGGAGGTT |
| | | CTGGTGGCGGTGGATCAGGCGGTGGCGGATCTGGA |
| | | TCTATGGATATCCAGATGACCCAGTCCCCGAGCTC |
| | | CCTGTCCGCCTCTGTGGGCGATAGGGTCACCATCA |
| | | CCTGCCGTGCCAGTCAGGACATCCGTAATTATCTG |
| | | AACTGGTATCAACAGAAACCAGGAAAAGCTCCGAA |
| | | ACTACTGATTTACTATACCTCCCGCCTGGAGTCTG |
| | | GAGTCCCTTCTCGCTTCTCTGGTTCTGGTTCTGGG |
| | | ACGGATTACACTCTGACCATCAGCAGTCTGCAACC |
| | | GGAAGACTTCGCAACTTATTACTGTCAGCAAGGTA |
| | | ATACTCTGCCGTGGACGTTCGGACAGGGCACCAAG |
| | | GTGGAGATCAAAGGCGGCGGCGGAAGTGGAGGAGG |
| | | AGGCTCAGGCGGAGGAGGGAGCGAGGTTCAGCTGG |
| | | TGGAGTCTGGCGGTGGCCTGGTGCAGCCAGGGGGC |
| | | TCACTCCGTTTGTCCTGTGCAGCTTCTGGCTACTC |
| | | CTTTACCGGCTACACTATGAACTGGGTGCGTCAGG |
| | | CCCCAGGTAAGGGCCTGGAATGGGTTGCACTGATT |
| | | AATCCTACCAAAGGTGTTAGTACCTACAACCAGAA |
| | | GTTCAAGGACCGTTTCACTATAAGCGTAGATAAAT |
| | | CCAAAAACACAGCCTACCTGCAAATGAACAGCCTG |
| | | CGTGCTGAGGACACTGCCGTCTATTATTGTGCTAG |
| | | AAGCGGATACTACGGCGATAGTGACTGGTATTTTG |
| | | ACGTGTGGGGTCAAGGAACCCTGGTCACCGTCTCC |
| | | TCGACATCTGGCGGCGGAGGATCTCTGGAATCTGG |
| | | ACAGGTGCTGCTGGAAAGCAACATCAAGGTGCTGC |
| | | CCACCTGGTCTACCCCAGTTCAGCCTATGGCTCTG |
| | | ATTGTGCTTGGCGGAGTTGCCGGCCTGCTGCTCTT |
| | | TATCGGCCTGGGCATCTTCTTTTGCGTGCGGTGCA |
| | | GACATCGGCGGAGACAGGCTGAGAGAATGAGCCAG |
| | | ATCAAGCGGCTGCTGAGCGAGAAGAAAACCTGTCA |
| | | GTGCCCTCACCGGTTCCAGAAAACATGCAGCCCCA |
| | | TC |
| 644 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGCAGGAGAGCGGCGGCGGCCTGGTGCAGCCC |
| | | GGCGGCAGCCTGAAGCTGAGCTGCGCCGCCAGCGG |
| | | CAGCATCTTCAGCATCGGCGCCATGAGGTGGTACA |
| | | GGCAGGTGCCCGGCAACGAGAGGGAGCTGGTGGCC |
| | | GGCATCACCAACGGCGGCAACACCAACTACGCCGA |
| | | CAGCGTGAAGGCCAGGTTCACCATCAGCAGGGACA |
| | | ACGCCAAGAACACCGTGTACCTGCAGATGAACAGC |
| | | CTGAAGCCCGAGGACACCACCGTGTACTTCTGCAA |
| | | CGCCGACGTGCAGAACAGCGGCTACGTGTGGGGCA |
| | | ACTACTGGGGCCAGGGCACCCAGGTGACCGTGAGC |
| | | AGCGAGCAGAAACTGATCAGCGAGGAAGATCTGAA |
| | | TCCTGGCGGAGGCGGAGGAAGTGGTGGCGGAGGTT |
| | | CTGGTGGCGGTGGATCAGGCGGTGGCGGATCTGGA |
| | | TCTATGGATATCCAGATGACCCAGTCCCCGAGCTC |
| | | CCTGTCCGCCTCTGTGGGCGATAGGGTCACCATCA |
| | | CCTGCCGTGCCAGTCAGGACATCCGTAATTATCTG |
| | | AACTGGTATCAACAGAAACCAGGAAAAGCTCCGAA |
| | | ACTACTGATTTACTATACCTCCCGCCTGGAGTCTG |
| | | GAGTCCCTTCTCGCTTCTCTGGTTCTGGTTCTGGG |
| | | ACGGATTACACTCTGACCATCAGCAGTCTGCAACC |
| | | GGAAGACTTCGCAACTTATTACTGTCAGCAAGGTA |
| | | ATACTCTGCCGTGGACGTTCGGACAGGGCACCAAG |
| | | GTGGAGATCAAAGGCGGCGGCGGAAGTGGAGGAGG |
| | | AGGCTCAGGCGGAGGAGGGAGCGAGGTTCAGCTGG |
| | | TGGAGTCTGGCGGTGGCCTGGTGCAGCCAGGGGGC |
| | | TCACTCCGTTTGTCCTGTGCAGCTTCTGGCTACTC |
| | | CTTTACCGGCTACACTATGAACTGGGTGCGTCAGG |
| | | CCCCAGGTAAGGGCCTGGAATGGGTTGCACTGATT |
| | | AATCCTACCAAAGGTGTTAGTACCTACAACCAGAA |
| | | GTTCAAGGACCGTTTCACTATAAGCGTAGATAAAT |
| | | CCAAAAACACAGCCTACCTGCAAATGAACAGCCTG |
| | | CGTGCTGAGGACACTGCCGTCTATTATTGTGCTAG |
| | | AAGCGGATACTACGGCGATAGTGACTGGTATTTTG |
| | | ACGTGTGGGGTCAAGGAACCCTGGTCACCGTCTCC |
| | | TCGACATCTGGCGGCGGAGGATCTCTGGAATCTGG |
| | | ACAGGTGCTGCTGGAAAGCAACATCAAGGTGCTGC |
| | | CCACCTGGTCTACCCCAGTTCAGCCTATGGCTCTG |
| | | ATTGTGCTTGGCGGAGTTGCCGGCCTGCTGCTCTT |
| | | TATCGGCCTGGGCATCTTCTTTTGCGTGCGGTGCA |
| | | GACATCGGCGGAGACAGGCTGAGAGAATGAGCCAG |
| | | ATCAAGCGGCTGCTGAGCGAGAAGAAAACCTGTCA |
| | | GTGCCCTCACCGGTTCCAGAAAACATGCAGCCCCA |
| | | TC |
| 645 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGAGCGGCGGCGGCCTGGTGCAGCCC |
| | | GGCGGCAGCCTGAGGCTGAGCTGCGCCGCCAGCAG |
| | | GAGCATCTTCGTGATCGCCTTCATGGACTGGTACA |
| | | GGCAGGCCCCCGGCAAGCAGAGGGAGTTCGTGGCC |
| | | GGCATCAGCAGCAGCGGCGCCACCAGCTACGCCGA |
| | | CAGCGTGAAGGGCAGGTTCACCGTGAGCAGGGACA |
| | | CCGCCAAGAACACCATGTACCTGCAGATGAACAAC |
| | | CTGCTGCCCGAGGACAGCGCCGTGTACTACTGCAA |
| | | GGCCGACACCGCCACCAACACCGACTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 646 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGAGCGGCGGCGGCCTGGTGCAGCCC |
| | | GGCGGCAGCCTGAGGCTGAGCTGCGCCACCAGCGG |
| | | CATCATCTTCCACATCTACACCATGGGCTGGTACA |
| | | GGGGCGCCCCCGGCAAGCAGAGGGAGCTGGTGGCC |
| | | AGGATCACCAGCGGCGGCGACACCAACTACGCCGA |
| | | CAGCGTGAAGGGCAGGTTCACCATCAGCAGGGACA |
| | | ACGTGAAGAACACCATGTACCTGCAGATGAACAGC |
| | | CTGAAGCCCGAGGACACCGCCGTGTACTACTGCAA |
| | | CAGGTTCCCCGGCGCCACCTTCAGCTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTACCAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 647 | Nucleotide | ATGGCTCTGCCTGTGACAGCTCTGTTGCTGCCTCT |
| | | GGCTCTGCTGCTGCATGCTGCTAGACCTCAGCTGC |
| | | AGCTGGTGGAGAGCGGCGGCGGCCTGGTGCAGGCC |
| | | GGCGGCAGCCTGAGGCTGAGCTGCGCCGTGAGCGG |
| | | CAACATCTGGGTGTTCACCGTGATGGCCTGGTACA |
| | | GGCAGGTGCCCGGCAAGCAGAGGGAGCTGGTGGCC |
| | | GCCAGCACCAACGGCGGCAGCACCAACTACGCCGA |
| | | CAGCGTGAAGGGCAGGTTCACCATCAGCAGGGACA |
| | | ACGTGAAGAACACCGTGTACCTGCAGATGAACAGC |
| | | CTGAAGCCCGAGGACACCGCCGTGTACTACTGCAA |
| | | CAGGCAGAGGACCGTGATCGGCATGAACCCCCTGG |
| | | CCCCCTGGGGCCAGGGCACCCAGGTGACCGTGAGC |
| | | AGCGAGCAGAAACTGATCAGCGAGGAAGATCTGAA |
| | | TCCTGGCGGAGGCGGAGGAAGTGGTGGCGGAGGTT |
| | | CTGGTGGCGGTGGATCAGGCGGTGGCGGATCTGGA |
| | | TCTATGGATATCCAGATGACCCAGTCCCCGAGCTC |
| | | CCTGTCCGCCTCTGTGGGCGATAGGGTCACCATCA |
| | | CCTGCCGTGCCAGTCAGGACATCCGTAATTATCTG |
| | | AACTGGTATCAACAGAAACCAGGAAAAGCTCCGAA |
| | | ACTACTGATTTACTATACCTCCCGCCTGGAGTCTG |
| | | GAGTCCCTTCTCGCTTCTCTGGTTCTGGTTCTGGG |
| | | ACGGATTACACTCTGACCATCAGCAGTCTGCAACC |
| | | GGAAGACTTCGCAACTTATTACTGTCAGCAAGGTA |
| | | ATACTCTGCCGTGGACGTTCGGACAGGGCACCAAG |
| | | GTGGAGATCAAAGGCGGCGGCGGAAGTGGAGGAGG |
| | | AGGCTCAGGCGGAGGAGGGAGCGAGGTTCAGCTGG |
| | | TGGAGTCTGGCGGTGGCCTGGTGCAGCCAGGGGGC |
| | | TCACTCCGTTTGTCCTGTGCAGCTTCTGGCTACTC |
| | | CTTTACCGGCTACACTATGAACTGGGTGCGTCAGG |
| | | CCCCAGGTAAGGGCCTGGAATGGGTTGCACTGATT |
| | | AATCCTACCAAAGGTGTTAGTACCTACAACCAGAA |
| | | GTTCAAGGACCGTTTCACTATAAGCGTAGATAAAT |
| | | CCAAAAACACAGCCTACCTGCAAATGAACAGCCTG |
| | | CGTGCTGAGGACACTGCCGTCTATTATTGTGCTAG |
| | | AAGCGGATACTACGGCGATAGTGACTGGTATTTTG |
| | | ACGTGTGGGGTCAAGGAACCCTGGTCACCGTCTCC |
| | | TCGACATCTGGCGGCGGAGGATCTCTGGAATCTGG |
| | | ACAGGTGCTGCTGGAAAGCAACATCAAGGTGCTGC |
| | | CCACCTGGTCTACCCCAGTTCAGCCTATGGCTCTG |
| | | ATTGTGCTTGGCGGAGTTGCCGGCCTGCTGCTCTT |
| | | TATCGGCCTGGGCATCTTCTTTTGCGTGCGGTGCA |
| | | GACATCGGCGGAGACAGGCTGAGAGAATGAGCCAG |
| | | ATCAAGCGGCTGCTGAGCGAGAAGAAAACCTGTCA |
| | | GTGCCCTCACCGGTTCCAGAAAACATGCAGCCCCA |
| | | TC |
| 648 | Nucleotide | ATGGCTCTGCCTGTGACAGCTCTGTTGCTGCCTCT |
| | | GGCTCTGCTGCTGCATGCTGCTAGACCTGAGGTGA |
| | | AGCTGGTGGAGAGCGGCGGCGGCCTGGTGCAGCCC |
| | | GGCGGCAGCCTGAAGCTGAGCTGCGCCGCCAGCGG |
| | | CTTCACCTTCAGCAGCTACGGCCTGAGCTGGGTGA |
| | | GGCAGACCCCCGACAAGGGCCTGGAGCTGGTGGCC |
| | | AGCATCAACAGGAACGGCGGCAACACCTACTACAC |
| | | CGACAGCGTGAAGGGCAGGTTCACCATCAGCAGGG |
| | | ACAACGCCAAGAACACCCTGAACCTGCAGATGAGC |
| | | AGCCTGAAGAGCGAGGACACCGCCATGTACTACTG |
| | | CGCCAGGGGCGGCTTCACCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCTTCCCCGGCGAGAAGGTGACCATGACC |
| | | TGCAGCGCCAGCAGCAGCGTGGGCTACATGTACTG |
| | | GTACCAGCAGAAGCCCGGCAGCAGCCCCAGGCTGC |
| | | TGATCTACGACACCAGCAACCTGGCCAGCGGCGTG |
| | | CCCGTGAGGTTCAGCGGCAGCGGCAGCGGCACCGC |
| | | CTACAGCCTGACCATCAGCAGGATGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTTATAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 649 | Nucleotide | ATGGCTCTGCCTGTGACAGCTCTGTTGCTGCCTCT |
| | | GGCTCTGCTGCTGCATGCTGCTAGACCTCAGATCG |
| | | TGCTGACCCAGAGCCCCGCCATCATGAGCGCCTTC |
| | | CCCGGCGAGAAGGTGACCATGACCTGCAGCGCCAG |
| | | CAGCAGCGTGGGCTACATGTACTGGTACCAGCAGA |
| | | AGCCCGGCAGCAGCCCCAGGCTGCTGATCTACGAC |
| | | ACCAGCAACCTGGCCAGCGGCGTGCCCGTGAGGTT |
| | | CAGCGGCAGCGGCAGCGGCACCGCCTACAGCCTGA |
| | | CCATCAGCAGGATGGAGGCCGAGGACGCCGCCACC |
| | | TACTACTGCCAGCAGTGGAGCGGCTACCCCCCCAT |
| | | CACCTTCGGCGCCGGCACCAAGCTGGAGCTGAAGG |
| | | GCAGCACCAGCGGCAGCGGCAAGCCCGGCAGCGGC |
| | | GAGGGCAGCACCAAGGGCGAGGTGAAGCTGGTGGA |
| | | GAGCGGCGGCGGCCTGGTGCAGCCCGGCGGCAGCC |
| | | TGAAGCTGAGCTGCGCCGCCAGCGGCTTCACCTTC |
| | | AGCAGCTACGGCCTGAGCTGGGTGAGGCAGACCCC |
| | | CGACAAGGGCCTGGAGCTGGTGGCCAGCATCAACA |
| | | GGAACGGCGGCAACACCTACTACACCGACAGCGTG |
| | | AAGGGCAGGTTCACCATCAGCAGGGACAACGCCAA |
| | | GAACACCCTGAACCTGCAGATGAGCAGCCTGAAGA |
| | | GCGAGGACACCGCCATGTACTACTGCGCCAGGGGC |
| | | GGCTTCACCTACTGGGGCCAGGGCACCCTGGTGAC |
| | | CGTGAGCGCCGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTTATAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 650 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGAAGGAGAGCGGCGGCGGCCTGGTGCAGCCC |
| | | GGCGGCAGCCTGAAGCTGAGCTGCACCACCAGCGG |
| | | CTTCACCTTCAGCAGCTACGGCCTGAGCTGGATCA |
| | | GGCAGACCCCCGACAAGAGGCTGGAGCTGGTGGCC |
| | | AGCATCAACAGGAACGGCGACAACACCTACTACAC |
| | | CGACAGCGTGAGGGGCAGGTTCACCATCAGCAGGG |
| | | ACAACGCCAAGAGCACCCTGAACCTGCAGATGAGC |
| | | AGCCTGAAGAGCGAGGACACCGCCATGTACTACTG |
| | | CACCAGGGGCGGCTTCAGCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCCCCCTGA |
| | | TGAGCGCCAGCCCCGGCGAGAAGGTGACCATGACC |
| | | TGCAGCGCCAGCAGCAGCGTGGGCTACATGTACTG |
| | | GTTCCAGCAGAGGCCCGGCAGCAGCCCCAGGCTGC |
| | | TGATCTACGACACCTACAACCTGGCCAGCGGCGTG |
| | | CCCGTGAGGTTCAGCGGCAGCGGCAGCGGCACCAG |
| | | CTACAGCCTGACCATCAGCAGGCTGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAGGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTTATAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 651 | Nucleotide | ATGGCTCTGCCTGTGACAGCTCTGTTGCTGCCTCT |
| | | GGCTCTGCTGCTGCATGCTGCTAGACCTCAGATCG |
| | | TGCTGACCCAGAGCCCCCCCCTGATGAGCGCCAGC |
| | | CCCGGCGAGAAGGTGACCATGACCTGCAGCGCCAG |
| | | CAGCAGCGTGGGCTACATGTACTGGTTCCAGCAGA |
| | | GGCCCGGCAGCAGCCCCAGGCTGCTGATCTACGAC |
| | | ACCTACAACCTGGCCAGCGGCGTGCCCGTGAGGTT |
| | | CAGCGGCAGCGGCAGCGGCACCAGCTACAGCCTGA |
| | | CCATCAGCAGGCTGGAGGCCGAGGACGCCGCCACC |
| | | TACTACTGCCAGCAGTGGAGCGGCTACCCCCCCAT |
| | | CACCTTCGGCGCCGGCACCAAGCTGGAGCTGAGGG |
| | | GCAGCACCAGCGGCAGCGGCAAGCCCGGCAGCGGC |
| | | GAGGGCAGCACCAAGGGCCAGGTGCAGCTGAAGGA |
| | | GAGCGGCGGCGGCCTGGTGCAGCCCGGCGGCAGCC |
| | | TGAAGCTGAGCTGCACCACCAGCGGCTTCACCTTC |
| | | AGCAGCTACGGCCTGAGCTGGATCAGGCAGACCCC |
| | | CGACAAGAGGCTGGAGCTGGTGGCCAGCATCAACA |
| | | GGAACGGCGACAACACCTACTACACCGACAGCGTG |
| | | AGGGGCAGGTTCACCATCAGCAGGGACAACGCCAA |
| | | GAGCACCCTGAACCTGCAGATGAGCAGCCTGAAGA |
| | | GCGAGGACACCGCCATGTACTACTGCACCAGGGGC |
| | | GGCTTCAGCTACTGGGGCCAGGGCACCCTGGTGAC |
| | | CGTGAGCGCCGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTTATAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 652 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGAAGGAGAGCGGCGGCGGCCTGGTGAGGCCC |
| | | GGCGGCAGCCTGAAGCTGAGCTGCGCCGCCAGCGG |
| | | CTTCACCTTCAGCAACTACGGCATGAGCTGGATCA |
| | | GGCAGACCCCCGACAAGAACCTGGAGCTGGTGGCC |
| | | AGCATCAACACCAGCGGCGGCAACACCTACTACCC |
| | | CGACAGCGTGAAGGGCAGGTTCACCATCAGCAGGG |
| | | ACAACGCCAGGGCCACCCTGAACCTGCAGATGAGC |
| | | AACCTGAAGAGCGAGGACACCGCCATCTACTACTG |
| | | CACCAGGGGCGGCTTCACCCACTGGGGCCAGGGCA |
| | | CCCTGGTGGCCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCAGCCCCGGCGAGAAGGTGACCATGACC |
| | | TGCAGCGCCAGCAGCAGCGTGAGCTACATGTACTG |
| | | GTACCAGCAGAAGCCCGGCAGCAGCCCCAGGCTGC |
| | | TGATCTACGACACCAGCAACCTGGCCAGCGGCGTG |
| | | CCCGTGAGGTTCAGCGGCAGCGGCAGCGGCACCAG |
| | | CTACTTCCTGACCATCAGCAGGATGGAGGCCGAGG |
| | | ACGCCGCCACCTACTACTGCCAGCAGTGGACCGGC |
| | | TACCCCCCCATCACCTTCGGCGCCGGCACCAAGCT |
| | | GGAGCTGAGGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTTATAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 653 | Nucleotide | ATGGCTCTGCCTGTGACAGCTCTGTTGCTGCCTCT |
| | | GGCTCTGCTGCTGCATGCTGCTAGACCTCAGATCG |
| | | TGCTGACCCAGAGCCCCGCCATCATGAGCGCCAGC |
| | | CCCGGCGAGAAGGTGACCATGACCTGCAGCGCCAG |
| | | CAGCAGCGTGAGCTACATGTACTGGTACCAGCAGA |
| | | AGCCCGGCAGCAGCCCCAGGCTGCTGATCTACGAC |
| | | ACCAGCAACCTGGCCAGCGGCGTGCCCGTGAGGTT |
| | | CAGCGGCAGCGGCAGCGGCACCAGCTACTTCCTGA |
| | | CCATCAGCAGGATGGAGGCCGAGGACGCCGCCACC |
| | | TACTACTGCCAGCAGTGGACCGGCTACCCCCCCAT |
| | | CACCTTCGGCGCCGGCACCAAGCTGGAGCTGAGGG |
| | | GCAGCACCAGCGGCAGCGGCAAGCCCGGCAGCGGC |
| | | GAGGGCAGCACCAAGGGCCAGGTGCAGCTGAAGGA |
| | | GAGCGGCGGCGGCCTGGTGAGGCCCGGCGGCAGCC |
| | | TGAAGCTGAGCTGCGCCGCCAGCGGCTTCACCTTC |
| | | AGCAACTACGGCATGAGCTGGATCAGGCAGACCCC |
| | | CGACAAGAACCTGGAGCTGGTGGCCAGCATCAACA |
| | | CCAGCGGCGGCAACACCTACTACCCCGACAGCGTG |
| | | AAGGGCAGGTTCACCATCAGCAGGGACAACGCCAG |
| | | GGCCACCCTGAACCTGCAGATGAGCAACCTGAAGA |
| | | GCGAGGACACCGCCATCTACTACTGCACCAGGGGC |
| | | GGCTTCACCCACTGGGGCCAGGGCACCCTGGTGGC |
| | | CGTGAGCGCCGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTTATAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 654 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGAAGGAGAGCGGCGGCGGCCTGGTGCAGCCC |
| | | GGCGGCAGCCTGAAGCTGAGCTGCGCCGCCAGCGG |
| | | CTTCCCCTTCAGCAGCTACGGCCTGAGCTGGGTGA |
| | | GGCAGACCCCCGACAAGAGGCTGGAGCTGGTGGCC |
| | | ACCATCAACAGGAACGGCGACAGCGCCTACTACCC |
| | | CGACAGCGTGAAGGGCAGGTTCACCATCAGCAGGG |
| | | ACGACGCCAAGAACACCCTGTACCTGCAGATGAGC |
| | | AGCCTGAAGAGCGAGGACACCGCCATGTACTACTG |
| | | CACCAGGGGCGGCTTCGCCTACTGGGGCCAGGGCA |
| | | CCCTGGTGACCGTGAGCGCCGGCAGCACCAGCGGC |
| | | AGCGGCAAGCCCGGCAGCGGCGAGGGCAGCACCAA |
| | | GGGCCAGATCGTGCTGACCCAGAGCCCCGCCATCA |
| | | TGAGCGCCAGCCCCGGCGAGAAGGTGACCATGACC |
| | | TGCAGCGGCAGCAGCAGCGTGGGCTACATGTACTG |
| | | GTACCAGCAGAAGCCCGGCTTCAGCCCCAGGCTGC |
| | | TGATCTACGACACCAGCAACCTGGCCAGCGGCGTG |
| | | CCCGTGAGGTTCAGCGGCAGCGGCAGCGGCACCAG |
| | | CTACAGCCTGACCATCAGCAGGATGGAGGCCGAGG |
| | | ACGCCGCCACCTACTACTGCCAGCAGTGGAGCGGC |
| | | TACCCCCCCATCACCTTCGGCGCCGGCACCAAGCT |
| | | GGAGCTGAAGGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTTATAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 655 | Nucleotide | ATGGCTCTGCCTGTGACAGCTCTGTTGCTGCCTCT |
| | | GGCTCTGCTGCTGCATGCTGCTAGACCTCAGATCG |
| | | TGCTGACCCAGAGCCCCGCCATCATGAGCGCCAGC |
| | | CCCGGCGAGAAGGTGACCATGACCTGCAGCGGCAG |
| | | CAGCAGCGTGGGCTACATGTACTGGTACCAGCAGA |
| | | AGCCCGGCTTCAGCCCCAGGCTGCTGATCTACGAC |
| | | ACCAGCAACCTGGCCAGCGGCGTGCCCGTGAGGTT |
| | | CAGCGGCAGCGGCAGCGGCACCAGCTACAGCCTGA |
| | | CCATCAGCAGGATGGAGGCCGAGGACGCCGCCACC |
| | | TACTACTGCCAGCAGTGGAGCGGCTACCCCCCCAT |
| | | CACCTTCGGCGCCGGCACCAAGCTGGAGCTGAAGG |
| | | GCAGCACCAGCGGCAGCGGCAAGCCCGGCAGCGGC |
| | | GAGGGCAGCACCAAGGGCCAGGTGCAGCTGAAGGA |
| | | GAGCGGCGGCGGCCTGGTGCAGCCCGGCGGCAGCC |
| | | TGAAGCTGAGCTGCGCCGCCAGCGGCTTCCCCTTC |
| | | AGCAGCTACGGCCTGAGCTGGGTGAGGCAGACCCC |
| | | CGACAAGAGGCTGGAGCTGGTGGCCACCATCAACA |
| | | GGAACGGCGACAGCGCCTACTACCCCGACAGCGTG |
| | | AAGGGCAGGTTCACCATCAGCAGGGACGACGCCAA |
| | | GAACACCCTGTACCTGCAGATGAGCAGCCTGAAGA |
| | | GCGAGGACACCGCCATGTACTACTGCACCAGGGGC |
| | | GGCTTCGCCTACTGGGGCCAGGGCACCCTGGTGAC |
| | | CGTGAGCGCCGAGCAGAAACTGATCAGCGAGGAAG |
| | | ATCTGAATCCTGGCGGAGGCGGAGGAAGTGGTGGC |
| | | GGAGGTTCTGGTGGCGGTGGATCAGGCGGTGGCGG |
| | | ATCTGGATCTATGGATATCCAGATGACCCAGTCCC |
| | | CGAGCTCCCTGTCCGCCTCTGTGGGCGATAGGGTC |
| | | ACCATCACCTGCCGTGCCAGTCAGGACATCCGTAA |
| | | TTATCTGAACTGGTATCAACAGAAACCAGGAAAAG |
| | | CTCCGAAACTACTGATTTACTATACCTCCCGCCTG |
| | | GAGTCTGGAGTCCCTTCTCGCTTCTCTGGTTCTGG |
| | | TTCTGGGACGGATTACACTCTGACCATCAGCAGTC |
| | | TGCAACCGGAAGACTTCGCAACTTATTACTGTCAG |
| | | CAAGGTAATACTCTGCCGTGGACGTTCGGACAGGG |
| | | CACCAAGGTGGAGATCAAAGGCGGCGGCGGAAGTG |
| | | GAGGAGGAGGCTCAGGCGGAGGAGGGAGCGAGGTT |
| | | CAGCTGGTGGAGTCTGGCGGTGGCCTGGTGCAGCC |
| | | AGGGGGCTCACTCCGTTTGTCCTGTGCAGCTTCTG |
| | | GCTACTCCTTTACCGGCTACACTATGAACTGGGTG |
| | | CGTCAGGCCCCAGGTAAGGGCCTGGAATGGGTTGC |
| | | ACTGATTAATCCTTATAAAGGTGTTAGTACCTACA |
| | | ACCAGAAGTTCAAGGACCGTTTCACTATAAGCGTA |
| | | GATAAATCCAAAAACACAGCCTACCTGCAAATGAA |
| | | CAGCCTGCGTGCTGAGGACACTGCCGTCTATTATT |
| | | GTGCTAGAAGCGGATACTACGGCGATAGTGACTGG |
| | | TATTTTGACGTGTGGGGTCAAGGAACCCTGGTCAC |
| | | CGTCTCCTCGACATCTGGCGGCGGAGGATCTCTGG |
| | | AATCTGGACAGGTGCTGCTGGAAAGCAACATCAAG |
| | | GTGCTGCCCACCTGGTCTACCCCAGTTCAGCCTAT |
| | | GGCTCTGATTGTGCTTGGCGGAGTTGCCGGCCTGC |
| | | TGCTCTTTATCGGCCTGGGCATCTTCTTTTGCGTG |
| | | CGGTGCAGACATCGGCGGAGACAGGCTGAGAGAAT |
| | | GAGCCAGATCAAGCGGCTGCTGAGCGAGAAGAAAA |
| | | CCTGTCAGTGCCCTCACCGGTTCCAGAAAACATGC |
| | | AGCCCCATC |
| 656 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGCAGGAGAGCGGCGGCGGCCTGGTGCAGCCC |
| | | GGCGGCAGCCTGAGGCTGAGCTGCGCCGCCAGCAG |
| | | GAGCATCTTCGGCATCGCCTTCATGGACTGGTACA |
| | | GGCAGGCCCCCGGCAAGCAGAGGGAGTTCGTGGCC |
| | | GGCATCAGCCACAGCGGCGCCACCAGCTACGCCGA |
| | | CAGCGTGAAGGGCAGGTTCACCGTGAGCAGGGACA |
| | | ACGCCAAGAACACCATGTACCTGCAGATGAACAAC |
| | | CTGCTGCCCGAGGACAGCGCCGTGTACTACTGCAA |
| | | GGCCGACACCGCCACCAACACCGACTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTTATAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 657 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGAGCGGCGGCGGCCTGGTGCAGCCC |
| | | GGCGGCAGCCTGAGGCTGAGCTGCGCCGCCAGCAG |
| | | GAGCATCTTCGTGATCGCCTTCATGGACTGGTACA |
| | | GGCAGGCCCCCGGCAAGCAGAGGGAGTTCGTGGCC |
| | | GGCATCAGCAGCAGCGGCGCCACCAGCTACGCCAG |
| | | CAGCGTGAAGGGCAGGTTCACCGTGAGCAGGGACA |
| | | CCGCCAAGAACACCATGTACCTGCAGATGAACAAC |
| | | CTGCTGCCCGAGGACAGCGCCGTGTACTACTGCAA |
| | | GGCCGACACCGCCACCACCACCGACTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTTATAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 658 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGAGCGGCGGCGGCCTGGTGCAGCCC |
| | | GGCGGCAGCCTGAGGCTGAGCTGCGCCGCCAGCGG |
| | | CATCATCTTCAGCATCAACACCATGGCCTGGTACA |
| | | GGCAGGGCCCCGGCAAGGAGAGGGACCTGGTGGCC |
| | | GTGATCAAGGGCGACGGCAGCACCAGCTACGCCGA |
| | | CAGCGTGAACGGCAGGTTCACCATCAGCAGGGACA |
| | | ACGCCAAGAACACCGTGTACCTGAGGATGAACGGC |
| | | CTGAAGCCCGAGGACACCGCCGTGTACTACTGCAA |
| | | CAGCGCCGGCAGGAGCTACAGCGGCGTGTACGGCG |
| | | CCTACTGGGGCCAGGGCACCCAGGTGACCGTGAGC |
| | | AGCGAGCAGAAACTGATCAGCGAGGAAGATCTGAA |
| | | TCCTGGCGGAGGCGGAGGAAGTGGTGGCGGAGGTT |
| | | CTGGTGGCGGTGGATCAGGCGGTGGCGGATCTGGA |
| | | TCTATGGATATCCAGATGACCCAGTCCCCGAGCTC |
| | | CCTGTCCGCCTCTGTGGGCGATAGGGTCACCATCA |
| | | CCTGCCGTGCCAGTCAGGACATCCGTAATTATCTG |
| | | AACTGGTATCAACAGAAACCAGGAAAAGCTCCGAA |
| | | ACTACTGATTTACTATACCTCCCGCCTGGAGTCTG |
| | | GAGTCCCTTCTCGCTTCTCTGGTTCTGGTTCTGGG |
| | | ACGGATTACACTCTGACCATCAGCAGTCTGCAACC |
| | | GGAAGACTTCGCAACTTATTACTGTCAGCAAGGTA |
| | | ATACTCTGCCGTGGACGTTCGGACAGGGCACCAAG |
| | | GTGGAGATCAAAGGCGGCGGCGGAAGTGGAGGAGG |
| | | AGGCTCAGGCGGAGGAGGGAGCGAGGTTCAGCTGG |
| | | TGGAGTCTGGCGGTGGCCTGGTGCAGCCAGGGGGC |
| | | TCACTCCGTTTGTCCTGTGCAGCTTCTGGCTACTC |
| | | CTTTACCGGCTACACTATGAACTGGGTGCGTCAGG |
| | | CCCCAGGTAAGGGCCTGGAATGGGTTGCACTGATT |
| | | AATCCTTATAAAGGTGTTAGTACCTACAACCAGAA |
| | | GTTCAAGGACCGTTTCACTATAAGCGTAGATAAAT |
| | | CCAAAAACACAGCCTACCTGCAAATGAACAGCCTG |
| | | CGTGCTGAGGACACTGCCGTCTATTATTGTGCTAG |
| | | AAGCGGATACTACGGCGATAGTGACTGGTATTTTG |
| | | ACGTGTGGGGTCAAGGAACCCTGGTCACCGTCTCC |
| | | TCGACATCTGGCGGCGGAGGATCTCTGGAATCTGG |
| | | ACAGGTGCTGCTGGAAAGCAACATCAAGGTGCTGC |
| | | CCACCTGGTCTACCCCAGTTCAGCCTATGGCTCTG |
| | | ATTGTGCTTGGCGGAGTTGCCGGCCTGCTGCTCTT |
| | | TATCGGCCTGGGCATCTTCTTTTGCGTGCGGTGCA |
| | | GACATCGGCGGAGACAGGCTGAGAGAATGAGCCAG |
| | | ATCAAGCGGCTGCTGAGCGAGAAGAAAACCTGTCA |
| | | GTGCCCTCACCGGTTCCAGAAAACATGCAGCCCCA |
| | | TC |
| 659 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGCAGGAGAGCGGCGGCGGCCTGGTGCAGCCC |
| | | GGCGGCAGCCTGAAGCTGAGCTGCGCCGCCAGCGG |
| | | CAGCATCTTCAGCATCGGCGCCATGAGGTGGTACA |
| | | GGCAGGTGCCCGGCAACGAGAGGGAGCTGGTGGCC |
| | | GGCATCACCAACGGCGGCAACACCAACTACGCCGA |
| | | CAGCGTGAAGGCCAGGTTCACCATCAGCAGGGACA |
| | | ACGCCAAGAACACCGTGTACCTGCAGATGAACAGC |
| | | CTGAAGCCCGAGGACACCACCGTGTACTTCTGCAA |
| | | CGCCGACGTGCAGAACAGCGGCTACGTGTGGGGCA |
| | | ACTACTGGGGCCAGGGCACCCAGGTGACCGTGAGC |
| | | AGCGAGCAGAAACTGATCAGCGAGGAAGATCTGAA |
| | | TCCTGGCGGAGGCGGAGGAAGTGGTGGCGGAGGTT |
| | | CTGGTGGCGGTGGATCAGGCGGTGGCGGATCTGGA |
| | | TCTATGGATATCCAGATGACCCAGTCCCCGAGCTC |
| | | CCTGTCCGCCTCTGTGGGCGATAGGGTCACCATCA |
| | | CCTGCCGTGCCAGTCAGGACATCCGTAATTATCTG |
| | | AACTGGTATCAACAGAAACCAGGAAAAGCTCCGAA |
| | | ACTACTGATTTACTATACCTCCCGCCTGGAGTCTG |
| | | GAGTCCCTTCTCGCTTCTCTGGTTCTGGTTCTGGG |
| | | ACGGATTACACTCTGACCATCAGCAGTCTGCAACC |
| | | GGAAGACTTCGCAACTTATTACTGTCAGCAAGGTA |
| | | ATACTCTGCCGTGGACGTTCGGACAGGGCACCAAG |
| | | GTGGAGATCAAAGGCGGCGGCGGAAGTGGAGGAGG |
| | | AGGCTCAGGCGGAGGAGGGAGCGAGGTTCAGCTGG |
| | | TGGAGTCTGGCGGTGGCCTGGTGCAGCCAGGGGGC |
| | | TCACTCCGTTTGTCCTGTGCAGCTTCTGGCTACTC |
| | | CTTTACCGGCTACACTATGAACTGGGTGCGTCAGG |
| | | CCCCAGGTAAGGGCCTGGAATGGGTTGCACTGATT |
| | | AATCCTTATAAAGGTGTTAGTACCTACAACCAGAA |
| | | GTTCAAGGACCGTTTCACTATAAGCGTAGATAAAT |
| | | CCAAAAACACAGCCTACCTGCAAATGAACAGCCTG |
| | | CGTGCTGAGGACACTGCCGTCTATTATTGTGCTAG |
| | | AAGCGGATACTACGGCGATAGTGACTGGTATTTTG |
| | | ACGTGTGGGGTCAAGGAACCCTGGTCACCGTCTCC |
| | | TCGACATCTGGCGGCGGAGGATCTCTGGAATCTGG |
| | | ACAGGTGCTGCTGGAAAGCAACATCAAGGTGCTGC |
| | | CCACCTGGTCTACCCCAGTTCAGCCTATGGCTCTG |
| | | ATTGTGCTTGGCGGAGTTGCCGGCCTGCTGCTCTT |
| | | TATCGGCCTGGGCATCTTCTTTTGCGTGCGGTGCA |
| | | GACATCGGCGGAGACAGGCTGAGAGAATGAGCCAG |
| | | ATCAAGCGGCTGCTGAGCGAGAAGAAAACCTGTCA |
| | | GTGCCCTCACCGGTTCCAGAAAACATGCAGCCCCA |
| | | TC |
| 660 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGAGCGGCGGCGGCCTGGTGCAGCCC |
| | | GGCGGCAGCCTGAGGCTGAGCTGCGCCGCCAGCAG |
| | | GAGCATCTTCGTGATCGCCTTCATGGACTGGTACA |
| | | GGCAGGCCCCCGGCAAGCAGAGGGAGTTCGTGGCC |
| | | GGCATCAGCAGCAGCGGCGCCACCAGCTACGCCGA |
| | | CAGCGTGAAGGGCAGGTTCACCGTGAGCAGGGACA |
| | | CCGCCAAGAACACCATGTACCTGCAGATGAACAAC |
| | | CTGCTGCCCGAGGACAGCGCCGTGTACTACTGCAA |
| | | GGCCGACACCGCCACCAACACCGACTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTTATAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 661 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGAGCGGCGGCGGCCTGGTGCAGCCC |
| | | GGCGGCAGCCTGAGGCTGAGCTGCGCCACCAGCGG |
| | | CATCATCTTCCACATCTACACCATGGGCTGGTACA |
| | | GGGGCGCCCCCGGCAAGCAGAGGGAGCTGGTGGCC |
| | | AGGATCACCAGCGGCGGCGACACCAACTACGCCGA |
| | | CAGCGTGAAGGGCAGGTTCACCATCAGCAGGGACA |
| | | ACGTGAAGAACACCATGTACCTGCAGATGAACAGC |
| | | CTGAAGCCCGAGGACACCGCCGTGTACTACTGCAA |
| | | CAGGTTCCCCGGCGCCACCTTCAGCTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTTATAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 662 | Nucleotide | ATGGCTCTGCCTGTGACAGCTCTGTTGCTGCCTCT |
| | | GGCTCTGCTGCTGCATGCTGCTAGACCTCAGCTGC |
| | | AGCTGGTGGAGAGCGGCGGCGGCCTGGTGCAGGCC |
| | | GGCGGCAGCCTGAGGCTGAGCTGCGCCGTGAGCGG |
| | | CAACATCTGGGTGTTCACCGTGATGGCCTGGTACA |
| | | GGCAGGTGCCCGGCAAGCAGAGGGAGCTGGTGGCC |
| | | GCCAGCACCAACGGCGGCAGCACCAACTACGCCGA |
| | | CAGCGTGAAGGGCAGGTTCACCATCAGCAGGGACA |
| | | ACGTGAAGAACACCGTGTACCTGCAGATGAACAGC |
| | | CTGAAGCCCGAGGACACCGCCGTGTACTACTGCAA |
| | | CAGGCAGAGGACCGTGATCGGCATGAACCCCCTGG |
| | | CCCCCTGGGGCCAGGGCACCCAGGTGACCGTGAGC |
| | | AGCGAGCAGAAACTGATCAGCGAGGAAGATCTGAA |
| | | TCCTGGCGGAGGCGGAGGAAGTGGTGGCGGAGGTT |
| | | CTGGTGGCGGTGGATCAGGCGGTGGCGGATCTGGA |
| | | TCTATGGATATCCAGATGACCCAGTCCCCGAGCTC |
| | | CCTGTCCGCCTCTGTGGGCGATAGGGTCACCATCA |
| | | CCTGCCGTGCCAGTCAGGACATCCGTAATTATCTG |
| | | AACTGGTATCAACAGAAACCAGGAAAAGCTCCGAA |
| | | ACTACTGATTTACTATACCTCCCGCCTGGAGTCTG |
| | | GAGTCCCTTCTCGCTTCTCTGGTTCTGGTTCTGGG |
| | | ACGGATTACACTCTGACCATCAGCAGTCTGCAACC |
| | | GGAAGACTTCGCAACTTATTACTGTCAGCAAGGTA |
| | | ATACTCTGCCGTGGACGTTCGGACAGGGCACCAAG |
| | | GTGGAGATCAAAGGCGGCGGCGGAAGTGGAGGAGG |
| | | AGGCTCAGGCGGAGGAGGGAGCGAGGTTCAGCTGG |
| | | TGGAGTCTGGCGGTGGCCTGGTGCAGCCAGGGGGC |
| | | TCACTCCGTTTGTCCTGTGCAGCTTCTGGCTACTC |
| | | CTTTACCGGCTACACTATGAACTGGGTGCGTCAGG |
| | | CCCCAGGTAAGGGCCTGGAATGGGTTGCACTGATT |
| | | AATCCTTATAAAGGTGTTAGTACCTACAACCAGAA |
| | | GTTCAAGGACCGTTTCACTATAAGCGTAGATAAAT |
| | | CCAAAAACACAGCCTACCTGCAAATGAACAGCCTG |
| | | CGTGCTGAGGACACTGCCGTCTATTATTGTGCTAG |
| | | AAGCGGATACTACGGCGATAGTGACTGGTATTTTG |
| | | ACGTGTGGGGTCAAGGAACCCTGGTCACCGTCTCC |
| | | TCGACATCTGGCGGCGGAGGATCTCTGGAATCTGG |
| | | ACAGGTGCTGCTGGAAAGCAACATCAAGGTGCTGC |
| | | CCACCTGGTCTACCCCAGTTCAGCCTATGGCTCTG |
| | | ATTGTGCTTGGCGGAGTTGCCGGCCTGCTGCTCTT |
| | | TATCGGCCTGGGCATCTTCTTTTGCGTGCGGTGCA |
| | | GACATCGGCGGAGACAGGCTGAGAGAATGAGCCAG |
| | | ATCAAGCGGCTGCTGAGCGAGAAGAAAACCTGTCA |
| | | GTGCCCTCACCGGTTCCAGAAAACATGCAGCCCCA |
| | | TC |
| 663 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGAGCGGCGGCGGCCTGGTGCAGCCC |
| | | GGCGGCAGCCTGAGGCTGAGCTGCGCCGTGAGCAG |
| | | GAACATCGCCAGCCTGTACAGGGTGGACTGGTACA |
| | | GGCAGGCCCCCGGCAAGCAGAGGGAGCTGGTGGCC |
| | | GGCAGGACCAGCGGCGGCACCACCACCTACCTGGA |
| | | CGCCGTGGAGGGCAGGTTCACCATCAGCAGGGACA |
| | | ACGTGAAGGACACCGTGTACCTGCAGATGAACAGC |
| | | CTGACCCCCGAGGACACCGCCGTGTACTACTGCCA |
| | | CGCCCACGACCACTGGAGGGACAGCTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTTATAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 664 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGGAGAGCGGCGGCGGCCTGGTGCAGCCC |
| | | GGCGGCAGCCTGAGGCTGAGCTGCGCCGCCAGCAG |
| | | GAACATCTTCAGCCTGTACAGGGTGGACTGGTACA |
| | | GGCAGGCCCCCGGCAAGCAGAGGGAGCTGGTGGCC |
| | | GGCAGCACCAGCGGCGGCACCACCACCTACGCCGA |
| | | CGCCGTGAAGGGCAGGTTCACCATCAGCACCGACA |
| | | ACGTGAAGGACACCGTGTACCTGCAGATGAACAGC |
| | | CTGACCCCCGAGGACACCGCCGTGTACTACTGCCA |
| | | CGCCCACGACCACTGGAGGGACAGCTGGGGCCAGG |
| | | GCACCCAGGTGACCGTGAGCAGCGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTTATAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 665 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGACATATTACGATTGAGCGTGTCCTCGG |
| | | GTATGGACGTCTGGGGCCAAGGGACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGCTGACTCAGCCACCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGACTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGCTGTGGTATTCGGCGGAGGGACCAAGCTGA |
| | | CCGTCCTAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTTATAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 666 | Nucleotide | ATGGCTCTGCCTGTGACAGCTCTGTTGCTGCCTCT |
| | | GGCTCTGCTGCTGCATGCTGCTAGACCTCAGTCTG |
| | | TGCTGACTCAGCCACCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGACTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGCTGTGGTATTCGGCGGAGGGACCAAGCTGA |
| | | CCGTCCTAGGGTCAACCTCTGGTAGCGGTAAGCCT |
| | | GGCTCCGGCGAAGGCTCCACAAAGGGTCAGGTGCA |
| | | GCTGGTGCAGTCTGGGGCTGAGGTGAAGAAGCCTG |
| | | GGTCCTCGGTGAAGGTCTCCTGCAAGGCTTCTGGA |
| | | GGCACCTTCAGCAGCTATGCTATCAGCTGGGTGCG |
| | | ACAGGCCCCTGGACAAGGGCTTGAGTGGATGGGAG |
| | | GGATCATCCCTATCTTTGGTACAGCAAACTACGCA |
| | | CAGAAGTTCCAGGGCAGAGTCACGATTACCGCGGA |
| | | CGAATCCACGAGCACAGCCTACATGGAGCTGAGCA |
| | | GCCTGAGGAGCGAGGACACGGCCGTGTATTACTGT |
| | | GCGAGAGACATATTACGATTGAGCGTGTCCTCGGG |
| | | TATGGACGTCTGGGGCCAAGGGACCCTGGTCACCG |
| | | TGTCCTCAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTTATAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 667 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGCAGGAGTCGGGGGGAGGCGTGGTCCAGCCT |
| | | GGGAGGTCCCTGAGACTCTCCTGTGCAGCCTCTGG |
| | | ATTCACCCTCAGTAGCTATGGCATGCACTGGGTCC |
| | | GCCAGGCTCCAGGCAAGGGGCTGGAGTGGGTGGCA |
| | | GTTATATCATATGATGGAAGTAATAAATACTATGC |
| | | AGACTCCGTGAAGGGCCGATTCACCATCTCCAGAG |
| | | ACAATTCCAAGAACACGCTGTATCTGCAAATGAAC |
| | | AGCCTGAGAGCTGAGGACACGGCTGTGTATTACTG |
| | | TGCGAAGAGCGGCTGGAGATATTACTACTACTACG |
| | | GTATGGACGTCTGGGGCCAAGGGACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGTTGACGCAGCCGCCCTCAGTGTCTGGGGCCCCA |
| | | GGGCAGAGGGTCACCATCTCCTGCACTGGGAGCAG |
| | | CTCCAACATCGGGGCAGGTTATGATGTACACTGGT |
| | | ACCAGCAGCTTCCAGGAACAGCCCCCAAACTCCTC |
| | | ATCTATGGTAACAGCAATCGGCCCTCAGGGGTCCC |
| | | TGACCGATTCTCTGGCTCCAAGTCTGGCACCTCAG |
| | | CCTCCCTGGCCATCACTGGGCTCCAGGCTGAGGAT |
| | | GAGGCTGATTATTACTGCCAGTCCTATGACAGCAG |
| | | CCTGAGTGGTTCGGTATTCGGCGGAGGGACCAAGC |
| | | TGACCGTCCTAGAGCAGAAACTGATCAGCGAGGAA |
| | | GATCTGAATCCTGGCGGAGGCGGAGGAAGTGGTGG |
| | | CGGAGGTTCTGGTGGCGGTGGATCAGGCGGTGGCG |
| | | GATCTGGATCTATGGATATCCAGATGACCCAGTCC |
| | | CCGAGCTCCCTGTCCGCCTCTGTGGGCGATAGGGT |
| | | CACCATCACCTGCCGTGCCAGTCAGGACATCCGTA |
| | | ATTATCTGAACTGGTATCAACAGAAACCAGGAAAA |
| | | GCTCCGAAACTACTGATTTACTATACCTCCCGCCT |
| | | GGAGTCTGGAGTCCCTTCTCGCTTCTCTGGTTCTG |
| | | GTTCTGGGACGGATTACACTCTGACCATCAGCAGT |
| | | CTGCAACCGGAAGACTTCGCAACTTATTACTGTCA |
| | | GCAAGGTAATACTCTGCCGTGGACGTTCGGACAGG |
| | | GCACCAAGGTGGAGATCAAAGGCGGCGGCGGAAGT |
| | | GGAGGAGGAGGCTCAGGCGGAGGAGGGAGCGAGGT |
| | | TCAGCTGGTGGAGTCTGGCGGTGGCCTGGTGCAGC |
| | | CAGGGGGCTCACTCCGTTTGTCCTGTGCAGCTTCT |
| | | GGCTACTCCTTTACCGGCTACACTATGAACTGGGT |
| | | GCGTCAGGCCCCAGGTAAGGGCCTGGAATGGGTTG |
| | | CACTGATTAATCCTTATAAAGGTGTTAGTACCTAC |
| | | AACCAGAAGTTCAAGGACCGTTTCACTATAAGCGT |
| | | AGATAAATCCAAAAACACAGCCTACCTGCAAATGA |
| | | ACAGCCTGCGTGCTGAGGACACTGCCGTCTATTAT |
| | | TGTGCTAGAAGCGGATACTACGGCGATAGTGACTG |
| | | GTATTTTGACGTGTGGGGTCAAGGAACCCTGGTCA |
| | | CCGTCTCCTCGACATCTGGCGGCGGAGGATCTCTG |
| | | GAATCTGGACAGGTGCTGCTGGAAAGCAACATCAA |
| | | GGTGCTGCCCACCTGGTCTACCCCAGTTCAGCCTA |
| | | TGGCTCTGATTGTGCTTGGCGGAGTTGCCGGCCTG |
| | | CTGCTCTTTATCGGCCTGGGCATCTTCTTTTGCGT |
| | | GCGGTGCAGACATCGGCGGAGACAGGCTGAGAGAA |
| | | TGAGCCAGATCAAGCGGCTGCTGAGCGAGAAGAAA |
| | | ACCTGTCAGTGCCCTCACCGGTTCCAGAAAACATG |
| | | CAGCCCCATC |
| 668 | Nucleotide | ATGGCTCTGCCTGTGACAGCTCTGTTGCTGCCTCT |
| | | GGCTCTGCTGCTGCATGCTGCTAGACCTCAGTCTG |
| | | TGTTGACGCAGCCGCCCTCAGTGTCTGGGGCCCCA |
| | | GGGCAGAGGGTCACCATCTCCTGCACTGGGAGCAG |
| | | CTCCAACATCGGGGCAGGTTATGATGTACACTGGT |
| | | ACCAGCAGCTTCCAGGAACAGCCCCCAAACTCCTC |
| | | ATCTATGGTAACAGCAATCGGCCCTCAGGGGTCCC |
| | | TGACCGATTCTCTGGCTCCAAGTCTGGCACCTCAG |
| | | CCTCCCTGGCCATCACTGGGCTCCAGGCTGAGGAT |
| | | GAGGCTGATTATTACTGCCAGTCCTATGACAGCAG |
| | | CCTGAGTGGTTCGGTATTCGGCGGAGGGACCAAGC |
| | | TGACCGTCCTAGGGTCAACCTCTGGTAGCGGTAAG |
| | | CCTGGCTCCGGCGAAGGCTCCACAAAGGGTCAGGT |
| | | GCAGCTGCAGGAGTCGGGGGGAGGCGTGGTCCAGC |
| | | CTGGGAGGTCCCTGAGACTCTCCTGTGCAGCCTCT |
| | | GGATTCACCCTCAGTAGCTATGGCATGCACTGGGT |
| | | CCGCCAGGCTCCAGGCAAGGGGCTGGAGTGGGTGG |
| | | CAGTTATATCATATGATGGAAGTAATAAATACTAT |
| | | GCAGACTCCGTGAAGGGCCGATTCACCATCTCCAG |
| | | AGACAATTCCAAGAACACGCTGTATCTGCAAATGA |
| | | ACAGCCTGAGAGCTGAGGACACGGCTGTGTATTAC |
| | | TGTGCGAAGAGCGGCTGGAGATATTACTACTACTA |
| | | CGGTATGGACGTCTGGGGCCAAGGGACCCTGGTCA |
| | | CCGTGTCCTCAGAGCAGAAACTGATCAGCGAGGAA |
| | | GATCTGAATCCTGGCGGAGGCGGAGGAAGTGGTGG |
| | | CGGAGGTTCTGGTGGCGGTGGATCAGGCGGTGGCG |
| | | GATCTGGATCTATGGATATCCAGATGACCCAGTCC |
| | | CCGAGCTCCCTGTCCGCCTCTGTGGGCGATAGGGT |
| | | CACCATCACCTGCCGTGCCAGTCAGGACATCCGTA |
| | | ATTATCTGAACTGGTATCAACAGAAACCAGGAAAA |
| | | GCTCCGAAACTACTGATTTACTATACCTCCCGCCT |
| | | GGAGTCTGGAGTCCCTTCTCGCTTCTCTGGTTCTG |
| | | GTTCTGGGACGGATTACACTCTGACCATCAGCAGT |
| | | CTGCAACCGGAAGACTTCGCAACTTATTACTGTCA |
| | | GCAAGGTAATACTCTGCCGTGGACGTTCGGACAGG |
| | | GCACCAAGGTGGAGATCAAAGGCGGCGGCGGAAGT |
| | | GGAGGAGGAGGCTCAGGCGGAGGAGGGAGCGAGGT |
| | | TCAGCTGGTGGAGTCTGGCGGTGGCCTGGTGCAGC |
| | | CAGGGGGCTCACTCCGTTTGTCCTGTGCAGCTTCT |
| | | GGCTACTCCTTTACCGGCTACACTATGAACTGGGT |
| | | GCGTCAGGCCCCAGGTAAGGGCCTGGAATGGGTTG |
| | | CACTGATTAATCCTTATAAAGGTGTTAGTACCTAC |
| | | AACCAGAAGTTCAAGGACCGTTTCACTATAAGCGT |
| | | AGATAAATCCAAAAACACAGCCTACCTGCAAATGA |
| | | ACAGCCTGCGTGCTGAGGACACTGCCGTCTATTAT |
| | | TGTGCTAGAAGCGGATACTACGGCGATAGTGACTG |
| | | GTATTTTGACGTGTGGGGTCAAGGAACCCTGGTCA |
| | | CCGTCTCCTCGACATCTGGCGGCGGAGGATCTCTG |
| | | GAATCTGGACAGGTGCTGCTGGAAAGCAACATCAA |
| | | GGTGCTGCCCACCTGGTCTACCCCAGTTCAGCCTA |
| | | TGGCTCTGATTGTGCTTGGCGGAGTTGCCGGCCTG |
| | | CTGCTCTTTATCGGCCTGGGCATCTTCTTTTGCGT |
| | | GCGGTGCAGACATCGGCGGAGACAGGCTGAGAGAA |
| | | TGAGCCAGATCAAGCGGCTGCTGAGCGAGAAGAAA |
| | | ACCTGTCAGTGCCCTCACCGGTTCCAGAAAACATG |
| | | CAGCCCCATC |
| 669 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGTTGGAGTCTGGGGGAGGCTTGGTCCAGCCT |
| | | GGGGGGTCCCTGAGACTCTCCTGTGCAGCCTCTGG |
| | | ATTCACCTTTAGTAGCTATTGGATGAGCTGGGTCC |
| | | GCCAGGCTCCAGGGAAGGGGCTGGAGTGGGTGGCC |
| | | AACATAAAGCAAGATGGAAGTGAGAAATACTATGT |
| | | GGACTCTGTGAAGGGCCGATTCACCATCTCCAGAG |
| | | ACAACGCCAAGAACTCACTGTATCTGCAAATGAAC |
| | | AGCCTGAGAGCCGAGGACACGGCTGTGTATTACTG |
| | | TGCGAGACCGGCGGGCCAGCTGCTCTACGGTATGG |
| | | ACGTCTGGGGCCAAGGGACCCTGGTCACCGTGTCC |
| | | TCAGGGTCAACCTCTGGTAGCGGTAAGCCTGGCTC |
| | | CGGCGAAGGCTCCACAAAGGGTTCCTATGTGCTGA |
| | | CTCAGCCACCCTCGGTGTCAGTGTCCCCAGGACAG |
| | | ACGGCCAGGATCACCTGCTCTGGAGATGCATTGCC |
| | | AAAGCAATATGCTTATTGGTACCAGCAGAAGCCAG |
| | | GCCAGGCCCCTGTGCTGGTGATATATAAAGACAGT |
| | | GAGAGGCCCTCAGGGATCCCTGAGCGATTCTCTGG |
| | | CTCCAGCTCAGGGACAACAGTCACGTTGACCATCA |
| | | GTGGAGTCCAGGCAGAAGACGAGGCTGACTATTAC |
| | | TGTCAATCAGCAGACAGCAGTGGTACTTGGGTGTT |
| | | CGGCGGAGGCACCAAGCTGACCGTCCTCGAGCAGA |
| | | AACTGATCAGCGAGGAAGATCTGAATCCTGGCGGA |
| | | GGCGGAGGAAGTGGTGGCGGAGGTTCTGGTGGCGG |
| | | TGGATCAGGCGGTGGCGGATCTGGATCTATGGATA |
| | | TCCAGATGACCCAGTCCCCGAGCTCCCTGTCCGCC |
| | | TCTGTGGGCGATAGGGTCACCATCACCTGCCGTGC |
| | | CAGTCAGGACATCCGTAATTATCTGAACTGGTATC |
| | | AACAGAAACCAGGAAAAGCTCCGAAACTACTGATT |
| | | TACTATACCTCCCGCCTGGAGTCTGGAGTCCCTTC |
| | | TCGCTTCTCTGGTTCTGGTTCTGGGACGGATTACA |
| | | CTCTGACCATCAGCAGTCTGCAACCGGAAGACTTC |
| | | GCAACTTATTACTGTCAGCAAGGTAATACTCTGCC |
| | | GTGGACGTTCGGACAGGGCACCAAGGTGGAGATCA |
| | | AAGGCGGCGGCGGAAGTGGAGGAGGAGGCTCAGGC |
| | | GGAGGAGGGAGCGAGGTTCAGCTGGTGGAGTCTGG |
| | | CGGTGGCCTGGTGCAGCCAGGGGGCTCACTCCGTT |
| | | TGTCCTGTGCAGCTTCTGGCTACTCCTTTACCGGC |
| | | TACACTATGAACTGGGTGCGTCAGGCCCCAGGTAA |
| | | GGGCCTGGAATGGGTTGCACTGATTAATCCTTATA |
| | | AAGGTGTTAGTACCTACAACCAGAAGTTCAAGGAC |
| | | CGTTTCACTATAAGCGTAGATAAATCCAAAAACAC |
| | | AGCCTACCTGCAAATGAACAGCCTGCGTGCTGAGG |
| | | ACACTGCCGTCTATTATTGTGCTAGAAGCGGATAC |
| | | TACGGCGATAGTGACTGGTATTTTGACGTGTGGGG |
| | | TCAAGGAACCCTGGTCACCGTCTCCTCGACATCTG |
| | | GCGGCGGAGGATCTCTGGAATCTGGACAGGTGCTG |
| | | CTGGAAAGCAACATCAAGGTGCTGCCCACCTGGTC |
| | | TACCCCAGTTCAGCCTATGGCTCTGATTGTGCTTG |
| | | GCGGAGTTGCCGGCCTGCTGCTCTTTATCGGCCTG |
| | | GGCATCTTCTTTTGCGTGCGGTGCAGACATCGGCG |
| | | GAGACAGGCTGAGAGAATGAGCCAGATCAAGCGGC |
| | | TGCTGAGCGAGAAGAAAACCTGTCAGTGCCCTCAC |
| | | CGGTTCCAGAAAACATGCAGCCCCATC |
| 670 | Nucleotide | ATGGCTCTGCCTGTGACAGCTCTGTTGCTGCCTCT |
| | | GGCTCTGCTGCTGCATGCTGCTAGACCTTCCTATG |
| | | TGCTGACTCAGCCACCCTCGGTGTCAGTGTCCCCA |
| | | GGACAGACGGCCAGGATCACCTGCTCTGGAGATGC |
| | | ATTGCCAAAGCAATATGCTTATTGGTACCAGCAGA |
| | | AGCCAGGCCAGGCCCCTGTGCTGGTGATATATAAA |
| | | GACAGTGAGAGGCCCTCAGGGATCCCTGAGCGATT |
| | | CTCTGGCTCCAGCTCAGGGACAACAGTCACGTTGA |
| | | CCATCAGTGGAGTCCAGGCAGAAGACGAGGCTGAC |
| | | TATTACTGTCAATCAGCAGACAGCAGTGGTACTTG |
| | | GGTGTTCGGCGGAGGCACCAAGCTGACCGTCCTCG |
| | | GGTCAACCTCTGGTAGCGGTAAGCCTGGCTCCGGC |
| | | GAAGGCTCCACAAAGGGTGAGGTGCAGCTGTTGGA |
| | | GTCTGGGGGAGGCTTGGTCCAGCCTGGGGGGTCCC |
| | | TGAGACTCTCCTGTGCAGCCTCTGGATTCACCTTT |
| | | AGTAGCTATTGGATGAGCTGGGTCCGCCAGGCTCC |
| | | AGGGAAGGGGCTGGAGTGGGTGGCCAACATAAAGC |
| | | AAGATGGAAGTGAGAAATACTATGTGGACTCTGTG |
| | | AAGGGCCGATTCACCATCTCCAGAGACAACGCCAA |
| | | GAACTCACTGTATCTGCAAATGAACAGCCTGAGAG |
| | | CCGAGGACACGGCTGTGTATTACTGTGCGAGACCG |
| | | GCGGGCCAGCTGCTCTACGGTATGGACGTCTGGGG |
| | | CCAAGGGACCCTGGTCACCGTGTCCTCAGAGCAGA |
| | | AACTGATCAGCGAGGAAGATCTGAATCCTGGCGGA |
| | | GGCGGAGGAAGTGGTGGCGGAGGTTCTGGTGGCGG |
| | | TGGATCAGGCGGTGGCGGATCTGGATCTATGGATA |
| | | TCCAGATGACCCAGTCCCCGAGCTCCCTGTCCGCC |
| | | TCTGTGGGCGATAGGGTCACCATCACCTGCCGTGC |
| | | CAGTCAGGACATCCGTAATTATCTGAACTGGTATC |
| | | AACAGAAACCAGGAAAAGCTCCGAAACTACTGATT |
| | | TACTATACCTCCCGCCTGGAGTCTGGAGTCCCTTC |
| | | TCGCTTCTCTGGTTCTGGTTCTGGGACGGATTACA |
| | | CTCTGACCATCAGCAGTCTGCAACCGGAAGACTTC |
| | | GCAACTTATTACTGTCAGCAAGGTAATACTCTGCC |
| | | GTGGACGTTCGGACAGGGCACCAAGGTGGAGATCA |
| | | AAGGCGGCGGCGGAAGTGGAGGAGGAGGCTCAGGC |
| | | GGAGGAGGGAGCGAGGTTCAGCTGGTGGAGTCTGG |
| | | CGGTGGCCTGGTGCAGCCAGGGGGCTCACTCCGTT |
| | | TGTCCTGTGCAGCTTCTGGCTACTCCTTTACCGGC |
| | | TACACTATGAACTGGGTGCGTCAGGCCCCAGGTAA |
| | | GGGCCTGGAATGGGTTGCACTGATTAATCCTTATA |
| | | AAGGTGTTAGTACCTACAACCAGAAGTTCAAGGAC |
| | | CGTTTCACTATAAGCGTAGATAAATCCAAAAACAC |
| | | AGCCTACCTGCAAATGAACAGCCTGCGTGCTGAGG |
| | | ACACTGCCGTCTATTATTGTGCTAGAAGCGGATAC |
| | | TACGGCGATAGTGACTGGTATTTTGACGTGTGGGG |
| | | TCAAGGAACCCTGGTCACCGTCTCCTCGACATCTG |
| | | GCGGCGGAGGATCTCTGGAATCTGGACAGGTGCTG |
| | | CTGGAAAGCAACATCAAGGTGCTGCCCACCTGGTC |
| | | TACCCCAGTTCAGCCTATGGCTCTGATTGTGCTTG |
| | | GCGGAGTTGCCGGCCTGCTGCTCTTTATCGGCCTG |
| | | GGCATCTTCTTTTGCGTGCGGTGCAGACATCGGCG |
| | | GAGACAGGCTGAGAGAATGAGCCAGATCAAGCGGC |
| | | TGCTGAGCGAGAAGAAAACCTGTCAGTGCCCTCAC |
| | | CGGTTCCAGAAAACATGCAGCCCCATC |
| 671 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGTTGGAGTCTGGGGGAGGCGTGGTCCAGCCT |
| | | GGGAGGTCCCTGAGACTCTCCTGTGCAGCCTCTGG |
| | | ATTCACCTTCAGTAGCTATGGCATGCACTGGGTCC |
| | | GCCAGGCTCCAGGCAAGGGGCTGGAGTGGGTGGCA |
| | | GTTATATCATATGATGGAAGTAATAAATACTATGC |
| | | AGACTCCGTGAAGGGCCGATTCACCATCTCCAGAG |
| | | ACAATTCCAAGAACACGCTGTATCTGCAAATGAAC |
| | | AGCCTGAGAGCTGAGGACACGGCTGTGTATTACTG |
| | | TGCGAAAGATGCCGCGGATATGGGGGCCTTTGACT |
| | | ACTGGGGCCAGGGAACCCTGGTCACCGTGTCCTCA |
| | | GGGTCAACCTCTGGTAGCGGTAAGCCTGGCTCCGG |
| | | CGAAGGCTCCACAAAGGGTGACATCCAGATGACCC |
| | | AGTCTCCAGACTCCCTGGCTGTGTCTCTGGGCGAG |
| | | AGGGCCACCATGAACTGCAAGTCCAGCCAGAGTGT |
| | | TTTATACAGCTCCAACAATAAGAACTACTTAGCTT |
| | | GGTACCAGCAGAAACCAGGACAGCCTCCTAAGCTG |
| | | CTCATTTACTGGGCATCTGCCCGGGAATCTGGGGT |
| | | CCCTGACCGATTCAGTGGCAGCGGGTCTGGGACAG |
| | | ATTTCACTCTCACCATCAGCAGCCTGCAGGCTGAA |
| | | GATGTGGCAGTTTATTACTGTCAGCAATATTATAG |
| | | TACTCCTCTCACTTTTGGCCAGGGGACCAAGGTGG |
| | | AAATCAAAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTTATAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 672 | Nucleotide | ATGGCTCTGCCTGTGACAGCTCTGTTGCTGCCTCT |
| | | GGCTCTGCTGCTGCATGCTGCTAGACCTGACATCC |
| | | AGATGACCCAGTCTCCAGACTCCCTGGCTGTGTCT |
| | | CTGGGCGAGAGGGCCACCATGAACTGCAAGTCCAG |
| | | CCAGAGTGTTTTATACAGCTCCAACAATAAGAACT |
| | | ACTTAGCTTGGTACCAGCAGAAACCAGGACAGCCT |
| | | CCTAAGCTGCTCATTTACTGGGCATCTGCCCGGGA |
| | | ATCTGGGGTCCCTGACCGATTCAGTGGCAGCGGGT |
| | | CTGGGACAGATTTCACTCTCACCATCAGCAGCCTG |
| | | CAGGCTGAAGATGTGGCAGTTTATTACTGTCAGCA |
| | | ATATTATAGTACTCCTCTCACTTTTGGCCAGGGGA |
| | | CCAAGGTGGAAATCAAAGGGTCAACCTCTGGTAGC |
| | | GGTAAGCCTGGCTCCGGCGAAGGCTCCACAAAGGG |
| | | TGAGGTGCAGCTGTTGGAGTCTGGGGGAGGCGTGG |
| | | TCCAGCCTGGGAGGTCCCTGAGACTCTCCTGTGCA |
| | | GCCTCTGGATTCACCTTCAGTAGCTATGGCATGCA |
| | | CTGGGTCCGCCAGGCTCCAGGCAAGGGGCTGGAGT |
| | | GGGTGGCAGTTATATCATATGATGGAAGTAATAAA |
| | | TACTATGCAGACTCCGTGAAGGGCCGATTCACCAT |
| | | CTCCAGAGACAATTCCAAGAACACGCTGTATCTGC |
| | | AAATGAACAGCCTGAGAGCTGAGGACACGGCTGTG |
| | | TATTACTGTGCGAAAGATGCCGCGGATATGGGGGC |
| | | CTTTGACTACTGGGGCCAGGGAACCCTGGTCACCG |
| | | TGTCCTCAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTTATAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 673 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGTTGGAGTCTGGGGGAGGCCTGGTCAAGCCT |
| | | GGGGGGTCCCTGAGACTCTCCTGTGCAGCCTCTGG |
| | | ATTCACCTTCAGTAGCTATAGCATGAACTGGGTCC |
| | | GCCAGGCTCCAGGGAAGGGGCTGGAGTGGGTCTCA |
| | | GCTATTAGTGGTAGTGGTGGTAGCACATACTACGC |
| | | AGACTCCGTGAAGGGCCGGTTCACCATCTCCAGAG |
| | | ACAATTCCAAGAACACGCTGTATCTGCAAATGAAC |
| | | AGCCTGAGAGCCGAGGACACGGCTGTGTATTACTG |
| | | TGCGAGAGCACGTGGATACAGCTATGGCTCTGATG |
| | | CTTTTGATATCTGGGGCCAAGGGACAATGGTCACC |
| | | GTGTCTTCAGGGTCAACCTCTGGTAGCGGTAAGCC |
| | | TGGCTCCGGCGAAGGCTCCACAAAGGGTCAGTCTG |
| | | TGCTGACTCAGCCACCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGCGTGGTATTCGGCGGAGGGACCAAGCTGA |
| | | CCGTCCTAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTTATAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 674 | Nucleotide | ATGGCTCTGCCTGTGACAGCTCTGTTGCTGCCTCT |
| | | GGCTCTGCTGCTGCATGCTGCTAGACCTCAGTCTG |
| | | TGCTGACTCAGCCACCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGCGTGGTATTCGGCGGAGGGACCAAGCTGA |
| | | CCGTCCTAGGGTCAACCTCTGGTAGCGGTAAGCCT |
| | | GGCTCCGGCGAAGGCTCCACAAAGGGTGAGGTGCA |
| | | GCTGTTGGAGTCTGGGGGAGGCCTGGTCAAGCCTG |
| | | GGGGGTCCCTGAGACTCTCCTGTGCAGCCTCTGGA |
| | | TTCACCTTCAGTAGCTATAGCATGAACTGGGTCCG |
| | | CCAGGCTCCAGGGAAGGGGCTGGAGTGGGTCTCAG |
| | | CTATTAGTGGTAGTGGTGGTAGCACATACTACGCA |
| | | GACTCCGTGAAGGGCCGGTTCACCATCTCCAGAGA |
| | | CAATTCCAAGAACACGCTGTATCTGCAAATGAACA |
| | | GCCTGAGAGCCGAGGACACGGCTGTGTATTACTGT |
| | | GCGAGAGCACGTGGATACAGCTATGGCTCTGATGC |
| | | TTTTGATATCTGGGGCCAAGGGACAATGGTCACCG |
| | | TGTCTTCAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTTATAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 675 | Nucleotide | ATGGCTCTGCCTGTGACAGCTCTGTTGCTGCCTCT |
| | | GGCTCTGCTGCTGCATGCTGCTAGACCTGAGGTGC |
| | | AGCTGGTGCAGTCTGGGGCTGAGGTGAAGAAGCCT |
| | | GGGTCCTCGGTGAAGGTCTCCTGCAAGGCTTCCGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGATCGGGGGAGAAGATATTGTAGTGGTG |
| | | GTAGCTGCCCTAATGCTTTTGATATCTGGGGCCAA |
| | | GGGACAATGGTCACCGTGTCTTCAGGGTCAACCTC |
| | | TGGTAGCGGTAAGCCTGGCTCCGGCGAAGGCTCCA |
| | | CAAAGGGTGAAATTGTGTTGACACAGTCTCCAGCC |
| | | ACCCTGTCTGTGTCTCCAGGGGAAAGAGCCACCCT |
| | | CTCCTGCAGGGCCAGTCAGAGTGTTAGCAGCAACT |
| | | TAGCCTGGTACCAGCAGAAACCTGGCCAGGCTCCC |
| | | AGGCTCCTCATCTATGGTGCATCCACCAGGGCCAC |
| | | TGGTATCCCAGCCAGGTTCAGTGGCAGTGGGTCTG |
| | | GGACAGAGTTCACTCTCACCATCAGCAGCCTGCAG |
| | | TCTGAAGATTTTGCAGTTTATTACTGTCAGCAGTA |
| | | TAATAACTGGCCCCCGGCCCTCACTTTCGGCGGAG |
| | | GGACCAAGGTGGAAATCAAAGAGCAGAAACTGATC |
| | | AGCGAGGAAGATCTGAATCCTGGCGGAGGCGGAGG |
| | | AAGTGGTGGCGGAGGTTCTGGTGGCGGTGGATCAG |
| | | GCGGTGGCGGATCTGGATCTATGGATATCCAGATG |
| | | ACCCAGTCCCCGAGCTCCCTGTCCGCCTCTGTGGG |
| | | CGATAGGGTCACCATCACCTGCCGTGCCAGTCAGG |
| | | ACATCCGTAATTATCTGAACTGGTATCAACAGAAA |
| | | CCAGGAAAAGCTCCGAAACTACTGATTTACTATAC |
| | | CTCCCGCCTGGAGTCTGGAGTCCCTTCTCGCTTCT |
| | | CTGGTTCTGGTTCTGGGACGGATTACACTCTGACC |
| | | ATCAGCAGTCTGCAACCGGAAGACTTCGCAACTTA |
| | | TTACTGTCAGCAAGGTAATACTCTGCCGTGGACGT |
| | | TCGGACAGGGCACCAAGGTGGAGATCAAAGGCGGC |
| | | GGCGGAAGTGGAGGAGGAGGCTCAGGCGGAGGAGG |
| | | GAGCGAGGTTCAGCTGGTGGAGTCTGGCGGTGGCC |
| | | TGGTGCAGCCAGGGGGCTCACTCCGTTTGTCCTGT |
| | | GCAGCTTCTGGCTACTCCTTTACCGGCTACACTAT |
| | | GAACTGGGTGCGTCAGGCCCCAGGTAAGGGCCTGG |
| | | AATGGGTTGCACTGATTAATCCTTATAAAGGTGTT |
| | | AGTACCTACAACCAGAAGTTCAAGGACCGTTTCAC |
| | | TATAAGCGTAGATAAATCCAAAAACACAGCCTACC |
| | | TGCAAATGAACAGCCTGCGTGCTGAGGACACTGCC |
| | | GTCTATTATTGTGCTAGAAGCGGATACTACGGCGA |
| | | TAGTGACTGGTATTTTGACGTGTGGGGTCAAGGAA |
| | | CCCTGGTCACCGTCTCCTCGACATCTGGCGGCGGA |
| | | GGATCTCTGGAATCTGGACAGGTGCTGCTGGAAAG |
| | | CAACATCAAGGTGCTGCCCACCTGGTCTACCCCAG |
| | | TTCAGCCTATGGCTCTGATTGTGCTTGGCGGAGTT |
| | | GCCGGCCTGCTGCTCTTTATCGGCCTGGGCATCTT |
| | | CTTTTGCGTGCGGTGCAGACATCGGCGGAGACAGG |
| | | CTGAGAGAATGAGCCAGATCAAGCGGCTGCTGAGC |
| | | GAGAAGAAAACCTGTCAGTGCCCTCACCGGTTCCA |
| | | GAAAACATGCAGCCCCATC |
| 676 | Nucleotide | ATGGCTCTGCCTGTGACAGCTCTGTTGCTGCCTCT |
| | | GGCTCTGCTGCTGCATGCTGCTAGACCTGAAATTG |
| | | TGTTGACACAGTCTCCAGCCACCCTGTCTGTGTCT |
| | | CCAGGGGAAAGAGCCACCCTCTCCTGCAGGGCCAG |
| | | TCAGAGTGTTAGCAGCAACTTAGCCTGGTACCAGC |
| | | AGAAACCTGGCCAGGCTCCCAGGCTCCTCATCTAT |
| | | GGTGCATCCACCAGGGCCACTGGTATCCCAGCCAG |
| | | GTTCAGTGGCAGTGGGTCTGGGACAGAGTTCACTC |
| | | TCACCATCAGCAGCCTGCAGTCTGAAGATTTTGCA |
| | | GTTTATTACTGTCAGCAGTATAATAACTGGCCCCC |
| | | GGCCCTCACTTTCGGCGGAGGGACCAAGGTGGAAA |
| | | TCAAAGGGTCAACCTCTGGTAGCGGTAAGCCTGGC |
| | | TCCGGCGAAGGCTCCACAAAGGGTGAGGTGCAGCT |
| | | GGTGCAGTCTGGGGCTGAGGTGAAGAAGCCTGGGT |
| | | CCTCGGTGAAGGTCTCCTGCAAGGCTTCCGGAGGC |
| | | ACCTTCAGCAGCTATGCTATCAGCTGGGTGCGACA |
| | | GGCCCCTGGACAAGGGCTTGAGTGGATGGGAGGGA |
| | | TCATCCCTATCTTTGGTACAGCAAACTACGCACAG |
| | | AAGTTCCAGGGCAGAGTCACGATTACCGCGGACGA |
| | | ATCCACGAGCACAGCCTACATGGAGCTGAGCAGCC |
| | | TGAGGAGCGAGGACACGGCCGTGTATTACTGTGCG |
| | | AGAGATCGGGGGAGAAGATATTGTAGTGGTGGTAG |
| | | CTGCCCTAATGCTTTTGATATCTGGGGCCAAGGGA |
| | | CAATGGTCACCGTGTCTTCAGAGCAGAAACTGATC |
| | | AGCGAGGAAGATCTGAATCCTGGCGGAGGCGGAGG |
| | | AAGTGGTGGCGGAGGTTCTGGTGGCGGTGGATCAG |
| | | GCGGTGGCGGATCTGGATCTATGGATATCCAGATG |
| | | ACCCAGTCCCCGAGCTCCCTGTCCGCCTCTGTGGG |
| | | CGATAGGGTCACCATCACCTGCCGTGCCAGTCAGG |
| | | ACATCCGTAATTATCTGAACTGGTATCAACAGAAA |
| | | CCAGGAAAAGCTCCGAAACTACTGATTTACTATAC |
| | | CTCCCGCCTGGAGTCTGGAGTCCCTTCTCGCTTCT |
| | | CTGGTTCTGGTTCTGGGACGGATTACACTCTGACC |
| | | ATCAGCAGTCTGCAACCGGAAGACTTCGCAACTTA |
| | | TTACTGTCAGCAAGGTAATACTCTGCCGTGGACGT |
| | | TCGGACAGGGCACCAAGGTGGAGATCAAAGGCGGC |
| | | GGCGGAAGTGGAGGAGGAGGCTCAGGCGGAGGAGG |
| | | GAGCGAGGTTCAGCTGGTGGAGTCTGGCGGTGGCC |
| | | TGGTGCAGCCAGGGGGCTCACTCCGTTTGTCCTGT |
| | | GCAGCTTCTGGCTACTCCTTTACCGGCTACACTAT |
| | | GAACTGGGTGCGTCAGGCCCCAGGTAAGGGCCTGG |
| | | AATGGGTTGCACTGATTAATCCTTATAAAGGTGTT |
| | | AGTACCTACAACCAGAAGTTCAAGGACCGTTTCAC |
| | | TATAAGCGTAGATAAATCCAAAAACACAGCCTACC |
| | | TGCAAATGAACAGCCTGCGTGCTGAGGACACTGCC |
| | | GTCTATTATTGTGCTAGAAGCGGATACTACGGCGA |
| | | TAGTGACTGGTATTTTGACGTGTGGGGTCAAGGAA |
| | | CCCTGGTCACCGTCTCCTCGACATCTGGCGGCGGA |
| | | GGATCTCTGGAATCTGGACAGGTGCTGCTGGAAAG |
| | | CAACATCAAGGTGCTGCCCACCTGGTCTACCCCAG |
| | | TTCAGCCTATGGCTCTGATTGTGCTTGGCGGAGTT |
| | | GCCGGCCTGCTGCTCTTTATCGGCCTGGGCATCTT |
| | | CTTTTGCGTGCGGTGCAGACATCGGCGGAGACAGG |
| | | CTGAGAGAATGAGCCAGATCAAGCGGCTGCTGAGC |
| | | GAGAAGAAAACCTGTCAGTGCCCTCACCGGTTCCA |
| | | GAAAACATGCAGCCCCATC |
| 677 | Nucleotide | ATGGCTCTGCCTGTGACAGCTCTGTTGCTGCCTCT |
| | | GGCTCTGCTGCTGCATGCTGCTAGACCTCAGGTTC |
| | | AGCTGGTGCAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGAGAGGGGGTTACGTTTGGGGGAGTTATCGTC |
| | | AGAACTCCAACTGGTTCGACCCCTGGGGCCAGGGA |
| | | ACCCTGGTCACCGTGTCCTCAGGGTCAACCTCTGG |
| | | TAGCGGTAAGCCTGGCTCCGGCGAAGGCTCCACAA |
| | | AGGGTTCCTATGTGCTGACTCAGCCACCCTCAGCG |
| | | TCTGGGACCCCCGGGCAGAGGGTCACCATCTCTTG |
| | | TTCTGGAAGCAGCTCCAACATCGGAAGTAATTATG |
| | | TATACTGGTACCAGCAGCTCCCAGGAACGGCCCCC |
| | | AAACTCCTCATCTATAGGAATAATCAGCGGCCCTC |
| | | AGGGGTCCCTGACCGATTCTCTGGCTCCAAGTCTG |
| | | GCACCTCAGCCTCCCTGGCCATCAGTGGGCTCCGG |
| | | TCCGAGGATGAGGCTGATTATTACTGTGCAGCATG |
| | | GGATGACAGCCTGAGTGGTTGGGTGTTCGGCGGAG |
| | | GGACCAAGCTGACCGTCCTAGAGCAGAAACTGATC |
| | | AGCGAGGAAGATCTGAATCCTGGCGGAGGCGGAGG |
| | | AAGTGGTGGCGGAGGTTCTGGTGGCGGTGGATCAG |
| | | GCGGTGGCGGATCTGGATCTATGGATATCCAGATG |
| | | ACCCAGTCCCCGAGCTCCCTGTCCGCCTCTGTGGG |
| | | CGATAGGGTCACCATCACCTGCCGTGCCAGTCAGG |
| | | ACATCCGTAATTATCTGAACTGGTATCAACAGAAA |
| | | CCAGGAAAAGCTCCGAAACTACTGATTTACTATAC |
| | | CTCCCGCCTGGAGTCTGGAGTCCCTTCTCGCTTCT |
| | | CTGGTTCTGGTTCTGGGACGGATTACACTCTGACC |
| | | ATCAGCAGTCTGCAACCGGAAGACTTCGCAACTTA |
| | | TTACTGTCAGCAAGGTAATACTCTGCCGTGGACGT |
| | | TCGGACAGGGCACCAAGGTGGAGATCAAAGGCGGC |
| | | GGCGGAAGTGGAGGAGGAGGCTCAGGCGGAGGAGG |
| | | GAGCGAGGTTCAGCTGGTGGAGTCTGGCGGTGGCC |
| | | TGGTGCAGCCAGGGGGCTCACTCCGTTTGTCCTGT |
| | | GCAGCTTCTGGCTACTCCTTTACCGGCTACACTAT |
| | | GAACTGGGTGCGTCAGGCCCCAGGTAAGGGCCTGG |
| | | AATGGGTTGCACTGATTAATCCTTATAAAGGTGTT |
| | | AGTACCTACAACCAGAAGTTCAAGGACCGTTTCAC |
| | | TATAAGCGTAGATAAATCCAAAAACACAGCCTACC |
| | | TGCAAATGAACAGCCTGCGTGCTGAGGACACTGCC |
| | | GTCTATTATTGTGCTAGAAGCGGATACTACGGCGA |
| | | TAGTGACTGGTATTTTGACGTGTGGGGTCAAGGAA |
| | | CCCTGGTCACCGTCTCCTCGACATCTGGCGGCGGA |
| | | GGATCTCTGGAATCTGGACAGGTGCTGCTGGAAAG |
| | | CAACATCAAGGTGCTGCCCACCTGGTCTACCCCAG |
| | | TTCAGCCTATGGCTCTGATTGTGCTTGGCGGAGTT |
| | | GCCGGCCTGCTGCTCTTTATCGGCCTGGGCATCTT |
| | | CTTTTGCGTGCGGTGCAGACATCGGCGGAGACAGG |
| | | CTGAGAGAATGAGCCAGATCAAGCGGCTGCTGAGC |
| | | GAGAAGAAAACCTGTCAGTGCCCTCACCGGTTCCA |
| | | GAAAACATGCAGCCCCATC |
| 678 | Nucleotide | ATGGCTCTGCCTGTGACAGCTCTGTTGCTGCCTCT |
| | | GGCTCTGCTGCTGCATGCTGCTAGACCTTCCTATG |
| | | TGCTGACTCAGCCACCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGTTGGGTGTTCGGCGGAGGGACCAAGCTGA |
| | | CCGTCCTAGGGTCAACCTCTGGTAGCGGTAAGCCT |
| | | GGCTCCGGCGAAGGCTCCACAAAGGGTCAGGTTCA |
| | | GCTGGTGCAGTCTGGGGCTGAGGTGAAGAAGCCTG |
| | | GGTCCTCGGTGAAGGTCTCCTGCAAGGCTTCTGGA |
| | | GGCACCTTCAGCAGCTATGCTATCAGCTGGGTGCG |
| | | ACAGGCCCCTGGACAAGGGCTTGAGTGGATGGGAG |
| | | GGATCATCCCTATCTTTGGTACAGCAAACTACGCA |
| | | CAGAAGTTCCAGGGCAGAGTCACGATTACCGCGGA |
| | | CGAATCCACGAGCACAGCCTACATGGAGCTGAGCA |
| | | GCCTGAGGAGCGAGGACACGGCCGTGTATTACTGT |
| | | GCGAGAGGGGGTTACGTTTGGGGGAGTTATCGTCA |
| | | GAACTCCAACTGGTTCGACCCCTGGGGCCAGGGAA |
| | | CCCTGGTCACCGTGTCCTCAGAGCAGAAACTGATC |
| | | AGCGAGGAAGATCTGAATCCTGGCGGAGGCGGAGG |
| | | AAGTGGTGGCGGAGGTTCTGGTGGCGGTGGATCAG |
| | | GCGGTGGCGGATCTGGATCTATGGATATCCAGATG |
| | | ACCCAGTCCCCGAGCTCCCTGTCCGCCTCTGTGGG |
| | | CGATAGGGTCACCATCACCTGCCGTGCCAGTCAGG |
| | | ACATCCGTAATTATCTGAACTGGTATCAACAGAAA |
| | | CCAGGAAAAGCTCCGAAACTACTGATTTACTATAC |
| | | CTCCCGCCTGGAGTCTGGAGTCCCTTCTCGCTTCT |
| | | CTGGTTCTGGTTCTGGGACGGATTACACTCTGACC |
| | | ATCAGCAGTCTGCAACCGGAAGACTTCGCAACTTA |
| | | TTACTGTCAGCAAGGTAATACTCTGCCGTGGACGT |
| | | TCGGACAGGGCACCAAGGTGGAGATCAAAGGCGGC |
| | | GGCGGAAGTGGAGGAGGAGGCTCAGGCGGAGGAGG |
| | | GAGCGAGGTTCAGCTGGTGGAGTCTGGCGGTGGCC |
| | | TGGTGCAGCCAGGGGGCTCACTCCGTTTGTCCTGT |
| | | GCAGCTTCTGGCTACTCCTTTACCGGCTACACTAT |
| | | GAACTGGGTGCGTCAGGCCCCAGGTAAGGGCCTGG |
| | | AATGGGTTGCACTGATTAATCCTTATAAAGGTGTT |
| | | AGTACCTACAACCAGAAGTTCAAGGACCGTTTCAC |
| | | TATAAGCGTAGATAAATCCAAAAACACAGCCTACC |
| | | TGCAAATGAACAGCCTGCGTGCTGAGGACACTGCC |
| | | GTCTATTATTGTGCTAGAAGCGGATACTACGGCGA |
| | | TAGTGACTGGTATTTTGACGTGTGGGGTCAAGGAA |
| | | CCCTGGTCACCGTCTCCTCGACATCTGGCGGCGGA |
| | | GGATCTCTGGAATCTGGACAGGTGCTGCTGGAAAG |
| | | CAACATCAAGGTGCTGCCCACCTGGTCTACCCCAG |
| | | TTCAGCCTATGGCTCTGATTGTGCTTGGCGGAGTT |
| | | GCCGGCCTGCTGCTCTTTATCGGCCTGGGCATCTT |
| | | CTTTTGCGTGCGGTGCAGACATCGGCGGAGACAGG |
| | | CTGAGAGAATGAGCCAGATCAAGCGGCTGCTGAGC |
| | | GAGAAGAAAACCTGTCAGTGCCCTCACCGGTTCCA |
| | | GAAAACATGCAGCCCCATC |
| 679 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACAAATCCACGAGCACAGCCTACATGGAGCTGAGC |
| | | AGCCTGAGGAGCGAGGACACGGCCGTGTATTACTG |
| | | TGCGAGGACCCATCTACCATACAGCTATGGTTTAG |
| | | GCGGGTTTGACTACTGGGGCCAGGGAACCCTGGTC |
| | | ACCGTGTCCTCAGGGTCAACCTCTGGTAGCGGTAA |
| | | GCCTGGCTCCGGCGAAGGCTCCACAAAGGGTCAGT |
| | | CTGTGCTGACGCAGCCGCCCTCAGTGTCTGGGGCC |
| | | CCAGGGCAGAGGGTCACCATCTCCTGCACTGGGAG |
| | | CAGCTCCAACATCGGGGCAGGTTATGATGTACACT |
| | | GGTACCAGCAGCTTCCAGGAACAGCCCCCAAACTC |
| | | CTCATCTATGGTAACAGCAATCGGCCCTCAGGGGT |
| | | CCCTGACCGATTCTCTGGCTCCAAGTCTGGCACCT |
| | | CAGCCTCCCTGGCCATCACTGGGCTCCAGGCTGAG |
| | | GATGAGGCTGATTATTACTGCCAGTCCTATGACAG |
| | | CAGCCTGAGTGGCCCGGTGGTATTCGGCGGAGGGA |
| | | CCAAGGTCACCGTCCTAGAGCAGAAACTGATCAGC |
| | | GAGGAAGATCTGAATCCTGGCGGAGGCGGAGGAAG |
| | | TGGTGGCGGAGGTTCTGGTGGCGGTGGATCAGGCG |
| | | GTGGCGGATCTGGATCTATGGATATCCAGATGACC |
| | | CAGTCCCCGAGCTCCCTGTCCGCCTCTGTGGGCGA |
| | | TAGGGTCACCATCACCTGCCGTGCCAGTCAGGACA |
| | | TCCGTAATTATCTGAACTGGTATCAACAGAAACCA |
| | | GGAAAAGCTCCGAAACTACTGATTTACTATACCTC |
| | | CCGCCTGGAGTCTGGAGTCCCTTCTCGCTTCTCTG |
| | | GTTCTGGTTCTGGGACGGATTACACTCTGACCATC |
| | | AGCAGTCTGCAACCGGAAGACTTCGCAACTTATTA |
| | | CTGTCAGCAAGGTAATACTCTGCCGTGGACGTTCG |
| | | GACAGGGCACCAAGGTGGAGATCAAAGGCGGCGGC |
| | | GGAAGTGGAGGAGGAGGCTCAGGCGGAGGAGGGAG |
| | | CGAGGTTCAGCTGGTGGAGTCTGGCGGTGGCCTGG |
| | | TGCAGCCAGGGGGCTCACTCCGTTTGTCCTGTGCA |
| | | GCTTCTGGCTACTCCTTTACCGGCTACACTATGAA |
| | | CTGGGTGCGTCAGGCCCCAGGTAAGGGCCTGGAAT |
| | | GGGTTGCACTGATTAATCCTTATAAAGGTGTTAGT |
| | | ACCTACAACCAGAAGTTCAAGGACCGTTTCACTAT |
| | | AAGCGTAGATAAATCCAAAAACACAGCCTACCTGC |
| | | AAATGAACAGCCTGCGTGCTGAGGACACTGCCGTC |
| | | TATTATTGTGCTAGAAGCGGATACTACGGCGATAG |
| | | TGACTGGTATTTTGACGTGTGGGGTCAAGGAACCC |
| | | TGGTCACCGTCTCCTCGACATCTGGCGGCGGAGGA |
| | | TCTCTGGAATCTGGACAGGTGCTGCTGGAAAGCAA |
| | | CATCAAGGTGCTGCCCACCTGGTCTACCCCAGTTC |
| | | AGCCTATGGCTCTGATTGTGCTTGGCGGAGTTGCC |
| | | GGCCTGCTGCTCTTTATCGGCCTGGGCATCTTCTT |
| | | TTGCGTGCGGTGCAGACATCGGCGGAGACAGGCTG |
| | | AGAGAATGAGCCAGATCAAGCGGCTGCTGAGCGAG |
| | | AAGAAAACCTGTCAGTGCCCTCACCGGTTCCAGAA |
| | | AACATGCAGCCCCATC |
| 680 | Nucleotide | ATGGCTCTGCCTGTGACAGCTCTGTTGCTGCCTCT |
| | | GGCTCTGCTGCTGCATGCTGCTAGACCTCAGTCTG |
| | | TGCTGACGCAGCCGCCCTCAGTGTCTGGGGCCCCA |
| | | GGGCAGAGGGTCACCATCTCCTGCACTGGGAGCAG |
| | | CTCCAACATCGGGGCAGGTTATGATGTACACTGGT |
| | | ACCAGCAGCTTCCAGGAACAGCCCCCAAACTCCTC |
| | | ATCTATGGTAACAGCAATCGGCCCTCAGGGGTCCC |
| | | TGACCGATTCTCTGGCTCCAAGTCTGGCACCTCAG |
| | | CCTCCCTGGCCATCACTGGGCTCCAGGCTGAGGAT |
| | | GAGGCTGATTATTACTGCCAGTCCTATGACAGCAG |
| | | CCTGAGTGGCCCGGTGGTATTCGGCGGAGGGACCA |
| | | AGGTCACCGTCCTAGGGTCAACCTCTGGTAGCGGT |
| | | AAGCCTGGCTCCGGCGAAGGCTCCACAAAGGGTCA |
| | | GGTGCAGCTGGTGGAGTCTGGGGCTGAGGTGAAGA |
| | | AGCCTGGGTCCTCGGTGAAGGTCTCCTGCAAGGCT |
| | | TCTGGAGGCACCTTCAGCAGCTATGCTATCAGCTG |
| | | GGTGCGACAGGCCCCTGGACAAGGGCTTGAGTGGA |
| | | TGGGAGGGATCATCCCTATCTTTGGTACAGCAAAC |
| | | TACGCACAGAAGTTCCAGGGCAGAGTCACGATTAC |
| | | CGCGGACAAATCCACGAGCACAGCCTACATGGAGC |
| | | TGAGCAGCCTGAGGAGCGAGGACACGGCCGTGTAT |
| | | TACTGTGCGAGGACCCATCTACCATACAGCTATGG |
| | | TTTAGGCGGGTTTGACTACTGGGGCCAGGGAACCC |
| | | TGGTCACCGTGTCCTCAGAGCAGAAACTGATCAGC |
| | | GAGGAAGATCTGAATCCTGGCGGAGGCGGAGGAAG |
| | | TGGTGGCGGAGGTTCTGGTGGCGGTGGATCAGGCG |
| | | GTGGCGGATCTGGATCTATGGATATCCAGATGACC |
| | | CAGTCCCCGAGCTCCCTGTCCGCCTCTGTGGGCGA |
| | | TAGGGTCACCATCACCTGCCGTGCCAGTCAGGACA |
| | | TCCGTAATTATCTGAACTGGTATCAACAGAAACCA |
| | | GGAAAAGCTCCGAAACTACTGATTTACTATACCTC |
| | | CCGCCTGGAGTCTGGAGTCCCTTCTCGCTTCTCTG |
| | | GTTCTGGTTCTGGGACGGATTACACTCTGACCATC |
| | | AGCAGTCTGCAACCGGAAGACTTCGCAACTTATTA |
| | | CTGTCAGCAAGGTAATACTCTGCCGTGGACGTTCG |
| | | GACAGGGCACCAAGGTGGAGATCAAAGGCGGCGGC |
| | | GGAAGTGGAGGAGGAGGCTCAGGCGGAGGAGGGAG |
| | | CGAGGTTCAGCTGGTGGAGTCTGGCGGTGGCCTGG |
| | | TGCAGCCAGGGGGCTCACTCCGTTTGTCCTGTGCA |
| | | GCTTCTGGCTACTCCTTTACCGGCTACACTATGAA |
| | | CTGGGTGCGTCAGGCCCCAGGTAAGGGCCTGGAAT |
| | | GGGTTGCACTGATTAATCCTTATAAAGGTGTTAGT |
| | | ACCTACAACCAGAAGTTCAAGGACCGTTTCACTAT |
| | | AAGCGTAGATAAATCCAAAAACACAGCCTACCTGC |
| | | AAATGAACAGCCTGCGTGCTGAGGACACTGCCGTC |
| | | TATTATTGTGCTAGAAGCGGATACTACGGCGATAG |
| | | TGACTGGTATTTTGACGTGTGGGGTCAAGGAACCC |
| | | TGGTCACCGTCTCCTCGACATCTGGCGGCGGAGGA |
| | | TCTCTGGAATCTGGACAGGTGCTGCTGGAAAGCAA |
| | | CATCAAGGTGCTGCCCACCTGGTCTACCCCAGTTC |
| | | AGCCTATGGCTCTGATTGTGCTTGGCGGAGTTGCC |
| | | GGCCTGCTGCTCTTTATCGGCCTGGGCATCTTCTT |
| | | TTGCGTGCGGTGCAGACATCGGCGGAGACAGGCTG |
| | | AGAGAATGAGCCAGATCAAGCGGCTGCTGAGCGAG |
| | | AAGAAAACCTGTCAGTGCCCTCACCGGTTCCAGAA |
| | | AACATGCAGCCCCATC |
| 681 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGCAGTCTGGGGCTGAGGTGAAGAAGCCT |
| | | GGGGCCTCAGTGAAGGTCTCCTGCAAGGCTTCTGG |
| | | TTACACCTTTACCAGCTATGGTATCAGCTGGGTGC |
| | | GACAGGCCCCTGGACAAGGGCTTGAGTGGATGGGA |
| | | TGGATCAGCGCTTACAATGGTAACACAAACTATGC |
| | | ACAGAAGCTCCAGGGCAGAGTCACCATGACCACAG |
| | | ACACATCCACGAGCACAGCCTACATGGAGCTGAGG |
| | | AGCCTGAGGAGCGACGACACGGCCGTGTATTACTG |
| | | TGCGAGAGATATAATACGATATTGTAGTAGTACCA |
| | | GCTGCTATAGAGGGATTGACTACTGGGGCCAGGGA |
| | | ACCCTGGTCACCGTGTCCTCAGGGTCAACCTCTGG |
| | | TAGCGGTAAGCCTGGCTCCGGCGAAGGCTCCACAA |
| | | AGGGTCAGTCTGCCCTGACTCAGCCTGCCTCCGTG |
| | | TCTGGGTCTCCTGGACAGTCGATCACCATCTCCTG |
| | | CACTGGAACCAGCAGTGACGTTGGTGGTTATAACT |
| | | ATGTCTCCTGGTACCAACAGCACCCAGGCAAAGCC |
| | | CCCAAACTCATGATTTATGATGTCAGTAATCGGCC |
| | | CTCAGGGGTTTCTAATCGCTTCTCTGGCTCCAAGT |
| | | CTGGCAACACGGCCTCCCTGACCATCTCTGGGCTC |
| | | CAGGCTGAGGACGAGGCTGATTATTACTGCAGCTC |
| | | ATATACAAGCAGCAGCCCCCATGTGGTATCCGGCG |
| | | GAGGGACCAAGCTGACGGTCCTAGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTTATAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 682 | Nucleotide | ATGGCTCTGCCTGTGACAGCTCTGTTGCTGCCTCT |
| | | GGCTCTGCTGCTGCATGCTGCTAGACCTCAGTCTG |
| | | CCCTGACTCAGCCTGCCTCCGTGTCTGGGTCTCCT |
| | | GGACAGTCGATCACCATCTCCTGCACTGGAACCAG |
| | | CAGTGACGTTGGTGGTTATAACTATGTCTCCTGGT |
| | | ACCAACAGCACCCAGGCAAAGCCCCCAAACTCATG |
| | | ATTTATGATGTCAGTAATCGGCCCTCAGGGGTTTC |
| | | TAATCGCTTCTCTGGCTCCAAGTCTGGCAACACGG |
| | | CCTCCCTGACCATCTCTGGGCTCCAGGCTGAGGAC |
| | | GAGGCTGATTATTACTGCAGCTCATATACAAGCAG |
| | | CAGCCCCCATGTGGTATCCGGCGGAGGGACCAAGC |
| | | TGACGGTCCTAGGGTCAACCTCTGGTAGCGGTAAG |
| | | CCTGGCTCCGGCGAAGGCTCCACAAAGGGTCAGGT |
| | | GCAGCTGGTGCAGTCTGGGGCTGAGGTGAAGAAGC |
| | | CTGGGGCCTCAGTGAAGGTCTCCTGCAAGGCTTCT |
| | | GGTTACACCTTTACCAGCTATGGTATCAGCTGGGT |
| | | GCGACAGGCCCCTGGACAAGGGCTTGAGTGGATGG |
| | | GATGGATCAGCGCTTACAATGGTAACACAAACTAT |
| | | GCACAGAAGCTCCAGGGCAGAGTCACCATGACCAC |
| | | AGACACATCCACGAGCACAGCCTACATGGAGCTGA |
| | | GGAGCCTGAGGAGCGACGACACGGCCGTGTATTAC |
| | | TGTGCGAGAGATATAATACGATATTGTAGTAGTAC |
| | | CAGCTGCTATAGAGGGATTGACTACTGGGGCCAGG |
| | | GAACCCTGGTCACCGTGTCCTCAGAGCAGAAACTG |
| | | ATCAGCGAGGAAGATCTGAATCCTGGCGGAGGCGG |
| | | AGGAAGTGGTGGCGGAGGTTCTGGTGGCGGTGGAT |
| | | CAGGCGGTGGCGGATCTGGATCTATGGATATCCAG |
| | | ATGACCCAGTCCCCGAGCTCCCTGTCCGCCTCTGT |
| | | GGGCGATAGGGTCACCATCACCTGCCGTGCCAGTC |
| | | AGGACATCCGTAATTATCTGAACTGGTATCAACAG |
| | | AAACCAGGAAAAGCTCCGAAACTACTGATTTACTA |
| | | TACCTCCCGCCTGGAGTCTGGAGTCCCTTCTCGCT |
| | | TCTCTGGTTCTGGTTCTGGGACGGATTACACTCTG |
| | | ACCATCAGCAGTCTGCAACCGGAAGACTTCGCAAC |
| | | TTATTACTGTCAGCAAGGTAATACTCTGCCGTGGA |
| | | CGTTCGGACAGGGCACCAAGGTGGAGATCAAAGGC |
| | | GGCGGCGGAAGTGGAGGAGGAGGCTCAGGCGGAGG |
| | | AGGGAGCGAGGTTCAGCTGGTGGAGTCTGGCGGTG |
| | | GCCTGGTGCAGCCAGGGGGCTCACTCCGTTTGTCC |
| | | TGTGCAGCTTCTGGCTACTCCTTTACCGGCTACAC |
| | | TATGAACTGGGTGCGTCAGGCCCCAGGTAAGGGCC |
| | | TGGAATGGGTTGCACTGATTAATCCTTATAAAGGT |
| | | GTTAGTACCTACAACCAGAAGTTCAAGGACCGTTT |
| | | CACTATAAGCGTAGATAAATCCAAAAACACAGCCT |
| | | ACCTGCAAATGAACAGCCTGCGTGCTGAGGACACT |
| | | GCCGTCTATTATTGTGCTAGAAGCGGATACTACGG |
| | | CGATAGTGACTGGTATTTTGACGTGTGGGGTCAAG |
| | | GAACCCTGGTCACCGTCTCCTCGACATCTGGCGGC |
| | | GGAGGATCTCTGGAATCTGGACAGGTGCTGCTGGA |
| | | AAGCAACATCAAGGTGCTGCCCACCTGGTCTACCC |
| | | CAGTTCAGCCTATGGCTCTGATTGTGCTTGGCGGA |
| | | GTTGCCGGCCTGCTGCTCTTTATCGGCCTGGGCAT |
| | | CTTCTTTTGCGTGCGGTGCAGACATCGGCGGAGAC |
| | | AGGCTGAGAGAATGAGCCAGATCAAGCGGCTGCTG |
| | | AGCGAGAAGAAAACCTGTCAGTGCCCTCACCGGTT |
| | | CCAGAAAACATGCAGCCCCATC |
| 683 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGGTGGAGTCTGGGGCTGAGGTGAAGAAGCCT |
| | | GGGTCCTCGGTGAAGGTCTCCTGCAAGGCTTCTGG |
| | | AGGCACCTTCAGCAGCTATGCTATCAGCTGGGTGC |
| | | GACAGGCCCCTGGACAAGGGCTTGAGTGGATGGGA |
| | | GGGATCATCCCTATCTTTGGTACAGCAAACTACGC |
| | | ACAGAAGTTCCAGGGCAGAGTCACGATTACCGCGG |
| | | ACGAATCCACGAGCACAGCCTACATGGAGCTGAGC |
| | | AGCCTGAGGAGCGAGGACACGGCCGTGTATTACTG |
| | | TGCGCGCCCCCTGTATGCCCCTCGCTTCGGATACG |
| | | GTATGGACGTCTGGGGCCAAGGGACCCTGGTCACC |
| | | GTGTCCTCAGGGTCAACCTCTGGTAGCGGTAAGCC |
| | | TGGCTCCGGCGAAGGCTCCACAAAGGGTCAGTCTG |
| | | TGTTGACGCAGCCGCCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGTTGGGTGTTCGGCGGAGGCACCCAGCTGA |
| | | CCGTCCTCGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTTATAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 684 | Nucleotide | ATGGCTCTGCCTGTGACAGCTCTGTTGCTGCCTCT |
| | | GGCTCTGCTGCTGCATGCTGCTAGACCTCAGTCTG |
| | | TGTTGACGCAGCCGCCCTCAGCGTCTGGGACCCCC |
| | | GGGCAGAGGGTCACCATCTCTTGTTCTGGAAGCAG |
| | | CTCCAACATCGGAAGTAATTATGTATACTGGTACC |
| | | AGCAGCTCCCAGGAACGGCCCCCAAACTCCTCATC |
| | | TATAGGAATAATCAGCGGCCCTCAGGGGTCCCTGA |
| | | CCGATTCTCTGGCTCCAAGTCTGGCACCTCAGCCT |
| | | CCCTGGCCATCAGTGGGCTCCGGTCCGAGGATGAG |
| | | GCTGATTATTACTGTGCAGCATGGGATGACAGCCT |
| | | GAGTGGTTGGGTGTTCGGCGGAGGCACCCAGCTGA |
| | | CCGTCCTCGGGTCAACCTCTGGTAGCGGTAAGCCT |
| | | GGCTCCGGCGAAGGCTCCACAAAGGGTCAGGTGCA |
| | | GCTGGTGGAGTCTGGGGCTGAGGTGAAGAAGCCTG |
| | | GGTCCTCGGTGAAGGTCTCCTGCAAGGCTTCTGGA |
| | | GGCACCTTCAGCAGCTATGCTATCAGCTGGGTGCG |
| | | ACAGGCCCCTGGACAAGGGCTTGAGTGGATGGGAG |
| | | GGATCATCCCTATCTTTGGTACAGCAAACTACGCA |
| | | CAGAAGTTCCAGGGCAGAGTCACGATTACCGCGGA |
| | | CGAATCCACGAGCACAGCCTACATGGAGCTGAGCA |
| | | GCCTGAGGAGCGAGGACACGGCCGTGTATTACTGT |
| | | GCGCGCCCCCTGTATGCCCCTCGCTTCGGATACGG |
| | | TATGGACGTCTGGGGCCAAGGGACCCTGGTCACCG |
| | | TGTCCTCAGAGCAGAAACTGATCAGCGAGGAAGAT |
| | | CTGAATCCTGGCGGAGGCGGAGGAAGTGGTGGCGG |
| | | AGGTTCTGGTGGCGGTGGATCAGGCGGTGGCGGAT |
| | | CTGGATCTATGGATATCCAGATGACCCAGTCCCCG |
| | | AGCTCCCTGTCCGCCTCTGTGGGCGATAGGGTCAC |
| | | CATCACCTGCCGTGCCAGTCAGGACATCCGTAATT |
| | | ATCTGAACTGGTATCAACAGAAACCAGGAAAAGCT |
| | | CCGAAACTACTGATTTACTATACCTCCCGCCTGGA |
| | | GTCTGGAGTCCCTTCTCGCTTCTCTGGTTCTGGTT |
| | | CTGGGACGGATTACACTCTGACCATCAGCAGTCTG |
| | | CAACCGGAAGACTTCGCAACTTATTACTGTCAGCA |
| | | AGGTAATACTCTGCCGTGGACGTTCGGACAGGGCA |
| | | CCAAGGTGGAGATCAAAGGCGGCGGCGGAAGTGGA |
| | | GGAGGAGGCTCAGGCGGAGGAGGGAGCGAGGTTCA |
| | | GCTGGTGGAGTCTGGCGGTGGCCTGGTGCAGCCAG |
| | | GGGGCTCACTCCGTTTGTCCTGTGCAGCTTCTGGC |
| | | TACTCCTTTACCGGCTACACTATGAACTGGGTGCG |
| | | TCAGGCCCCAGGTAAGGGCCTGGAATGGGTTGCAC |
| | | TGATTAATCCTTATAAAGGTGTTAGTACCTACAAC |
| | | CAGAAGTTCAAGGACCGTTTCACTATAAGCGTAGA |
| | | TAAATCCAAAAACACAGCCTACCTGCAAATGAACA |
| | | GCCTGCGTGCTGAGGACACTGCCGTCTATTATTGT |
| | | GCTAGAAGCGGATACTACGGCGATAGTGACTGGTA |
| | | TTTTGACGTGTGGGGTCAAGGAACCCTGGTCACCG |
| | | TCTCCTCGACATCTGGCGGCGGAGGATCTCTGGAA |
| | | TCTGGACAGGTGCTGCTGGAAAGCAACATCAAGGT |
| | | GCTGCCCACCTGGTCTACCCCAGTTCAGCCTATGG |
| | | CTCTGATTGTGCTTGGCGGAGTTGCCGGCCTGCTG |
| | | CTCTTTATCGGCCTGGGCATCTTCTTTTGCGTGCG |
| | | GTGCAGACATCGGCGGAGACAGGCTGAGAGAATGA |
| | | GCCAGATCAAGCGGCTGCTGAGCGAGAAGAAAACC |
| | | TGTCAGTGCCCTCACCGGTTCCAGAAAACATGCAG |
| | | CCCCATC |
| 685 | Nucleotide | ATGGCTCTGCCTGTGACAGCTCTGTTGCTGCCTCT |
| | | GGCTCTGCTGCTGCATGCTGCTAGACCTCAGGTGC |
| | | AGCTGCAGGAGAGCGGCGGCGGCCTGGTGCAGCCC |
| | | GGCGGCAGCCTGAGGCTGAGCTGCGCCGCCAGCGG |
| | | CATCATCTTCAGCATCAACACCATGGCCTGGTACA |
| | | GGCAGGGCCCCGGCAAGCAGAGGGACCTGGTGGCC |
| | | CTGATCAGCAGCGGCGGCAACACCAGCTACGCCGA |
| | | CAGCGTGAACGGCAGGTTCACCATCAGCAGGGACA |
| | | ACGCCAAGAACACCGTGTACCTGCAGATGAACGGC |
| | | CTGAAGCCCGAGGACACCGCCGTGTACTACTGCAA |
| | | CAGCGCCGGCAGGAGCTACAGCGGCAGCTACGGCG |
| | | CCTACTGGGGCCAGGGCACCCAGGTGACCGTGAGC |
| | | AGCGAGCAGAAACTGATCAGCGAGGAAGATCTGAA |
| | | TCCTGGCGGAGGCGGAGGAAGTGGTGGCGGAGGTT |
| | | CTGGTGGCGGTGGATCAGGCGGTGGCGGATCTGGA |
| | | TCTATGGATATCCAGATGACCCAGTCCCCGAGCTC |
| | | CCTGTCCGCCTCTGTGGGCGATAGGGTCACCATCA |
| | | CCTGCCGTGCCAGTCAGGACATCCGTAATTATCTG |
| | | AACTGGTATCAACAGAAACCAGGAAAAGCTCCGAA |
| | | ACTACTGATTTACTATACCTCCCGCCTGGAGTCTG |
| | | GAGTCCCTTCTCGCTTCTCTGGTTCTGGTTCTGGG |
| | | ACGGATTACACTCTGACCATCAGCAGTCTGCAACC |
| | | GGAAGACTTCGCAACTTATTACTGTCAGCAAGGTA |
| | | ATACTCTGCCGTGGACGTTCGGACAGGGCACCAAG |
| | | GTGGAGATCAAAGGCGGCGGCGGAAGTGGAGGAGG |
| | | AGGCTCAGGCGGAGGAGGGAGCGAGGTTCAGCTGG |
| | | TGGAGTCTGGCGGTGGCCTGGTGCAGCCAGGGGGC |
| | | TCACTCCGTTTGTCCTGTGCAGCTTCTGGCTACTC |
| | | CTTTACCGGCTACACTATGAACTGGGTGCGTCAGG |
| | | CCCCAGGTAAGGGCCTGGAATGGGTTGCACTGATT |
| | | AATCCTTATAAAGGTGTTAGTACCTACAACCAGAA |
| | | GTTCAAGGACCGTTTCACTATAAGCGTAGATAAAT |
| | | CCAAAAACACAGCCTACCTGCAAATGAACAGCCTG |
| | | CGTGCTGAGGACACTGCCGTCTATTATTGTGCTAG |
| | | AAGCGGATACTACGGCGATAGTGACTGGTATTTTG |
| | | ACGTGTGGGGTCAAGGAACCCTGGTCACCGTCTCC |
| | | TCGACATCTGGCGGCGGAGGATCTCTGGAATCTGG |
| | | ACAGGTGCTGCTGGAAAGCAACATCAAGGTGCTGC |
| | | CCACCTGGTCTACCCCAGTTCAGCCTATGGCTCTG |
| | | ATTGTGCTTGGCGGAGTTGCCGGCCTGCTGCTCTT |
| | | TATCGGCCTGGGCATCTTCTTTTGCGTGCGGTGCA |
| | | GACATCGGCGGAGACAGGCTGAGAGAATGAGCCAG |
| | | ATCAAGCGGCTGCTGAGCGAGAAGAAAACCTGTCA |
| | | GTGCCCTCACCGGTTCCAGAAAACATGCAGCCCCA |
| | | TC |
| 686 | AminoAcid | MALPVTALLLPLALLLHAARPQVQLQESGGGLVQP |
| | | GGSLRLSCAASGIIFSINTMAWYRQGPGKQRDLVA |
| | | LISSGGNTSYADSVNGRFTISRDNAKNTVYLQMNG |
| | | LKPEDTAVYYCNSAGRSYSGSYGAYWGQGTQVTVS |
| | | SEQKLISEEDLNPGGGGGSGGGGSGGGGSGGGGSG |
| | | SMDIQMTQSPSSLSASVGDRVTITCRASQDIRNYL |
| | | NWYQQKPGKAPKLLIYYTSRLESGVPSRFSGSGSG |
| | | TDYTLTISSLQPEDFATYYCQQGNTLPWTFGQGTK |
| | | VEIKGGGGSGGGGSGGGGSEVQLVESGGGLVQPGG |
| | | SLRLSCAASGYSFTGYTMNWVRQAPGKGLEWVALI |
| | | NPYKGVSTYNQKFKDRFTISVDKSKNTAYLQMNSL |
| | | RAEDTAVYYCARSGYYGDSDWYFDVWGQGTLVTVS |
| | | STSGGGGSLESGQVLLESNIKVLPTWSTPVQPMAL |
| | | IVLGGVAGLLLFIGLGIFFCVRCRHRRRQAERMSQ |
| | | IKRLLSEKKTCQCPHRFQKTCSPI |

What is claimed is:

1. A Guanylate Cyclase 2C (GUCY2C) T cell-antigen coupler (GUCY2C-TAC) protein, comprising:
(a) a first polypeptide encoding an antigen-binding domain that binds GUCY2C;
(b) a second polypeptide encoding an antigen-binding domain that binds a protein associated with a TCR complex; and
(c) a third polypeptide encoding a TCR co-receptor cytosolic domain and transmembrane domain;
wherein components encoded by (a), components encoded by (b), and components encoded by (c) are fused directly to each other, or joined by at least one linker.

2. The GUCY2C-TAC protein of claim 1, wherein the first polynucleotide, the second polynucleotide, and the third polynucleotide are in order.

3. The GUCY2C-TAC protein of claim 1 or 2, wherein the antigen-binding domain that binds GUCY2C is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody.

4. The GUCY2C-TAC protein of any one of claims 1-3, wherein the antigen-binding domain that binds GUCY2C is a designed ankyrin repeat (DARPin) polypeptide, a single chain variable fragment (scFv), or a nanobody.

5. The GUCY2C-TAC protein of any one of claims 1-4, wherein the antigen-binding domain that binds GUCY2C is a nanobody.

6. The GUCY2C-TAC protein of any one of claims 1-5, wherein the protein associated with the TCR complex is a CD3 protein.

7. The GUCY2C-TAC protein of claim 6, wherein the CD3 protein is a CD3γ protein, CD3δ protein and/or CD3ε protein. In some embodiments, the CD3 protein is a CD3ε protein.

8. The GUCY2C-TAC protein of claim 7, wherein the CD3 protein is a CD3ε protein.

9. The GUCY2C-TAC protein of any one of claims 1-8, wherein the antigen-binding domain that binds the protein associated with the TCR complex is a designed ankyrin repeat (DARPin) polypeptide, single chain variable fragment (scFv), single domain antibody, diabody, affibody, adnectin, affilin, phylomer; fynomer, affimer, peptide aptamer, knottin, centyrin, anticalin, or nanobody.

10. The GUCY2C-TAC protein of any one of claims 1-9, wherein the antigen-binding domain that binds the protein associated with the TCR complex is derived from an antibody selected from UCHT1 OKT3, F6A, and L2K.

11. The GUCY2C-TAC protein of claim 10, wherein the antigen-binding domain that binds the protein associated with the TCR complex is a UCHT1 antigen-binding domain.

12. The GUCY2C-TAC protein of claim 11, wherein the UCHT1 antigen-binding domain is an scFv of UCHT1.

13. The GUCY2C-TAC protein of claim 11 or 12, wherein the UCHT1 antigen-binding domain comprises a Y to T mutation at a position corresponding to amino acid 182 of SEQ ID NO: 32 (Y182T).

14. The GUCY2C-TAC protein of any one of claims 10-12, wherein the UCHT1 antigen-binding domain comprises a humanized variant of UCHT1 (huUCHT1).

15. The GUCY2C-TAC protein of claim 14, wherein the UCHT1 antigen-binding domain comprises a humanized variant of UCHT1 comprising a Y to T mutation at a position corresponding to amino acid 177 of SEQ ID NO: 40 (huUCHT1 (Y177T)).

16. The GUCY2C-TAC protein of any one of claims 10-12, wherein antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)).

17. The GUCY2C-TAC protein of any one of claims 10-12, wherein the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)).

18. The GUCY2C-TAC protein of any one of claims 10-12, wherein the CDR sequences of the antigen-binding domain that binds the protein associated with the TCR complex have 100% identity with the CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 32 (UCHT1), SEQ ID NO: 44 (UCHT1 (Y182T)), SEQ ID NO: 40 (huUCHT1), or SEQ ID NO: 42 (huUCHT1 (Y177T)).

19. The GUCY2C-TAC protein of claim 10, wherein the antigen-binding domain that binds the protein associated with the TCR complex is an OKT3 antigen-binding domain.

20. The GUCY2C-TAC protein of claim 19, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 34 (OKT3).

21. The GUCY2C-TAC protein of claim 19 or 20, wherein the CDR sequences of the antigen-binding domain that binds the protein associated with the TCR complex have 100% identity with the CDR sequences of the amino acid sequence of SEQ ID NO: 34 (OKT3).

22. The GUCY2C-TAC protein of claim 19 or claim 20, wherein the CDR sequences of the antigen-binding domain that binds the protein associated with the TCR complex have 100% identity with the CDR sequences of the amino acid sequence of SEQ ID NO: 34 (OKT3), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 34 (OKT3).

23. The GUCY2C-TAC protein of claim 10, wherein the antigen-binding domain that binds the protein associated with the TCR complex is a F6A antigen-binding domain.

24. The GUCY2C-TAC protein of claim 23, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 36 (F6A).

25. The GUCY2C-TAC protein of claim 23 or 24, wherein the CDR sequences of the antigen-binding domain that binds the protein associated with the TCR complex have 100% identity with the CDR sequences of the amino acid sequence of SEQ ID NO: 36 (F6A).

26. The GUCY2C-TAC protein of claim 23 or claim 24, wherein the CDR sequences of the antigen-binding domain that binds the protein associated with the TCR complex have 100% identity with the CDR sequences of the amino acid sequence of SEQ ID NO: 36 (F6A), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 36 (F6A).

27. The GUCY2C-TAC protein of claim 10, wherein the antigen-binding domain that binds the protein associated with the TCR complex is a L2K antigen-binding domain.

28. The GUCY2C-TAC protein of claim 27, wherein the antigen-binding domain that binds the protein associated with the TCR complex comprises an amino acid sequence having at least 80%, at least 85%, at least 90%, at least 95%, at least 96%, at least 97%, at least 98%, at least 99%, or 100% sequence identity with SEQ ID NO: 38 (L2K).

29. The GUCY2C-TAC protein of claim 27 or 28, wherein the CDR sequences of the antigen-binding domain that binds the protein associated with the TCR complex have 100% identity with the CDR sequences of the amino acid sequence of SEQ ID NO: 38 (L2K).

30. The GUCY2C-TAC protein of claim 27 or 28, wherein the CDR sequences of the antigen-binding domain that binds the protein associated with the TCR complex have 100% identity with the CDR sequences of the amino acid sequence of SEQ ID NO: 38 (L2K), and the non-CDR sequences of the antigen-binding domain that binds the protein associated with the TCR complex have at least 80%, at least 85%, at least 90%, at least 95%, at least 96%, at least 97%, at least 98%, or at least 99% sequence identity with the non-CDR sequences of the amino acid sequence of SEQ ID NO: 38 (L2K).

31. The GUCY2C-TAC protein of any one of claims 1-30, wherein the transmembrane domain is a CD4 transmembrane domain and the cytosolic domain is a CD4 cytosolic domain.

32. The GUCY2C-TAC protein of claim 31, wherein the transmembrane and cytosolic domain comprise an amino acid sequence having at least 80%, at least 85%, at least 90%, at least 95%, at least 96%, at least 97%, at least 98%, at least 99%, or 100% sequence identity with the amino acid sequence of SEQ ID NO: 46 (CD4 transmembrane and cytosolic domain).

33. The GUCY2C-TAC protein of any one of claims 1-32, wherein the transmembrane domain is a CD8 transmembrane domain and the cytosolic domain is a CD8 cytosolic domain.

34. The GUCY2C-TAC protein of any one of claims 1-33, wherein the component encoded by (a) and the component encoded by (c) are fused to the component encoded by (b).

35. The GUCY2C-TAC protein of any one of claims 1-33, wherein the component encoded by (b) and the component encoded by (c) are fused to the component encoded by (a).

36. The GUCY2C-TAC protein of any one of claims 1-35, wherein at least one linker joins the component encoded by (a) to the component encoded by (b).

37. The GUCY2C-TAC protein of claim 36, wherein the at least one linker is a glycine and/or serine-rich linker, a large protein domain, a long helix structure, or a short helix structure.

38. The GUCY2C-TAC protein of claim 36 or 37, wherein the at least one linker comprises an amino acid sequence having at least 80%, at least 85%, at least 90%, at least 95%, at least 96%, at least 97%, at least 98%, at least 99%, or 100% sequence identity with the amino acid sequence of SEQ ID NO: 26 ((G4S)4-based linker), SEQ ID NO: 28 (G4S-based linker), SEQ ID NO: 14 (CD4 based linker), SEQ ID NO: 12 (short helix connector), SEQ ID NO: 14 (long helix connector), SEQ ID NO: 16 (large domain connector), or SEQ ID NO: 24 ((G4S)3 flexible linker).

39. The GUCY2C-TAC protein of any one of claims 1-38, wherein the GUYC2C antigen binding domain is selected from an amino acid sequence according to any one of SEQ ID NOs: 53-127 or 514-521.

40. The GUCY2C-TAC protein of any one of claims 1-38, wherein the GUCY2C antigen-binding domain comprises a heavy chain variable region having an amino acid sequence having at least 80% sequence identity with an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence having at least 80% sequence identity with an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203.

41. The GUCY2C-TAC protein of any one of claims 1-38, wherein the GUYC2C antigen binding domain comprises a heavy chain variable region having an amino acid sequence according to any one of SEQ ID NOs: 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, and 202; and a light chain variable region having an amino acid sequence according to any one of SEQ ID NOs: 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, and 203.

42. The GUCY2C-TAC protein of any one of claims 1-38, wherein the GUCY2C antigen-binding domain comprises a heavy chain variable region comprising (a) a CDR1 having an amino acid selected from the group consisting of SEQ ID NO: 204, 210, 216, 222, 228, 234, 240, 246, 252, 258, 264, 270, 276, 282, 288, 294, 300, 306, 312, 318, 324, 330, 336, 342, 348, 354, 393, 399, 405, 411, 417, 423, 429, 435, 441, 447, 453, 459, 522, 525, 528, 531, 534, 537, 540, and 543, (b) a CDR2 having an amino acid selected from the group consisting of SEQ ID NO: 205, 211, 217, 223, 229, 235, 241, 247, 253, 259, 265, 271, 277, 283, 289, 295, 301, 307, 313, 319, 325, 331, 337, 343, 349, 355, 394, 400, 406, 412, 418, 424, 430, 436, 442, 448, 454, 460, 523, 526, 529, 532, 535, 538, 541, and 544, and (c) a CDR3 having an amino acid selected from the group consisting of SEQ ID NO: 206, 212, 218, 224, 230, 236, 242, 248, 254, 260, 266, 272, 278, 284, 290, 296, 302, 308, 314, 320, 326, 332, 338, 344, 350, 356, 395, 401, 407, 413, 419, 425, 431, 437, 443, 449, 455, 461, 524, 527, 530, 533, 536, 539, 542, and 545; and a light chain variable region comprising (a) a CDR1 having an amino acid selected from the group consisting of SEQ ID NO: 207, 213, 219, 225, 231, 237, 243, 249, 255, 261, 267, 273, 279, 285, 291, 297, 303, 309, 315, 321, 327, 333, 339, 345, 351, 357, 396, 402, 408, 414, 420, 426, 432, 438, 444, 450, 456 and 462, (b) a CDR2 having an amino acid selected from the group consisting of SEQ ID NO: 208, 214, 220, 226, 232, 238, 244, 250, 256, 262, 268, 274, 280, 286, 292, 298, 304, 310, 316, 322, 328, 334, 340, 346, 352, 358, 397, 403, 409, 415, 421, 427, 433, 439, 445, 451, 457 and 463, and (c) a CDR3 having an amino acid selected from the group consisting of SEQ ID NO: 209, 215, 221, 227, 233, 239, 245, 251, 257, 263, 269, 275, 281, 287, 293, 299, 305, 311, 317, 323, 329, 335, 341, 347, 353, 359, 398, 404, 410, 416, 422, 428, 434, 440, 446, 452, 458 and 464.

43. The GUCY2C-TAC protein of any one of claims 1-38, wherein the GUCY2C antigen-binding domain is a nanobody and comprises (a) a VHH CDR1 having an amino acid selected from the group consisting of SEQ ID NO: 360, 363, 366, 369, 372, 375, 378, 381, 384, 387, and 390; (b) a VHH CDR2 having an amino acid selected from the group consisting of SEQ ID NO: 361, 364, 367, 370, 373, 376, 379, 382, 385, 388, and 391; and (c) a VHH CDR3 having an amino acid selected from the group consisting of SEQ ID NO: 362, 365, 368, 371, 374, 377, 380, 383, 386, 389, and 392.

44. The GUCY2C-TAC protein of any one of claims 1-43, wherein the GUCY2C-TAC protein does not comprise a co-stimulatory domain.

45. The GUCY2C-TAC protein of any one of claims 1-44, wherein the GUCY2C-TAC protein does not comprise an activation domain.

46. The GUCY2C-TAC protein of any one of claims 1-45, wherein the GUCY2C-TAC protein further comprises a leader sequence.

47. The GUCY2C-TAC protein of claim 46, wherein the leader sequence comprises an amino acid sequence having at least 80%, at least 85%, at least 90%, at least 95%, at least 96%, at least 97%, at least 98%, at least 99%, or 100% sequence identity with the amino acid sequence of SEQ ID NO: 2 (muIgG leader), SEQ ID NO: 18 (huIgG leader), SEQ ID NO: 20 (huCD8a-1 leader) or SEQ ID NO: 30 (huCD8a-2 leader).

48. A GUCY2C TAC protein comprising an amino acid sequence having at least 80% sequence identity with the amino acid sequence of any one of SEQ ID NOs: 465-513, 546-590, or 686.

49. A GUCY2C TAC protein comprising an amino acid sequence according to the amino acid sequence of any one of SEQ ID NOs: 465-513, 546-590, or 686.

50. A nucleic acid sequence encoding the GUCY2C TAC protein of any one of claims 1-49.

51. The nucleic acid sequence of claim 50, wherein the nucleic acid sequence has at least 80% sequence identity with the nucleic acid sequence of any one of SEQ ID NOs: 591-685.

52. The nucleic acid sequence of claim 50, wherein the nucleic acid sequence comprises the nucleic acid sequence of any one of SEQ ID NOs: 591-685.

53. A T cell expressing the GUCY2C-TAC protein of any one of claims 1-49.

54. A T cell comprising the nucleic acid sequence of any one of claims 50-52.

55. A pharmaceutical composition comprising the T cell of claim 53 or 54, and a pharmaceutically acceptable excipient.

56. A method of treating a GUCY2C-expressing cancer in an individual in need thereof, comprising administering to the individual the pharmaceutical composition of claim 55.

57. The method of claim 56, wherein the cancer is a solid cancer.

58. The method of claim 56 or 57, wherein the cancer is a primary colorectal cancer, a primary gastric cancer, a primary gastroesophageal junction cancer, a primary esophageal cancer, or a primary pancreatic cancer.

59. The method of claim 56 or 57, wherein the cancer is a metastatic colorectal cancer, a metastatic gastric cancer, a metastatic gastroesophageal junction cancer, a metastatic esophageal cancer, or a metastatic pancreatic cancer.
